(19) United States
(12) Patent Application Publication
(10) Pub. No.: US 2019/0360051 A1
(43) Pub. Date: Nov. 28, 2019

(54) SWARM INTELLIGENCE-ENHANCED DIAGNOSIS AND THERAPY SELECTION FOR CANCER USING TUMOR- EDUCATED PLATELETS

(71) Applicant: Stichting VUmc, Amsterdam (NL)

(72) Inventors: Thomas Würdinger, Amsterdam (NL); Myron Ghislain Best, Amsterdam (NL)

(21) Appl. No.: 16/313,231

(22) PCT Filed: Feb. 19, 2018
(86) PCT No.: PCT/NL2018/050110

§ 371 Date: Dec. 26, 2018

(30) Foreign Application Priority Data
Feb. 17, 2017 (NL) 2018391
Mar. 23, 2017 (NL) 2018567

(51) Int. Cl. C12Q 1/6886; C07K 16/28

(52) U.S. Cl. CPC C12Q 1/6886; C07K 16/2818; C07K 2317/21; C12Q 2600/158; C12Q 2600/106

(57) ABSTRACT

The invention provides methods of administering immunotherapy that modulates an interaction between PD-i and its ligand, to a cancer patient, based on tumor-educated gene expression profiles obtained from anucleated cells. The invention further provides methods of typing a sample of a subject for the presence or absence of a cancer, based on tumor-educated gene expression profiles obtained from anucleated cells. The invention further provides a method for obtaining a biomarker panel for typing of a sample from a subject using particle swarm optimization-based algorithms.

The invention is in the field of medical diagnostics, in particular in the field of disease diagnostics and monitoring. The invention is directed to markers for the detection of disease, to methods for detecting disease, and to a method for determining the efficacy of a disease treatment.

### BACKGROUND OF THE INVENTION

Cancer is one of the leading causes of death in developed countries. Studies have revealed that many cancer patients are diagnosed at a late stage, when they are more difficult to treat. Cancer is mainly driven by successive mutations in normal cells, resulting in DNA damages and ultimately causing significant gene alterations that contribute to a cancerous state.

Cancer is often diagnosed on the basis of tumor markers. Tumor markers are substances that are present in a cancer cell or that is produced in another cell in response to a cancer. Some tumor markers are also present in normal cells but, for example, in an alternative form of at higher levels, in a cancerous cell. Tumor markers can often be identified in a liquid sample, such as blood, urine, stool, or bodily fluids.

Most presently used tumor markers are proteins. An example is prostate-specific antigen (PSA), which is used as a tumor marker for prostate cancer. Most single tumor markers are not reliable to be useful in the management of an individual patient with cancer. Alternative markers, such as gene expression levels and DNA alterations such as DNA methylation, have begun to be used as tumor markers. The identification of alterations in expression levels and/or genomic DNA of multiple genes may improve detection, diagnosis, prognosis and treatment of cancer. Extensive data mining and statistical analysis is required to discover combinations of tumor markers that can differentiate between normal variation and a cancerous state.

Blood-based liquid biopsies, including tumor-educated blood platelets (TEPs; Nilsson et al., 2011. Blood 118: 3680-3683; Best et al., 2015. Cancer Cell 28: 666-676; Nilsson et al., 2015. Oncotarget 7: 1066-1075), have emerged as promising biomarker sources for non-invasive detection of cancer and therapy selection. A notorious challenge is the identification of optimal biomarker panels from such liquid biosources. To select robust biomarker panels for disease classification the use of ‘swarm intelligence’ was proposed, especially particle swarm optimization (PSO) (Kennedy et al., 2001. The Morgan Kaufmann Series in Evolutionary Computation. Ed: David B. Fogel; Bonyadi and Michalewicz 2016. Evolutionary computation: 1-54; Kennedy and Eberhart, 1995. Proceedings of IEEE International Conference on Neural Networks: 1942-1948).

PSO-driven algorithms are inspired by the concomitant swarm of birds and schools of fish that by self-organisation efficiently adapt to their environment or identify sources of food. Bioinformatically, PSO algorithms are exploited for the identification of optimal solutions for complex parameter selection procedures, including the selection of biomarker gene lists (Alshamlan et al., 2015. Computational Biol Chem 56: 49-60; Martinez et al., 2010. Computational Biol Chem 34: 244-250).

### SUMMARY OF THE INVENTION

Targeted therapy and personalized medicine are critically depending on disease profiling and the development of companion diagnostics. Mutations in disease-derived nucleic acids can be highly predictive for the response to targeted treatment. However, obtaining easily accessible high-quality nucleic acids remains a significant developmental hurdle. Blood generally contains 150,000-350,000 thrombocytes (platelets) per microliter, providing a highly available biomarker source for research and clinical use. Moreover, thrombocyte isolation is relatively simple and is a standard procedure in blood bank/hematology labs. Since platelets do not contain a nucleus, their RNA transcripts—needed for functional maintenance—are derived from bone marrow megakaryocytes during thrombocyte origination. In addition, thrombocytes may take up RNA and/or DNA from other cells during circulation via various transfer mechanisms. Tumor cells for instance release an abundant collection of genetic material, some of which is secreted by microvesicles in the form of mutant RNA During circulation in the blood stream thrombocytes may absorb the genetic material secreted by cancer cells and other diseased cells, serving as an attractive platform for the companion diagnostics of cancer, specifically in the context of personalized medicine.

The present invention provides a method of administering immunotherapy that modulates an interaction between programmed death protein 1 (PD-1) and its ligand, to a cancer patient, comprising the steps of providing a sample from the patient, the sample comprising mRNA products that are obtained from anucleated cells of said patient; determining a gene expression level for at least four genes, more preferred at least five genes, more preferred at least six genes listed in Table 1 in said sample; comparing said determined gene expression level to a reference expression level of said genes in a reference sample; typing the patient as a positive responder to said immunotherapy, or as a not-positive responder, based on the comparison with the reference; and administering immunotherapy to a cancer patient that is typed as a positive responder.

In a preferred method of the invention, a gene expression level is determined for at least four genes listed in Table 1, more preferred at least five genes, more preferred at least six genes, more preferred at least ten genes, more preferred at least fifty genes, more preferred all genes, listed in Table 1.

Said immunotherapy that modulates an interaction between PD-1 and its ligand, PD-L1 or PD-L2, is aimed at activating the immune system to attack the cancer of the patient. Known modulators that inhibit interaction between PD-1 and its ligand include monoclonal antibodies such as atezolizumab (Genentech Oncology/Roche), avelumab (Merck/Pfizer), durvalumab (AstraZeneca/MedImmune), nivolumab (Bristol-Myers Squibb), lambrolizumab (Merck), pidilizumab (CureTech) and pembrolizumab (Merck), and fusion proteins such as AMP-224 (GlaxoSmithKline). A preferred immunotherapy comprises nivolumab.

In another embodiment, the invention provides a method of typing a sample of a subject for the presence or absence of a lung cancer, comprising the steps of providing a sample from the subject, whereby the sample comprises mRNA products that are obtained from anucleated cells of said subject; determining a gene expression level for at least five genes listed in Table 2; comparing said determined gene expression level to a reference expression level of said genes in a reference sample; and typing said sample for the presence or absence of a lung cancer on the basis of the comparison between the determined gene expression level and the reference gene expression level.

Said subject, a mammalian, preferably a human, is not known to suffer from lung cancer. Said lung cancer preferably is a non-small cell lung cancer.

In a preferred method of the invention, a gene expression level is determined for at least ten genes listed in Table 2, more preferred at least forty five genes, more preferred at least fifty genes, more preferred all genes, listed in Table 2.

Anucleated cells, as referred to herein above, may act as local and systemic responders during tumorigenesis and cancer metastasis, thereby being exposed to tumor-mediated education, and resulting in altered behaviour. Anucleated cells, such as thrombocytes can function as a RNA biomarker trove to detect and classify cancer from diverse sources. Said RNA present in anucleated cells preferably originates from tumor cells, and is transferred from tumor cells to anucleated cells. These anucleated cells can be easily isolated from a liquid biopsy such as blood and may contain RNA from nucleated tumor cells.

Said sample comprising mRNA products is preferably obtained from a liquid biopsy, preferably blood. Said anucleated cells preferably are or comprise thrombocytes. In a preferred embodiment, thrombocytes are isolated from a blood sample and mRNA is subsequently isolated from said isolated thrombocytes.

A gene expression level for at least four genes listed in Table 1, more preferred at least five genes listed in Table 1, and/or for at least five genes listed in Table 2, in said sample may be determined by any method known in the art, including micro-array-based analyses, serial analysis of gene expression (SAGE), multiplex Polymerase Chain Reaction (PCR), multiplex Ligation-dependent Probe Amplification (MLPA), bead based multiplexing such as Luminex/XMAP, and high-throughput sequencing including next generation sequencing. The gene expression level is preferably determined by next generation sequencing.

The invention further provides a method of treating a cancer patient, preferably a lung cancer patient, by assigning immunotherapy that modulates an interaction between PD-1 and its ligand to said patient, wherein said cancer patient is selected by typing a sample from the patient, the sample comprising mRNA products that are obtained from anucleated cells of said subject; determining a gene expression level for at least four genes listed in Table 1, more preferred at least five genes listed in Table 1; comparing said determined gene expression level to an expression level of said genes in a reference sample; typing the patient as a positive responder to said immunotherapy, or as a not-positive responder, based on the comparison with the reference; and assigning immunotherapy to a cancer patient that is selected as a positive responder.

Further provided is immunotherapy that modulates an interaction between PD-1 and its ligand, for use in a method of treating a cancer patient, preferably a lung cancer patient, wherein said cancer patient is selected by typing a sample from the patient, the sample comprising mRNA products that are obtained from anucleated cells of said subject; determining a gene expression level for at least four genes listed in Table 1, more preferred at least five genes listed in Table 1; comparing said determined gene expression level to an expression level of said genes in a reference sample; typing the patient as a positive responder to said immunotherapy, or as a not-positive responder, based on the comparison with the reference; and assigning immunotherapy to a cancer patient that is selected as a positive responder.

As is indicated herein above, said immunotherapy that modulates an interaction between PD-1 and its ligand. PD-L1 or PD-L2, is aimed at activating the immune system to attack the cancer of the patient. Known modulators that inhibit interaction between PD-1 and its ligand include monoclonal antibodies such as atezolizumab (Genentech Oncology/Roche), avelumab (Merck/Pfizer), durvalumab (AstraZeneca/MedImmune), nivolumab (Bristol-Myers Squibb), lambrolizumab (Merck), pidilizumab (CureTech) and pembrolizumab (Merck), and fusion proteins such as AMP-224 (GlaxoSmithKline). A preferred immunotherapy comprises nivolumab.

The invention further provides a method for obtaining a biomarker panel for typing of a sample from a subject, the method comprising isolating anucleated cells, preferably thrombocytes, from a liquid sample of a subject having condition A; isolating RNA from said isolated cells; determining RNA expression levels for at least 100 genes in said isolated RNA; determining RNA expression levels for said at least 100 genes in a control sample from a subject not having condition A; and using particle swarm optimization-based algorithms to obtain a biomarker panel that discriminates between a subject having condition A and a subject not having condition A.

It is preferred that the subject having condition A is suffering from a cancer, preferably a lung cancer, or had a known, positive response to a cancer treatment, while a subject not having condition A is not suffering from a cancer, or had a known, negative response to a cancer treatment.

### FIGURE LEGENDS

FIG. 1. PSO-enhanced thromboSeq for NSCLC diagnostics.

(a) Overview of Non-cancer and NSCLC platelet samples (total of 728) included in this study for thromboSeq. (b) Overview of alternative splicing analyses, the estimated contribution to the TEP signatures, and additional Figures related to these analyses. RBP=HNA-binding protein (c) Schematic representation of the particle-swarm intelligence approach. Light to dark grey colored dots represent AUC-values of 38 samples classified using a thromboSeq classification algorithm, with use of 100 randomly selected parameters (left) or 100 parameters selected by swarm-intelligence (right). Dots were mirrored twice for visualization purposes. Most optimal AUC-value reached by swarm-enhanced thromboSeq is indicated in both plots with an asterisk. (d) ROC analysis of swarm-enhanced thromboSeq classifications using Non-cancer and NSCLC cohorts matched for patient age and blood storage time. Grey dashed line indicates ROC evaluation of the training cohort assessed by LOOCV, red line indicates ROC evaluation of the evaluation cohort (n=40), blue line indicates ROC evaluation of the validation cohort (n=130). Indicated are cohort size, most optimal accuracy, and AUC-value. Acc.=accuracy. (e) Performance of the swarm-enhanced thromboSeq algorithm evaluated in the full 728-samples cohort summarized in a ROC curve. Swarm intelligence made use of the evaluation cohort (red line, n=88 samples) to optimize the classification performance of the 120-samples training cohort by selection of the biomarker gene panel. The swarm-enhanced thromboSeq NSCLC diagnostics algorithm was validated using a patient age and/or blood storage time-unmatched cohort (n=520, blue line). Performance of the training cohort, assessed by LOOCV, is indicated with a grey dashed line. Indicated are cohort, size, most optimal accuracy, and AUC-value. Acc.=accuracy.

FIG. 2—TEP-based nivolumab response prediction.

(a) Schematic overview of the experimental setup. Blood of patients eligible for treatment with PD-1 inhibitor nivolumab was included from one month before till start of treatment (baseline, t=0). Tumor response read-out based on CT-imaging and according to the RECIST 1.1, criteria were performed at 6-8 weeks, 3 months, and 6 months, after start of nivolumab therapy. Best response was selected as overall tumor response (see Example 1). (b) Heatmap of unsupervised clustering of platelet mRNAs following swarm-intelligence driven gene panel selection of responders (blue, n=44) and non-responders (red, n=60). (c) ROC analysis of the swarm-enhanced thromboSeq nivolumab response prediction algorithm of 104 nivolumab baseline samples. Training cohort performance as measured by LOOCV approach is indicated by a red line, dependent evaluation cohort by a black line, and independent validation cohort by a blue line. Grey solid (upper bound) and dotted (lower bound) lines indicate the ROC curve resulting from a randomly trained algorithm. The black dot indicates a potential clinical threshold of the algorithm for optimal therapy selection and non-responder rule-out. (d) A 2×2 cross-table indicating the classification accuracies of the independent validation cohort, with the thromboSeq classification read-out optimized towards a rule-out value. A 100% sensitivity results in 53% specificity. Indicated are sample numbers and percentages.

FIG. 3—Experimental approach thromboSeq.

(a) Schematic representation of thromboSeq machine learning-based liquid biopsies for cancer diagnostics and therapy monitoring. A library of RNA-seq data generated from platelets of individuals with different diseases and healthy individuals served as input for thromboSeq algorithm development. Following algorithm optimization using the swarm-module and model validation, the platform enables RNA signature-based disease classification and therapy monitoring. (b) Schematic representation and sample cohort details of the training, evaluation, and validation cohorts. Cohorts are used for assessing the analytical performance of swarm-enhanced thromboSeq and to investigate the diagnostic classification power in patient age- and blood storage time-matched cohorts. The patient age and blood storage time-matched cohort was validated on a 130-samples training cohort, optimized using a 40-samples evaluation cohort.

FIG. 4—Technical performance parameters of thromboSeq.

(a) Overview of the demographic characteristics of the platelet sample cohort (n=263) matched for patient age and blood storage time. Characteristics are shown for both Non-cancer (n=104) and NSCLC (n=159) individuals. Indicated per clinical group are number of male individuals and percentage of total, median age (including interquartile range (IQR) and minimal and maximum age, in years), smoking status and percentage of total, and metastasis of the primary NSCLC towards other organs (yes/no). n.a.=not applicable. (b) Overview of platelet activation markers as measured by flow cytometric analysis of n=3 (8 hours time point) or n=6 (other time points) platelet samples collected from healthy donors and isolated using the thromboSeq platelet isolation protocol. Light and dark grey boxes represent average percentage of platelets expressing respectively P-selectin or CD-63 on the surface. The box indicates the interquartile range (IQR), black line represents the median, and the whiskers indicate 1.5×IQR. Dots represent expression of these surface markers after platelet activation with TRAP (see Example 1). Platelet samples are only minimally activated using the thromboSeq platelet isolation protocol. (c) Summary of the platelet total RNA yield in nanograms per microliter isolated from 6 mL whole blood in EDTA-coated Vacutainers tubes. The RNA concentration and quality was measured by Bioanalyzer RNA Picochip analysis. Total RNA yield was summarized in boxplots for both Non-cancer (n=86) and NSCLC (n=151) separately. The box indicates the interquartile range (IQR), black line represents the median, and the whiskers indicate 1.5×IQR. Platelets of NSCLC patients had a significantly higher RNA yield as compared to Non-cancer patients (p=0.0014, two-sided independent Student's t-test). (d) Linearity and efficiency of SMARTer cDNA synthesis and amplification using the thromboSeq protocol. Correlation plot of estimated RNA input (x-axis, in pg/μL) to the output SMARTer cDNA yield (y-axis, in nM, n=177 observations in total). Each dot represents a sample, color-coded by clinical group. An average RNA input, as measured by Bioanalyzer Picochip RNA, of ˜500 pg was used for SMARTer cDNA synthesis and PCR amplification. The RNA input and cDNA output showed a positive correlation (r=0.23, p=0.003, Pearson's correlation). (e) Linearity and efficiency of Truseq cDNA library preparation and PCR amplification using the thromboSeq protocol. Correlation plot of SMARTer cDNA yield used as input (x-axis, in nM) to the outputted Truseq platelet cDNA sequence library yield (y-axis, in nM, n=177 observations in total). Each dot represents a sample, color-coded by clinical group. All SMARTer cDNA output, except a 1.5 μL purification buffer aliquot for Bioanalyzer analysis, was used as input for the Truseq Library Preparation. The SMARTer cDNA yield and Truseq platelet cDNA library output showed a positive correlation (r=0.44, p<0.0001, Pearson's correlation). (f) Bioanalyzer traces of samples with spiked, smooth, and intermediate spiked/smooth profiles. For each example, the total RNA on Picochip Bioanalyzer, the SMARTer amplified cDNA on DNA High Sensitivity Bioanalyzer, and Truseq cDNA library on DNA 7500 Bioanalyzer are shown. X-axes indicate the length of the product (in nucleotides (nt) for RNA, and base pairs (bp) for cDNA), while y-axes indicate the relative fluorescence as measured by the Bioanalyzer 2100. From spiked towards smooth SMARTer cDNA samples, a gradual increase of smoothness of the SMARTer cDNA Bioanalyzer slopes was observed, while the total RNA and Truseq cDNA show non-distinguishable profiles. (g) Overview of the relative cDNA yield in nM resulting from the SMARTer amplification (top), relative cDNA length in bp of the spiked, smooth, and intermediate spiked/smooth SMARTer cDNA groups (middle), and number of intron-spanning spliced RNA reads (bottom). cDNA concentrations were measured by the area-under-the-graph on a Bioanalyzer cDNA High Sensitivity chip. cDNA yield is comparable among the three distinct SMARTer profiles. The relative cDNA length was measured by selection of a 200-9000 bp region in the Bioanalyzer software. The SMARTer cDNA slopes are strongly correlated to the average cDNA length. The contribution of reads mapping to intergenic regions do negatively influence the number of intron-spanning reads eligible for thromboSeq analysis. Number of samples per SMARTer slope and clinical group is shown below the graph. The box indicates the interquartile range (IQR), black line represents the median, and the whiskers indicate 1.5×IQR. (h) Histogram of the average fragment length of reads mapped to intergenic regions for both spiked (upper) and smooth (bottom) samples (n=50 each, randomly sampled). Overlapping reads mapping to intergenic regions were merged (see Online Methods), and total resulting fragment sizes were quantified. Both spiked and smooth samples contain primarily fragments of <250 nt, with a peak in the 100-200 nt region. (i) Selection of intron-spanning spliced RNA reads for thromboSeq analysis. Stackplot indicates the distribution of reads for each sample, subspecified from intron-spanning, exonic, intronic, intergenic, and mitochondrial regions. Of note, the intron-spanning reads were subtracted from the reads mapping to the exonic regions. Samples (n=263) were sorted according to the proportion (y-axis) of intron-spanning reads. (j) Selection of samples with >3000 genes detected for thromboSeq analysis. Plot indicates for 740 platelet RNA samples subjected to thromboSeq the total number of intron-spanning reads (x-axis), and the number of genes detected (y-axis), with at least one intron-spanning read. The number of detected genes is partially correlated to the total number of intron-spanning reads yielded per sample. Samples with less than 3000 genes detected (n=10) were excluded from analyses. (k) Summary of the number of genes detected with confidence (i.e. >30 spliced RNA reads) in the platelet RNA samples using shallow thromboSeq (10-20 million reads on average), shown for both Non-cancer (n=377) and NSCLC (n=353) cohorts. The box indicates the interquartile range (IQR), black line represents the median, and the whiskers indicate 1.5×IQR. The average detection of genes per samples is ˜4500 different RNAs, and slightly decreased on average in platelets of NSCLC patients as compared to Non-cancer individuals. (l) Comparison of shallow versus deep thromboSeq. A total of 12 platelet RNA samples collected from healthy controls were subjected to deep thromboSeq (median 59.7 (min-max: 43.2-96.2) million raw reads counts per sample) and compared with the matched shallow thromboSeq RNA-seq data. For the deep thromboSeq, platelet samples were reprepared for sequencing, starting from platelet total RNA, with comparable input concentrations. Plot indicates the raw read counts for each gene (log-transformed y-axis) sorted by median read counts of all samples (x-axis). The three genes with highest expression in deep thromboSeq are highlighted. (m) Leave-one-sample-out cross-correlation. To investigate the comparability of one sample (test case) to all other sample (reference cohort), we performed cross-correlations, during which counts of each sample was correlated to the median counts of all other samples. This step was included as a quality control step (see Online Methods) following the selection for samples with sufficient number of genes detected (see also (j)). The cross-correlation was calculated 730 times, i.e. all samples were left out of the reference cohort, once. Results indicate that all samples show high inter-sample Pearson's correlations. Samples with a inter-sample-correlation <0.5 (n=2) were excluded from analyses.

FIG. 5—Differential spliced RNAs in TEPs of NSCLC patients.

(a) Unsupervised hierarchical clustering of differentially spliced RNAs between Non-cancer (n=104) and NSCLC (n=159) individuals. A total of 1625 genes (698 up, 927 down) showed a significance with a False Discovery Rate <0.01 (see Example 3). Columns indicate samples, rows indicate genes, and color intensity represent the z-score transformed RNA expression values (prior to visualization subjected to the RUV-based iteration correction-module). Clustering of samples showed non-random partitioning (p<0.0001, Fisher's exact test). (b) PAGODA gene ontology analysis (see Example 1). Significantly enriched genes were subjected to unbiased gene cluster identification and gene ontology analysis. Most significant results by adjusted Z-score, indicating high statistical significance, were clustered and visualized. Grey code indicates a dark to light (low-to-high) score per sample per gene cluster. The most significant biological group (maximum adjusted Z-score of 13.9) includes gene ontologies related to translation, RNA binding proteins (RBPs), and signaling, with a low splicing score in NSCLC samples compared to Non-cancer samples. The most significantly enriched gene cluster in NSCLC patients compared to Non-cancer individuals is related to signaling and immune response (maximum adjusted Z-score of 5.3). This clustering analysis identified correlations between platelet homeostasis gene signatures in platelets of Non-cancer individuals and specific immune signaling pathways in TEPs of NSCLC patients. RBP=RNA binding proteins.

FIG. 6—thromboSplicing.

(a) Schematic figure represents the read distribution analyses procedure. From the patient age- and blood storage time-matched cohort, we mapped 100 bp reads to the human genome and quantified the number of reads mapping to four distinct regions (see Example 3). i.e. exonic, intronic, and intergenic regions (together the ‘genomic regions’) and the mitochondrial genome (abbreviated as mtDNA). Of note, the intron-spanning spliced reads were included in the exonic regions. (b) Boxplots indicate for Non-cancer (light grey, n=104) and NSCLC (dark grey, n=159) the median and spread of reads mapping to mitochondrial (mtDNA), exonic, intronic, or intergenic regions, and the median and spread of intron-spanning and exon boundary reads. The box indicates the interquartile range (IQR), black line represents the median, and the whiskers indicate 1.5×IQR. Intron-spanning reads are defined as reads that start on exon a and end on exon b. Exon boundary reads are defined as reads that overlay a neighbouring exon-intron boundary. The exonic, intronic, intergenic, intron-spanning, and exon boundary reads were normalized to one million total genomic reads. (c) Summary figure of the analysis of alternative RNA isoforms. Schematic figure represents the development of an isoform count matrix. For this, 92 bp trimmed RNA-seq reads were mapped to the human genome and following subjected to the MISO algorithm. The MISO algorithm allows for inferring expressed RNA isoforms from single read RNA-seq data. MISO output data was deconvoluted into a count matrix that contains per sample for each expressed RNA isoform the number of reads supporting that particular isoform. The count matrix of 104 Non-cancer individuals and 159 NSCLC patients was used for differential expression analysis. Isoforms with a significance value (FDR)<0.01 were selected. Piechart of the total number of differentially spliced RNA isoforms (FDR<0.01, n=743, summarized in color codes) per gene (n=571, summarized in the pies of the piechart), indicating the distribution of significantly altered isoforms between Non-cancer and NSCLC per parent gene. In 38% of the significantly altered RNA isoforms multiple isoforms belonged to the same parent gene, supporting the notion that some genes show co-regulation of multiple RNA isoforms. Pie chart of total number of genes (n=571 in total) that shows for all RNA isoforms co-increased expression levels ( 277/571, 49%), co-decreased expression levels ( 281/571, 49%), or alternative splicing ( 13/571, 2%). Additional details are provided in Example 2. (d) Summarizing figure of the exon skipping events analysis. Schematic figure represent the experimental approach for detection of exon skipping events. Reads were mapped and analyzed using the MISO algorithm, which infers reads favouring either inclusion (on top of the schematic figure) or exclusion (below of schematic figure) of the specific exon. For this, the algorithm does also takes reads mapping to neighbouring exons into account. After filtering for average read coverage in the majority of the sample cohort (see Online Methods), a total 230 exons remained eligible for analysis. Percent spliced in (PSI)-values, as outputted by MISO, were used for differential ANOVA statistics. A total of 27 exons were identified as potentially skipped in either Non-cancer or NSCLC samples (FDR<0.01). The histogram shows the direction of the PSI-value, where positive PSI-values favour exclusion in Non-cancer, and negative PSI-values favour exclusion in NSCLC. The gene names of the annotated events, sorted by FDR-value and filtered for unique gene names, are listed in the box. Additional details are provided in Example 2.

FIG. 7—P-selectin signature.

(a) Correlation plot of proportion of reads mapping to exonic coordinates (x-axis) versus the log-transformed. RUV-corrected, and counts-per-million of P-selectin. Each dot represent a sample, coded by clinical group (NSCLC, n=159, dark grey, and Non-cancer, n=104, light grey). The exonic reads correlate with the expression levels of P-selectin (r=0.51, p<0.001). (b) Distribution of correlation coefficients of the correlation between log-transformed counts-per-million levels of 4722 genes and the log-transformed counts-per-million of P-selectin. A subset of the genes show a strong correlation with P-selectin (r approximates −1 or 1), whereas other do not (r approximates 0). For the histogram, a bin size of 0.05 was used. (c) Venn-diagram overlay of genes upregulated in the NSCLC TEP signature (698 genes, see also FIG. 5a), and genes with a significant positive correlation (FDR<0.01) towards P-selectin (SELP signature, 1820 genes), 77% ( 536/698) of genes increased in the TEP signature are also present in the SELP signature, suggesting that the SELP signature might partially contribute to the TEP signature.

FIG. 8—RNA-binding protein (RBP) analysis of TEP-derived RNA signatures.

(a) Schematic biological model highlighting the difference between nucleated cells and anucleated platelets in the context of regulation of translation. Nucleated cells (left) are able to regulate and maintain the transcriptome by transcription factor (TF)-mediated DNA transcription, resulting in protein translation. Anucleated platelets lack genomic DNA, and thus the ability to regulate the RNA content by TFs. Circulating platelets retain the ability to selectively splice the pre-mRNA repertoire, suggesting a key regulatory function during the induction of splicing events. (b) Schematic representation of the RBP-thromboSearch engine algorithm. The algorithm is designed to identify correlations between the presence of RBP motif sequences in specific genomic regions of the genome, here applied to 5′-UTRs and 3′-UTRs. At start, the algorithm extracts the reference sequence of the regions of interest from the human genome (hg19). In addition, the algorithm was complemented with validated RBP binding sites motif sequences that were previously identified (Ray et al., 2013. Nature 499: 172-177). By reduction of the motif sequences, 547 non-redundant oligonucleotide sequences were matched with the UTR reference sequences, and all matched counts (ranging 0 to 460) were summarized in a UTR-to-motif matrix, used for downstream analyses. For further details of the RBP-thromboSearch engine algorithm, see Example 1. (c) UTR-read coverage filter. UTR regions (n=19180, x-axis) included in this analysis were quantified for number of mapping reads (y-axis). UTRs with more than five (5′-UTRs) or three (3′-UTRs) mapped reads were considered present in platelets. Blue dots represent mean counts across all samples, grey shade indicates the respective standard deviations. (d) Enrichment of identified RBP binding sites per UTR region. The x- and y-axes represent the mean binding sites for the 5′ and 3′-UTR per RBP (dots, n=102). Several RBPs are specifically enriched in the 3′-UTR, whereas others are enriched in the 5′-UTR (see also Example 4). (e and f) Heatmap of all RBPs (n=80, rows) and all 5′-UTR (e) and 3′-UTH (f) regions detected with sufficient coverage in platelets (n=3210 for 5′-UTR, and n=3720 for 3′UTR, columns, see Example 4). Number of binding sites is reflected by the heatmaps colors (see grey scale). UTR regulation by RBPs seems to be mediated by presence/absence of RBP binding sites. (g) Correlation analysis between n binding sites of an RBP and the logarithmic fold-change (log FC) of genes (n=4722) in the NSCLC/Non-cancer differential splicing analysis (see also FIG. 5a). Positive correlations indicate an enrichment in binding sites with an increase of the log FC, whereas negative correlations indicate the opposite. Plots indicate the relation between the Spearman's correlation coefficient (x-axis) and the concomitant p-value adjusted for multiple hypothesis testing (FDR). Results suggest that RBP docking sites are implicated in the log FC of genes between NSCLC and Non-cancer.

FIG. 9—Schematic overview of PSO-enhanced thromboSeq classification algorithm and application to NSCLC and Non-cancer cohorts matched for patient age and blood storage time.

(a) Schematic overview of the iterative correction module as implemented in thromboSeq. The RNA-seq data correction procedure includes multiple steps, i.e. 1) filtering of low abundant genes, 2) determination of stable genes among confounding variables, 3) raw-read counts Remove Unwanted Variation (RUV)-based factor analysis and correction, and 4) reference group-mediated counts-per-million and TMM-normalisation (see also Example 1). In detail, in step 1 genes with low confidence of detection, i.e. less than 30 intron-spanning spliced RNA reads in more than 90% of the sample cohort, are excluded. In the schematic example, the two upper genes (rows) contain in >90% of the samples (in this schematic example n=10 in total) sufficient numbers of reads, as indicated by the light grey boxes. Thus, these genes will be included for analysis. The lower two boxes indicate insufficient numbers of samples with sufficient numbers of genes, thus prompting the algorithm to remove these particular genes from the downstream analyses. Secondly, the algorithm searches for genes that show a stable expression pattern among all other samples. For this, the algorithm performs multiple Pearson's correlation analyses among a (potential confounding) variable and raw read counts, resulting in a distribution of the correlation coefficients. In the schematic figure, this is shown for intron-spanning reads library size (left) and patient age (right). The correlation distribution is shown below, and the putative thresholds (also subjected to PSO selection, see (e)) are indicated by black lines. Of note, as the raw intron-spanning read counts are normalised by counts-per-million normalization afterwards, stable genes have to approximate a correlation coefficient of one (see also FIG. 9b-c). During the third step, the algorithm first identifies factors contributing to the data in an unbiased way, using the RUVseq-correction module (RUVg-function). The RUVSeq correction approach estimates and corrects based on a generalized linear model of a subset of genes and by singular value decomposition the contribution of covariates of interest and unwanted variation. Secondly, the algorithm iteratively correlates the variable of interest (group) and potentially confounding variables (patient age and blood storage time) to the factors identified by RUVSeq. If a factor is determined to be correlated to a confounding factor (e.g. intron-spanning reads library size in ‘Factor 1’), the factor will be marked for removal (‘Remove’). Alternatively, if a factor is determined to be correlated to the factor of interest (e.g. group in ‘Factor 2’) or to none of the factors identified as involved factors (e.g. ‘Factor 3’), the factor will not be removed (‘Keep’). Finally, in the fourth step, default counts-per-million normalization and Trimmed Mean of M-values (TMM)-correction is performed using only the samples from the training cohort as eligible samples to calculate the TMM-correction factor. (b) Same example for correlation intron-spanning library size as shown in A.2 (left), but here y-axis indicates counts-per-million (CPM) normalized counts. This graph emphasizes that, for this particular variable, a correlation coefficient up to 1 has to be selected, resulting in selection of genes stable after CPM-normalization. (c) Interquartile range distribution of all genes after CPM-normalization ordered by correlation with library size. Highly correlated genes (right of black line, example threshold r>0.8) show a minimal interquartile range after CPM-normalization as compared to the samples with a diminished correlation coefficient (left of the black line). (d) Relative log expression (RLE) plots of 263 samples normalized using our previous approach (upper plot) and the novel approach (current study, lower plot). The RLE plot indicates the log-ratio of a read count to the median count across samples, and should show for a well-normalized datasets a similar distribution centered around zero. The correction module reduces the intersample variability significantly (p<0.0001, two-sided Student's t-test). (e) Schematic overview of the swarm-enhanced thromboSeq classification module. Multiple steps and filters of the algorithm are swarm-optimized, as indicated by the ‘bird’-sign. First, the dataset is subjected to the iterative correction module (see FIG. 9a). Second, most differentially spliced genes are calculated and selected (see Example 1). Third, highly correlated genes among genes selected in the second step are removed. Fourth, an SVM model is built using the training cohort, optimizing the gamma (g) and cost (c) parameters by a grid search (see Online Methods). Fifth, all genes selected for classification are recursively ranked according to the contribution to the SVM model, resulting in a ranked classification gene list. This list is subjected to swarm-based filtering. Sixth, using the reduced gene list an updated SVM model, again with gamma (g) and cost (c) optimization by grid search, is built. Seventh, the gamma (g) and cost (c) values are further optimized by a second particle-swarm optimization algorithm (see Online Methods). Finally, using the reduced gene list and optimized gamma (g) and cost (c) parameters the final SVM model is built.

FIG. 10—Comparative analysis of TEP RNA profiles of NSCLC patients at 2-4 weeks after start of nivolumab treatment. (a) Differential splicing analysis of n=17 Responders and n=11 Non-responders of which blood was collected at 2-4 weeks following start of treatment. An 195-gene panel shows significant separation between Responders and Non-responders (gene panel optimized by swarm-intelligence, p<0.0001 by Fisher's exact test). Venn diagram shows that a 1246-gene baseline response prediction signature and a 195-gene baseline follow-up response prediction signature have minimal overlay. (b) Differential splicing analysis of n=61 Responders and n=72 Non-responders of which blood was collected at baseline and during 2-4 weeks following start of treatment. (c) 378 altered RNAs were identified in TEPs of Responders and 107 altered RNAs in TEPs of Non-responders that were on treatment (genes panel optimized by swarm-intelligence, p<0.0001 by Fishers exact test). Venn diagram shows that both signatures have minimal overlay.

### DETAILED DESCRIPTION

### (1) Abbreviations

As used herein, the term “cancer” refers to a disease or disorder resulting from the proliferation of oncogenically transformed cells. “Cancer” shall be taken to include any one or more of a wide range of benign or malignant tumours, including those that are capable of invasive growth and metastasis through a human or animal body or a part thereof, such as, for example, via the lymphatic system and/or the blood stream. As used herein, the term “tumour” includes both benign and malignant tumours or solid growths, notwithstanding that the present invention is particularly directed to the diagnosis or detection of malignant tumours and solid cancers. Cancers further include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukaemia. Hodgkin's disease, small cell carcinoma of the lung, melanoma, neuroblastoma, glioma (e.g. glioblastoma), and soft tissue sarcoma, lymphoma, melanoma, sarcoma, and adenocarcinoma. In preferred embodiments of aspects of the present invention, thrombocyte cancer is disclaimed.

The term “liquid biopsy”, as is used herein, refers to a liquid sample that is obtained from a subject. Said liquid biopsy is preferably selected from blood, urine, milk. cerebrospinal fluid, interstitial fluid, lymph, amniotic fluid, bile, cerumen, feces, female ejaculate, gastric juice, mucus pericardial fluid, pleural fluid, pus, saliva, semen, smegma, sputum, synovial fluid, sweat, tears, vaginal secretion, and vomit. A preferred liquid biopsy is blood.

The term “blood”, as is used herein, refers to whole blood (including plasma and cells) and includes arterial, capillary and venous blood.

The term “anucleated blood cell”, as used herein, refers to a cell that lacks a nucleus. The term includes reference to both erythrocyte and thrombocyte. Preferred embodiments of anucleated cells according to this invention are thrombocytes. The term “anucleated blood cell” preferably does not include reference to cells that lack a nucleus as a result of faulty cell division.

The term “thrombocyte”, as used herein, refers to blood platelets. i.e. small, irregularly-shaped cell fragments that do not have a DNA-containing nucleus and that circulate in the blood of mammals. Thrombocytes are 2-3 μm in diameter, and are derived from fragmentation of precursor megakaryocytes. Platelets or thrombocytes lack nuclear DNA, although they retain some megakaryocyte-derived mRNAs as part of their lineal origin. The average lifespan of a thrombocyte is 5 to 9 days. Thrombocytes are involved and play an essential role in hemostasis, leading to the formation of blood clots.

### (2) Determining Gene Expression Levels

The present invention describes methods of diagnosing, prognosticating or predicting a response to treatment, based on analyzing gene expression levels in anucleated cells such as thrombocytes extracted from blood. This approach is robust and easy. This is attributed to the rapid and straight forward extraction procedures and the quality of the extracted nucleic acid. Within the clinical setting, thrombocytes extraction from blood samples is implemented in general biological sample collection and therefore it is foreseen that the implementation into the clinic is relatively easy.

The present invention provides general methods of diagnosing, prognosticating or predicting treatment response of a subject using said general methods. When reference is herein made to a method of the invention, any and all of these embodiments are referred to, except if explicitly indicated otherwise.

A method of the invention can be performed on any suitable body sample comprising anucleated blood cells, such as for instance a tissue sample comprising blood, but preferably said sample is whole blood.

A blood sample of a subject can be obtained by any standard method, for instance by venous extraction.

The amount of blood that is required is not limited. Depending on the methods employed, the skilled person will be capable of establishing the amount of sample required to perform the various steps of the methods of the present invention and obtain sufficient nucleic acid for genetic analysis. Generally, such amounts will comprise a volume ranging from 0.01 μl to 100 ml, preferably between 1 μl to 10 ml, more preferably about 1 ml.

The body fluid, preferably blood sample, may be analyzed immediately following collection of the sample. Alternatively, analysis according to the method of the present invention can be performed on a stored body fluid or on a stored fraction of anucleated blood cells thereof, preferably thrombocytes. The body fluid for testing, or the fraction of anucleated blood cells thereof, can be preserved using methods and apparatuses known in the art. In an anucleated blood cell fraction, the thrombocytes are preferably maintained in inactivated state (i.e. in non-activated state). In that way, the cellular integrity and the disease-derived nucleic acids are best preserved. A thrombocyte-containing sample from a body fluid preferably does not include platelet poor plasma or platelet rich plasma (PRP). Further isolation of the platelets is preferred for optimal resolution.

A body fluid, preferably blood sample, may suitably be processed, for instance, it may be purified, or digested, or specific compounds may be extracted therefrom. Anucleated cells may be extracted from the sample by methods known to the skilled person and be transferred to any suitable medium for extraction of nucleic acid. The subject's body fluid may be treated to remove nucleic acid degrading enzymes like RNases and DNases, in order to prevent destruction of the nucleic acids.

Thrombocyte extraction from the body sample of the subject may involve any available method. In transfusion medicine, thrombocytes are often collected by apheresis, a medical technology in which the blood of a donor or patient is passed through an apparatus that separates out one particular constituent and returns the remainder to the circulation. The separation of individual blood components is done with a specialized centrifuge. Plateletpheresis (also called thrombopheresis or thrombocytapheresis) is an apheresis process of collecting thrombocytes. Modern automatic plateletpheresis allows blood donors to give a portion of their thrombocytes, while keeping their red blood cells and at least a portion of blood plasma. Although it is possible to provide the body fluid comprising thrombocytes as envisioned herein by apheresis, it is often easier to collect whole blood and isolate the thrombocyte fraction therefrom by centrifugation. Generally, in such a protocol, the thrombocytes are first separated from other blood cells by a centrifugation step of about 120×g for about 20 minutes at room temperature to obtain a platelet rich plasma (PRP) fraction. The thrombocytes are then washed, for example in phosphate-buffered saline/ethylenediaminetetraacetic acid, to remove plasma proteins and enrich for thrombocytes. Wash steps are generally followed by centrifugation at 850-1000×g for about 10 min at room temperature. Further enrichments can be carried out to yield more pure thrombocyte fractions.

Platelet isolation generally involves blood sample collection in Vacutainer tubes containing anticoagulant citrate dextrose (e.g. 36 ml citric acid, 5 mmol KCl, 90 mmol/l NaCl, 5 mmol/l glucose, 10 mmol/l EDTA pH 6.8). A suitable protocol for platelet isolation is described in Ferretti et al. (Ferretti et al., 2002. J Clin Endocrinol Metab 87: 2180-2184). This method involves a preliminary centrifugation step (1,300 rpm per 10 min) to obtain platelet-rich plasma (PRP). Platelets may then be washed three times in an anti-aggregation buffer (Tris-HCl 10 mmol/l; NaCl 150 mmol/l; EDTA 1 mmol/l; glucose 5 mmol/l; pH 7.4) and centrifuged as above, to avoid any contamination with plasma proteins and to remove any residual erythrocytes. A final centrifugation at 4,000 rpm for 20 min may then be performed to isolate platelets. For quantitative determination, the protein concentration of platelet membranes may be used as internal reference. Such protein concentrations may be determined by the method of Bradford (Bradford, 1976. Anal Biochem 72: 248-254), using serum albumin as a standard.

A sample comprising anucleated cells can be freshly prepared at the moment of harvesting, or can be prepared and stored at −70° C. until processed for sample preparation. Preferably, storage is performed under conditions that preserve the quality of the nucleic acid content of the anucleated cells. Examples of preservative conditions are fixation using e.g. formaline and paraffin embedding, the addition of RNase inhibitors such as RNAsin (Pharmingen) or RNasecure (Ambion), the addition of aqueous solutions such as RNAlater (Assuragen; U.S. Ser. No. 06/204,375), Hepes-Glutamic acid buffer mediated Organic solvent Protection Effect (HOPE; DE10021390), and RCL2 (Alphelys; WO04083369), and the addition of non-aquous solutions such as Universal Molecular Fixative (Sakura Finetek USA Inc.; U.S. Pat. No. 7,138,226).

Methods to determine gene expression levels are known to a skilled person and include, but are not limited to, Northern blotting, quantitative PCR, microarray analysis and RNA sequencing. It is preferred that said gene expression levels are determined simultaneously. Simultaneous analyses can be performed, for example, by multiplex qPCR, RNA sequencing procedures, and microarray analysis. Microarray analysis allow the simultaneous determination of gene expression levels of expression of a large number of genes, such as more than 50 genes, more than 100 genes, more than 1000 genes, more than 10,000 genes, or even whole-genome based, allowing the use of a large set of gene expression data for normalization of the determined gene expression levels in a method of the invention.

Microarray-based analysis involves the use of selected biomolecules that are immobilized on a solid surface, an array. A microarray usually comprises nucleic acid molecules, termed probes, which are able to hybridize to gene expression products. The probes are exposed to labeled sample nucleic acid, hybridized, and the abundance of gene expression products in the sample that are complementary to a probe is determined. The probes on a microarray may comprise DNA sequences. RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

A probe preferably is specific for a gene expression product of a gene as listed in Tables 1-3. A probe is specific when it comprises a continuous stretch of nucleotides that are completely complementary to a nucleotide sequence of a gene expression product, or a cDNA product thereof. A probe can also be specific when it comprises a continuous stretch of nucleotides that are partially complementary to a nucleotide sequence of a gene expression product of said gene, or a cDNA product thereof. Partially means that a maximum of 5% from the nucleotides in a continuous stretch of at least 20 nucleotides differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe. It is preferred that the probe is, or mimics, a single stranded nucleic acid molecule. The length of said complementary continuous stretch of nucleotides can vary between 15 bases and several kilo bases, and is preferably between 20 bases and 1 kilobase, more preferred between 40 and 100 bases, and most preferred about 60 nucleotides. A most preferred probe comprises about 60 nucleotides that are identical to a nucleotide sequence of a gene expression product of a gene, or a cDNA product thereof. In a method of the invention, probes comprising probe sequences as indicated in Tables 1-3 and 5-7 can be employed.

To determine the gene expression level by micro-arraying, the gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

A preferred method for determining gene expression levels is by sequencing techniques, preferably next-generation sequencing (NGS) techniques of RNA samples. Sequencing techniques for sequencing RNA have been developed. Such sequencing techniques include, for example, sequencing-by-synthesis. Sequencing-by-synthesis or cycle sequencing can be accomplished by stepwise addition of nucleotides containing, for example, a cleavable or photobleachable dye label as described, for example, in U.S. Pat. Nos. 7,427,673; 7,414,116; WO 04/018497 WO 91/06678; WO 07/123744; and U.S. Pat. No. 7,057,026. Alternatively, pyrosequencing techniques may be employed. Pyrosequencing detects the release of inorganic pyrophosphate (PPi) as particular nucleotides are incorporated into the nascent strand (Ronaghi et al., 1996, Analytical Biochemistry 242: 84-89; Ronaghi, 2001. Genome Res 11: 3-11; Ronaghi et al., 1998. Science 281: 363; U.S. Pat. Nos. 6,210,891; 6,258,568; and 6,274,320. In pyrosequencing, released PPi can be detected as it is immediately converted to adenosine triphosphate (ATP) by ATP sulfurylase, and the level of ATP generated is detected via luciferase-produced photons.

Sequencing techniques also include sequencing by ligation techniques. Such techniques use DNA ligase to incorporate oligonucleotides and identify the incorporation of such oligonucleotides and are inter alia described in U.S. Pat. Nos. 6,969,488; 6,172,218; and 6,306,597. Other sequencing techniques include, for example, fluorescent in situ sequencing (FISSEQ), and Massively Parallel Signature Sequencing (MPSS).

Sequencing techniques can be performed by directly sequencing RNA, or by sequencing a RNA-to-cDNA converted nucleic acid library. Most protocols for sequencing RNA samples employ a sample preparation method that converts the RNA in the sample into a double-stranded cDNA format prior to sequencing.

The determined gene expression levels are preferably normalized. Normalization refers to a method for adjusting or correcting a systematic error in the measurements for determining gene expression levels. Systemic bias may result from variation by differences in overall performance, differences in isolation efficiency of anucleated cells resulting in differences in purity of the isolated anucleated cells, and to variation between RNA samples, which can be due for example to variations in purity. Systemic bias can be introduced during the handling of the sample during the determination of gene expression levels.

### (3) Comparison of Determined Gene Expression Levels

The determined levels of expression of genes from Tables 1-3 in a sample are compared to the levels of expression of the same genes in a reference sample. Said comparison may result in an index score indicating a similarity of the determined expression levels in a sample of an individual, subject or patient, with the expression levels in the reference sample. For example, an index can be generated by determining a fold change/ratio between the median value of gene expression across samples that have been typed as being obtained from individuals suffering from cancer and the median value of gene expression across samples that are typed as being obtained from individuals not suffering from cancer. The relevance of this fold change/ratio as being significant between the two respective groups can be tested, for example, in an ANOVA (Analysis of variance) model. Univariate p-values can be calculated in the model and after multiple correction testing (Benjamini & Hochberg, 1995. JRSS, B, 57: 289-300) can be used as a threshold for determining significance that the gene expression shows a clear difference between the groups. Multivariate analysis may also be performed in adding covariates such as tumor stage/grade/size into the ANOVA model.

Similarly, an index can be determined by Pearson correlation between the expression levels of the genes in a sample of a patient and the average or mean of expression levels in one or more cancer samples that are known to respond to immunotherapy that modulates an interaction between PD-1 and its ligand, and the average or mean expression levels in one or more cancer samples that are known not to respond to immunotherapy that modulates an interaction between PD-1 and its ligand. The resultant Pearson scores can be used to provide an index score. Said score may vary between +1, indicating a prefect similarity, and −1, indicating a reverse similarity. Preferably, an arbitrary threshold is used to type samples as being responsive or as not being responsive. More preferably, samples are classified as responsive or as not responsive based on the respective highest similarity measurement. A similarity score is preferably displayed or outputted to a user interface device, a computer readable storage medium, or a local or remote computer system.

For predicting a response to immunotherapy that modulates an interaction between PD-1 and its ligand, said reference sample preferably comprises gene expression products that are obtained from anucleated cells of an individual known to respond positive to said immunotherapy, and/or of an individual known not to respond positive to said immunotherapy. Similarly, for typing of a sample of a subject for the presence or absence of a cancer, said reference sample preferably comprises gene expression products that are obtained from anucleated cells of an individual known to suffer from a cancer, and/or known not to suffer from a cancer.

Said reference sample preferably provides a measure of the average or mean level of expression of genes in anucleated cells of at least 2 independent individuals, more preferred at least 5 independent individuals, more preferred at least 10 independent individuals, such as between 10 and 100 individuals.

Said average or mean level of expression of genes in anucleated cells of the reference sample is preferably presented on a user interface device, a computer readable storage medium, or a local or remote computer system. The storage medium may include, but is not limited to, a floppy disk, an optical disk, a compact disk read-only memory (CD-ROM), a compact disk rewritable (CD-RW), a memory stick, and a magneto-optical disk.

### (4) Predicting Response to Administration of Immunotherapy that Modulates an Interaction Between PD-1 and its Ligand

The gene expression level for at least four genes listed in Table 1, more preferred at least five genes listed in Table 1 can be used to predict a response to immunotherapy that modulates an interaction between PD-1 and its ligand, to a cancer patient, prior to administering said therapy.

For this, anucleated cells, preferably thrombocytes, are isolated from a patient known to suffer from a cancer, such as a lung cancer. A sample comprising ribonucleic acid (RNA), preferably messenger RNA (mRNA), is isolated from the isolated anucleated cells. Following reverse transcription of the RNA into copy desoxyribonucleic acid (cDNA) using any method known to the skilled person, the resulting cDNA is labelled and gene expression levels are quantified, for example by next generation sequencing, for example on an Illumina sequencing platform.

Based on the sequencing results, the gene expression level for at least four genes listed in Table 1, more preferred at least five genes listed in Table 1 is determined in the sample comprising ribonucleic acid (RNA), from said cancer patient and preferably normalized. The normalized expression levels are compared to the level of expression of the same at least four genes listed in Table 1, more preferred at least five genes in a reference sample. Said reference sample is obtained from one or more cancer patients with a known, positive response to immunotherapy that modulates an interaction between PD-1 and its ligand, and/or obtained from one or more cancer patients with a known, negative response to immunotherapy that modulates an interaction between PD-1 and its ligand. From said comparison, a predicted response efficacy to administration of immunotherapy that modulates an interaction between PD-1 and its ligand such as, for example, administration of nivolumab, is obtained.

Contemplated herein is a method of typing a sample of a subject known to suffer from a cancer, especially a lung cancer, comprising the steps of providing a sample from the subject, whereby the sample comprises mRNA products that are obtained from anucleated cells of said subject; determining a gene expression level for at least four genes listed in Table 1, more preferred at least five genes listed in Table 1; comparing said determined gene expression level to a reference expression level of said genes in a reference sample; and typing said sample for a likelihood of responding to immunotherapy that modulates an interaction between PD-1 and its ligand such as, for example, administration of nivolumab, on the basis of the comparison between the determined gene expression level and the reference gene expression level.

In a preferred method according to the invention, a level of expression of at least four genes listed in Table 1, more preferred at least five genes from Table 1 is determined, more preferred a level of expression of at least ten genes from Table 1, more preferred a level of expression of at least twenty genes from Table 1, more preferred a level of expression of at least thirty genes from Table 1, more preferred a level of expression of at least forty genes from Table 1, more preferred a level of expression of at least fifty genes from Table 1, more preferred a level of RNA expression of all five hundred thirty two genes from Table 1.

It is further preferred that said at least five genes from Table 1 comprise the first four genes listed in Table 1, more preferred the first five genes with the lowest P-value, as is indicated in Table 1, more preferred the first ten genes with the lowest P-value, as is indicated in Table 1, more preferred the first twenty genes with the lowest P-value, as is indicated in Table 1, more preferred the first thirty genes with the lowest P-value, as is indicated in Table 1, more preferred the first forty genes with the lowest P-value, as is indicated in Table 1, more preferred the first fifty genes with the lowest P-value, as is indicated in Table 1.

In a further preferred embodiment, said at least four genes listed in Table 1, more preferred at least five genes from Table 1 comprise ENSG00000084234 (APLP2), ENSG00000165071 (TMEM71), ENSG00000143515 (ATP8B2), ENSG00000119314 (PTBP3) and ENSG00000126698 (DNAJC8); more preferably ENSG000000084234 (APLP2), ENSG00000165071 (TMEM71), ENSG00000143515 (ATP8B2), ENSG00000119314 (PTBP3), ENSG00000126698 (DNAJC8) and ENSG00000121879 (PIK3CA); more preferably ENSG0000000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA) and ENSG00000174238 (PITPNA); more preferably ENSG0000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA) and ENSG0000084754 (HADHA); more preferably ENSG0000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA), ENSG00000084754 (HADHA) and ENSG00000272369); more preferably ENSG0000084234 (APLP2), ENSG00000165071 (TMEM71), ENSG00000143515 (ATP8B2), ENSG0000019314 (PTBP3), ENSG0000126698 (DNAJC8), ENSG00000121879 (PIK3CA), ENSG00000174238 (PITPNA), ENSG00000084754 (HADHA), ENSG00000272369) and ENSG00000073111 (MCM2); more preferably ENSG00000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA), ENSG00000084754 (HADHA), ENSG00000272369), ENSG00000073111 (MCM2), ENSG00000137073 (UBAP2), ENSG00000115866 (DARS), ENSG00000229474 (PATL2), ENSG00000086589 (RBM22), ENSG00000145675 (PIK3R1), ENSG00000088833 (NSFL1C), ENSG00000267243, ENSG00000260661, ENSG00000144747 (TMF1) and ENSG00000158578 (ALAS2), more preferably ENSG00000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA), ENSG00000084754 (HADHA), ENSG00000272369), ENSG00000073111 (MCM2), ENSG00000137073 (UBAP2), ENSG00000115866 (DARS), ENSG0000229474 (PATL2), ENSG00000086589 (RBM22), ENSG00000145675 (PIK3R1), ENSG00000088833 (NSFL1C), ENSG000000267243, ENSG00000260661, ENSG0000144747 (TMF1), ENSG00000158578 (ALAS2), ENSG00000083642 (PDS5B), ENSG00000142089 (IFITM3), ENSG00000107175 (CREB3), ENSG00000162585 (C1orf86), ENSG00000142687 (KIAA0319L), ENSG00000100796 (SMEK1), ENSG00000142856 (ITGB3BP), ENSG00000103479 (RBL2), ENSG00000048471 (SNX29), ENSG00000196233 (LCOR) and ENSG00000068120 (COASY); more preferably ENSG00000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA), ENSG00000084754 (HADHA), ENSG00000272369), ENSG00000073111 (MCM2), ENSG00000137073 (UBAP2), ENSG00000115866 (DARS), ENSG00000229474 (PATL2), ENSG00000086589 (RBM22), ENSG00000145675 (PIK3R1), ENSG00000088833 (NSFL1C), ENSG00000267243, ENSG00000260661, ENSG00000144747 (TMF1), ENSG00000158578 (ALAS2), ENSG000000083642 (PDS5B), ENSG00000142089 (IFITM3), ENSG00000107175 (CREB3), ENSG00000162585 (C1orf86), ENSG000000142687 (KIAA0319L), ENSG00000100796 (SMEK1), ENSG00000142856 (ITGB3BP), ENSG00000103479 (RBL2), ENSG00000048471 (SNX29), ENSG00000196233 (LCOR), ENSG00000068120 (COASY), ENSG00000120868 (APAF1), ENSG00000198265 (HELZ), ENSG00000162688 (AGL), ENSG00000228215, ENSG00000147457 (CHMP7), ENSG00000129187 (DCTD), ENSG00000141644 (MBD1), ENSG00000172172 (MRPL13), ENSG0000011097 (PITPNM1) and ENSG00000102054 (RBBP7); more preferably ENSG00000084234 (APLP2), ENSG00000165071 (TMEM71), ENSG00000143515 (ATP8B2), ENSG00000119314 (PTBP3), ENSG00000126698 (DNAJC8), ENSG00000121879 (PIK3CA), ENSG00000174238 (PITPNA), ENSG0000084754 (HADHA), ENSG00000272369), ENSG00000073111 (MCM2), ENSG00000137073 (UBAP2), ENSG00000115866 (DARS), ENSG00000229474 (PATL2), ENSG00000086589 (RBM22), ENSG00000145675 (PIK3R1), ENSG0000088833 (NSFL1C), ENSG00000267243, ENSG00000260661, ENSG000000144747 (TMF1), ENSG00000158578 (ALAS2), ENSG00000083642 (PDS5B), ENSG00000142089 (IFITM3), ENSG00000107175 (CREB3), ENSG00000162585 (C1orf86), ENSG00000142687 (KIAA0319L), ENSG00000100796 (SMEK1), ENSG00000142856 (ITGB3BP), ENSG00000103479 (RBL2), ENSG00000048471 (SNX29), ENSG00000196233 (LCOR), ENSG00000068120 (COASY), ENSG00000120868 (APAF1), ENSG00000198265 (HELZ), ENSG00000162688 (AGL), ENSG00000228215, ENSG00000147457 (CHMP7), ENSG00000129187 (DCTD), ENSG00000141644 (MBD1), ENSG00000172172 (MRPL13), ENSG00000110697 (PITPNM1), ENSG00000102054 (RBBP7), ENSG000153214 (TMEM87B), ENSG0000150054 (MPP7), ENSG00000122008 (POLK), ENSG00000151150 (ANK3), ENSG00000165970 (SLC6A5), ENSG00000100811 (YY1), ENSG00000152127 (MGAT5), ENSG00000172493 (AFF1), ENSG00000213722 (DDAH2), ENSG00000177425 (PAWR), ENSG00000260017, ENSG0000141429 (GALNT1), ENSG00000119979 (FAM45A), ENSG00000136167 (LCP1), ENSG00000244734 (HBB), ENSG00000143569 (UBAP2L), ENSG00000079459 (FDFT1), ENSG00000197459 (HIST1H2BH) and ENSG00000080371 (RAB21).

In a most preferred embodiment, a set of at least four genes from Table 1 comprises ENSG00000164985 (PSIP1), ENSG00000114316 (USP4), ENSG00000103091 (WDR59) and ENSG00000140564 (FURIN), which resulted in an AUC-value of 0.70 (95%-CI: 0.47-0.94) and an classification accuracy of 73%.

### (5) Typing Presence or Absence of a Cancer

The gene expression level for at least five genes listed in Table 2 can be used to type a sample from a subject for the presence or absence of a cancer in said subject.

For this, anucleated cells, preferably thrombocytes, are isolated from a subject not known to suffer from a cancer, such as a lung cancer. A sample comprising ribonucleic acid (RNA), preferably messenger RNA (mRNA), is isolated from said isolated anucleated cells. Following reverse transcription of the RNA into copy desoxyribonucleic acid (cDNA) using any method known to the skilled person, the resulting cDNA is labelled and gene expression levels are quantified, for example by next generation sequencing, for example on an Illumina sequencing platform.

Based on the sequencing results, the gene expression level for at least five genes listed in Table 2 is determined in the sample comprising ribonucleic acid (RNA), from said subject and preferably normalized. The normalized expression levels are compared to the level of expression of the same at least five genes in a reference sample. Said reference sample is obtained from one or more cancer patients, and/or obtained from one or more subjects that are known not to suffer from a cancer. From said comparison, said subject can be types for a likelihood of having a cancer such as a lung cancer, or not having a cancer.

In a preferred method according to the invention, a level of expression of at least five genes from Table 2 is determined, more preferred a level of expression of at least ten genes from Table 2, more preferred a level of expression of at least twenty genes from Table 2, more preferred a level of expression of at least thirty genes from Table 2, more preferred a level of expression of at least forty genes from Table 2, more preferred a level of expression of at least fifty genes from Table 2, more preferred a level of RNA expression of all thousand genes from Table 2.

It is further preferred that said at least five genes from Table 2 comprise the first five genes with the lowest P-value, as is indicated in Table 2, more preferred the first ten genes with the lowest P-value, as is indicated in Table 2, more preferred the first twenty genes with the lowest P-value, as is indicated in Table 2, more preferred the first thirty genes with the lowest P-value, as is indicated in Table 2, more preferred the first forty genes with the lowest P-value, as is indicated in Table 2, more preferred the first fifty genes with the lowest P-value, as is indicated in Table 2.

In a further preferred embodiment, said at least five genes from Table 2 comprise HBB, EIF1, CAPNS1, NDUFAF3 and OTUD5, more preferred HBB, EIF1, CAPNS1, NDUFAF3, OTUD5, SRSF2, ANP32B, KIFAP3, ATOX1 and BCAP31, more preferred HBB, EF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM and DSTN, more preferred HBB, EIF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM, DSTN, ALDH9A1, ZNF346, LMAN1, EEF1B2, AP2S1, HSPB1, HBQ1, HTATIP2, PTMS and TPM2, more preferred HBB, EIF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM, DSTN, ALDH9A1, ZNF346, LMAN1, EEF1B2, AP2S1, HSPB1, HBQ1, HTATIP2, PTMS, TPM2, DESI1, RHOC, YWHAH, CPQ, FMTPN, ISCU, MRPL37, MGST3, CMTM5 and ACTG1, more preferred HBB, EIF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM, DSTN, ALDH9A1, ZNF346, LMAN1, EEF1B2, AP2S1, HSPB1, HBQ1, HTATIP2, PTMS, TPM2, DESI1, RHOC, YWHAH, CPQ, MTPN, ISCU, MRPL37, MGST3, CMTM5, ACTG1, ITGA2B, HPSE, KLHDC8B, CDC37, HLA-DRA, KSR1, ACOT7, PRKAR1B, MAOB and ZDHHC12, more preferred HBB, EIF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM, DSTN, ALDH9A1, ZNF346, LMAN1, EEF1B2, AP2S1, HSPB1, HBQ1, HTATIP2, PTMS, TPM2, DESI1, RHOC, YWHAH, CPQ, MTPN, ISCU, MRPL37, MGST3, CMTM5, ACTG1, ITGA2B, HPSE, KLHDC8B, CDC37, HLA-DRA, KSR1, ACOT7, PRKAR1B, MAOB, ZDHHC12, SNX3, YIF1B, PRDX5, HDAC8, DDX5, TPM1, SVIP, PDAP1, CD79B and PRSS50, more preferred HBB, EIF1, CAPNS1, NDUFAF3, OTUD5, SRSF2, ANP32B, KIFAP3, ATOX1, BCAP31, NAP1L1, TIMP1, POLR2E, CD74, POLR2G, RPS5, GPI, GSTM4, IGHM, DSTN, ALDH9A1, ZNF346, LMAN1, EEF1B2, AP2S1, HSPB1, HBQ1, HTATIP2, PTMS, TPM2, DESI1, RHOC, YWLAH, CPQ, MTPN, ISCU, MRPL37, MGST3, CMTM5, ACTG1, ITGA2B, HPSE, KLHDC8B, CDC37, HLA-DRA, KSR1, ACOT7, PRKAR1B, MAOB, ZDHHC12, SNX3, YIF1B, PRDX5, HDAC8, DDX5, TPM1, SVIP, PDAP1, CD79B, PRSS50, GPX1, IFITM3, SAIMD14, FUNDC2, BRIX1, CFL1, AKIRIN2, NAPSB, GPAA1, TRIM28, CMTM3 and MMP1.

In a most preferred embodiment, said at least 10 genes from Table 2 comprise ENSG00000168765 (GSTM4), ENSG00000206549 (PRSS50), ENSG00000106211 (HSPB1), ENSG00000185909 (IKLHDC8B), ENSG00000097021 (ACOT7), ENSG00000105401 (CDC37), ENSG00000099817 (POLR2E), ENSG00000105220 (GPI), ENSG00000075945 (KIFAP3), ENSG00000100418 (DESI1). The 10 genes resulted in an AUC-value of 0.74 (95%-CI: 0.70-0.77) and a classification accuracy of 68%) in an independent, late stage validation set (n=518 samples). The AUC-value was 0.69 (95%-CI: 0.59-0.79), with a classification accuracy of 65% in an early stage validation set (n=106 samples).

In a most preferred embodiment, a set of at least 45 genes from Table 2 is used to type a sample from a subject for the presence or absence of a cancer, especially a lung cancer, in said subject. Said at least 45 genes comprise ENSG00000023191 (RNH1), ENSG00000142089 (IFITM3), ENSG00000097021 (ACOT7), ENSG00000172757 (CFL1), ENSG00000213465 (ARL2), ENSG00000136938 (ANP32B), ENSG00000067365 (METTL22), ENSG00000130429 (ARPC1B), ENSG00000116221 (MRPL37), ENSG00000177556 (ATOX1), ENSG00000074695 (LMAN1), ENSG00000198467 (TPM2), ENSG00000188191 (PRKAR1B), ENSG00000126247 (CAPNS1), ENSG00000159335 (PTMS), ENSG00000113761 (ZNF346), ENSG00000102265 (TIMP1), ENSG00000168002 (POLR2G), ENSG00000185825 (BCAP31), ENSG00000155366 (RHOC), ENSG00000099817 (POLR2E), ENSG00000125868 (DSTN), ENSG00000160446 (ZDHHC12), ENSG00000100418 (DESI1), ENSG00000109854 (HTATIP2), ENSG00000161547 (SRSF2), ENSG000068308 (OTUD5), ENSG00000206549 (PRSS50), ENSG00000178057 (NDUFAF3), ENSG00000042753 (AP2S1), ENSG00000168765 (GSTM4), ENSG00000075945 (KIFAP3), ENSG00000173812 (EIF1), ENSG00000086506 (HBQ1), ENSG00000106244 (PDAP1), ENSG00000187109 (NAP1L1), ENSG00000106211 (HSPB1), ENSG00000105220 (GPI), ENSG00000105401 (CDC37), ENSG00000128245 (YWHAH), ENSG00000173083 (HPSE), ENSG00000185909 (KLHDC8B), ENSG00000126432 (PRDX5), ENSG00000166091 (CMTM5) and ENSG00000069535 (MAOB). The 45 genes resulted in an AUC-value of 0.77 (95%-CI: 0.73-0.81) and a classification accuracy of 77%) in an independent, late stage validation set (n=518 samples). The AUC-value was 0.74 (95%-CI: 0.65-0.83), with a classification accuracy of 70% in an early stage validation set (n=106 samples)

### (6) Additional P-Selectin Profile

P selectin protein (SELP, CD62) is stored in platelet alpha-granules and released upon platelet activation. P-selectin levels are enriched in younger, reticulated platelets. The platelet RNA gene panel selected for NSCLC diagnostics depicted in Table 2 contains genes that are co-regulated with p-selectin RNA expression in platelets. Hence, the NSCLC diagnostic signature may be enriched for reticulated platelets, expressing high levels of p-selectin RNA. Said P-selectin signature may have help in predicting therapy response, in case the platelet population of responding patients shifts during treatment from reticulated platelets to mature platelets. This shift might also be observed for other treatment modules including chemotherapy, targeted therapies, radiotherapy, surgery or immunotherapy.

Therefore, the gene expression level for at least five genes listed in Table 3 can be used to assist in predicting a response to immunotherapy that modulates an interaction between PD-1 and its ligand, to a cancer patient, prior to administering said therapy.

Hence, the invention provides a method of administering immunotherapy that modulates an interaction between PD-1 and its ligand, to a cancer patient, comprising the steps of providing a sample from the patient, the sample comprising mRNA products that are obtained from anucleated cells of said patient; determining a gene expression level for at least four genes listed in Table 1, more preferred at least five genes listed in Table 1, and at least five genes listed in Table 3; comparing said determined gene expression levels to reference expression levels of said genes in a reference sample; typing the patient as a positive responder to said immunotherapy, or as a not-positive responder, based on the comparison with the reference; and administering immunotherapy to a cancer patient that is typed as a positive responder.

For this, anucleated cells, preferably thrombocytes, are isolated from a patient known to suffer from a cancer, such as a lung cancer. A sample comprising ribonucleic acid (RNA), preferably messenger RNA (mRNA), is isolated from the isolated anucleated cells. Following reverse transcription of the RNA into copy desoxyribonucleic acid (cDNA) using any method known to the skilled person, the resulting cDNA is labelled and gene expression levels are quantified, for example by next generation sequencing, for example on an Illumina sequencing platform.

Based on the sequencing results, the gene expression level for at least five genes listed in Table 3 is determined in the sample comprising ribonucleic acid (RNA), from said cancer patient and preferably normalized. The normalized expression levels are compared to the level of expression of the same at least five genes in a reference sample. Said reference sample is obtained from one or more cancer patients with a known, positive response to immunotherapy that modulates an interaction between PD-1 and its ligand, and/or obtained from one or more cancer patients with a known, negative response to immunotherapy that modulates an interaction between PD-1 and its ligand. From said comparison, a predicted response efficacy to administration of immunotherapy that modulates an interaction between PD-1 and its ligand such as, for example, administration of nivolumab, is obtained.

In a preferred method according to the invention, a level of expression of at least five genes from Table 3 is determined, more preferred a level of expression of at least ten genes from Table 3, more preferred a level of expression of at least twenty genes from Table 3, more preferred a level of expression of at least thirty genes from Table 3, more preferred a level of expression of at least forty genes from Table 3, more preferred a level of expression of at least fifty genes from Table 3, more preferred a level of RNA expression of all thousand eight hundred twenty genes from Table 3.

It is further preferred that said at least five genes from Table 3 comprise the first five genes with the lowest P-value, as is indicated in Table 3, more preferred the first ten genes with the lowest P-value, as is indicated in Table 3, more preferred the first twenty genes with the lowest P-value, as is indicated in Table 3, more preferred the first thirty genes with the lowest P-value, as is indicated in Table 3, more preferred the first forty genes with the lowest P-value, as is indicated in Table 3, more preferred the first fifty genes with the lowest P-value, as is indicated in Table 3.

In a further preferred embodiment, said at least five genes from Table 3 comprise SELP, ITGA2B, AP2S1, OTUD5 and MAOB from Table 3, more preferred SELP, ITGA2B, AP2S1, OTUD5, MAOB, KIFAP3, HBQ1, ACOT7, POLR2E and DESI1, more preferred SELP, ITGA2B, AP2S, OTUD5, MAOB, KIFAP3, HBQ1, ACOT7, POLR2E, DESI1, TIMP1, CPQ, GPI, CDC37, MTPN, HSPB1, PDAP1, HTATIP2, SNX3 and ZNF346, more preferred SELP, ITGA2B, AP2S1, OTUD5, MAOB, KIFAP3, HBQ1, ACOT7, POLR2E, DESI1, TIMP1, CPQ, GPI, CDC37, MTPN, HSPB1, PDAP1, HTATIP2, SNX3, ZNF346, DSTN, CAPNS1, PRDX5, YWHAH, AKIRIN2, ISCU, TPM1, CMTM3, ALDH9A1 and RHOC, more preferred SELP, ITGA2B, AP2S1, OTUD5, MAOB, KIFAP3, HBQ1, ACOT7, POLR2E, DESI1, TIMP1, CPQ, GPI, CDC37, MTPN, HSPB1, PDAP1, HTATIP2, SNX3, ZNF346, DSTN, CAPNS1, PRDX5, YWHAH, AKIRIN2, ISCU, TPM1, CMTM3, ALDH9A1, RHOC, PTMS, ZDHHC12, SRSF2, FUNDC2, CMTM5, SAMD14, YIF1B, POLR2G, GSTM4 and CFL1, more preferred SELP, ITGA2B, AP2S1, OTUD5, MAOB, KIFAP3, HBQ1, ACOT7, POLR2E, DESI1, TIMP1, CPQ, GPI, CDC37, MTPN, HSPB1, PDAP1, HTATIP2, SNX3, ZNF346, DSTN, CAPNS1, PRDX5, YWHAH, AKIRIN2, ISCU, TPM1, CMTM3, ALDH9A1, RHOC, PTMS, ZDHHC12, SRSF2, FUNDC2, CMTM5, SAMD14, YIF1B, POLR2G, GSTM4, CFL1, HPSE, EIF1, NDUFAF3, ACTG1, BCAP31, KLHDC8B, NAP1L1, PRKAR1B, MMP1, GPAA1, SVIP, TPM2, PRSS50 and GPX1.

A most preferred set of at least five genes from Table 3 comprises ENSG00000161203 (AP2M1), ENSG00000204420 (C6orf25), ENSG00000204592 (HLA-E), ENSG00000064601 (CTSA), and ENSG00000005961 (ITGA2B). Use of this additional set of genes, besides the most preferred set of at least ten genes, resulted in classification of early-stage NSCLC with an AUC-value of 0.66 (95%-CI: 0.55-0.76) and an accuracy of 65% (n=106 samples).

### (7) Definition Particle Swarm Optimization

Several bioinformatic optimization algorithms can be exploited for solving mathematical problems regarding parameter selection. These optimization processes iteratively seek most optimal parameter settings of parameters that determine the mathematical problem. This iterative process is guided by the optimization algorithm, effectively and efficiently. We claim the use of particle swarm intelligence optimization (PSO), the mathematical approach for parameter selection, including its subvariants and hybridization/combination with other mathematical optimization algorithms for gene panel selection in liquid biopsies. We define PSO as a meta-algorithm exploiting particle position and particle velocity using iterative repositioning in a high-dimensional space for efficient and optimized parameter selection, i.e. gene panel selection. PSO also includes other optimization meta-algorithms that can be applied for automated and enhanced gene panel selection. We tested the particle swarm optimization algorithm, and demonstrate that PSO-enhanced algorithms enable efficient selection of spliced RNA biomarker panels from platelet RNA-seq libraries (n=728). This resulted in accurate TEP-based detection of stage IV non-small cell lung cancer (NSCLC) (n=520 independent validation cohort, accuracy: 89%, AUC: 0.94, 95%-CI: 0.93-0.96, p<0.001), independent of age of the individuals, whole blood storage time, and various inflammatory conditions. In addition, we employed swarm intelligence to explore spliced RNA biomarker profiles for the blood-based therapeutic response prediction of stage IV NSCLC patients at moment of baseline for anti-PD-1 nivolumab immunotherapy (n=64). The nivolumab response prediction algorithm resulted in an accuracy of 88% (AUC 0.89, 95%-CI: 0.8-1.0, p<0.01). To our knowledge this is the first demonstration of PSO for selection of biomarker gene panels to diagnose cancer and predict therapy response from TEPs. The PSO-algorithm was exploited for optimization of four parameters that determined the gene panel used for support vector machine training. As aside analyzing RNA molecules from TEPs. PSO can also be applied for analysis of small RNAs, RNA rearrangements. DNA single nucleotide alterations, protein levels, metabolomic levels, which constituents are isolated from TEPs, plasma, serum, circulating tumor cells, or extracellular vesicles, by subjecting similar or combined data input to the PSO-algorithm.

For the purpose of clarity and a concise description, features are described herein as part of the same or separate embodiments, however, it will be appreciated that the scope of the invention may include embodiments having combinations of all or some of the features described.

TABLE 1

| Ensembl_gene_id | hgnc_symbol | logFC | p-value |
|---|---|---|---|
| ENSG00000084234 | APLP2 | −4.42 | 0.000 |
| ENSG00000163359 | COL6A3 | 1.93 | 0.001 |
| ENSG00000147099 | HDAC8 | 1.47 | 0.001 |
| ENSG00000165970 | SLC6A5 | 1.24 | 0.003 |
| ENSG00000164068 | RNF123 | 1.88 | 0.003 |
| ENSG00000238683 | | 1.12 | 0.004 |
| ENSG00000248538 | | −2.46 | 0.004 |
| ENSG00000110422 | HIPK3 | −1.19 | 0.006 |
| ENSG00000005486 | RHBDD2 | 0.99 | 0.007 |
| ENSG00000065833 | ME1 | 1.53 | 0.008 |
| ENSG00000109790 | KLHL5 | −1.31 | 0.009 |
| ENSG00000175634 | RPS6KB2 | −0.66 | 0.010 |
| ENSG00000095319 | NUP188 | −1.50 | 0.013 |
| ENSG00000112992 | NNT | −0.97 | 0.013 |
| ENSG00000166164 | BRD7 | −0.73 | 0.013 |
| ENSG00000137075 | RNF38 | −1.38 | 0.014 |
| ENSG00000100612 | DHRS7 | −0.72 | 0.014 |
| ENSG00000198176 | TFDP1 | 1.20 | 0.014 |
| ENSG00000169967 | MAP3K2 | −1.06 | 0.015 |
| ENSG00000233369 | | 1.14 | 0.015 |
| ENSG00000107937 | GTPBP4 | −1.16 | 0.017 |
| ENSG00000035681 | NSMAF | −1.46 | 0.018 |
| ENSG00000111231 | GPN3 | −1.17 | 0.018 |
| ENSG00000060688 | SNRNP40 | −0.81 | 0.019 |
| ENSG00000206549 | PRSS50 | −2.05 | 0.020 |
| ENSG00000172869 | DMXL1 | −0.88 | 0.021 |
| ENSG00000070610 | GBA2 | 0.78 | 0.021 |
| ENSG00000143569 | UBAP2L | 0.73 | 0.022 |
| ENSG00000136824 | SMC2 | 1.04 | 0.022 |
| ENSG00000163220 | S100A9 | −1.05 | 0.023 |
| ENSG00000077380 | DYNC1I2 | 0.62 | 0.023 |
| ENSG00000151465 | CDC123 | −0.86 | 0.025 |
| ENSG00000182463 | TSHZ2 | 0.82 | 0.025 |
| ENSG00000106211 | HSPB1 | 0.61 | 0.025 |
| ENSG00000122299 | ZC3H7A | −0.67 | 0.026 |
| ENSG00000112118 | MCM3 | −1.22 | 0.027 |
| ENSG00000092964 | DPYSL2 | −1.70 | 0.027 |
| ENSG00000144283 | PKP4 | 1.04 | 0.028 |
| ENSG00000134242 | PTPN22 | 1.23 | 0.028 |
| ENSG00000198467 | TPM2 | 0.87 | 0.028 |
| ENSG00000067704 | IARS2 | 1.29 | 0.030 |
| ENSG00000198502 | HLA-DRB5 | −1.52 | 0.030 |
| ENSG00000114383 | TUSC2 | 0.79 | 0.031 |
| ENSG00000167414 | GNG8 | 0.90 | 0.031 |
| ENSG00000138029 | HADHB | 0.71 | 0.031 |
| ENSG00000168944 | CEP120 | 1.32 | 0.031 |
| ENSG00000183401 | CCDC159 | −0.80 | 0.031 |
| ENSG00000132950 | ZMYM5 | 0.99 | 0.031 |
| ENSG00000072518 | MARK2 | −0.85 | 0.032 |
| ENSG00000060138 | YBX3 | 0.71 | 0.032 |
| ENSG00000231389 | HLA-DPA1 | −0.62 | 0.032 |
| ENSG00000180370 | PAK2 | −0.67 | 0.033 |
| ENSG00000165113 | GKAP1 | 1.10 | 0.034 |
| ENSG00000205744 | DENND1C | −0.85 | 0.034 |
| ENSG00000166938 | DIS3L | −1.39 | 0.035 |
| ENSG00000114316 | USP4 | −0.48 | 0.035 |
| ENSG00000104660 | LEPROTL1 | −0.71 | 0.035 |
| ENSG00000148248 | SURF4 | −0.81 | 0.036 |
| ENSG00000213889 | PPM1N | 0.87 | 0.037 |
| ENSG00000007168 | PAFAH1B1 | −0.63 | 0.037 |
| ENSG00000185187 | SIGIRR | −0.76 | 0.039 |
| ENSG00000082397 | EPB41L3 | −0.99 | 0.039 |
| ENSG00000083937 | CHMP2B | −0.78 | 0.040 |
| ENSG00000141644 | MBD1 | −0.79 | 0.040 |
| ENSG00000198873 | GRK5 | 0.54 | 0.041 |
| ENSG00000049860 | HEXB | −0.89 | 0.042 |
| ENSG00000129071 | MBD4 | −0.63 | 0.043 |
| ENSG00000138085 | ATRAID | −0.56 | 0.043 |
| ENSG00000131467 | PSME3 | −1.02 | 0.044 |
| ENSG00000120688 | WBP4 | −0.55 | 0.045 |
| ENSG00000118260 | CREB1 | −0.91 | 0.047 |
| ENSG00000103544 | C16orf62 | −0.76 | 0.047 |
| ENSG00000114867 | EIF4G1 | −0.51 | 0.047 |
| ENSG00000106012 | IQCE | 1.06 | 0.048 |
| ENSG00000117475 | BLZF1 | −0.97 | 0.048 |
| ENSG00000075856 | SART3 | −0.69 | 0.049 |
| ENSG00000107874 | CUEDC2 | −0.65 | 0.049 |
| ENSG00000170525 | PFKFB3 | 0.98 | 0.049 |
| ENSG00000051382 | PIK3CB | −0.73 | 0.050 |
| ENSG00000131508 | UBE2D2 | −0.42 | 0.050 |
| ENSG00000196975 | ANXA4 | 0.64 | 0.051 |
| ENSG00000196396 | PTPN1 | 0.63 | 0.051 |
| ENSG00000155729 | KCTD18 | −1.15 | 0.052 |
| ENSG00000153066 | TXNDC11 | 0.78 | 0.052 |
| ENSG00000132305 | IMMT | 0.72 | 0.052 |
| ENSG00000107077 | KDM4C | −1.07 | 0.052 |
| ENSG00000143546 | S100A8 | −0.85 | 0.053 |
| ENSG00000163513 | TGFBR2 | −0.68 | 0.053 |
| ENSG00000108344 | PSMD3 | −0.89 | 0.054 |
| ENSG00000129103 | SUMF2 | 0.81 | 0.054 |
| ENSG00000179912 | R3HDM2 | −0.61 | 0.054 |
| ENSG00000138767 | CNOT6L | −0.65 | 0.054 |
| ENSG00000076513 | ANKRD13A | 0.80 | 0.055 |
| ENSG00000128708 | HAT1 | −0.69 | 0.055 |
| ENSG00000101161 | PRPF6 | −0.70 | 0.055 |
| ENSG00000140612 | SEC11A | −0.69 | 0.055 |
| ENSG00000138802 | SEC24B | 1.16 | 0.056 |
| ENSG00000068028 | RASSF1 | −0.79 | 0.056 |
| ENSG00000167996 | FTH1 | 0.62 | 0.057 |
| ENSG00000198336 | MYL4 | −1.50 | 0.057 |
| ENSG00000160551 | TAOK1 | 0.61 | 0.057 |
| ENSG00000165949 | IFI27 | 1.24 | 0.057 |
| ENSG00000163221 | S100A12 | −0.98 | 0.057 |
| ENSG00000103811 | CTSH | −1.01 | 0.058 |
| ENSG00000113240 | CLK4 | −0.82 | 0.059 |
| ENSG00000126217 | MCF2L | 0.75 | 0.059 |
| ENSG00000115020 | PIKFYVE | −0.95 | 0.059 |
| ENSG00000264538 | | −0.66 | 0.060 |
| ENSG00000113312 | TTC1 | 0.65 | 0.060 |
| ENSG00000171206 | TRIM8 | −0.84 | 0.061 |
| ENSG00000163781 | TOPBP1 | 1.02 | 0.062 |
| ENSG00000112234 | FBXL4 | 0.85 | 0.063 |
| ENSG00000178425 | NT5DC1 | −0.89 | 0.063 |
| ENSG00000137100 | DCTN3 | 0.67 | 0.064 |
| ENSG00000109854 | HTATIP2 | 0.68 | 0.064 |
| ENSG00000179115 | FARSA | −0.75 | 0.065 |
| ENSG00000138434 | SSFA2 | 0.54 | 0.065 |
| ENSG00000101220 | C20orf27 | −0.60 | 0.065 |
| ENSG00000135040 | NAA35 | −0.96 | 0.065 |
| ENSG00000184203 | PPP1R2 | −0.51 | 0.066 |
| ENSG00000182093 | WRB | 0.71 | 0.066 |
| ENSG00000161813 | LARP4 | 0.74 | 0.067 |
| ENSG00000012124 | CD22 | −0.90 | 0.068 |
| ENSG00000196407 | THEM5 | 0.78 | 0.069 |
| ENSG00000102145 | GATA1 | 0.46 | 0.069 |
| ENSG00000260017 | | 0.75 | 0.070 |
| ENSG00000172922 | RNASEH2C | 0.57 | 0.070 |
| ENSG00000027075 | PRKCH | −0.93 | 0.070 |
| ENSG00000103005 | USB1 | 0.62 | 0.071 |
| ENSG00000138449 | SLC40A1 | −0.76 | 0.072 |
| ENSG00000204428 | LY6G5C | 0.67 | 0.072 |
| ENSG00000067334 | DNTTIP2 | −0.75 | 0.072 |
| ENSG00000104133 | SPG11 | −0.76 | 0.073 |
| ENSG00000125977 | EIF2S2 | 0.47 | 0.073 |
| ENSG00000171566 | PLRG1 | 0.75 | 0.073 |
| ENSG00000173852 | DPY19L1 | −1.19 | 0.074 |
| ENSG00000109272 | PF4V1 | 0.63 | 0.074 |
| ENSG00000167261 | DPEP2 | −0.84 | 0.075 |
| ENSG00000143553 | SNAPIN | 0.67 | 0.075 |
| ENSG00000119900 | OGFRL1 | −0.61 | 0.075 |
| ENSG00000132676 | DAP3 | −0.59 | 0.076 |
| ENSG00000136044 | APPL2 | 0.85 | 0.076 |
| ENSG00000086189 | DIMT1 | −1.03 | 0.077 |
| ENSG00000042493 | CAPG | −0.78 | 0.077 |
| ENSG00000130313 | PGLS | −0.69 | 0.077 |
| ENSG00000204209 | DAXX | −0.49 | 0.077 |
| ENSG00000055070 | SZRD1 | 0.58 | 0.078 |
| ENSG00000065518 | NDUFB4 | −0.47 | 0.078 |
| ENSG00000102531 | FNDC3A | −0.80 | 0.079 |
| ENSG00000121879 | PIK3CA | 0.98 | 0.080 |
| ENSG00000113387 | SUB1 | −0.40 | 0.080 |
| ENSG00000141968 | VAV1 | 0.71 | 0.081 |
| ENSG00000109536 | FRG1 | −0.52 | 0.081 |
| ENSG00000128915 | NARG2 | 0.96 | 0.082 |
| ENSG00000144802 | NFKBIZ | 1.20 | 0.082 |
| ENSG00000089154 | GCN1L1 | −1.06 | 0.082 |
| ENSG00000148481 | FAM188A | 1.07 | 0.083 |
| ENSG00000023228 | NDUFS1 | −0.79 | 0.083 |
| ENSG00000165629 | ATP5C1 | −0.35 | 0.084 |
| ENSG00000135506 | OS9 | −0.56 | 0.084 |
| ENSG00000109919 | MTCH2 | −0.52 | 0.085 |
| ENSG00000026297 | RNASET2 | −0.57 | 0.086 |
| ENSG00000166508 | MCM7 | −0.54 | 0.086 |
| ENSG00000109113 | RAB34 | −0.68 | 0.086 |
| ENSG00000102103 | PQBP1 | −0.61 | 0.086 |
| ENSG00000184205 | TSPYL2 | 0.70 | 0.087 |
| ENSG00000105185 | PDCD5 | −0.66 | 0.087 |
| ENSG00000109736 | MFSD10 | −0.65 | 0.087 |
| ENSG00000161204 | ABCF3 | −0.79 | 0.087 |
| ENSG00000159335 | PTMS | 0.47 | 0.087 |
| ENSG00000145996 | CDKAL1 | 0.88 | 0.087 |
| ENSG00000100811 | YY1 | 0.45 | 0.088 |
| ENSG00000115234 | SNX17 | −0.36 | 0.088 |
| ENSG00000151136 | BTBD11 | −1.63 | 0.088 |
| ENSG00000169621 | APLF | 1.02 | 0.088 |
| ENSG00000180190 | TDRP | −0.80 | 0.089 |
| ENSG00000100079 | LGALS2 | −1.06 | 0.089 |
| ENSG00000167085 | PHB | −0.71 | 0.089 |
| ENSG00000013275 | PSMC4 | −0.46 | 0.091 |
| ENSG00000159658 | EFCAB14 | −0.60 | 0.091 |
| ENSG00000155366 | RHOC | 0.59 | 0.092 |
| ENSG00000113013 | HSPA9 | −0.60 | 0.092 |
| ENSG00000168090 | COPS6 | −0.45 | 0.092 |
| ENSG00000133742 | CA1 | 1.38 | 0.093 |
| ENSG00000064687 | ABCA7 | −0.73 | 0.093 |
| ENSG00000181704 | YIPF6 | 0.62 | 0.093 |
| ENSG00000169891 | REPS2 | −0.63 | 0.093 |
| ENSG00000175567 | UCP2 | −0.44 | 0.093 |
| ENSG00000223553 | SMPD4P1 | −1.25 | 0.093 |
| ENSG00000164031 | DNAJB14 | −0.73 | 0.095 |
| ENSG00000257261 | | 0.80 | 0.095 |
| ENSG00000036257 | CUL3 | −0.52 | 0.095 |
| ENSG00000170315 | UBB | 0.60 | 0.095 |
| ENSG00000143515 | ATP8B2 | −0.76 | 0.095 |
| ENSG00000151893 | CACUL1 | −0.90 | 0.096 |
| ENSG00000135930 | EIF4E2 | −0.64 | 0.096 |
| ENSG00000100299 | ARSA | 0.55 | 0.097 |
| ENSG00000127084 | FGD3 | −0.48 | 0.098 |
| ENSG00000132842 | AP3B1 | 0.58 | 0.098 |
| ENSG00000109466 | KLHL2 | −0.97 | 0.099 |
| ENSG00000167986 | DDB1 | 0.67 | 0.099 |
| ENSG00000108523 | RNF167 | −0.35 | 0.100 |
| ENSG00000143149 | ALDH9A1 | −0.60 | 0.100 |
| ENSG00000197555 | SIPA1L1 | −0.84 | 0.100 |
| ENSG00000101335 | MYL9 | 0.56 | 0.100 |
| ENSG00000138757 | G3BP2 | −0.51 | 0.100 |
| ENSG00000104960 | PTOV1 | 0.52 | 0.100 |
| ENSG00000130402 | ACTN4 | −0.65 | 0.101 |
| ENSG00000163444 | TMEM183A | 0.48 | 0.101 |
| ENSG00000136709 | WDR33 | −0.72 | 0.101 |
| ENSG00000103342 | GSPT1 | 0.75 | 0.102 |
| ENSG00000115520 | COQ10B | −0.75 | 0.102 |
| ENSG00000237854 | LINC00674 | 0.41 | 0.102 |
| ENSG00000064225 | ST3GAL6 | −0.70 | 0.102 |
| ENSG00000108582 | CPD | −1.33 | 0.103 |
| ENSG00000105404 | RABAC1 | 0.44 | 0.103 |
| ENSG00000113318 | MSH3 | 0.65 | 0.103 |
| ENSG00000196683 | TOMM7 | −0.56 | 0.103 |
| ENSG00000092199 | HNRNPC | −0.31 | 0.104 |
| ENSG00000021574 | SPAST | 0.71 | 0.104 |
| ENSG00000110711 | AIP | −0.56 | 0.105 |
| ENSG00000022277 | RTFDC1 | 0.35 | 0.105 |
| ENSG00000114439 | BBX | 0.38 | 0.106 |
| ENSG00000035687 | ADSS | −0.65 | 0.106 |
| ENSG00000100353 | EIF3D | −0.50 | 0.106 |
| ENSG00000103202 | NME4 | 0.53 | 0.107 |
| ENSG00000183386 | FHL3 | 0.70 | 0.107 |
| ENSG00000240356 | RPL23AP7 | 0.89 | 0.107 |
| ENSG00000113269 | RNF130 | −0.33 | 0.107 |
| ENSG00000130638 | ATXN10 | −0.51 | 0.107 |
| ENSG00000215302 | | 0.61 | 0.107 |
| ENSG00000179051 | RCC2 | −0.76 | 0.108 |
| ENSG00000236397 | DDX11L2 | 0.76 | 0.108 |
| ENSG00000183258 | DDX41 | 0.49 | 0.109 |
| ENSG00000122257 | RBBP6 | 0.50 | 0.109 |
| ENSG00000113638 | TTC33 | −0.61 | 0.110 |
| ENSG00000141068 | KSR1 | 0.60 | 0.110 |
| ENSG00000110768 | GTF2H1 | 0.73 | 0.110 |
| ENSG00000070413 | DGCR2 | −0.63 | 0.111 |
| ENSG00000033050 | ABCF2 | −0.91 | 0.111 |
| ENSG00000111667 | USP5 | −0.72 | 0.111 |
| ENSG00000163703 | CRELD1 | 0.60 | 0.112 |
| ENSG00000138031 | ADCY3 | −0.77 | 0.113 |
| ENSG00000078747 | ITCH | −0.63 | 0.113 |
| ENSG00000160221 | C21orf33 | 0.60 | 0.113 |
| ENSG00000197386 | HTT | 0.46 | 0.113 |
| ENSG00000085719 | CPNE3 | −0.66 | 0.114 |
| ENSG00000185909 | KLHDC8B | 0.78 | 0.115 |
| ENSG00000015133 | CCDC88C | −0.47 | 0.115 |
| ENSG00000184319 | RPL23AP82 | 0.80 | 0.115 |
| ENSG00000090905 | TNRC6A | 0.57 | 0.116 |
| ENSG00000165169 | DYNLT3 | 0.61 | 0.116 |
| ENSG00000102908 | NFAT5 | −0.52 | 0.116 |
| ENSG00000145685 | LHFPL2 | −0.96 | 0.116 |
| ENSG00000108179 | PPIF | 0.51 | 0.117 |
| ENSG00000102226 | USP11 | −0.46 | 0.117 |
| ENSG00000178537 | SLC25A20 | −0.69 | 0.117 |
| ENSG00000109685 | WHSC1 | 0.66 | 0.118 |
| ENSG00000112159 | MDN1 | −0.79 | 0.118 |
| ENSG00000165119 | HNRNPK | −0.41 | 0.119 |
| ENSG00000054523 | KIF1B | −0.78 | 0.119 |
| ENSG00000107262 | BAG1 | 0.38 | 0.120 |
| ENSG00000034053 | APBA2 | −0.96 | 0.120 |
| ENSG00000080189 | SLC35C2 | 0.50 | 0.120 |
| ENSG00000143033 | MTF2 | −0.63 | 0.121 |
| ENSG00000053900 | ANAPC4 | −0.84 | 0.121 |
| ENSG00000130706 | ADRM1 | 0.42 | 0.121 |
| ENSG00000172046 | USP19 | −0.72 | 0.121 |
| ENSG00000133302 | ANKRD32 | −0.45 | 0.122 |
| ENSG00000100997 | ABHD12 | 0.57 | 0.122 |
| ENSG00000139168 | ZCRB1 | −0.46 | 0.123 |
| ENSG00000136527 | TRA2B | 0.37 | 0.123 |
| ENSG00000067208 | EVI5 | 0.79 | 0.123 |
| ENSG00000239839 | DEFA3 | 1.07 | 0.123 |
| ENSG00000140264 | SERF2 | 0.54 | 0.123 |
| ENSG00000226824 | | 0.98 | 0.124 |
| ENSG00000160710 | ADAR | −0.62 | 0.124 |
| ENSG00000117450 | PRDX1 | −0.51 | 0.124 |
| ENSG00000164975 | SNAPC3 | −0.74 | 0.124 |
| ENSG00000147874 | HAUS6 | −0.73 | 0.125 |
| ENSG00000106245 | BUD31 | 0.44 | 0.125 |
| ENSG00000130935 | NOL11 | 0.85 | 0.125 |
| ENSG00000008018 | PSMB1 | −0.51 | 0.125 |
| ENSG00000124491 | F13A1 | −0.67 | 0.126 |
| ENSG00000136732 | GYPC | 0.70 | 0.126 |
| ENSG00000107959 | PITRM1 | −0.61 | 0.127 |
| ENSG00000198833 | UBE2J1 | −0.54 | 0.127 |
| ENSG00000135387 | CAPRIN1 | 0.69 | 0.128 |
| ENSG00000136381 | IREB2 | 1.01 | 0.128 |
| ENSG00000111796 | KLRB1 | −0.52 | 0.128 |
| ENSG00000175582 | RAB6A | −0.58 | 0.128 |
| ENSG00000006712 | PAF1 | −0.57 | 0.129 |
| ENSG00000137804 | NUSAP1 | 0.78 | 0.129 |
| ENSG00000140368 | PSTPIP1 | −0.64 | 0.129 |
| ENSG00000131652 | THOC6 | −0.73 | 0.129 |
| ENSG00000132970 | WASF3 | −0.64 | 0.130 |
| ENSG00000079277 | MKNK1 | 0.55 | 0.132 |
| ENSG00000047249 | ATP6V1H | −0.64 | 0.132 |
| ENSG00000119314 | PTBP3 | −0.69 | 0.133 |
| ENSG00000066027 | PPP2R5A | −0.40 | 0.134 |
| ENSG00000106605 | BLVRA | −0.70 | 0.134 |
| ENSG00000107175 | CREB3 | 0.70 | 0.134 |
| ENSG00000166979 | EVA1C | 0.54 | 0.135 |
| ENSG00000112303 | VNN2 | −0.89 | 0.136 |
| ENSG00000136819 | C9orf78 | 0.45 | 0.136 |
| ENSG00000138663 | COPS4 | 0.51 | 0.137 |
| ENSG00000090487 | SPG21 | −0.43 | 0.137 |
| ENSG00000155508 | CNOT8 | 0.53 | 0.137 |
| ENSG00000167522 | ANKRD11 | −0.34 | 0.137 |
| ENSG00000117528 | ABCD3 | 1.03 | 0.138 |
| ENSG00000003402 | CFLAR | 0.34 | 0.138 |
| ENSG00000166266 | CUL5 | −0.60 | 0.139 |
| ENSG00000175193 | PARL | −0.60 | 0.139 |
| ENSG00000169727 | GPS1 | 0.57 | 0.139 |
| ENSG00000211456 | SACM1L | −0.56 | 0.139 |
| ENSG00000176542 | KIAA2018 | 0.54 | 0.140 |
| ENSG00000135723 | FHOD1 | −0.62 | 0.140 |
| ENSG00000125351 | UPF3B | 0.66 | 0.140 |
| ENSG00000135269 | TES | −0.55 | 0.140 |
| ENSG00000131373 | HACL1 | 0.75 | 0.141 |
| ENSG00000105438 | KDELR1 | −0.47 | 0.141 |
| ENSG00000135913 | USP37 | 0.93 | 0.141 |
| ENSG00000131748 | STARD3 | −0.49 | 0.143 |
| ENSG00000183576 | SETD3 | −0.40 | 0.143 |
| ENSG00000164961 | KIAA0196 | −0.94 | 0.143 |
| ENSG00000151779 | NBAS | −0.37 | 0.143 |
| ENSG00000118507 | AKAP7 | −0.53 | 0.143 |
| ENSG00000136522 | MRPL47 | −0.53 | 0.144 |
| ENSG00000136631 | VPS45 | 0.70 | 0.144 |
| ENSG00000102786 | INTS6 | −0.60 | 0.145 |
| ENSG00000137947 | GTF2B | 0.45 | 0.145 |
| ENSG00000197858 | GPAA1 | −0.41 | 0.145 |
| ENSG00000147535 | PPAPDC1B | 0.48 | 0.145 |
| ENSG00000157601 | MX1 | 0.74 | 0.145 |
| ENSG00000100596 | SPTLC2 | 0.62 | 0.146 |
| ENSG00000170004 | CHD3 | −0.41 | 0.146 |
| ENSG00000153250 | RBMS1 | −0.45 | 0.146 |
| ENSG00000164307 | ERAP1 | −0.77 | 0.146 |
| ENSG00000131725 | WDR44 | −0.57 | 0.146 |
| ENSG00000166128 | RAB8B | −0.47 | 0.147 |
| ENSG00000140694 | PARN | −0.68 | 0.147 |
| ENSG00000170581 | STAT2 | 0.84 | 0.148 |
| ENSG00000104522 | TSTA3 | 0.42 | 0.149 |
| ENSG00000108349 | CASC3 | −0.65 | 0.149 |
| ENSG00000132965 | ALOX5AP | −0.54 | 0.150 |
| ENSG00000156587 | UBE2L6 | −0.44 | 0.150 |
| ENSG00000100100 | PIK3IP1 | 0.87 | 0.151 |
| ENSG00000126934 | MAP2K2 | −0.33 | 0.151 |
| ENSG00000135940 | COX5B | −0.35 | 0.151 |
| ENSG00000178950 | GAK | 0.45 | 0.151 |
| ENSG00000018699 | TTC27 | −0.92 | 0.151 |
| ENSG00000087502 | ERGIC2 | −0.53 | 0.152 |
| ENSG00000143545 | RAB13 | 0.44 | 0.152 |
| ENSG00000103657 | HERC1 | −0.35 | 0.152 |
| ENSG00000074842 | C19orf10 | −0.58 | 0.152 |
| ENSG00000079616 | KIF22 | −0.55 | 0.152 |
| ENSG00000169826 | CSGALNACT2 | 0.67 | 0.152 |
| ENSG00000172661 | FAM21C | 0.48 | 0.153 |
| ENSG00000161638 | ITGA5 | 0.80 | 0.153 |
| ENSG00000134294 | SLC38A2 | −0.81 | 0.153 |
| ENSG00000172572 | PDE3A | −0.95 | 0.153 |
| ENSG00000174442 | ZWILCH | 1.10 | 0.153 |
| ENSG00000106537 | TSPAN13 | −0.73 | 0.153 |
| ENSG00000168785 | TSPAN5 | 0.69 | 0.153 |
| ENSG00000108384 | RAD51C | −0.66 | 0.153 |
| ENSG00000196230 | TUBB | −0.55 | 0.155 |
| ENSG00000101294 | HM13 | −0.65 | 0.155 |
| ENSG00000135624 | CCT7 | −0.43 | 0.155 |
| ENSG00000177030 | DEAF1 | 0.66 | 0.155 |
| ENSG00000110321 | EIF4G2 | −0.46 | 0.155 |
| ENSG00000132300 | PTCD3 | −0.78 | 0.155 |
| ENSG00000114446 | IFT57 | −0.70 | 0.155 |
| ENSG00000102710 | SUPT20H | −0.58 | 0.156 |
| ENSG00000115919 | KYNU | 0.79 | 0.156 |
| ENSG00000138378 | STAT4 | 0.65 | 0.156 |
| ENSG00000152234 | ATP5A1 | −0.38 | 0.156 |
| ENSG00000182923 | CEP63 | −0.52 | 0.156 |
| ENSG00000198130 | HIBCH | −0.67 | 0.156 |
| ENSG00000124302 | CHST8 | −0.72 | 0.157 |
| ENSG00000130734 | ATG4D | 0.58 | 0.157 |
| ENSG00000008952 | SEC62 | 0.29 | 0.157 |
| ENSG00000111906 | HDDC2 | −0.59 | 0.158 |
| ENSG00000176986 | SEC24C | −0.73 | 0.158 |
| ENSG00000160446 | ZDHHC12 | 0.39 | 0.159 |
| ENSG00000198055 | GRK6 | −0.34 | 0.159 |
| ENSG00000142694 | EVA1B | 0.58 | 0.159 |
| ENSG00000144579 | CTDSP1 | 0.46 | 0.159 |
| ENSG00000013306 | SLC25A39 | 0.63 | 0.159 |
| ENSG00000253819 | LINC01151 | 0.46 | 0.160 |
| ENSG00000133193 | FAM104A | 0.45 | 0.160 |
| ENSG00000126698 | DNAJC8 | −0.29 | 0.160 |
| ENSG00000198814 | GK | 0.48 | 0.161 |
| ENSG00000171055 | FEZ2 | 0.45 | 0.161 |
| ENSG00000122986 | HVCN1 | −0.67 | 0.161 |
| ENSG00000185507 | IRF7 | 0.59 | 0.161 |
| ENSG00000204472 | AIF1 | −0.53 | 0.161 |
| ENSG00000149187 | CELF1 | 0.72 | 0.162 |
| ENSG00000013364 | MVP | −0.54 | 0.162 |
| ENSG00000112893 | MAN2A1 | 0.43 | 0.162 |
| ENSG00000162688 | AGL | 0.87 | 0.164 |
| ENSG00000131100 | ATP6V1E1 | 0.36 | 0.164 |
| ENSG00000158552 | ZFAND2B | 0.35 | 0.165 |
| ENSG00000011260 | UTP18 | −0.44 | 0.165 |
| ENSG00000160190 | SLC37A1 | −0.58 | 0.165 |
| ENSG00000164032 | H2AFZ | 0.41 | 0.165 |
| ENSG00000136807 | CDK9 | 0.52 | 0.165 |
| ENSG00000125844 | RRBP1 | −0.60 | 0.166 |
| ENSG00000159023 | EPB41 | 0.42 | 0.166 |
| ENSG00000116678 | LEPR | −0.60 | 0.166 |
| ENSG00000133318 | RTN3 | −0.48 | 0.167 |
| ENSG00000077097 | TOP2B | −0.36 | 0.167 |
| ENSG00000107890 | ANKRD26 | 0.75 | 0.167 |
| ENSG00000197771 | MCMBP | −0.60 | 0.167 |
| ENSG00000129562 | DAD1 | 0.39 | 0.169 |
| ENSG00000116717 | GADD45A | 0.55 | 0.169 |
| ENSG00000125779 | PANK2 | −0.43 | 0.169 |
| ENSG00000211899 | IGHM | −0.51 | 0.169 |
| ENSG00000150316 | CWC15 | −0.48 | 0.169 |
| ENSG00000125970 | RALY | 0.28 | 0.169 |
| ENSG00000175756 | AURKAIP1 | −0.41 | 0.169 |
| ENSG00000180776 | ZDHHC20 | −0.70 | 0.170 |
| ENSG00000125124 | BBS2 | −0.69 | 0.170 |
| ENSG00000033170 | FUT8 | −0.48 | 0.170 |
| ENSG00000134265 | NAPG | 0.54 | 0.170 |
| ENSG00000144381 | HSPD1 | −0.50 | 0.171 |
| ENSG00000137628 | DDX60 | 0.64 | 0.171 |
| ENSG00000088992 | TESC | −0.46 | 0.173 |
| ENSG00000131795 | RBM8A | −0.42 | 0.173 |
| ENSG00000134516 | DOCK2 | −0.45 | 0.173 |
| ENSG00000198301 | SDAD1 | −0.59 | 0.173 |
| ENSG00000116962 | NID1 | −1.01 | 0.173 |
| ENSG00000170471 | RALGAPB | 0.71 | 0.173 |
| ENSG00000164934 | DCAF13 | −0.67 | 0.174 |
| ENSG00000124784 | RIOK1 | −0.68 | 0.174 |
| ENSG00000175471 | MCTP1 | −0.47 | 0.174 |
| ENSG00000167641 | PPP1R14A | 0.47 | 0.174 |
| ENSG00000165732 | DDX21 | 0.42 | 0.175 |
| ENSG00000173692 | PSMD1 | −0.38 | 0.175 |
| ENSG00000101079 | NDRG3 | −0.72 | 0.176 |
| ENSG00000082805 | ERC1 | 0.65 | 0.176 |
| ENSG00000120063 | GNA13 | −0.61 | 0.176 |
| ENSG00000104998 | IL27RA | −0.51 | 0.176 |
| ENSG00000132589 | FLOT2 | 0.41 | 0.176 |
| ENSG00000086666 | ZFAND6 | 0.41 | 0.176 |
| ENSG00000117360 | PRPF3 | 0.59 | 0.176 |
| ENSG00000140455 | USP3 | −0.51 | 0.176 |
| ENSG00000136319 | TTC5 | −0.75 | 0.177 |
| ENSG00000133246 | PRAM1 | −0.62 | 0.177 |
| ENSG00000120129 | DUSP1 | 0.76 | 0.177 |
| ENSG00000197969 | VPS13A | −0.41 | 0.178 |
| ENSG00000100097 | LGALS1 | −0.44 | 0.178 |
| ENSG00000083457 | ITGAE | 0.51 | 0.179 |
| ENSG00000172965 | MIR4435-1HG | 0.35 | 0.179 |
| ENSG00000135605 | TEC | 0.74 | 0.179 |
| ENSG00000108604 | SMARCD2 | −0.48 | 0.179 |
| ENSG00000115966 | ATF2 | −0.57 | 0.180 |
| ENSG00000106028 | SSBP1 | −0.45 | 0.180 |
| ENSG00000030582 | GRN | −0.48 | 0.180 |
| ENSG00000125868 | DSTN | 0.40 | 0.180 |
| ENSG00000116586 | LAMTOR2 | −0.37 | 0.180 |
| ENSG00000118564 | FBXL5 | 0.47 | 0.181 |
| ENSG00000177370 | TIMM22 | 0.52 | 0.181 |
| ENSG00000142794 | NBPF3 | 0.78 | 0.182 |
| ENSG00000101940 | WDR13 | 0.32 | 0.182 |
| ENSG00000117748 | RPA2 | 0.50 | 0.182 |
| ENSG00000139083 | ETV6 | 0.43 | 0.183 |
| ENSG00000023287 | RB1CC1 | 0.44 | 0.183 |
| ENSG00000143641 | GALNT2 | 0.60 | 0.183 |
| ENSG00000102897 | LYRM1 | 0.55 | 0.183 |
| ENSG00000114331 | ACAP2 | −0.37 | 0.183 |
| ENSG00000115875 | SRSF7 | −0.49 | 0.183 |
| ENSG00000092439 | TRPM7 | −0.57 | 0.184 |
| ENSG00000015153 | YAF2 | 0.58 | 0.185 |
| ENSG00000092203 | TOX4 | −0.40 | 0.186 |
| ENSG00000135070 | ISCA1 | 0.50 | 0.186 |
| ENSG00000101158 | NELFCD | 0.41 | 0.186 |
| ENSG00000136490 | LIMD2 | −0.45 | 0.187 |
| ENSG00000138834 | MAPK8IP3 | −0.64 | 0.187 |
| ENSG00000164543 | STK17A | 0.39 | 0.187 |
| ENSG00000068400 | GRIPAP1 | −0.32 | 0.187 |
| ENSG00000140395 | WDR61 | −0.55 | 0.187 |
| ENSG00000166311 | SMPD1 | 0.51 | 0.188 |
| ENSG00000086758 | HUWE1 | −0.42 | 0.188 |
| ENSG00000196591 | HDAC2 | 0.38 | 0.188 |
| ENSG00000114861 | FOXP1 | 0.37 | 0.189 |
| ENSG00000197111 | PCBP2 | 0.30 | 0.189 |
| ENSG00000081087 | OSTM1 | −0.59 | 0.189 |
| ENSG00000080371 | RAB21 | −0.57 | 0.189 |
| ENSG00000116984 | MTR | −0.47 | 0.189 |
| ENSG00000151651 | ADAM8 | −0.60 | 0.189 |
| ENSG00000103051 | COG4 | −0.62 | 0.189 |
| ENSG00000100359 | SGSM3 | −0.38 | 0.189 |
| ENSG00000081377 | CDC14B | −0.54 | 0.190 |
| ENSG00000129925 | TMEM8A | 0.44 | 0.190 |
| ENSG00000106771 | TMEM245 | 0.77 | 0.190 |
| ENSG00000255079 | | −0.57 | 0.191 |
| ENSG00000182551 | ADI1 | −0.42 | 0.191 |
| ENSG00000119203 | CPSF3 | −0.64 | 0.191 |
| ENSG00000078596 | ITM2A | −0.41 | 0.191 |
| ENSG00000074706 | IPCEF1 | 0.52 | 0.192 |
| ENSG00000198356 | ASNA1 | 0.36 | 0.192 |
| ENSG00000117505 | DR1 | −0.47 | 0.192 |
| ENSG00000146918 | NCAPG2 | −0.73 | 0.193 |
| ENSG00000234456 | MAGI2-AS3 | 0.70 | 0.193 |
| ENSG00000163931 | TKT | −0.51 | 0.193 |
| ENSG00000153786 | ZDHHC7 | −0.67 | 0.194 |
| ENSG00000156170 | NDUFAF6 | −0.53 | 0.194 |
| ENSG00000229474 | PATL2 | −0.50 | 0.195 |
| ENSG00000170776 | AKAP13 | −0.37 | 0.195 |
| ENSG00000092847 | AGO1 | −0.83 | 0.195 |
| ENSG00000160703 | NLRX1 | −0.67 | 0.195 |
| ENSG00000101162 | TUBB1 | −0.52 | 0.196 |
| ENSG00000186314 | PRELID2 | 0.69 | 0.196 |
| ENSG00000133106 | EPSTI1 | 0.36 | 0.197 |
| ENSG00000260032 | LINC00657 | 0.52 | 0.197 |
| ENSG00000100365 | NCF4 | −0.49 | 0.197 |
| ENSG00000080815 | PSEN1 | 0.75 | 0.198 |
| ENSG00000167210 | LOXHD1 | −0.61 | 0.198 |
| ENSG00000104946 | TBC1D17 | −0.49 | 0.198 |
| ENSG00000117676 | RPS6KA1 | −0.57 | 0.199 |
| ENSG00000090054 | SPTLC1 | 0.71 | 0.199 |
| ENSG00000074054 | CLASP1 | 0.68 | 0.199 |
| ENSG00000084090 | STARD7 | −0.63 | 0.200 |
| ENSG00000188906 | LRRK2 | 0.75 | 0.200 |
| ENSG00000130826 | DKC1 | −0.61 | 0.200 |
| ENSG00000091640 | SPAG7 | −0.45 | 0.201 |
| ENSG00000163412 | EIF4E3 | −0.57 | 0.201 |
| ENSG00000138073 | PREB | −0.53 | 0.201 |
| ENSG00000138768 | USO1 | −0.50 | 0.202 |
| ENSG00000012223 | LTF | −0.98 | 0.202 |
| ENSG00000136167 | LCP1 | −0.41 | 0.202 |
| ENSG00000138709 | LARP1B | −0.39 | 0.203 |

TABLE 2

| Ensembl_gene_id | hgnc_symbol | logFC | FDR |
|---|---|---|---|
| ENSG00000023191 | RNH1 | 0.73 | 0.000 |
| ENSG00000172757 | CFL1 | 0.79 | 0.000 |
| ENSG00000097021 | ACOT7 | 1.10 | 0.000 |
| ENSG00000147099 | HDAC8 | −1.14 | 0.000 |
| ENSG00000142089 | IFITM3 | 1.61 | 0.000 |
| ENSG00000130429 | ARPC1B | 0.85 | 0.000 |
| ENSG00000156738 | MS4A1 | −2.57 | 0.000 |
| ENSG00000213465 | ARL2 | 1.10 | 0.000 |
| ENSG00000067365 | METTL22 | 1.10 | 0.000 |
| ENSG00000116221 | MRPL37 | 1.06 | 0.000 |
| ENSG00000141068 | KSR1 | −1.66 | 0.000 |
| ENSG00000188191 | PRKAR1B | 1.06 | 0.000 |
| ENSG00000185825 | BCAP31 | 0.65 | 0.000 |
| ENSG00000109854 | HTATIP2 | 1.00 | 0.000 |
| ENSG00000211899 | IGHM | −1.92 | 0.000 |
| ENSG00000074695 | LMAN1 | 0.84 | 0.000 |
| ENSG00000102265 | TIMP1 | 0.87 | 0.000 |
| ENSG00000125868 | DSTN | 0.71 | 0.000 |
| ENSG00000168002 | POLR2G | 0.70 | 0.000 |
| ENSG00000161547 | SRSF2 | 0.88 | 0.000 |
| ENSG00000068308 | OTUD5 | 0.62 | 0.000 |
| ENSG00000126247 | CAPNS1 | 0.75 | 0.000 |
| ENSG00000173812 | EIF1 | 0.73 | 0.000 |
| ENSG00000244734 | HBB | −2.45 | 0.000 |
| ENSG00000136938 | ANP32B | 0.91 | 0.000 |
| ENSG00000075945 | KIFAP3 | 0.84 | 0.000 |
| ENSG00000178057 | NDUFAF3 | 0.74 | 0.000 |
| ENSG00000177556 | ATOX1 | 1.09 | 0.000 |
| ENSG00000099817 | POLR2E | 0.69 | 0.000 |
| ENSG00000019582 | CD74 | −1.28 | 0.000 |
| ENSG00000159335 | PTMS | 0.89 | 0.000 |
| ENSG00000113761 | ZNF346 | 1.59 | 0.000 |
| ENSG00000155366 | RHOC | 0.73 | 0.000 |
| ENSG00000114942 | EEF1B2 | −1.01 | 0.000 |
| ENSG00000198467 | TPM2 | 1.35 | 0.000 |
| ENSG00000105220 | GPI | 0.66 | 0.000 |
| ENSG00000168765 | GSTM4 | 0.82 | 0.000 |
| ENSG00000128245 | YWHAH | 0.65 | 0.000 |
| ENSG00000143149 | ALDH9A1 | 0.66 | 0.000 |
| ENSG00000042753 | AP2S1 | 0.67 | 0.000 |
| ENSG00000187109 | NAP1L1 | 0.70 | 0.000 |
| ENSG00000083845 | RPS5 | −1.26 | 0.000 |
| ENSG00000100418 | DESI1 | 0.87 | 0.000 |
| ENSG00000173083 | HPSE | 1.23 | 0.000 |
| ENSG00000143198 | MGST3 | 0.64 | 0.000 |
| ENSG00000069535 | MAOB | 1.47 | 0.000 |
| ENSG00000104324 | CPQ | 0.72 | 0.000 |
| ENSG00000166091 | CMTM5 | 0.75 | 0.000 |
| ENSG00000184009 | ACTG1 | 0.53 | 0.000 |
| ENSG00000185909 | KLHDC8B | 1.15 | 0.000 |
| ENSG00000136003 | ISCU | 0.64 | 0.000 |
| ENSG00000105401 | CDC37 | 0.58 | 0.000 |
| ENSG00000108654 | DDX5 | −1.06 | 0.000 |
| ENSG00000106211 | HSPB1 | 0.88 | 0.000 |
| ENSG00000086506 | HBQ1 | 0.99 | 0.000 |
| ENSG00000105887 | MTPN | 0.65 | 0.000 |
| ENSG00000140416 | TPM1 | 0.63 | 0.000 |
| ENSG00000204287 | HLA-DRA | −1.38 | 0.000 |
| ENSG00000160446 | ZDHHC12 | 0.61 | 0.000 |
| ENSG00000126432 | PRDX5 | 0.53 | 0.000 |
| ENSG00000005961 | ITGA2B | 0.70 | 0.000 |
| ENSG00000105711 | SCN1B | 0.73 | 0.000 |
| ENSG00000206549 | PRSS50 | 1.80 | 0.000 |
| ENSG00000112335 | SNX3 | 0.49 | 0.000 |
| ENSG00000198168 | SVIP | 0.71 | 0.000 |
| ENSG00000171159 | C9orf16 | 0.68 | 0.000 |
| ENSG00000143226 | FCGR2A | 0.82 | 0.000 |
| ENSG00000106537 | TSPAN13 | 0.91 | 0.000 |
| ENSG00000167100 | SAMD14 | 0.89 | 0.000 |
| ENSG00000111348 | ARHGDIB | 0.47 | 0.000 |
| ENSG00000095585 | BLNK | −1.68 | 0.000 |
| ENSG00000128272 | ATF4 | 0.57 | 0.000 |
| ENSG00000165775 | FUNDC2 | 0.53 | 0.000 |
| ENSG00000106244 | PDAP1 | 0.54 | 0.000 |
| ENSG00000124486 | USP9X | −1.08 | 0.000 |
| ENSG00000135334 | AKIRIN2 | 0.58 | 0.000 |
| ENSG00000233276 | GPX1 | 0.57 | 0.000 |
| ENSG00000167645 | YIF1B | 0.59 | 0.000 |
| ENSG00000102172 | SMS | 0.52 | 0.000 |
| ENSG00000013306 | SLC25A39 | −1.34 | 0.000 |
| ENSG00000163041 | H3F3A | 0.73 | 0.000 |
| ENSG00000162894 | FAIM3 | −1.64 | 0.000 |
| ENSG00000112977 | DAP | 0.68 | 0.000 |
| ENSG00000106565 | TMEM176B | −2.34 | 0.000 |
| ENSG00000053371 | AKR7A2 | 0.86 | 0.000 |
| ENSG00000087237 | CETP | 0.70 | 0.000 |
| ENSG00000163466 | ARPC2 | 0.32 | 0.000 |
| ENSG00000166848 | TERF2IP | 0.46 | 0.000 |
| ENSG00000100906 | NFKBIA | −1.30 | 0.000 |
| ENSG00000101940 | WDR13 | 0.44 | 0.000 |
| ENSG00000176407 | KCMF1 | 0.55 | 0.000 |
| ENSG00000144381 | HSPD1 | −1.00 | 0.000 |
| ENSG00000131401 | NAPSB | −1.53 | 0.000 |
| ENSG00000197858 | GPAA1 | 0.63 | 0.001 |
| ENSG00000160789 | LMNA | 0.48 | 0.001 |
| ENSG00000144560 | VGLL4 | 0.59 | 0.001 |
| ENSG00000147454 | SLC25A37 | −1.35 | 0.001 |
| ENSG00000101439 | CST3 | 0.47 | 0.001 |
| ENSG00000096384 | HSP90AB1 | −0.81 | 0.001 |
| ENSG00000006125 | AP2B1 | 0.54 | 0.001 |
| ENSG00000101460 | MAP1LC3A | 0.86 | 0.001 |
| ENSG00000168374 | ARF4 | 0.47 | 0.001 |
| ENSG00000168918 | INPP5D | −1.31 | 0.001 |
| ENSG00000007312 | CD79B | −1.75 | 0.001 |
| ENSG00000147206 | NXF3 | 1.11 | 0.001 |
| ENSG00000152952 | PLOD2 | 0.61 | 0.001 |
| ENSG00000174915 | PTDSS2 | 0.76 | 0.001 |
| ENSG00000104341 | LAPTM4B | 0.66 | 0.001 |
| ENSG00000196154 | S100A4 | −0.97 | 0.001 |
| ENSG00000145088 | EAF2 | −0.75 | 0.001 |
| ENSG00000126267 | COX6B1 | 0.46 | 0.001 |
| ENSG00000198034 | RPS4X | −0.76 | 0.001 |
| ENSG00000143761 | ARF1 | 0.41 | 0.001 |
| ENSG00000105193 | RPS16 | −0.85 | 0.001 |
| ENSG00000249684 | | 0.87 | 0.001 |
| ENSG00000158062 | UBXN11 | 0.61 | 0.001 |
| ENSG00000107341 | UBE2R2 | 0.47 | 0.001 |
| ENSG00000116288 | PARK7 | 0.48 | 0.001 |
| ENSG00000021776 | AQR | −1.31 | 0.001 |
| ENSG00000064601 | CTSA | 0.46 | 0.001 |
| ENSG00000161911 | TREML1 | 0.48 | 0.001 |
| ENSG00000188404 | SELL | −1.09 | 0.002 |
| ENSG00000111640 | GAPDH | 0.40 | 0.002 |
| ENSG00000180596 | HIST1H2BC | 0.60 | 0.002 |
| ENSG00000130592 | LSP1 | −0.80 | 0.002 |
| ENSG00000198668 | CALM1 | 0.42 | 0.002 |
| ENSG00000244509 | APOBEC3C | 0.39 | 0.002 |
| ENSG00000113732 | ATP6V0E1 | 0.40 | 0.002 |
| ENSG00000196531 | NACA | −0.61 | 0.002 |
| ENSG00000149476 | DAK | 0.67 | 0.002 |
| ENSG00000214941 | ZSWIM7 | 0.48 | 0.002 |
| ENSG00000119705 | SLIRP | −1.10 | 0.002 |
| ENSG00000255002 | | 1.36 | 0.002 |
| ENSG00000125354 | SEPT6 | 0.37 | 0.002 |
| ENSG00000169100 | SLC25A6 | −0.67 | 0.002 |
| ENSG00000166311 | SMPD1 | 0.83 | 0.002 |
| ENSG00000117691 | NENF | 0.51 | 0.002 |
| ENSG00000112799 | LY86 | −1.01 | 0.003 |
| ENSG00000146247 | PHIP | −0.48 | 0.003 |
| ENSG00000142168 | SOD1 | 0.37 | 0.003 |
| ENSG00000117118 | SDHB | −0.90 | 0.003 |
| ENSG00000084207 | GSTP1 | −0.60 | 0.003 |
| ENSG00000178980 | SEPW1 | 0.44 | 0.003 |
| ENSG00000148346 | LCN2 | 0.59 | 0.003 |
| ENSG00000107816 | LZTS2 | 0.63 | 0.003 |
| ENSG00000102901 | CENPT | 0.40 | 0.003 |
| ENSG00000172795 | DCP2 | 0.58 | 0.003 |
| ENSG00000206503 | HLA-A | 0.64 | 0.003 |
| ENSG00000167674 | | 0.64 | 0.003 |
| ENSG00000067082 | KLF6 | −0.61 | 0.003 |
| ENSG00000160991 | ORAI2 | 0.49 | 0.003 |
| ENSG00000204308 | RNF5 | 0.39 | 0.004 |
| ENSG00000125870 | SNRPB2 | −0.67 | 0.004 |
| ENSG00000159363 | ATP13A2 | 0.70 | 0.004 |
| ENSG00000169057 | MECP2 | 0.49 | 0.004 |
| ENSG00000145979 | TBC1D7 | 0.75 | 0.004 |
| ENSG00000109971 | HSPA8 | −0.66 | 0.004 |
| ENSG00000140968 | IRF8 | −1.23 | 0.004 |
| ENSG00000073009 | IKBKG | 0.47 | 0.004 |
| ENSG00000211895 | IGHA1 | −1.25 | 0.004 |
| ENSG00000102898 | NUTF2 | 0.42 | 0.004 |
| ENSG00000140854 | KATNB1 | 0.45 | 0.004 |
| ENSG00000136490 | LIMD2 | −0.72 | 0.004 |
| ENSG00000128731 | HERC2 | 0.58 | 0.005 |
| ENSG00000167460 | TPM4 | 0.40 | 0.005 |
| ENSG00000180879 | SSR4 | 0.40 | 0.005 |
| ENSG00000134297 | PLEKHA8P1 | 0.51 | 0.005 |
| ENSG00000213445 | SIPA1 | −0.72 | 0.005 |
| ENSG00000118508 | RAB32 | 0.38 | 0.005 |
| ENSG00000100280 | AP1B1 | 0.63 | 0.005 |
| ENSG00000145287 | PLAC8 | −0.78 | 0.005 |
| ENSG00000147526 | TACC1 | 0.40 | 0.005 |
| ENSG00000123908 | AGO2 | 0.83 | 0.005 |
| ENSG00000105677 | TMEM147 | −1.00 | 0.005 |
| ENSG00000100453 | GZMB | −1.07 | 0.005 |
| ENSG00000100353 | EIF3D | −0.69 | 0.005 |
| ENSG00000149100 | EIF3M | −0.73 | 0.006 |
| ENSG00000152795 | HNRNPDL | −0.85 | 0.006 |
| ENSG00000102781 | KATNAL1 | 0.86 | 0.006 |
| ENSG00000138376 | BARD1 | 0.63 | 0.006 |
| ENSG00000182087 | TMEM259 | −0.77 | 0.006 |
| ENSG00000141030 | COPS3 | 0.39 | 0.006 |
| ENSG00000101412 | E2F1 | 0.84 | 0.006 |
| ENSG00000156639 | ZFAND3 | 0.57 | 0.006 |
| ENSG00000143110 | C1orf162 | −0.89 | 0.006 |
| ENSG00000196405 | EVL | 0.41 | 0.006 |
| ENSG00000236875 | DDX11L5 | −0.69 | 0.007 |
| ENSG00000126457 | PRMT1 | −0.63 | 0.007 |
| ENSG00000084093 | REST | −0.35 | 0.007 |
| ENSG00000134440 | NARS | −0.69 | 0.007 |
| ENSG00000125107 | CNOT1 | −0.76 | 0.007 |
| ENSG00000156256 | USP16 | −0.78 | 0.007 |
| ENSG00000171700 | RGS19 | −0.81 | 0.007 |
| ENSG00000163344 | PMVK | 0.52 | 0.007 |
| ENSG00000149925 | ALDOA | 0.42 | 0.007 |
| ENSG00000142634 | EFHD2 | −0.79 | 0.007 |
| ENSG00000129083 | COPB1 | −0.69 | 0.007 |
| ENSG00000181704 | YIPF6 | 0.45 | 0.007 |
| ENSG00000070182 | SPTB | 0.54 | 0.008 |
| ENSG00000089327 | FXYD5 | 0.37 | 0.008 |
| ENSG00000091140 | DLD | −0.93 | 0.008 |
| ENSG00000114383 | TUSC2 | 0.47 | 0.008 |
| ENSG00000163479 | SSR2 | −0.77 | 0.008 |
| ENSG00000107099 | DOCK8 | −0.80 | 0.008 |
| ENSG00000127084 | FGD3 | −0.66 | 0.008 |
| ENSG00000133030 | MPRIP | 0.58 | 0.008 |
| ENSG00000089693 | MLF2 | −0.64 | 0.008 |
| ENSG00000158019 | BRE | 0.37 | 0.008 |
| ENSG00000163110 | PDLIM5 | 0.46 | 0.008 |
| ENSG00000154146 | NRGN | 0.49 | 0.008 |
| ENSG00000123338 | NCKAP1L | −1.06 | 0.009 |
| ENSG00000131795 | RBM8A | −0.55 | 0.009 |
| ENSG00000104805 | NUCB1 | 0.43 | 0.009 |
| ENSG00000069493 | CLEC2D | −1.13 | 0.009 |
| ENSG00000125347 | IRF1 | −0.94 | 0.009 |
| ENSG00000172057 | ORMDL3 | 0.47 | 0.009 |
| ENSG00000166452 | AKIP1 | 0.43 | 0.009 |
| ENSG00000072778 | ACADVL | 0.37 | 0.009 |
| ENSG00000125503 | PPP1R12C | 0.54 | 0.009 |
| ENSG00000196565 | HBG2 | 1.49 | 0.009 |
| ENSG00000198791 | CNOT7 | −0.52 | 0.009 |
| ENSG00000247774 | PCED1B-AS1 | −0.92 | 0.010 |
| ENSG00000044574 | HSPA5 | −1.11 | 0.010 |
| ENSG00000104522 | TSTA3 | 0.59 | 0.010 |
| ENSG00000110852 | CLEC2B | −1.02 | 0.010 |
| ENSG00000088726 | TMEM40 | 0.39 | 0.010 |
| ENSG00000103769 | RAB11A | 0.42 | 0.010 |
| ENSG00000149806 | FAU | −0.48 | 0.010 |
| ENSG00000138468 | SENP7 | −0.60 | 0.010 |
| ENSG00000077984 | CST7 | 0.88 | 0.010 |
| ENSG00000154518 | ATP5G3 | −0.65 | 0.010 |
| ENSG00000162819 | BROX | 0.57 | 0.010 |
| ENSG00000220804 | | 0.51 | 0.011 |
| ENSG00000165494 | PCF11 | −0.96 | 0.011 |
| ENSG00000143549 | TPM3 | 0.29 | 0.011 |
| ENSG00000103148 | NPRL3 | 0.52 | 0.011 |
| ENSG00000130726 | TRIM28 | −0.52 | 0.011 |
| ENSG00000196954 | CASP4 | 0.37 | 0.011 |
| ENSG00000185507 | IRF7 | −0.86 | 0.011 |
| ENSG00000113460 | BRIX1 | −1.08 | 0.011 |
| ENSG00000140931 | CMTM3 | 0.36 | 0.011 |
| ENSG00000127527 | EPS15L1 | 0.37 | 0.011 |
| ENSG00000196396 | PTPN1 | 0.47 | 0.011 |
| ENSG00000196611 | MMP1 | 0.90 | 0.011 |
| ENSG00000173221 | GLRX | −0.90 | 0.012 |
| ENSG00000111581 | NUP107 | −0.93 | 0.012 |
| ENSG00000156110 | ADK | −0.89 | 0.012 |
| ENSG00000140022 | STON2 | 0.42 | 0.012 |
| ENSG00000159753 | RLTPR | −1.36 | 0.012 |
| ENSG00000132965 | ALOX5AP | −1.06 | 0.012 |
| ENSG00000175221 | MED16 | 0.52 | 0.012 |
| ENSG00000185624 | P4HB | 0.40 | 0.012 |
| ENSG00000152127 | MGAT5 | 0.75 | 0.012 |
| ENSG00000129292 | PHF20L1 | 0.32 | 0.012 |
| ENSG00000172322 | CLEC12A | −1.45 | 0.012 |
| ENSG00000117289 | TXNIP | −0.58 | 0.012 |
| ENSG00000106635 | BCL7B | 0.34 | 0.012 |
| ENSG00000163359 | COL6A3 | 0.83 | 0.012 |
| ENSG00000169230 | PRELID1 | −0.67 | 0.013 |
| ENSG00000102879 | CORO1A | −0.57 | 0.013 |
| ENSG00000013441 | CLK1 | −0.77 | 0.013 |
| ENSG00000100129 | EIF3L | −0.65 | 0.013 |
| ENSG00000167996 | FTH1 | 0.49 | 0.013 |
| ENSG00000156304 | SCAF4 | −0.77 | 0.013 |
| ENSG00000104903 | LYL1 | 0.38 | 0.013 |
| ENSG00000147162 | OGT | −1.28 | 0.014 |
| ENSG00000137710 | RDX | 0.46 | 0.014 |
| ENSG00000011198 | ABHD5 | 0.50 | 0.014 |
| ENSG00000107438 | PDLIM1 | 0.34 | 0.014 |
| ENSG00000214026 | MRPL23 | −0.70 | 0.014 |
| ENSG00000134516 | DOCK2 | −0.95 | 0.014 |
| ENSG00000169718 | DUS1L | −0.79 | 0.014 |
| ENSG00000107021 | TBC1D13 | 0.53 | 0.014 |
| ENSG00000130638 | ATXN10 | −0.48 | 0.015 |
| ENSG00000075142 | SRI | −0.72 | 0.015 |
| ENSG00000129968 | ABHD17A | 0.33 | 0.015 |
| ENSG00000130303 | BST2 | −0.90 | 0.015 |
| ENSG00000134480 | CCNH | −0.60 | 0.015 |
| ENSG00000233093 | LINC00892 | 0.56 | 0.015 |
| ENSG00000163660 | CCNL1 | −1.04 | 0.015 |
| ENSG00000003436 | TFPI | 0.42 | 0.015 |
| ENSG00000114784 | EIF1B | 0.37 | 0.015 |
| ENSG00000204482 | LST1 | −0.64 | 0.015 |
| ENSG00000167110 | GOLGA2 | 0.37 | 0.015 |
| ENSG00000197766 | CFD | −1.37 | 0.015 |
| ENSG00000160255 | ITGB2 | −0.97 | 0.015 |
| ENSG00000143977 | SNRPG | −0.69 | 0.016 |
| ENSG00000117091 | CD48 | −0.88 | 0.016 |
| ENSG00000141027 | NCOR1 | 0.28 | 0.016 |
| ENSG00000087206 | UIMC1 | 0.30 | 0.016 |
| ENSG00000163382 | APOA1BP | −0.86 | 0.016 |
| ENSG00000163930 | BAP1 | 0.51 | 0.016 |
| ENSG00000101161 | PRPF6 | −0.68 | 0.016 |
| ENSG00000147471 | PROSC | −0.67 | 0.016 |
| ENSG00000139278 | GLIPR1 | −1.28 | 0.016 |
| ENSG00000034152 | MAP2K3 | 0.35 | 0.016 |
| ENSG00000158856 | DMTN | 0.53 | 0.017 |
| ENSG00000113328 | CCNG1 | 0.37 | 0.017 |
| ENSG00000162191 | UBXN1 | −0.50 | 0.017 |
| ENSG00000136167 | LCP1 | −0.69 | 0.017 |
| ENSG00000057663 | ATG5 | −0.82 | 0.017 |
| ENSG00000034713 | GABARAPL2 | 0.34 | 0.017 |
| ENSG00000176783 | RUFY1 | 0.45 | 0.017 |
| ENSG00000234745 | HLA-B | 0.44 | 0.017 |
| ENSG00000206560 | ANKRD28 | −0.57 | 0.017 |
| ENSG00000132824 | SERINC3 | 0.29 | 0.018 |
| ENSG00000100664 | EIF5 | 0.31 | 0.018 |
| ENSG00000116670 | MAD2L2 | −0.98 | 0.018 |
| ENSG00000134758 | RNF138 | −0.64 | 0.018 |
| ENSG00000154473 | BUB3 | −0.53 | 0.018 |
| ENSG00000104915 | STX10 | −0.53 | 0.018 |
| ENSG00000164754 | RAD21 | 0.41 | 0.018 |
| ENSG00000141401 | IMPA2 | 0.72 | 0.018 |
| ENSG00000084463 | WBP11 | −0.40 | 0.018 |
| ENSG00000248636 | | 0.49 | 0.018 |
| ENSG00000272168 | CASC15 | 0.52 | 0.019 |
| ENSG00000113384 | GOLPH3 | −0.50 | 0.019 |
| ENSG00000158710 | TAGLN2 | 0.35 | 0.019 |
| ENSG00000235162 | C12orf75 | 0.44 | 0.019 |
| ENSG00000143621 | ILF2 | −0.78 | 0.019 |
| ENSG00000178105 | DDX10 | −0.67 | 0.019 |
| ENSG00000163875 | MEAF6 | −0.46 | 0.019 |
| ENSG00000074800 | ENO1 | 0.27 | 0.019 |
| ENSG00000197956 | S100A6 | −0.66 | 0.019 |
| ENSG00000089009 | RPL6 | −0.57 | 0.019 |
| ENSG00000140307 | GTF2A2 | 0.50 | 0.019 |
| ENSG00000161999 | JMJD8 | 0.51 | 0.019 |
| ENSG00000198821 | CD247 | −0.95 | 0.019 |
| ENSG00000198160 | MIER1 | 0.44 | 0.019 |
| ENSG00000154001 | PPP2R5E | −0.62 | 0.019 |
| ENSG00000005022 | SLC25A5 | −0.38 | 0.020 |
| ENSG00000157045 | NTAN1 | 0.33 | 0.020 |
| ENSG00000184014 | DENND5A | −0.95 | 0.020 |
| ENSG00000143933 | CALM2 | 0.29 | 0.020 |
| ENSG00000055163 | CYFIP2 | −0.68 | 0.020 |
| ENSG00000172183 | ISG20 | −0.70 | 0.020 |
| ENSG00000130024 | PHF10 | −0.70 | 0.020 |
| ENSG00000023902 | PLEKHO1 | 0.34 | 0.020 |
| ENSG00000102145 | GATA1 | 0.39 | 0.020 |
| ENSG00000140386 | SCAPER | −0.78 | 0.020 |
| ENSG00000180353 | HCLS1 | −0.59 | 0.020 |
| ENSG00000143924 | EML4 | −0.70 | 0.020 |
| ENSG00000144677 | CTDSPL | 0.33 | 0.020 |
| ENSG00000110077 | MS4A6A | −1.31 | 0.020 |
| ENSG00000113269 | RNF130 | −0.26 | 0.020 |
| ENSG00000144283 | PKP4 | 0.61 | 0.021 |
| ENSG00000026508 | CD44 | −0.74 | 0.021 |
| ENSG00000176986 | SEC24C | −1.07 | 0.021 |
| ENSG00000163931 | TKT | −0.67 | 0.021 |
| ENSG00000085117 | CD82 | 0.36 | 0.021 |
| ENSG00000189403 | HMGB1 | 0.33 | 0.021 |
| ENSG00000124383 | MPHOSPH10 | −0.90 | 0.021 |
| ENSG00000132334 | PTPRE | −0.94 | 0.021 |
| ENSG00000150316 | CWC15 | −0.57 | 0.021 |
| ENSG00000101236 | RNF24 | 0.33 | 0.022 |
| ENSG00000072818 | ACAP1 | −0.82 | 0.022 |
| ENSG00000147274 | RBMX | −0.60 | 0.022 |
| ENSG00000178982 | EIF3K | −0.54 | 0.022 |
| ENSG00000065809 | FAM107B | −0.32 | 0.023 |
| ENSG00000134444 | KIAA1468 | −0.82 | 0.023 |
| ENSG00000143537 | ADAM15 | −1.41 | 0.023 |
| ENSG00000196914 | ARHGEF12 | 0.44 | 0.023 |
| ENSG00000101361 | NOP56 | −0.80 | 0.023 |
| ENSG00000167699 | GLOD4 | −0.74 | 0.023 |
| ENSG00000105926 | MPP6 | 0.50 | 0.023 |
| ENSG00000168028 | RPSA | −0.55 | 0.024 |
| ENSG00000119004 | CYP20A1 | −0.81 | 0.024 |
| ENSG00000124784 | RIOK1 | −1.15 | 0.024 |
| ENSG00000144028 | SNRNP200 | −0.77 | 0.024 |
| ENSG00000162231 | NXF1 | −1.02 | 0.024 |
| ENSG00000107551 | RASSF4 | −1.02 | 0.024 |
| ENSG00000148180 | GSN | 0.33 | 0.024 |
| ENSG00000150867 | PIP4K2A | 0.31 | 0.025 |
| ENSG00000132153 | DHX30 | −0.87 | 0.025 |
| ENSG00000172922 | RNASEH2C | 0.50 | 0.025 |
| ENSG00000027075 | PRKCH | −0.94 | 0.025 |
| ENSG00000115875 | SRSF7 | −0.71 | 0.025 |
| ENSG00000142694 | EVA1B | 0.56 | 0.025 |
| ENSG00000090382 | LYZ | −1.13 | 0.025 |
| ENSG00000112367 | FIG4 | −1.03 | 0.026 |
| ENSG00000182866 | LCK | −0.79 | 0.026 |
| ENSG00000164163 | ABCE1 | −0.99 | 0.026 |
| ENSG00000187097 | ENTPD5 | 0.63 | 0.026 |
| ENSG00000108798 | ABI3 | −0.63 | 0.026 |
| ENSG00000247627 | MTND4P12 | 0.67 | 0.026 |
| ENSG00000105355 | PLIN3 | 0.29 | 0.026 |
| ENSG00000118260 | CREB1 | −0.52 | 0.026 |
| ENSG00000127920 | GNG11 | 0.36 | 0.027 |
| ENSG00000178741 | COX5A | −0.57 | 0.027 |
| ENSG00000203747 | FCGR3A | −0.87 | 0.027 |
| ENSG00000197070 | ARRDC1 | −0.52 | 0.027 |
| ENSG00000111666 | CHPT1 | −0.81 | 0.027 |
| ENSG00000079257 | LXN | 0.64 | 0.028 |
| ENSG00000127252 | HRASLS | 0.42 | 0.028 |
| ENSG00000168904 | LRRC28 | 0.37 | 0.028 |
| ENSG00000197601 | FAR1 | −0.72 | 0.028 |
| ENSG00000163191 | S100A11 | −0.63 | 0.028 |
| ENSG00000121680 | PEX16 | −0.85 | 0.028 |
| ENSG00000139644 | TMBIM6 | 0.21 | 0.029 |
| ENSG00000052841 | TTC17 | −0.93 | 0.029 |
| ENSG00000163516 | ANKZF1 | −0.98 | 0.029 |
| ENSG00000151092 | NGLY1 | −0.74 | 0.029 |
| ENSG00000164022 | AIMP1 | −0.56 | 0.029 |
| ENSG00000120656 | TAF12 | −0.54 | 0.029 |
| ENSG00000206341 | HLA-H | 0.71 | 0.030 |
| ENSG00000119535 | CSF3R | −1.18 | 0.030 |
| ENSG00000011638 | TMEM159 | 0.50 | 0.030 |
| ENSG00000140612 | SEC11A | −0.48 | 0.030 |
| ENSG00000183137 | CEP57L1 | 0.53 | 0.030 |
| ENSG00000244038 | DDOST | −0.57 | 0.030 |
| ENSG00000115652 | UXS1 | 0.34 | 0.030 |
| ENSG00000198837 | DENND4B | −1.02 | 0.030 |
| ENSG00000102096 | PIM2 | −0.85 | 0.031 |
| ENSG00000158417 | EIF5B | −0.53 | 0.031 |
| ENSG00000114902 | SPCS1 | 0.33 | 0.031 |
| ENSG00000091164 | TXNL1 | 0.26 | 0.031 |
| ENSG00000100351 | GRAP2 | 0.30 | 0.031 |
| ENSG00000110799 | VWF | 0.45 | 0.031 |
| ENSG00000177575 | CD163 | −1.44 | 0.031 |
| ENSG00000156171 | DRAM2 | −0.64 | 0.032 |
| ENSG00000136104 | RNASEH2B | −0.61 | 0.032 |
| ENSG00000145649 | GZMA | −0.93 | 0.032 |
| ENSG00000116871 | MAP7D1 | −0.51 | 0.033 |
| ENSG00000124126 | PREX1 | −0.81 | 0.033 |
| ENSG00000122882 | ECD | −0.78 | 0.033 |
| ENSG00000184634 | MED12 | −0.62 | 0.033 |
| ENSG00000101605 | MYOM1 | 0.47 | 0.034 |
| ENSG00000168256 | NKIRAS2 | 0.39 | 0.034 |
| ENSG00000176390 | CRLF3 | −0.59 | 0.034 |
| ENSG00000239900 | ADSL | −0.73 | 0.034 |
| ENSG00000139436 | GIT2 | −0.52 | 0.035 |
| ENSG00000140575 | IQGAP1 | −0.81 | 0.035 |
| ENSG00000051620 | HEBP2 | −0.65 | 0.035 |
| ENSG00000134602 | | −0.37 | 0.035 |
| ENSG00000227165 | WDR11-AS1 | −0.41 | 0.035 |
| ENSG00000135905 | DOCK10 | −0.63 | 0.035 |
| ENSG00000110719 | TCIRG1 | −0.91 | 0.035 |
| ENSG00000138430 | OLA1 | −0.65 | 0.035 |
| ENSG00000164961 | KIAA0196 | −1.09 | 0.036 |
| ENSG00000155465 | SLC7A7 | −1.00 | 0.036 |
| ENSG00000205302 | SNX2 | −0.41 | 0.036 |
| ENSG00000023734 | STRAP | 0.31 | 0.036 |
| ENSG00000163956 | LRPAP1 | −0.68 | 0.036 |
| ENSG00000142192 | APP | 0.45 | 0.036 |
| ENSG00000064102 | ASUN | −0.99 | 0.036 |
| ENSG00000240065 | PSMB9 | −0.29 | 0.037 |
| ENSG00000147548 | WHSC1L1 | −0.31 | 0.037 |
| ENSG00000107077 | KDM4C | −0.89 | 0.037 |
| ENSG00000121774 | KHDRBS1 | −0.44 | 0.038 |
| ENSG00000100612 | DHRS7 | −0.50 | 0.038 |
| ENSG00000266714 | MYO15B | −0.95 | 0.038 |
| ENSG00000095564 | BTAF1 | −0.97 | 0.038 |
| ENSG00000188313 | PLSCR1 | −0.69 | 0.038 |
| ENSG00000126759 | CFP | −1.06 | 0.039 |
| ENSG00000155561 | NUP205 | −0.95 | 0.039 |
| ENSG00000020922 | MRE11A | −0.78 | 0.039 |
| ENSG00000070831 | CDC42 | 0.30 | 0.039 |
| ENSG00000151665 | PIGF | 0.38 | 0.039 |
| ENSG00000006114 | SYNRG | −0.63 | 0.040 |
| ENSG00000077232 | DNAJC10 | −0.94 | 0.040 |
| ENSG00000144895 | EIF2A | −0.70 | 0.040 |
| ENSG00000108270 | AATF | −0.60 | 0.040 |
| ENSG00000164896 | FASTK | −0.62 | 0.040 |
| ENSG00000115233 | PSMD14 | −0.81 | 0.040 |
| ENSG00000142227 | EMP3 | 0.31 | 0.040 |
| ENSG00000163513 | TGFBR2 | −0.44 | 0.040 |
| ENSG00000140105 | WARS | −0.63 | 0.040 |
| ENSG00000102699 | PARP4 | −0.67 | 0.040 |
| ENSG00000168894 | RNF181 | 0.39 | 0.041 |
| ENSG00000140740 | UQCRC2 | −0.49 | 0.041 |
| ENSG00000105669 | COPE | 0.22 | 0.041 |
| ENSG00000138964 | PARVG | −0.67 | 0.042 |
| ENSG00000150045 | KLRF1 | −0.84 | 0.042 |
| ENSG00000117868 | ESYT2 | −0.62 | 0.042 |
| ENSG00000055332 | EIF2AK2 | −0.65 | 0.042 |
| ENSG00000145833 | DDX46 | −0.60 | 0.043 |
| ENSG00000008988 | RPS20 | −0.41 | 0.043 |
| ENSG00000068831 | RASGRP2 | 0.23 | 0.043 |
| ENSG00000084070 | SMAP2 | −0.51 | 0.043 |
| ENSG00000129071 | MBD4 | −0.41 | 0.043 |
| ENSG00000107262 | BAG1 | −0.39 | 0.044 |
| ENSG00000138798 | EGF | 0.37 | 0.044 |
| ENSG00000030066 | NUP160 | −1.11 | 0.044 |
| ENSG00000138385 | SSB | −0.46 | 0.044 |
| ENSG00000250878 | METTL21EP | 0.53 | 0.044 |
| ENSG00000125257 | ABCC4 | 0.38 | 0.044 |
| ENSG00000100028 | SNRPD3 | −0.38 | 0.044 |
| ENSG00000111424 | VDR | −0.59 | 0.044 |
| ENSG00000140943 | MBTPS1 | −0.92 | 0.044 |
| ENSG00000108671 | PSMD11 | 0.31 | 0.044 |
| ENSG00000071994 | PDCD2 | −0.77 | 0.044 |
| ENSG00000093072 | CECR1 | −1.22 | 0.045 |
| ENSG00000004534 | RBM6 | −0.71 | 0.045 |
| ENSG00000136286 | MYO1G | −0.64 | 0.045 |
| ENSG00000060688 | SNRNP40 | −0.53 | 0.045 |
| ENSG00000115368 | WDR75 | −1.05 | 0.045 |
| ENSG00000103502 | CDIPT | 0.31 | 0.045 |
| ENSG00000112245 | PTP4A1 | −0.38 | 0.045 |
| ENSG00000119950 | MXI1 | −0.62 | 0.046 |
| ENSG00000147601 | TERF1 | −0.57 | 0.046 |
| ENSG00000113558 | SKP1 | 0.27 | 0.046 |
| ENSG00000100242 | SUN2 | −0.98 | 0.046 |
| ENSG00000014123 | UFL1 | −0.86 | 0.046 |
| ENSG00000123146 | CD97 | −0.55 | 0.046 |
| ENSG00000065978 | YBX1 | 0.33 | 0.046 |
| ENSG00000089022 | MAPKAPK5 | −0.92 | 0.046 |
| ENSG00000172992 | DCAKD | 0.37 | 0.046 |
| ENSG00000133789 | SWAP70 | −0.70 | 0.046 |
| ENSG00000205133 | TRIQK | 0.40 | 0.046 |
| ENSG00000161638 | ITGA5 | −0.82 | 0.047 |
| ENSG00000092108 | SCFD1 | −0.35 | 0.047 |
| ENSG00000135269 | TES | −0.75 | 0.047 |
| ENSG00000104517 | UBR5 | −0.48 | 0.047 |
| ENSG00000265148 | BZRAP1-AS1 | 0.32 | 0.047 |
| ENSG00000134308 | YWHAQ | 0.26 | 0.047 |
| ENSG00000067955 | CBFB | −0.68 | 0.047 |
| ENSG00000198301 | SDAD1 | −0.66 | 0.047 |
| ENSG00000165168 | CYBB | −0.68 | 0.048 |
| ENSG00000167895 | TMC8 | −1.06 | 0.048 |
| ENSG00000172164 | SNTB1 | −0.47 | 0.048 |
| ENSG00000130176 | CNN1 | 0.55 | 0.049 |
| ENSG00000166165 | CKB | 0.58 | 0.049 |
| ENSG00000070610 | GBA2 | −0.53 | 0.049 |
| ENSG00000010610 | CD4 | −1.28 | 0.049 |
| ENSG00000076108 | BAZ2A | −0.49 | 0.049 |
| ENSG00000122786 | CALD1 | 0.34 | 0.050 |
| ENSG00000139343 | SNRPF | −0.66 | 0.050 |
| ENSG00000141524 | TMC6 | −0.94 | 0.050 |
| ENSG00000100325 | ASCC2 | −0.93 | 0.050 |
| ENSG00000102908 | NFAT5 | 0.45 | 0.050 |
| ENSG00000151651 | ADAM8 | −1.10 | 0.050 |
| ENSG00000135317 | SNX14 | −0.70 | 0.050 |
| ENSG00000076662 | ICAM3 | −0.69 | 0.051 |
| ENSG00000116754 | SRSF11 | −0.37 | 0.051 |
| ENSG00000078124 | ACER3 | 0.36 | 0.051 |
| ENSG00000147202 | DIAPH2 | −1.02 | 0.051 |
| ENSG00000149499 | EML3 | −0.63 | 0.052 |
| ENSG00000162924 | REL | −0.63 | 0.052 |
| ENSG00000157216 | SSBP3 | 0.39 | 0.052 |
| ENSG00000167261 | DPEP2 | −0.91 | 0.052 |
| ENSG00000162517 | PEF1 | 0.32 | 0.052 |
| ENSG00000171735 | CAMTA1 | 0.32 | 0.053 |
| ENSG00000134333 | LDHA | −0.44 | 0.054 |
| ENSG00000108559 | NUP88 | −0.72 | 0.054 |
| ENSG00000249307 | LINC01088 | −0.59 | 0.054 |
| ENSG00000197872 | FAM49A | −1.09 | 0.054 |
| ENSG00000056097 | ZFR | −0.22 | 0.055 |
| ENSG00000110090 | CPT1A | 0.42 | 0.055 |
| ENSG00000013374 | NUB1 | −0.53 | 0.055 |
| ENSG00000186166 | CCDC84 | −0.85 | 0.055 |
| ENSG00000093167 | LRRFIP2 | 0.29 | 0.055 |
| ENSG00000204592 | HLA-E | 0.26 | 0.055 |
| ENSG00000176170 | SPHK1 | 0.31 | 0.055 |
| ENSG00000138834 | MAPK8IP3 | −0.73 | 0.055 |
| ENSG00000119396 | RAB14 | 0.31 | 0.055 |
| ENSG00000142507 | PSMB6 | −0.47 | 0.055 |
| ENSG00000091592 | NLRP1 | −0.99 | 0.056 |
| ENSG00000130560 | UBAC1 | 0.31 | 0.056 |
| ENSG00000014641 | MDH1 | −0.48 | 0.056 |
| ENSG00000110697 | PITPNM1 | −0.51 | 0.056 |
| ENSG00000174720 | LARP7 | −0.45 | 0.056 |
| ENSG00000168488 | ATXN2L | −0.45 | 0.056 |
| ENSG00000115935 | WIPF1 | 0.27 | 0.056 |
| ENSG00000138378 | STAT4 | −0.97 | 0.056 |
| ENSG00000166012 | TAF1D | −0.47 | 0.056 |
| ENSG00000163219 | ARHGAP25 | −0.59 | 0.056 |
| ENSG00000177868 | CCDC23 | 0.37 | 0.056 |
| ENSG00000000938 | FGR | −0.61 | 0.056 |
| ENSG00000148672 | GLUD1 | −0.55 | 0.056 |
| ENSG00000182473 | EXOC7 | −0.47 | 0.057 |
| ENSG00000176124 | DLEU1 | 0.41 | 0.058 |
| ENSG00000143995 | MEIS1 | −0.36 | 0.058 |
| ENSG00000131473 | ACLY | −0.69 | 0.058 |
| ENSG00000105971 | CAV2 | 0.49 | 0.058 |
| ENSG00000150593 | PDCD4 | −0.36 | 0.059 |
| ENSG00000160948 | VPS28 | 0.26 | 0.059 |
| ENSG00000141543 | EIF4A3 | −0.70 | 0.059 |
| ENSG00000105185 | PDCD5 | −0.69 | 0.059 |
| ENSG00000134186 | PRPF38B | −0.64 | 0.059 |
| ENSG00000239713 | APOBEC3G | −0.59 | 0.059 |
| ENSG00000099337 | KCNK6 | 0.31 | 0.059 |
| ENSG00000178952 | TUFM | −0.58 | 0.059 |
| ENSG00000131781 | FMO5 | 0.41 | 0.059 |
| ENSG00000109046 | WSB1 | −0.65 | 0.059 |
| ENSG00000129518 | EAPP | −0.44 | 0.060 |
| ENSG00000179950 | PUF60 | −0.65 | 0.060 |
| ENSG00000159128 | IFNGR2 | −0.88 | 0.060 |
| ENSG00000121064 | SCPEP1 | −0.86 | 0.061 |
| ENSG00000004700 | RECQL | −0.41 | 0.061 |
| ENSG00000189091 | SF3B3 | −0.70 | 0.061 |
| ENSG00000183486 | MX2 | −0.86 | 0.061 |
| ENSG00000059758 | CDK17 | −0.38 | 0.061 |
| ENSG00000143486 | EIF2D | −0.80 | 0.061 |
| ENSG00000168538 | TRAPPC11 | −0.94 | 0.062 |
| ENSG00000011485 | PPP5C | −0.62 | 0.062 |
| ENSG00000005007 | UPF1 | −0.46 | 0.063 |
| ENSG00000166986 | MARS | −0.64 | 0.063 |
| ENSG00000151422 | FER | 0.51 | 0.063 |
| ENSG00000118873 | RAB3GAP2 | −0.48 | 0.064 |
| ENSG00000065000 | AP3D1 | 0.35 | 0.064 |
| ENSG00000134759 | ELP2 | −0.88 | 0.065 |
| ENSG00000089127 | OAS1 | −0.82 | 0.066 |
| ENSG00000169583 | CLIC3 | −0.69 | 0.067 |
| ENSG00000117139 | KDM5B | −0.67 | 0.067 |
| ENSG00000120129 | DUSP1 | −1.06 | 0.067 |
| ENSG00000164483 | SAMD3 | −0.72 | 0.067 |
| ENSG00000101347 | SAMHD1 | −0.67 | 0.068 |
| ENSG00000163960 | UBXN7 | 0.54 | 0.068 |
| ENSG00000126524 | SBDS | −0.38 | 0.068 |
| ENSG00000124588 | NQO2 | 0.29 | 0.068 |
| ENSG00000123124 | WWP1 | −0.36 | 0.068 |
| ENSG00000041357 | PSMA4 | −0.58 | 0.068 |
| ENSG00000101367 | MAPRE1 | 0.25 | 0.068 |
| ENSG00000109320 | NFKB1 | −0.86 | 0.068 |
| ENSG00000091640 | SPAG7 | −0.49 | 0.068 |
| ENSG00000112308 | C6orf62 | −0.43 | 0.069 |
| ENSG00000155660 | PDIA4 | −0.84 | 0.069 |
| ENSG00000152332 | UHMK1 | 0.44 | 0.069 |
| ENSG00000115828 | QPCT | −1.30 | 0.070 |
| ENSG00000175166 | PSMD2 | 0.23 | 0.070 |
| ENSG00000090487 | SPG21 | −0.42 | 0.070 |
| ENSG00000080608 | KIAA0020 | −0.62 | 0.071 |
| ENSG00000102119 | EMD | 0.32 | 0.071 |
| ENSG00000224805 | LINC00853 | 0.35 | 0.071 |
| ENSG00000135899 | SP110 | −0.51 | 0.071 |
| ENSG00000204420 | C6orf25 | 0.33 | 0.072 |
| ENSG00000162613 | FUBP1 | −0.37 | 0.072 |
| ENSG00000158552 | ZFAND2B | 0.23 | 0.073 |
| ENSG00000204406 | MBD5 | 0.35 | 0.073 |
| ENSG00000112118 | MCM3 | −0.80 | 0.073 |
| ENSG00000163781 | TOPBP1 | −0.62 | 0.073 |
| ENSG00000131876 | SNRPA1 | −0.78 | 0.074 |
| ENSG00000164880 | INTS1 | −0.68 | 0.074 |
| ENSG00000177981 | ASB8 | 0.28 | 0.074 |
| ENSG00000005700 | IBTK | −0.65 | 0.074 |
| ENSG00000136247 | ZDHHC4 | 0.26 | 0.074 |
| ENSG00000151465 | CDC123 | −0.49 | 0.074 |
| ENSG00000089154 | GCN1L1 | −1.03 | 0.074 |
| ENSG00000174788 | PCP2 | 0.35 | 0.075 |
| ENSG00000165233 | C9orf89 | 0.27 | 0.075 |
| ENSG00000065154 | OAT | −0.78 | 0.075 |
| ENSG00000205744 | DENND1C | −0.73 | 0.075 |
| ENSG00000134539 | KLRD1 | −0.78 | 0.075 |
| ENSG00000122257 | RBBP6 | −0.26 | 0.075 |
| ENSG00000196924 | FLNA | 0.36 | 0.075 |
| ENSG00000148229 | POLE3 | 0.32 | 0.075 |
| ENSG00000162645 | GBP2 | −0.57 | 0.075 |
| ENSG00000103275 | UBE2I | 0.30 | 0.076 |
| ENSG00000175826 | CTDNEP1 | 0.31 | 0.076 |
| ENSG00000198382 | UVRAG | −0.60 | 0.076 |
| ENSG00000143321 | HDGF | 0.26 | 0.076 |
| ENSG00000145495 | MARCH6 | 0.25 | 0.076 |
| ENSG00000197102 | DYNC1H1 | −0.52 | 0.076 |
| ENSG00000056586 | RC3H2 | 0.30 | 0.077 |
| ENSG00000025293 | PHF20 | −0.29 | 0.078 |
| ENSG00000167705 | RILP | 0.29 | 0.079 |
| ENSG00000111596 | CNOT2 | −0.40 | 0.079 |
| ENSG00000111726 | CMAS | 0.26 | 0.079 |
| ENSG00000161526 | SAP30BP | −0.51 | 0.079 |
| ENSG00000173542 | MOB1B | 0.34 | 0.079 |
| ENSG00000170860 | LSM3 | −0.36 | 0.081 |
| ENSG00000173598 | NUDT4 | 0.42 | 0.081 |
| ENSG00000110324 | IL10RA | −0.85 | 0.082 |
| ENSG00000196576 | PLXNB2 | −0.77 | 0.082 |
| ENSG00000186470 | BTN3A2 | 0.31 | 0.082 |
| ENSG00000197081 | IGF2R | −0.91 | 0.082 |
| ENSG00000125304 | TM9SF2 | −0.37 | 0.083 |
| ENSG00000110031 | LPXN | −0.60 | 0.083 |
| ENSG00000143207 | RFWD2 | −0.46 | 0.083 |
| ENSG00000134548 | C12orf39 | 0.33 | 0.083 |
| ENSG00000103507 | BCKDK | 0.30 | 0.084 |
| ENSG00000038274 | MAT2B | −0.24 | 0.084 |
| ENSG00000196199 | MPHOSPH8 | −0.55 | 0.085 |
| ENSG00000122565 | CBX3 | −0.21 | 0.085 |
| ENSG00000101365 | IDH3B | −0.48 | 0.086 |
| ENSG00000124214 | STAU1 | 0.20 | 0.086 |
| ENSG00000215039 | CD27-AS1 | 0.28 | 0.087 |
| ENSG00000105717 | PBX4 | 0.39 | 0.087 |
| ENSG00000154582 | TCEB1 | 0.30 | 0.087 |
| ENSG00000177885 | GRB2 | 0.18 | 0.088 |
| ENSG00000107742 | SPOCK2 | −1.03 | 0.088 |
| ENSG00000119707 | RBM25 | −0.46 | 0.088 |
| ENSG00000100412 | ACO2 | −0.48 | 0.088 |
| ENSG00000138767 | CNOT6L | 0.37 | 0.088 |
| ENSG00000113649 | TCERG1 | −0.65 | 0.088 |
| ENSG00000123933 | MXD4 | −0.45 | 0.088 |
| ENSG00000134884 | ARGLU1 | −0.55 | 0.088 |
| ENSG00000116815 | CD58 | −0.53 | 0.089 |
| ENSG00000159840 | ZYX | 0.29 | 0.089 |
| ENSG00000162909 | CAPN2 | 0.29 | 0.089 |
| ENSG00000070540 | WIPI1 | 0.25 | 0.090 |
| ENSG00000198876 | DCAF12 | −0.45 | 0.090 |
| ENSG00000116701 | NCF2 | −0.60 | 0.090 |
| ENSG00000145725 | PPIP5K2 | −0.76 | 0.090 |
| ENSG00000131375 | CAPN7 | −0.69 | 0.090 |
| ENSG00000138795 | LEF1 | −0.80 | 0.090 |
| ENSG00000108846 | ABCC3 | 0.29 | 0.090 |
| ENSG00000185359 | HGS | −0.48 | 0.091 |
| ENSG00000253819 | LINC01151 | 0.35 | 0.091 |
| ENSG00000111716 | LDHB | −0.22 | 0.091 |
| ENSG00000161203 | AP2M1 | 0.24 | 0.091 |
| ENSG00000152256 | PDK1 | 0.36 | 0.091 |
| ENSG00000132906 | CASP9 | 0.40 | 0.092 |
| ENSG00000162777 | DENND2D | −0.36 | 0.092 |
| ENSG00000118680 | MYL12B | 0.21 | 0.092 |
| ENSG00000117676 | RPS6KA1 | −0.70 | 0.093 |
| ENSG00000164808 | SPIDR | −0.72 | 0.093 |
| ENSG00000149311 | ATM | −0.67 | 0.094 |
| ENSG00000023892 | DEF6 | −0.63 | 0.094 |
| ENSG00000185340 | GAS2L1 | 0.31 | 0.094 |
| ENSG00000119596 | YLPM1 | −0.60 | 0.095 |
| ENSG00000125652 | ALKBH7 | −0.44 | 0.096 |
| ENSG00000182173 | TSEN54 | −0.63 | 0.096 |
| ENSG00000141380 | SS18 | −0.41 | 0.097 |
| ENSG00000164329 | PAPD4 | −0.39 | 0.098 |
| ENSG00000151779 | NBAS | 0.35 | 0.098 |
| ENSG00000234810 | | 0.35 | 0.098 |
| ENSG00000197170 | PSMD12 | −0.49 | 0.098 |
| ENSG00000111670 | GNPTAB | −0.41 | 0.098 |
| ENSG00000114861 | FOXP1 | −0.30 | 0.098 |
| ENSG00000154359 | LONRF1 | −0.34 | 0.099 |
| ENSG00000010256 | UQCRC1 | −0.45 | 0.100 |
| ENSG00000158864 | NDUFS2 | −0.46 | 0.100 |
| ENSG00000072422 | RHOBTB1 | 0.26 | 0.101 |
| ENSG00000162598 | C1orf87 | −1.46 | 0.101 |
| ENSG00000166747 | AP1G1 | −0.52 | 0.102 |
| ENSG00000180182 | MED14 | −0.61 | 0.102 |
| ENSG00000161570 | CCL5 | 0.28 | 0.102 |
| ENSG00000111906 | HDDC2 | −0.57 | 0.102 |
| ENSG00000120903 | CHRNA2 | −0.55 | 0.102 |
| ENSG00000130177 | CDC16 | 0.27 | 0.103 |
| ENSG00000113712 | CSNK1A1 | 0.18 | 0.103 |
| ENSG00000263563 | UBBP4 | 0.28 | 0.104 |
| ENSG00000170638 | TRABD | −0.70 | 0.104 |
| ENSG00000145819 | ARHGAP26 | −0.77 | 0.105 |
| ENSG00000079134 | THOC1 | −0.88 | 0.105 |
| ENSG00000135596 | MICAL1 | −0.68 | 0.105 |
| ENSG00000110934 | BIN2 | 0.21 | 0.105 |
| ENSG00000072401 | UBE2D1 | −0.72 | 0.106 |
| ENSG00000172172 | MRPL13 | −0.56 | 0.107 |
| ENSG00000172053 | QARS | −0.56 | 0.107 |
| ENSG00000139350 | NEDD1 | −0.41 | 0.107 |
| ENSG00000170113 | NIPA1 | 0.51 | 0.107 |
| ENSG00000179344 | HLA-DQB1 | −0.92 | 0.108 |
| ENSG00000114626 | ABTB1 | 0.21 | 0.108 |
| ENSG00000033050 | ABCF2 | −0.81 | 0.108 |
| ENSG00000204371 | EHMT2 | −0.63 | 0.108 |
| ENSG00000128463 | EMC4 | −0.41 | 0.109 |
| ENSG00000146834 | MEPCE | 0.26 | 0.109 |
| ENSG00000080815 | PSEN1 | −0.81 | 0.109 |
| ENSG00000054523 | KIF1B | 0.50 | 0.109 |
| ENSG00000060237 | WNK1 | 0.35 | 0.110 |
| ENSG00000122705 | CLTA | 0.20 | 0.110 |
| ENSG00000067829 | IDH3G | 0.22 | 0.110 |
| ENSG00000046651 | OFD1 | −0.26 | 0.111 |
| ENSG00000103335 | PIEZO1 | −0.95 | 0.111 |
| ENSG00000125450 | NUP85 | −0.78 | 0.112 |
| ENSG00000146416 | AIG1 | 0.25 | 0.113 |
| ENSG00000163399 | ATP1A1 | 0.36 | 0.113 |
| ENSG00000125734 | GPR108 | 0.23 | 0.113 |
| ENSG00000196562 | SULF2 | −1.05 | 0.114 |
| ENSG00000128159 | TUBGCP6 | −0.83 | 0.114 |
| ENSG00000198851 | CD3E | −0.70 | 0.114 |
| ENSG00000131378 | RFTN1 | −0.64 | 0.115 |
| ENSG00000048707 | VPS13D | −0.53 | 0.117 |
| ENSG00000168056 | LTBP3 | −0.82 | 0.117 |
| ENSG00000148688 | RPP30 | −0.66 | 0.117 |
| ENSG00000183011 | LSMD1 | 0.27 | 0.117 |
| ENSG00000133872 | TMEM66 | −0.16 | 0.118 |
| ENSG00000026297 | RNASET2 | −0.37 | 0.118 |
| ENSG00000152942 | RAD17 | −0.67 | 0.118 |
| ENSG00000164332 | UBLCP1 | −0.58 | 0.118 |
| ENSG00000071189 | SNX13 | −0.44 | 0.119 |
| ENSG00000179115 | FARSA | −0.62 | 0.119 |
| ENSG00000136040 | PLXNC1 | −0.96 | 0.119 |
| ENSG00000105835 | NAMPT | −0.53 | 0.121 |
| ENSG00000164096 | C4orf3 | 0.23 | 0.121 |
| ENSG00000047644 | WWC3 | 0.30 | 0.122 |
| ENSG00000141298 | SSH2 | −0.29 | 0.123 |
| ENSG00000143156 | NME7 | 0.60 | 0.123 |
| ENSG00000197321 | SVIL | 0.24 | 0.124 |
| ENSG00000092330 | TINF2 | −0.30 | 0.124 |
| ENSG00000103740 | ACSBG1 | 0.30 | 0.125 |
| ENSG00000159210 | SNF8 | −0.40 | 0.126 |
| ENSG00000100461 | RBM23 | −0.33 | 0.127 |
| ENSG00000039123 | SKIV2L2 | −0.57 | 0.127 |
| ENSG00000188976 | NOC2L | −0.77 | 0.127 |
| ENSG00000106803 | SEC61B | −0.35 | 0.127 |
| ENSG00000106628 | POLD2 | −0.68 | 0.127 |
| ENSG00000125875 | TBC1D20 | 0.23 | 0.128 |
| ENSG00000156875 | HIAT1 | −0.35 | 0.128 |
| ENSG00000135968 | GCC2 | −0.33 | 0.128 |
| ENSG00000145241 | CENPC | −0.47 | 0.128 |
| ENSG00000075539 | FRYL | −0.40 | 0.128 |
| ENSG00000126709 | IFI6 | −0.64 | 0.129 |
| ENSG00000106153 | CHCHD2 | 0.19 | 0.130 |
| ENSG00000170873 | MTSS1 | 0.23 | 0.130 |
| ENSG00000197622 | CDC42SE1 | −0.42 | 0.131 |
| ENSG00000147036 | LANCL3 | 0.35 | 0.132 |
| ENSG00000135976 | ANKRD36 | −0.46 | 0.132 |
| ENSG00000143799 | PARP1 | −0.45 | 0.133 |
| ENSG00000163444 | TMEM183A | −0.22 | 0.133 |
| ENSG00000110395 | CBL | −0.36 | 0.133 |
| ENSG00000064419 | TNPO3 | −0.52 | 0.134 |
| ENSG00000163655 | GMPS | 0.24 | 0.135 |
| ENSG00000226777 | KIAA0125 | 0.31 | 0.135 |
| ENSG00000111669 | TPI1 | 0.14 | 0.135 |
| ENSG00000005175 | RPAP3 | −0.25 | 0.135 |
| ENSG00000107679 | PLEKHA1 | −0.57 | 0.135 |
| ENSG00000189067 | LITAF | −0.46 | 0.136 |
| ENSG00000144674 | GOLGA4 | −0.36 | 0.137 |
| ENSG00000189136 | UBE2Q2P1 | 0.43 | 0.137 |
| ENSG00000117592 | PRDX6 | 0.21 | 0.137 |
| ENSG00000138279 | ANXA7 | 0.18 | 0.137 |
| ENSG00000136754 | ABI1 | −0.22 | 0.137 |
| ENSG00000147457 | CHMP7 | −0.73 | 0.137 |
| ENSG00000162402 | USP24 | −0.68 | 0.138 |
| ENSG00000099246 | RAB18 | −0.27 | 0.138 |
| ENSG00000150681 | RGS18 | −0.28 | 0.139 |
| ENSG00000104907 | TRMT1 | −0.35 | 0.140 |
| ENSG00000197555 | SIPA1L1 | −0.73 | 0.140 |
| ENSG00000108094 | CUL2 | −0.57 | 0.140 |
| ENSG00000116688 | MFN2 | 0.42 | 0.140 |
| ENSG00000090060 | PAPOLA | 0.19 | 0.141 |
| ENSG00000054654 | SYNE2 | −0.65 | 0.141 |
| ENSG00000140750 | ARHGAP17 | −0.48 | 0.141 |
| ENSG00000140853 | NLRC5 | −0.40 | 0.141 |
| ENSG00000115808 | STRN | 0.32 | 0.143 |
| ENSG00000139083 | ETV6 | 0.24 | 0.144 |
| ENSG00000086589 | RBM22 | −0.53 | 0.144 |
| ENSG00000169129 | AFAP1L2 | 0.41 | 0.146 |
| ENSG00000118900 | UBN1 | 0.22 | 0.146 |
| ENSG00000148634 | HERC4 | −0.43 | 0.146 |
| ENSG00000205531 | NAP1L4 | 0.22 | 0.146 |
| ENSG00000164830 | OXR1 | −0.45 | 0.147 |
| ENSG00000139505 | MTMR6 | −0.69 | 0.147 |
| ENSG00000147050 | KDM6A | −0.46 | 0.147 |
| ENSG00000138496 | PARP9 | −0.55 | 0.148 |
| ENSG00000130935 | NOL11 | −0.83 | 0.149 |
| ENSG00000197969 | VPS13A | 0.26 | 0.149 |
| ENSG00000105698 | USF2 | 0.32 | 0.149 |
| ENSG00000132466 | ANKRD17 | −0.41 | 0.150 |
| ENSG00000178685 | PARP10 | −0.68 | 0.150 |
| ENSG00000137076 | TLN1 | 0.29 | 0.150 |
| ENSG00000152620 | NADK2 | −0.41 | 0.150 |
| ENSG00000198858 | R3HDM4 | 0.22 | 0.150 |
| ENSG00000163104 | SMARCAD1 | −0.53 | 0.151 |
| ENSG00000167286 | CD3D | −0.61 | 0.151 |
| ENSG00000114573 | ATP6V1A | −0.33 | 0.152 |
| ENSG00000078596 | ITM2A | 0.23 | 0.153 |
| ENSG00000136560 | TANK | −0.33 | 0.153 |
| ENSG00000174695 | TMEM167A | 0.23 | 0.154 |
| ENSG00000065150 | IPO5 | −0.50 | 0.154 |
| ENSG00000112031 | MTRF1L | 0.28 | 0.155 |
| ENSG00000182220 | ATP6AP2 | 0.20 | 0.155 |
| ENSG00000183172 | SMDT1 | 0.19 | 0.155 |
| ENSG00000103197 | TSC2 | −0.60 | 0.155 |
| ENSG00000103512 | NOMO1 | −0.39 | 0.156 |
| ENSG00000183291 | | −0.21 | 0.158 |
| ENSG00000115641 | FHL2 | 0.29 | 0.158 |
| ENSG00000162852 | CNST | −0.26 | 0.159 |
| ENSG00000110958 | PTGES3 | −0.17 | 0.159 |
| ENSG00000163947 | ARHGEF3 | −0.39 | 0.160 |
| ENSG00000168291 | PDHB | −0.28 | 0.160 |
| ENSG00000214078 | CPNE1 | 0.24 | 0.160 |
| ENSG00000087152 | ATXN7L3 | 0.32 | 0.160 |
| ENSG00000100297 | MCM5 | −0.48 | 0.160 |
| ENSG00000139626 | ITGB7 | −0.78 | 0.160 |
| ENSG00000140848 | CPNE2 | 0.29 | 0.162 |
| ENSG00000116514 | RNF19B | −0.46 | 0.162 |
| ENSG00000154122 | ANKH | −0.28 | 0.162 |
| ENSG00000134242 | PTPN22 | −0.71 | 0.162 |
| ENSG00000134779 | TPGS2 | 0.19 | 0.164 |
| ENSG00000101997 | CCDC22 | −0.62 | 0.165 |
| ENSG00000011243 | AKAP8L | −0.31 | 0.165 |
| ENSG00000079277 | MKNK1 | 0.28 | 0.165 |
| ENSG00000092929 | UNC13D | 0.26 | 0.165 |
| ENSG00000114416 | FXR1 | −0.36 | 0.167 |
| ENSG00000147872 | PLIN2 | −0.41 | 0.167 |
| ENSG00000002822 | MAD1L1 | −0.56 | 0.167 |
| ENSG00000169738 | DCXR | −0.45 | 0.167 |
| ENSG00000143643 | TTC13 | −0.88 | 0.167 |
| ENSG00000174837 | EMR1 | −0.34 | 0.167 |
| ENSG00000099995 | SF3A1 | −0.24 | 0.168 |
| ENSG00000131042 | LILRB2 | −0.81 | 0.168 |
| ENSG00000142208 | AKT1 | 0.25 | 0.169 |
| ENSG00000141959 | PFKL | 0.22 | 0.169 |
| ENSG00000119203 | CPSF3 | −0.63 | 0.170 |
| ENSG00000142546 | NOSIP | −0.29 | 0.170 |
| ENSG00000150347 | ARID5B | −0.56 | 0.171 |
| ENSG00000126261 | UBA2 | −0.34 | 0.172 |
| ENSG00000146463 | ZMYM4 | 0.32 | 0.173 |
| ENSG00000127511 | SIN3B | 0.23 | 0.174 |
| ENSG00000107625 | DDX50 | −0.51 | 0.174 |
| ENSG00000131165 | CHMP1A | 0.26 | 0.174 |
| ENSG00000103249 | CLCN7 | −0.53 | 0.175 |
| ENSG00000182732 | RGS6 | 0.20 | 0.175 |
| ENSG00000123064 | DDX54 | −0.62 | 0.176 |
| ENSG00000173113 | TRMT112 | −0.32 | 0.177 |
| ENSG00000100401 | RANGAP1 | 0.45 | 0.179 |
| ENSG00000111912 | NCOA7 | 0.21 | 0.180 |
| ENSG00000134824 | FADS2 | 0.39 | 0.180 |
| ENSG00000168439 | STIP1 | −0.28 | 0.181 |
| ENSG00000139624 | CERS5 | −0.34 | 0.182 |
| ENSG00000114388 | NPRL2 | −0.61 | 0.182 |
| ENSG00000101849 | TBL1X | 0.57 | 0.182 |
| ENSG00000145247 | OCIAD2 | −0.42 | 0.182 |
| ENSG00000198624 | CCDC69 | −0.46 | 0.183 |
| ENSG00000129933 | MAU2 | −0.60 | 0.184 |
| ENSG00000154217 | PITPNC1 | −0.36 | 0.184 |
| ENSG00000185418 | TARSL2 | 0.27 | 0.185 |
| ENSG00000124226 | RNF114 | −0.44 | 0.185 |
| ENSG00000073050 | XRCC1 | 0.23 | 0.186 |
| ENSG00000167978 | SRRM2 | −0.46 | 0.186 |
| ENSG00000122417 | ODF2L | −0.37 | 0.186 |
| ENSG00000102144 | PGK1 | 0.14 | 0.187 |
| ENSG00000160013 | PTGIR | 0.23 | 0.187 |
| ENSG00000166181 | API5 | −0.42 | 0.188 |
| ENSG00000182481 | KPNA2 | −0.50 | 0.189 |
| ENSG00000132792 | CTNNBL1 | −0.28 | 0.190 |
| ENSG00000171314 | PGAM1 | 0.18 | 0.191 |
| ENSG00000175054 | ATR | −0.75 | 0.192 |
| ENSG00000144649 | FAM198A | 0.29 | 0.192 |
| ENSG00000166888 | STAT6 | −0.33 | 0.192 |
| ENSG00000134748 | PRPF38A | −0.50 | 0.192 |
| ENSG00000092201 | SUPT16H | −0.37 | 0.193 |
| ENSG00000015153 | YAF2 | 0.30 | 0.194 |
| ENSG00000159625 | CCDC135 | 0.33 | 0.194 |
| ENSG00000166200 | COPS2 | −0.30 | 0.194 |
| ENSG00000116489 | CAPZA1 | 0.19 | 0.195 |
| ENSG00000116337 | AMPD2 | −0.28 | 0.195 |
| ENSG00000175416 | CLTB | 0.19 | 0.195 |
| ENSG00000018280 | SLC11A1 | −0.66 | 0.195 |
| ENSG00000272888 | | −0.20 | 0.195 |
| ENSG00000114446 | IFT57 | −0.41 | 0.195 |
| ENSG00000215845 | TSTD1 | −0.32 | 0.197 |
| ENSG00000119541 | VPS4B | −0.26 | 0.197 |
| ENSG00000062716 | VMP1 | −0.44 | 0.197 |
| ENSG00000151500 | THYN1 | −0.39 | 0.197 |
| ENSG00000205629 | LCMT1 | −0.40 | 0.197 |
| ENSG00000148362 | C9orf142 | −0.48 | 0.197 |
| ENSG00000204323 | SMIM5 | 0.27 | 0.198 |
| ENSG00000187257 | RSBN1L | −0.28 | 0.198 |
| ENSG00000134909 | ARHGAP32 | 0.24 | 0.198 |
| ENSG00000159176 | CSRP1 | 0.28 | 0.198 |
| ENSG00000120837 | NFYB | −0.39 | 0.200 |
| ENSG00000184602 | SNN | 0.23 | 0.200 |
| ENSG00000065357 | DGKA | −0.39 | 0.200 |
| ENSG00000237473 | | 0.30 | 0.200 |
| ENSG00000101158 | NELFCD | 0.18 | 0.201 |
| ENSG00000108651 | UTP6 | −0.62 | 0.201 |
| ENSG00000143641 | GALNT2 | 0.28 | 0.203 |
| ENSG00000102024 | PLS3 | −0.57 | 0.204 |
| ENSG00000104133 | SPG11 | −0.44 | 0.205 |
| ENSG00000069329 | VPS35 | −0.23 | 0.205 |
| ENSG00000125356 | NDUFA1 | 0.20 | 0.205 |
| ENSG00000120705 | ETF1 | −0.38 | 0.205 |
| ENSG00000103051 | COG4 | −0.59 | 0.205 |
| ENSG00000034053 | APBA2 | −0.39 | 0.206 |
| ENSG00000101040 | ZMYND8 | −0.24 | 0.207 |
| ENSG00000198162 | MAN1A2 | 0.25 | 0.207 |
| ENSG00000121892 | PDS5A | −0.26 | 0.208 |
| ENSG00000211896 | IGHG1 | −0.53 | 0.208 |
| ENSG00000175220 | ARHGAP1 | −0.34 | 0.208 |
| ENSG00000137210 | TMEM14B | −0.27 | 0.208 |
| ENSG00000166197 | NOLC1 | −0.47 | 0.210 |
| ENSG00000047365 | ARAP2 | −0.62 | 0.210 |
| ENSG00000148700 | ADD3 | 0.16 | 0.211 |
| ENSG00000124562 | SNRPC | −0.26 | 0.213 |
| ENSG00000033800 | PIAS1 | −0.25 | 0.213 |
| ENSG00000174953 | DHX36 | −0.36 | 0.213 |
| ENSG00000103415 | HMOX2 | −0.44 | 0.215 |
| ENSG00000136718 | IMP4 | −0.44 | 0.215 |
| ENSG00000197747 | S100A10 | 0.37 | 0.216 |
| ENSG00000138794 | CASP6 | 0.19 | 0.216 |
| ENSG00000204209 | DAXX | −0.23 | 0.216 |
| ENSG00000122223 | CD244 | −0.47 | 0.216 |
| ENSG00000149823 | VPS51 | −0.47 | 0.217 |
| ENSG00000166086 | JAM3 | 0.27 | 0.218 |
| ENSG00000132376 | INPP5K | 0.20 | 0.218 |
| ENSG00000151498 | ACAD8 | −0.29 | 0.219 |
| ENSG00000023318 | ERP44 | −0.34 | 0.219 |
| ENSG00000143183 | TMCO1 | −0.48 | 0.219 |
| ENSG00000125944 | HNRNPR | −0.16 | 0.219 |
| ENSG00000135604 | STX11 | −0.22 | 0.221 |
| ENSG00000128789 | PSMG2 | −0.27 | 0.225 |
| ENSG00000108349 | CASC3 | −0.29 | 0.225 |
| ENSG00000134294 | SLC38A2 | −0.35 | 0.226 |
| ENSG00000184319 | RPL23AP82 | 0.54 | 0.227 |
| ENSG00000185946 | RNPC3 | −0.36 | 0.227 |
| ENSG00000079246 | XRCC5 | −0.23 | 0.228 |
| ENSG00000104518 | GSDMD | −0.49 | 0.228 |
| ENSG00000089597 | GANAB | −0.34 | 0.229 |
| ENSG00000205250 | E2F4 | −0.47 | 0.230 |
| ENSG00000100938 | GMPR2 | 0.20 | 0.231 |
| ENSG00000157106 | SMG1 | −0.41 | 0.235 |
| ENSG00000006712 | PAF1 | −0.41 | 0.237 |
| ENSG00000123104 | ITPR2 | 0.23 | 0.240 |
| ENSG00000011405 | PIK3C2A | −0.46 | 0.242 |
| ENSG00000110330 | BIRC2 | −0.18 | 0.244 |

TABLE 3

| Gene | Gene_name | cor | FDR |
|---|---|---|---|
| ENSG00000001461 | NIPAL3 | 0.31 | 0.0000 |
| ENSG00000002330 | BAD | 0.37 | 0.0000 |
| ENSG00000002586 | CD99 | 0.52 | 0.0000 |
| ENSG00000002834 | LASP1 | 0.31 | 0.0000 |
| ENSG00000003436 | TFPI | 0.39 | 0.0000 |
| ENSG00000004059 | ARF5 | 0.46 | 0.0000 |
| ENSG00000004866 | ST7 | 0.65 | 0.0000 |
| ENSG00000005007 | UPF1 | 0.29 | 0.0000 |
| ENSG00000005020 | SKAP2 | 0.62 | 0.0000 |
| ENSG00000005238 | FAM214B | 0.62 | 0.0000 |
| ENSG00000005249 | PRKAR2B | 0.74 | 0.0000 |
| ENSG00000005486 | RHBDD2 | 0.21 | 0.0005 |
| ENSG00000005812 | FBXL3 | 0.24 | 0.0001 |
| ENSG00000005882 | PDK2 | 0.42 | 0.0000 |
| ENSG00000005893 | LAMP2 | 0.22 | 0.0004 |
| ENSG00000005961 | ITGA2B | 0.83 | 0.0000 |
| ENSG00000006007 | GDE1 | 0.69 | 0.0000 |
| ENSG00000006125 | AP2B1 | 0.47 | 0.0000 |
| ENSG00000006459 | KDM7A | 0.25 | 0.0000 |
| ENSG00000006576 | PHTF2 | 0.39 | 0.0000 |
| ENSG00000006638 | TBXA2R | 0.70 | 0.0000 |
| ENSG00000006652 | IFRD1 | 0.55 | 0.0000 |
| ENSG00000006715 | VPS41 | 0.53 | 0.0000 |
| ENSG00000008083 | JARID2 | 0.51 | 0.0000 |
| ENSG00000008513 | ST3GAL1 | 0.31 | 0.0000 |
| ENSG00000009307 | CSDE1 | 0.65 | 0.0000 |
| ENSG00000010017 | RANBP9 | 0.41 | 0.0000 |
| ENSG00000010270 | STARD3NL | 0.62 | 0.0000 |
| ENSG00000010278 | CD9 | 0.65 | 0.0000 |
| ENSG00000010404 | IDS | 0.57 | 0.0000 |
| ENSG00000010671 | BTK | 0.44 | 0.0000 |
| ENSG00000010810 | FYN | 0.52 | 0.0000 |
| ENSG00000011105 | TSPAN9 | 0.61 | 0.0000 |
| ENSG00000011198 | ABHD5 | 0.21 | 0.0008 |
| ENSG00000011258 | MBTD1 | 0.30 | 0.0000 |
| ENSG00000011304 | PTBP1 | 0.56 | 0.0000 |
| ENSG00000011454 | RABGAP1 | 0.19 | 0.0018 |
| ENSG00000011523 | CEP68 | 0.23 | 0.0002 |
| ENSG00000011638 | TMEM159 | 0.36 | 0.0000 |
| ENSG00000012822 | CALCOCO1 | 0.65 | 0.0000 |
| ENSG00000012983 | MAP4K5 | 0.54 | 0.0000 |
| ENSG00000013016 | EHD3 | 0.77 | 0.0000 |
| ENSG00000013561 | RNF14 | 0.30 | 0.0000 |
| ENSG00000014216 | CAPN1 | 0.84 | 0.0000 |
| ENSG00000015171 | ZMYND11 | 0.21 | 0.0008 |
| ENSG00000015479 | MATR3 | 0.26 | 0.0000 |
| ENSG00000015532 | XYLT2 | 0.39 | 0.0000 |
| ENSG00000017260 | ATP2C1 | 0.67 | 0.0000 |
| ENSG00000021355 | SERPINB1 | 0.44 | 0.0000 |
| ENSG00000022267 | FHL1 | 0.83 | 0.0000 |
| ENSG00000022840 | RNF10 | 0.71 | 0.0000 |
| ENSG00000023191 | RNH1 | 0.30 | 0.0000 |
| ENSG00000023697 | DERA | 0.61 | 0.0000 |
| ENSG00000023734 | STRAP | 0.64 | 0.0000 |
| ENSG00000023902 | PLEKHO1 | 0.54 | 0.0000 |
| ENSG00000023909 | GCLM | 0.68 | 0.0000 |
| ENSG00000028116 | VRK2 | 0.17 | 0.0046 |
| ENSG00000028203 | VEZT | 0.28 | 0.0000 |
| ENSG00000028528 | SNX1 | 0.34 | 0.0000 |
| ENSG00000028839 | TBPL1 | 0.40 | 0.0000 |
| ENSG00000029534 | ANK1 | 0.67 | 0.0000 |
| ENSG00000033170 | FUT8 | 0.39 | 0.0000 |
| ENSG00000033627 | ATP6V0A1 | 0.25 | 0.0000 |
| ENSG00000034053 | APBA2 | 0.17 | 0.0056 |
| ENSG00000034152 | MAP2K3 | 0.72 | 0.0000 |
| ENSG00000034713 | GABARAPL2 | 0.42 | 0.0000 |
| ENSG00000035403 | VCL | 0.74 | 0.0000 |
| ENSG00000036054 | TBC1D23 | 0.35 | 0.0000 |
| ENSG00000040341 | STAU2 | 0.36 | 0.0000 |
| ENSG00000040531 | CTNS | 0.54 | 0.0000 |
| ENSG00000041353 | RAB27B | 0.49 | 0.0000 |
| ENSG00000042062 | FAM65C | 0.36 | 0.0000 |
| ENSG00000042753 | AP2S1 | 0.37 | 0.0000 |
| ENSG00000043093 | DCUN1D1 | 0.22 | 0.0003 |
| ENSG00000044115 | CTNNA1 | 0.54 | 0.0000 |
| ENSG00000047597 | XK | 0.58 | 0.0000 |
| ENSG00000047617 | ANO2 | 0.36 | 0.0000 |
| ENSG00000047644 | WWC3 | 0.31 | 0.0000 |
| ENSG00000047648 | ARHGAP6 | 0.44 | 0.0000 |
| ENSG00000048740 | CELF2 | 0.56 | 0.0000 |
| ENSG00000048828 | FAM120A | 0.43 | 0.0000 |
| ENSG00000049245 | VAMP3 | 0.18 | 0.0042 |
| ENSG00000049323 | LTBP1 | 0.81 | 0.0000 |
| ENSG00000049541 | RFC2 | 0.22 | 0.0003 |
| ENSG00000049618 | ARID1B | 0.21 | 0.0006 |
| ENSG00000049656 | CLPTM1L | 0.24 | 0.0001 |
| ENSG00000050393 | MCUR1 | 0.37 | 0.0000 |
| ENSG00000051382 | PIK3CB | 0.61 | 0.0000 |
| ENSG00000052126 | PLEKHA5 | 0.27 | 0.0000 |
| ENSG00000053108 | FSTL4 | 0.22 | 0.0003 |
| ENSG00000053371 | AKR7A2 | 0.46 | 0.0000 |
| ENSG00000054356 | PTPRN | 0.50 | 0.0000 |
| ENSG00000055070 | SZRD1 | 0.42 | 0.0000 |
| ENSG00000055208 | TAB2 | 0.38 | 0.0000 |
| ENSG00000056586 | RC3H2 | 0.60 | 0.0000 |
| ENSG00000058091 | CDK14 | 0.31 | 0.0000 |
| ENSG00000058673 | ZC3H11A | 0.17 | 0.0050 |
| ENSG00000058866 | DGKG | 0.52 | 0.0000 |
| ENSG00000059377 | TBXAS1 | 0.47 | 0.0000 |
| ENSG00000059758 | CDK17 | 0.19 | 0.0019 |
| ENSG00000059804 | SLC2A3 | 0.53 | 0.0000 |
| ENSG00000060138 | YBX3 | 0.67 | 0.0000 |
| ENSG00000060558 | GNA15 | 0.69 | 0.0000 |
| ENSG00000061676 | NCKAP1 | 0.71 | 0.0000 |
| ENSG00000061918 | GUCY1B3 | 0.72 | 0.0000 |
| ENSG00000062598 | ELMO2 | 0.26 | 0.0000 |
| ENSG00000063245 | EPN1 | 0.38 | 0.0000 |
| ENSG00000064115 | TM7SF3 | 0.51 | 0.0000 |
| ENSG00000064201 | TSPAN32 | 0.37 | 0.0000 |
| ENSG00000064225 | ST3GAL6 | 0.26 | 0.0000 |
| ENSG00000064393 | HIPK2 | 0.38 | 0.0000 |
| ENSG00000064601 | CTSA | 0.84 | 0.0000 |
| ENSG00000064652 | SNX24 | 0.23 | 0.0002 |
| ENSG00000064666 | CNN2 | 0.36 | 0.0000 |
| ENSG00000064726 | BTBD1 | 0.46 | 0.0000 |
| ENSG00000064961 | HMG20B | 0.59 | 0.0000 |
| ENSG00000064999 | ANKS1A | 0.21 | 0.0006 |
| ENSG00000065060 | UHRF1BP1 | 0.30 | 0.0000 |
| ENSG00000065457 | ADAT1 | 0.29 | 0.0000 |
| ENSG00000065534 | MYLK | 0.71 | 0.0000 |
| ENSG00000065615 | CYB5R4 | 0.33 | 0.0000 |
| ENSG00000065675 | PRKCQ | 0.46 | 0.0000 |
| ENSG00000065833 | ME1 | 0.29 | 0.0000 |
| ENSG00000065911 | MTHFD2 | 0.50 | 0.0000 |
| ENSG00000065970 | FOXJ2 | 0.31 | 0.0000 |
| ENSG00000066027 | PPP2R5A | 0.42 | 0.0000 |
| ENSG00000066044 | ELAVL1 | 0.58 | 0.0000 |
| ENSG00000066136 | NFYC | 0.23 | 0.0002 |
| ENSG00000066185 | ZMYND12 | 0.23 | 0.0001 |
| ENSG00000066294 | CD84 | 0.57 | 0.0000 |
| ENSG00000066697 | MSANTD3 | 0.49 | 0.0000 |
| ENSG00000067057 | PFKP | 0.38 | 0.0000 |
| ENSG00000067167 | TRAM1 | 0.48 | 0.0000 |
| ENSG00000067225 | PKM | 0.80 | 0.0000 |
| ENSG00000067365 | METTL22 | 0.23 | 0.0002 |
| ENSG00000067560 | RHOA | 0.78 | 0.0000 |
| ENSG00000067836 | ROGDI | 0.33 | 0.0000 |
| ENSG00000067992 | PDK3 | 0.45 | 0.0000 |
| ENSG00000068308 | OTUD5 | 0.56 | 0.0000 |
| ENSG00000068354 | TBC1D25 | 0.46 | 0.0000 |
| ENSG00000068383 | INPP5A | 0.66 | 0.0000 |
| ENSG00000068400 | GRIPAP1 | 0.18 | 0.0032 |
| ENSG00000068650 | ATP11A | 0.22 | 0.0003 |
| ENSG00000068793 | CYFIP1 | 0.40 | 0.0000 |
| ENSG00000068796 | KIF2A | 0.34 | 0.0000 |
| ENSG00000068831 | RASGRP2 | 0.42 | 0.0000 |
| ENSG00000068903 | SIRT2 | 0.59 | 0.0000 |
| ENSG00000069020 | MAST4 | 0.35 | 0.0000 |
| ENSG00000069535 | MAOB | 0.60 | 0.0000 |
| ENSG00000069966 | GNB5 | 0.83 | 0.0000 |
| ENSG00000070010 | UFD1L | 0.25 | 0.0000 |
| ENSG00000070182 | SPTB | 0.59 | 0.0000 |
| ENSG00000070190 | DAPP1 | 0.38 | 0.0000 |
| ENSG00000070214 | SLC44A1 | 0.74 | 0.0000 |
| ENSG00000070413 | DGCR2 | 0.50 | 0.0000 |
| ENSG00000070540 | WIPI1 | 0.70 | 0.0000 |
| ENSG00000070614 | NDST1 | 0.63 | 0.0000 |
| ENSG00000071051 | NCK2 | 0.74 | 0.0000 |
| ENSG00000071127 | WDR1 | 0.86 | 0.0000 |
| ENSG00000071553 | ATP6AP1 | 0.67 | 0.0000 |
| ENSG00000071889 | FAM3A | 0.42 | 0.0000 |
| ENSG00000071909 | MYO3B | 0.22 | 0.0004 |
| ENSG00000072042 | RDH11 | 0.74 | 0.0000 |
| ENSG00000072110 | ACTN1 | 0.87 | 0.0000 |
| ENSG00000072135 | PTPN18 | 0.70 | 0.0000 |
| ENSG00000072422 | RHOBTB1 | 0.62 | 0.0000 |
| ENSG00000072778 | ACADVL | 0.19 | 0.0022 |
| ENSG00000072803 | FBXW11 | 0.33 | 0.0000 |
| ENSG00000072858 | SIDT1 | 0.39 | 0.0000 |
| ENSG00000072952 | MRVI1 | 0.46 | 0.0000 |
| ENSG00000073009 | IKBKG | 0.67 | 0.0000 |
| ENSG00000073111 | MCM2 | 0.18 | 0.0030 |
| ENSG00000073464 | CLCN4 | 0.46 | 0.0000 |
| ENSG00000073578 | SDHA | 0.49 | 0.0000 |
| ENSG00000073792 | IGF2BP2 | 0.53 | 0.0000 |
| ENSG00000073849 | ST6GAL1 | 0.39 | 0.0000 |
| ENSG00000074054 | CLASP1 | 0.25 | 0.0001 |
| ENSG00000074370 | ATP2A3 | 0.69 | 0.0000 |
| ENSG00000074416 | MGLL | 0.66 | 0.0000 |
| ENSG00000074603 | DPP8 | 0.28 | 0.0000 |
| ENSG00000074800 | ENO1 | 0.63 | 0.0000 |
| ENSG00000075151 | EIF4G3 | 0.62 | 0.0000 |
| ENSG00000075413 | MARK3 | 0.24 | 0.0001 |
| ENSG00000075624 | ACTB | 0.82 | 0.0000 |
| ENSG00000075711 | DLG1 | 0.20 | 0.0011 |
| ENSG00000075785 | RAB7A | 0.38 | 0.0000 |
| ENSG00000075790 | BCAP29 | 0.33 | 0.0000 |
| ENSG00000075945 | KIFAP3 | 0.43 | 0.0000 |
| ENSG00000076003 | MCM6 | 0.31 | 0.0000 |
| ENSG00000076043 | REXO2 | 0.19 | 0.0023 |
| ENSG00000076685 | NT5C2 | 0.41 | 0.0000 |
| ENSG00000076770 | MBNL3 | 0.27 | 0.0000 |
| ENSG00000076944 | STXBP2 | 0.61 | 0.0000 |
| ENSG00000077044 | DGKD | 0.49 | 0.0000 |
| ENSG00000077254 | USP33 | 0.44 | 0.0000 |
| ENSG00000077549 | CAPZB | 0.61 | 0.0000 |
| ENSG00000077585 | GPR137B | 0.39 | 0.0000 |
| ENSG00000077713 | SLC25A43 | 0.39 | 0.0000 |
| ENSG00000077809 | GTF2I | 0.19 | 0.0019 |
| ENSG00000078061 | ARAF | 0.53 | 0.0000 |
| ENSG00000078124 | ACER3 | 0.46 | 0.0000 |
| ENSG00000078369 | GNB1 | 0.75 | 0.0000 |
| ENSG00000078596 | ITM2A | 0.30 | 0.0000 |
| ENSG00000078618 | NRD1 | 0.81 | 0.0000 |
| ENSG00000078668 | VDAC3 | 0.38 | 0.0000 |
| ENSG00000078902 | TOLLIP | 0.54 | 0.0000 |
| ENSG00000079257 | LXN | 0.24 | 0.0001 |
| ENSG00000079277 | MKNK1 | 0.22 | 0.0003 |
| ENSG00000079308 | TNS1 | 0.29 | 0.0000 |
| ENSG00000079387 | SENP1 | 0.23 | 0.0002 |
| ENSG00000079482 | OPHN1 | 0.29 | 0.0000 |
| ENSG00000079739 | PGM1 | 0.28 | 0.0000 |
| ENSG00000079950 | STX7 | 0.22 | 0.0003 |
| ENSG00000080371 | RAB21 | 0.23 | 0.0002 |
| ENSG00000080503 | SMARCA2 | 0.34 | 0.0000 |
| ENSG00000081087 | OSTM1 | 0.43 | 0.0000 |
| ENSG00000081154 | PCNP | 0.43 | 0.0000 |
| ENSG00000081181 | ARG2 | 0.51 | 0.0000 |
| ENSG00000081377 | CDC14B | 0.77 | 0.0000 |
| ENSG00000082074 | FYB | 0.33 | 0.0000 |
| ENSG00000082146 | STRADB | 0.30 | 0.0000 |
| ENSG00000082397 | EPB41L3 | 0.38 | 0.0000 |
| ENSG00000082701 | GSK3B | 0.21 | 0.0008 |
| ENSG00000082781 | ITGB5 | 0.86 | 0.0000 |
| ENSG00000083312 | TNPO1 | 0.52 | 0.0000 |
| ENSG00000083444 | PLOD1 | 0.24 | 0.0001 |
| ENSG00000084072 | PPIE | 0.23 | 0.0001 |
| ENSG00000084073 | ZMPSTE24 | 0.18 | 0.0029 |
| ENSG00000084112 | SSH1 | 0.48 | 0.0000 |
| ENSG00000084676 | NCOA1 | 0.50 | 0.0000 |
| ENSG00000084693 | AGBL5 | 0.76 | 0.0000 |
| ENSG00000084731 | KIF3C | 0.69 | 0.0000 |
| ENSG00000084733 | RAB10 | 0.44 | 0.0000 |
| ENSG00000085117 | CD82 | 0.61 | 0.0000 |
| ENSG00000085449 | WDFY1 | 0.22 | 0.0004 |
| ENSG00000085733 | CTTN | 0.69 | 0.0000 |
| ENSG00000085832 | EPS15 | 0.27 | 0.0000 |
| ENSG00000086065 | CHMP5 | 0.26 | 0.0000 |
| ENSG00000086200 | IPO11 | 0.19 | 0.0015 |
| ENSG00000086232 | EIF2AK1 | 0.74 | 0.0000 |
| ENSG00000086506 | HBQ1 | 0.19 | 0.0019 |
| ENSG00000086666 | ZFAND6 | 0.19 | 0.0019 |
| ENSG00000087053 | MTMR2 | 0.68 | 0.0000 |
| ENSG00000087086 | FTL | 0.22 | 0.0003 |
| ENSG00000087095 | NLK | 0.40 | 0.0000 |
| ENSG00000087152 | ATXN7L3 | 0.37 | 0.0000 |
| ENSG00000087157 | PGS1 | 0.19 | 0.0018 |
| ENSG00000087206 | UIMC1 | 0.67 | 0.0000 |
| ENSG00000087237 | CETP | 0.63 | 0.0000 |
| ENSG00000087258 | GNAO1 | 0.38 | 0.0000 |
| ENSG00000087274 | ADD1 | 0.48 | 0.0000 |
| ENSG00000087303 | NID2 | 0.38 | 0.0000 |
| ENSG00000087460 | GNAS | 0.63 | 0.0000 |
| ENSG00000087470 | DNM1L | 0.49 | 0.0000 |
| ENSG00000088053 | GP6 | 0.84 | 0.0000 |
| ENSG00000088448 | ANKRD10 | 0.19 | 0.0021 |
| ENSG00000088726 | TMEM40 | 0.39 | 0.0000 |
| ENSG00000088766 | CRLS1 | 0.42 | 0.0000 |
| ENSG00000088826 | SMOX | 0.72 | 0.0000 |
| ENSG00000088832 | FKBP1A | 0.59 | 0.0000 |
| ENSG00000088888 | MAVS | 0.68 | 0.0000 |
| ENSG00000089006 | SNX5 | 0.17 | 0.0058 |
| ENSG00000089053 | ANAPC5 | 0.65 | 0.0000 |
| ENSG00000089063 | TMEM230 | 0.32 | 0.0000 |
| ENSG00000089327 | FXYD5 | 0.34 | 0.0000 |
| ENSG00000089351 | GRAMD1A | 0.35 | 0.0000 |
| ENSG00000089486 | CDIP1 | 0.58 | 0.0000 |
| ENSG00000089639 | GMIP | 0.24 | 0.0001 |
| ENSG00000090020 | SLC9A1 | 0.48 | 0.0000 |
| ENSG00000090372 | STRN4 | 0.80 | 0.0000 |
| ENSG00000090565 | RAB11FIP3 | 0.44 | 0.0000 |
| ENSG00000090975 | PITPNM2 | 0.66 | 0.0000 |
| ENSG00000091317 | CMTM6 | 0.41 | 0.0000 |
| ENSG00000091409 | ITGA6 | 0.23 | 0.0002 |
| ENSG00000091490 | SEL1L3 | 0.23 | 0.0002 |
| ENSG00000091542 | ALKBH5 | 0.37 | 0.0000 |
| ENSG00000092531 | SNAP23 | 0.75 | 0.0000 |
| ENSG00000092621 | PHGDH | 0.22 | 0.0002 |
| ENSG00000092841 | MYL6 | 0.32 | 0.0000 |
| ENSG00000092847 | AGO1 | 0.44 | 0.0000 |
| ENSG00000092929 | UNC13D | 0.77 | 0.0000 |
| ENSG00000092931 | MFSD11 | 0.17 | 0.0057 |
| ENSG00000093010 | COMT | 0.23 | 0.0001 |
| ENSG00000093167 | LRRFIP2 | 0.40 | 0.0000 |
| ENSG00000094631 | HDAC6 | 0.26 | 0.0000 |
| ENSG00000095303 | PTGS1 | 0.87 | 0.0000 |
| ENSG00000095321 | CRAT | 0.81 | 0.0000 |
| ENSG00000095794 | CREM | 0.28 | 0.0000 |
| ENSG00000096968 | JAK2 | 0.43 | 0.0000 |
| ENSG00000097021 | ACOT7 | 0.60 | 0.0000 |
| ENSG00000097033 | SH3GLB1 | 0.19 | 0.0024 |
| ENSG00000099204 | ABLIM1 | 0.50 | 0.0000 |
| ENSG00000099256 | PRTFDC1 | 0.60 | 0.0000 |
| ENSG00000099337 | KCNK6 | 0.50 | 0.0000 |
| ENSG00000099785 | MARCH2 | 0.74 | 0.0000 |
| ENSG00000099817 | POLR2E | 0.64 | 0.0000 |
| ENSG00000099940 | SNAP29 | 0.58 | 0.0000 |
| ENSG00000099942 | CRKL | 0.49 | 0.0000 |
| ENSG00000099995 | SF3A1 | 0.29 | 0.0000 |
| ENSG00000100030 | MAPK1 | 0.54 | 0.0000 |
| ENSG00000100060 | MFNG | 0.64 | 0.0000 |
| ENSG00000100075 | SLC25A1 | 0.26 | 0.0000 |
| ENSG00000100077 | ADRBK2 | 0.60 | 0.0000 |
| ENSG00000100181 | TPTEP1 | 0.48 | 0.0000 |
| ENSG00000100225 | FBXO7 | 0.70 | 0.0000 |
| ENSG00000100243 | CYB5R3 | 0.84 | 0.0000 |
| ENSG00000100266 | PACSIN2 | 0.71 | 0.0000 |
| ENSG00000100280 | AP1B1 | 0.58 | 0.0000 |
| ENSG00000100299 | ARSA | 0.30 | 0.0000 |
| ENSG00000100345 | MYH9 | 0.69 | 0.0000 |
| ENSG00000100351 | GRAP2 | 0.72 | 0.0000 |
| ENSG00000100359 | SGSM3 | 0.32 | 0.0000 |
| ENSG00000100418 | DESI1 | 0.24 | 0.0001 |
| ENSG00000100427 | MLC1 | 0.34 | 0.0000 |
| ENSG00000100439 | ABHD4 | 0.64 | 0.0000 |
| ENSG00000100490 | CDKL1 | 0.31 | 0.0000 |
| ENSG00000100503 | NIN | 0.28 | 0.0000 |
| ENSG00000100504 | PYGL | 0.57 | 0.0000 |
| ENSG00000100532 | CGRRF1 | 0.49 | 0.0000 |
| ENSG00000100554 | ATP6V1D | 0.23 | 0.0002 |
| ENSG00000100568 | VTI1B | 0.50 | 0.0000 |
| ENSG00000100592 | DAAM1 | 0.32 | 0.0000 |
| ENSG00000100600 | LGMN | 0.46 | 0.0000 |
| ENSG00000100614 | PPM1A | 0.63 | 0.0000 |
| ENSG00000100711 | ZFYVE21 | 0.54 | 0.0000 |
| ENSG00000100811 | YY1 | 0.17 | 0.0068 |
| ENSG00000100897 | DCAF11 | 0.26 | 0.0000 |
| ENSG00000100934 | SEC23A | 0.42 | 0.0000 |
| ENSG00000100979 | PLTP | 0.41 | 0.0000 |
| ENSG00000100982 | PCIF1 | 0.40 | 0.0000 |
| ENSG00000100983 | GSS | 0.18 | 0.0043 |
| ENSG00000100994 | PYGB | 0.75 | 0.0000 |
| ENSG00000101017 | CD40 | 0.17 | 0.0048 |
| ENSG00000101079 | NDRG3 | 0.26 | 0.0000 |
| ENSG00000101082 | SLA2 | 0.77 | 0.0000 |
| ENSG00000101109 | STK4 | 0.24 | 0.0001 |
| ENSG00000101150 | TPD52L2 | 0.25 | 0.0000 |
| ENSG00000101158 | NELFCD | 0.45 | 0.0000 |
| ENSG00000101162 | TUBB1 | 0.69 | 0.0000 |
| ENSG00000101236 | RNF24 | 0.65 | 0.0000 |
| ENSG00000101246 | ARFRP1 | 0.27 | 0.0000 |
| ENSG00000101290 | CDS2 | 0.50 | 0.0000 |
| ENSG00000101333 | PLCB4 | 0.24 | 0.0001 |
| ENSG00000101335 | MYL9 | 0.65 | 0.0000 |
| ENSG00000101367 | MAPRE1 | 0.65 | 0.0000 |
| ENSG00000101412 | E2F1 | 0.49 | 0.0000 |
| ENSG00000101439 | CST3 | 0.38 | 0.0000 |
| ENSG00000101460 | MAP1LC3A | 0.38 | 0.0000 |
| ENSG00000101473 | ACOT8 | 0.26 | 0.0000 |
| ENSG00000101558 | VAPA | 0.41 | 0.0000 |
| ENSG00000101605 | MYOM1 | 0.53 | 0.0000 |
| ENSG00000101608 | MYL12A | 0.28 | 0.0000 |
| ENSG00000101782 | RIOK3 | 0.55 | 0.0000 |
| ENSG00000101856 | PGRMC1 | 0.64 | 0.0000 |
| ENSG00000101940 | WDR13 | 0.53 | 0.0000 |
| ENSG00000102054 | RBBP7 | 0.25 | 0.0000 |
| ENSG00000102119 | EMD | 0.52 | 0.0000 |
| ENSG00000102144 | PGK1 | 0.56 | 0.0000 |
| ENSG00000102145 | GATA1 | 0.61 | 0.0000 |
| ENSG00000102172 | SMS | 0.52 | 0.0000 |
| ENSG00000102178 | UBL4A | 0.73 | 0.0000 |
| ENSG00000102225 | CDK16 | 0.64 | 0.0000 |
| ENSG00000102226 | USP11 | 0.17 | 0.0068 |
| ENSG00000102230 | PCYT1B | 0.45 | 0.0000 |
| ENSG00000102265 | TIMP1 | 0.39 | 0.0000 |
| ENSG00000102316 | MAGED2 | 0.83 | 0.0000 |
| ENSG00000102362 | SYTL4 | 0.55 | 0.0000 |
| ENSG00000102393 | GLA | 0.42 | 0.0000 |
| ENSG00000102401 | ARMCX3 | 0.55 | 0.0000 |
| ENSG00000102409 | BEX4 | 0.20 | 0.0015 |
| ENSG00000102572 | STK24 | 0.78 | 0.0000 |
| ENSG00000102753 | KPNA3 | 0.17 | 0.0067 |
| ENSG00000102781 | KATNAL1 | 0.36 | 0.0000 |
| ENSG00000102804 | TSC22D1 | 0.38 | 0.0000 |
| ENSG00000102893 | PHKB | 0.80 | 0.0000 |
| ENSG00000102897 | LYRM1 | 0.25 | 0.0000 |
| ENSG00000102898 | NUTF2 | 0.44 | 0.0000 |
| ENSG00000102901 | CENPT | 0.46 | 0.0000 |
| ENSG00000102908 | NFAT5 | 0.29 | 0.0000 |
| ENSG00000103148 | NPRL3 | 0.56 | 0.0000 |
| ENSG00000103160 | HSDL1 | 0.28 | 0.0000 |
| ENSG00000103184 | SEC14L5 | 0.49 | 0.0000 |
| ENSG00000103187 | COTL1 | 0.65 | 0.0000 |
| ENSG00000103194 | USP10 | 0.28 | 0.0000 |
| ENSG00000103202 | NME4 | 0.43 | 0.0000 |
| ENSG00000103222 | ABCC1 | 0.18 | 0.0037 |
| ENSG00000103266 | STUB1 | 0.25 | 0.0001 |
| ENSG00000103316 | CRYM | 0.28 | 0.0000 |
| ENSG00000103404 | USP31 | 0.54 | 0.0000 |
| ENSG00000103495 | MAZ | 0.49 | 0.0000 |
| ENSG00000103502 | CDIPT | 0.48 | 0.0000 |
| ENSG00000103507 | BCKDK | 0.27 | 0.0000 |
| ENSG00000103591 | AAGAB | 0.23 | 0.0002 |
| ENSG00000103657 | HERC1 | 0.23 | 0.0002 |
| ENSG00000103740 | ACSBG1 | 0.43 | 0.0000 |
| ENSG00000103769 | RAB11A | 0.32 | 0.0000 |
| ENSG00000103876 | FAH | 0.64 | 0.0000 |
| ENSG00000103942 | HOMER2 | 0.74 | 0.0000 |
| ENSG00000104164 | BLOC1S6 | 0.44 | 0.0000 |
| ENSG00000104219 | ZDHHC2 | 0.50 | 0.0000 |
| ENSG00000104231 | ZFAND1 | 0.22 | 0.0003 |
| ENSG00000104267 | CA2 | 0.25 | 0.0000 |
| ENSG00000104324 | CPQ | 0.51 | 0.0000 |
| ENSG00000104341 | LAPTM4B | 0.60 | 0.0000 |
| ENSG00000104687 | GSR | 0.41 | 0.0000 |
| ENSG00000104695 | PPP2CB | 0.25 | 0.0000 |
| ENSG00000104763 | ASAH1 | 0.58 | 0.0000 |
| ENSG00000104765 | BNIP3L | 0.44 | 0.0000 |
| ENSG00000104805 | NUCB1 | 0.69 | 0.0000 |
| ENSG00000104897 | SF3A2 | 0.36 | 0.0000 |
| ENSG00000104903 | LYL1 | 0.59 | 0.0000 |
| ENSG00000104904 | OAZ1 | 0.55 | 0.0000 |
| ENSG00000104946 | TBC1D17 | 0.39 | 0.0000 |
| ENSG00000105058 | FAM32A | 0.43 | 0.0000 |
| ENSG00000105063 | PPP6R1 | 0.71 | 0.0000 |
| ENSG00000105186 | ANKRD27 | 0.21 | 0.0005 |
| ENSG00000105220 | GPI | 0.66 | 0.0000 |
| ENSG00000105229 | PIAS4 | 0.25 | 0.0000 |
| ENSG00000105254 | TBCB | 0.22 | 0.0004 |
| ENSG00000105323 | HNRNPUL1 | 0.33 | 0.0000 |
| ENSG00000105329 | TGFB1 | 0.54 | 0.0000 |
| ENSG00000105355 | PLIN3 | 0.67 | 0.0000 |
| ENSG00000105401 | CDC37 | 0.41 | 0.0000 |
| ENSG00000105402 | NAPA | 0.72 | 0.0000 |
| ENSG00000105404 | RABAC1 | 0.35 | 0.0000 |
| ENSG00000105443 | CYTH2 | 0.42 | 0.0000 |
| ENSG00000105499 | PLA2G4C | 0.39 | 0.0000 |
| ENSG00000105507 | CABP5 | 0.35 | 0.0000 |
| ENSG00000105639 | JAK3 | 0.29 | 0.0000 |
| ENSG00000105698 | USF2 | 0.24 | 0.0001 |
| ENSG00000105700 | KXD1 | 0.39 | 0.0000 |
| ENSG00000105701 | FKBP8 | 0.57 | 0.0000 |
| ENSG00000105711 | SCN1B | 0.51 | 0.0000 |
| ENSG00000105717 | PBX4 | 0.31 | 0.0000 |
| ENSG00000105829 | BET1 | 0.27 | 0.0000 |
| ENSG00000105851 | PIK3CG | 0.20 | 0.0011 |
| ENSG00000105887 | MTPN | 0.27 | 0.0000 |
| ENSG00000105953 | OGDH | 0.17 | 0.0050 |
| ENSG00000105971 | CAV2 | 0.51 | 0.0000 |
| ENSG00000105993 | DNAJB6 | 0.55 | 0.0000 |
| ENSG00000106012 | IQCE | 0.22 | 0.0003 |
| ENSG00000106034 | CPED1 | 0.31 | 0.0000 |
| ENSG00000106070 | GRB10 | 0.42 | 0.0000 |
| ENSG00000106086 | PLEKHA8 | 0.38 | 0.0000 |
| ENSG00000106089 | STX1A | 0.58 | 0.0000 |
| ENSG00000106144 | CASP2 | 0.17 | 0.0070 |
| ENSG00000106211 | HSPB1 | 0.25 | 0.0000 |
| ENSG00000106244 | PDAP1 | 0.18 | 0.0035 |
| ENSG00000106290 | TAF6 | 0.25 | 0.0001 |
| ENSG00000106366 | SERPINE1 | 0.50 | 0.0000 |
| ENSG00000106392 | C1GALT1 | 0.32 | 0.0000 |
| ENSG00000106477 | CEP41 | 0.24 | 0.0001 |
| ENSG00000106484 | MEST | 0.41 | 0.0000 |
| ENSG00000106537 | TSPAN13 | 0.45 | 0.0000 |
| ENSG00000106609 | TMEM248 | 0.44 | 0.0000 |
| ENSG00000106615 | RHEB | 0.50 | 0.0000 |
| ENSG00000106635 | BCL7B | 0.60 | 0.0000 |
| ENSG00000106665 | CLIP2 | 0.57 | 0.0000 |
| ENSG00000106733 | NMRK1 | 0.21 | 0.0008 |
| ENSG00000106868 | SUSD1 | 0.83 | 0.0000 |
| ENSG00000106976 | DNM1 | 0.56 | 0.0000 |
| ENSG00000107021 | TBC1D13 | 0.57 | 0.0000 |
| ENSG00000107438 | PDLIM1 | 0.65 | 0.0000 |
| ENSG00000107521 | HPS1 | 0.54 | 0.0000 |
| ENSG00000107669 | ATE1 | 0.41 | 0.0000 |
| ENSG00000107738 | C10orf54 | 0.38 | 0.0000 |
| ENSG00000107745 | MICU1 | 0.69 | 0.0000 |
| ENSG00000107798 | LIPA | 0.65 | 0.0000 |
| ENSG00000107816 | LZTS2 | 0.52 | 0.0000 |
| ENSG00000107819 | SFXN3 | 0.57 | 0.0000 |
| ENSG00000107863 | ARHGAP21 | 0.48 | 0.0000 |
| ENSG00000108039 | XPNPEP1 | 0.80 | 0.0000 |
| ENSG00000108061 | SHOC2 | 0.25 | 0.0000 |
| ENSG00000108100 | CCNY | 0.79 | 0.0000 |
| ENSG00000108179 | PPIF | 0.49 | 0.0000 |
| ENSG00000108187 | PBLD | 0.47 | 0.0000 |
| ENSG00000108219 | TSPAN14 | 0.28 | 0.0000 |
| ENSG00000108370 | RGS9 | 0.27 | 0.0000 |
| ENSG00000108387 | SEPT4 | 0.52 | 0.0000 |
| ENSG00000108405 | P2RX1 | 0.83 | 0.0000 |
| ENSG00000108469 | RECQL5 | 0.30 | 0.0000 |
| ENSG00000108509 | CAMTA2 | 0.58 | 0.0000 |
| ENSG00000108523 | RNF167 | 0.18 | 0.0039 |
| ENSG00000108576 | SLC6A4 | 0.54 | 0.0000 |
| ENSG00000108622 | ICAM2 | 0.42 | 0.0000 |
| ENSG00000108679 | LGALS3BP | 0.39 | 0.0000 |
| ENSG00000108839 | ALOX12 | 0.83 | 0.0000 |
| ENSG00000108840 | HDAC5 | 0.64 | 0.0000 |
| ENSG00000108846 | ABCC3 | 0.79 | 0.0000 |
| ENSG00000108861 | DUSP3 | 0.44 | 0.0000 |
| ENSG00000108883 | EFTUD2 | 0.28 | 0.0000 |
| ENSG00000108946 | PRKAR1A | 0.66 | 0.0000 |
| ENSG00000108953 | YWHAE | 0.57 | 0.0000 |
| ENSG00000108960 | MMD | 0.62 | 0.0000 |
| ENSG00000109062 | SLC9A3R1 | 0.59 | 0.0000 |
| ENSG00000109066 | TMEM104 | 0.36 | 0.0000 |
| ENSG00000109171 | SLAIN2 | 0.18 | 0.0033 |
| ENSG00000109272 | PF4V1 | 0.29 | 0.0000 |
| ENSG00000109339 | MAPK10 | 0.27 | 0.0000 |
| ENSG00000109572 | CLCN3 | 0.55 | 0.0000 |
| ENSG00000109787 | KLF3 | 0.40 | 0.0000 |
| ENSG00000109854 | HTATIP2 | 0.36 | 0.0000 |
| ENSG00000110002 | VWA5A | 0.42 | 0.0000 |
| ENSG00000110011 | DNAJC4 | 0.49 | 0.0000 |
| ENSG00000110013 | SIAE | 0.77 | 0.0000 |
| ENSG00000110047 | EHD1 | 0.70 | 0.0000 |
| ENSG00000110080 | ST3GAL4 | 0.43 | 0.0000 |
| ENSG00000110090 | CPT1A | 0.33 | 0.0000 |
| ENSG00000110218 | PANX1 | 0.32 | 0.0000 |
| ENSG00000110321 | EIF4G2 | 0.78 | 0.0000 |
| ENSG00000110395 | CBL | 0.26 | 0.0000 |
| ENSG00000110422 | HIPK3 | 0.35 | 0.0000 |
| ENSG00000110455 | ACCS | 0.30 | 0.0000 |
| ENSG00000110514 | MADD | 0.65 | 0.0000 |
| ENSG00000110665 | C11orf21 | 0.29 | 0.0000 |
| ENSG00000110799 | VWF | 0.64 | 0.0000 |
| ENSG00000110851 | PRDM4 | 0.24 | 0.0001 |
| ENSG00000110880 | CORO1C | 0.69 | 0.0000 |
| ENSG00000110906 | KCTD10 | 0.27 | 0.0000 |
| ENSG00000110917 | MLEC | 0.33 | 0.0000 |
| ENSG00000110934 | BIN2 | 0.82 | 0.0000 |
| ENSG00000111145 | ELK3 | 0.70 | 0.0000 |
| ENSG00000111252 | SH2B3 | 0.46 | 0.0000 |
| ENSG00000111269 | CREBL2 | 0.32 | 0.0000 |
| ENSG00000111328 | CDK2AP1 | 0.66 | 0.0000 |
| ENSG00000111348 | ARHGDIB | 0.56 | 0.0000 |
| ENSG00000111424 | VDR | 0.43 | 0.0000 |
| ENSG00000111481 | COPZ1 | 0.29 | 0.0000 |
| ENSG00000111540 | RAB5B | 0.52 | 0.0000 |
| ENSG00000111554 | MDM1 | 0.49 | 0.0000 |
| ENSG00000111640 | GAPDH | 0.64 | 0.0000 |
| ENSG00000111644 | ACRBP | 0.33 | 0.0000 |
| ENSG00000111653 | ING4 | 0.28 | 0.0000 |
| ENSG00000111669 | TPI1 | 0.48 | 0.0000 |
| ENSG00000111674 | ENO2 | 0.61 | 0.0000 |
| ENSG00000111684 | LPCAT3 | 0.29 | 0.0000 |
| ENSG00000111711 | GOLT1B | 0.18 | 0.0034 |
| ENSG00000111726 | CMAS | 0.31 | 0.0000 |
| ENSG00000111790 | FGFR1OP2 | 0.24 | 0.0001 |
| ENSG00000111817 | DSE | 0.53 | 0.0000 |
| ENSG00000111885 | MAN1A1 | 0.38 | 0.0000 |
| ENSG00000111912 | NCOA7 | 0.25 | 0.0000 |
| ENSG00000112031 | MTRF1L | 0.61 | 0.0000 |
| ENSG00000112062 | MAPK14 | 0.48 | 0.0000 |
| ENSG00000112078 | KCTD20 | 0.70 | 0.0000 |
| ENSG00000112079 | STK38 | 0.18 | 0.0032 |
| ENSG00000112096 | SOD2 | 0.42 | 0.0000 |
| ENSG00000112146 | FBXO9 | 0.55 | 0.0000 |
| ENSG00000112234 | FBXL4 | 0.27 | 0.0000 |
| ENSG00000112242 | E2F3 | 0.28 | 0.0000 |
| ENSG00000112245 | PTP4A1 | 0.20 | 0.0014 |
| ENSG00000112290 | WASF1 | 0.48 | 0.0000 |
| ENSG00000112308 | C6orf62 | 0.54 | 0.0000 |
| ENSG00000112335 | SNX3 | 0.63 | 0.0000 |
| ENSG00000112531 | QKI | 0.29 | 0.0000 |
| ENSG00000112576 | CCND3 | 0.82 | 0.0000 |
| ENSG00000112655 | PTK7 | 0.40 | 0.0000 |
| ENSG00000112679 | DUSP22 | 0.48 | 0.0000 |
| ENSG00000112851 | ERBB2IP | 0.18 | 0.0030 |
| ENSG00000112893 | MAN2A1 | 0.22 | 0.0004 |
| ENSG00000112977 | DAP | 0.75 | 0.0000 |
| ENSG00000112992 | NNT | 0.57 | 0.0000 |
| ENSG00000113140 | SPARC | 0.81 | 0.0000 |
| ENSG00000113328 | CCNG1 | 0.80 | 0.0000 |
| ENSG00000113441 | LNPEP | 0.24 | 0.0001 |
| ENSG00000113558 | SKP1 | 0.31 | 0.0000 |
| ENSG00000113638 | TTC33 | 0.39 | 0.0000 |
| ENSG00000113712 | CSNK1A1 | 0.37 | 0.0000 |
| ENSG00000113732 | ATP6V0E1 | 0.36 | 0.0000 |
| ENSG00000113742 | CPEB4 | 0.31 | 0.0000 |
| ENSG00000113758 | DBN1 | 0.75 | 0.0000 |
| ENSG00000113761 | ZNF346 | 0.20 | 0.0013 |
| ENSG00000113851 | CRBN | 0.29 | 0.0000 |
| ENSG00000114098 | ARMC8 | 0.71 | 0.0000 |
| ENSG00000114166 | KAT2B | 0.23 | 0.0001 |
| ENSG00000114316 | USP4 | 0.21 | 0.0005 |
| ENSG00000114353 | GNAI2 | 0.62 | 0.0000 |
| ENSG00000114354 | TFG | 0.19 | 0.0026 |
| ENSG00000114383 | TUSC2 | 0.19 | 0.0015 |
| ENSG00000114541 | FRMD4B | 0.31 | 0.0000 |
| ENSG00000114573 | ATP6V1A | 0.26 | 0.0000 |
| ENSG00000114626 | ABTB1 | 0.68 | 0.0000 |
| ENSG00000114770 | ABCC5 | 0.24 | 0.0001 |
| ENSG00000114784 | EIF1B | 0.17 | 0.0050 |
| ENSG00000114805 | PLCH1 | 0.17 | 0.0048 |
| ENSG00000114904 | NEK4 | 0.58 | 0.0000 |
| ENSG00000114978 | MOB1A | 0.55 | 0.0000 |
| ENSG00000114982 | KANSL3 | 0.50 | 0.0000 |
| ENSG00000115159 | GPD2 | 0.50 | 0.0000 |
| ENSG00000115170 | ACVR1 | 0.75 | 0.0000 |
| ENSG00000115216 | NRBP1 | 0.46 | 0.0000 |
| ENSG00000115234 | SNX17 | 0.24 | 0.0001 |
| ENSG00000115290 | GRB14 | 0.26 | 0.0000 |
| ENSG00000115310 | RTN4 | 0.42 | 0.0000 |
| ENSG00000115318 | LOXL3 | 0.73 | 0.0000 |
| ENSG00000115457 | IGFBP2 | 0.36 | 0.0000 |
| ENSG00000115464 | USP34 | 0.18 | 0.0026 |
| ENSG00000115504 | EHBP1 | 0.19 | 0.0022 |
| ENSG00000115641 | FHL2 | 0.48 | 0.0000 |
| ENSG00000115649 | CNPPD1 | 0.31 | 0.0000 |
| ENSG00000115652 | UXS1 | 0.68 | 0.0000 |
| ENSG00000115677 | HDLBP | 0.32 | 0.0000 |
| ENSG00000115756 | HPCAL1 | 0.66 | 0.0000 |
| ENSG00000115758 | ODC1 | 0.66 | 0.0000 |
| ENSG00000115762 | PLEKHB2 | 0.46 | 0.0000 |
| ENSG00000115839 | RAB3GAP1 | 0.22 | 0.0002 |
| ENSG00000115935 | WIPF1 | 0.77 | 0.0000 |
| ENSG00000115956 | PLEK | 0.56 | 0.0000 |
| ENSG00000115966 | ATF2 | 0.27 | 0.0000 |
| ENSG00000115993 | TRAK2 | 0.19 | 0.0017 |
| ENSG00000116001 | TIA1 | 0.44 | 0.0000 |
| ENSG00000116171 | SCP2 | 0.43 | 0.0000 |
| ENSG00000116199 | FAM20B | 0.40 | 0.0000 |
| ENSG00000116260 | QSOX1 | 0.46 | 0.0000 |
| ENSG00000116288 | PARK7 | 0.34 | 0.0000 |
| ENSG00000116337 | AMPD2 | 0.34 | 0.0000 |
| ENSG00000116604 | MEF2D | 0.24 | 0.0001 |
| ENSG00000116678 | LEPR | 0.54 | 0.0000 |
| ENSG00000116679 | IVNS1ABP | 0.43 | 0.0000 |
| ENSG00000116685 | KIAA2013 | 0.28 | 0.0000 |
| ENSG00000116688 | MFN2 | 0.39 | 0.0000 |
| ENSG00000116711 | PLA2G4A | 0.25 | 0.0000 |
| ENSG00000116747 | TROVE2 | 0.41 | 0.0000 |
| ENSG00000116793 | PHTF1 | 0.65 | 0.0000 |
| ENSG00000116857 | TMEM9 | 0.24 | 0.0001 |
| ENSG00000116962 | NID1 | 0.50 | 0.0000 |
| ENSG00000116977 | LGALS8 | 0.49 | 0.0000 |
| ENSG00000116984 | MTR | 0.17 | 0.0062 |
| ENSG00000117153 | KLHL12 | 0.24 | 0.0001 |
| ENSG00000117155 | SSX2IP | 0.56 | 0.0000 |
| ENSG00000117298 | ECE1 | 0.64 | 0.0000 |
| ENSG00000117305 | HMGCL | 0.23 | 0.0002 |
| ENSG00000117362 | APH1A | 0.22 | 0.0002 |
| ENSG00000117400 | MPL | 0.47 | 0.0000 |
| ENSG00000117475 | BLZF1 | 0.19 | 0.0024 |
| ENSG00000117505 | DR1 | 0.37 | 0.0000 |
| ENSG00000117533 | VAMP4 | 0.32 | 0.0000 |
| ENSG00000117586 | TNFSF4 | 0.38 | 0.0000 |
| ENSG00000117592 | PRDX6 | 0.55 | 0.0000 |
| ENSG00000117625 | RCOR3 | 0.30 | 0.0000 |
| ENSG00000117640 | MTFR1L | 0.71 | 0.0000 |
| ENSG00000117691 | NENF | 0.38 | 0.0000 |
| ENSG00000117984 | CTSD | 0.53 | 0.0000 |
| ENSG00000118308 | LRMP | 0.25 | 0.0001 |
| ENSG00000118508 | RAB32 | 0.56 | 0.0000 |
| ENSG00000118705 | RPN2 | 0.22 | 0.0002 |
| ENSG00000118816 | CCNI | 0.39 | 0.0000 |
| ENSG00000118855 | MFSD1 | 0.79 | 0.0000 |
| ENSG00000118900 | UBN1 | 0.30 | 0.0000 |
| ENSG00000119139 | TJP2 | 0.64 | 0.0000 |
| ENSG00000119242 | CCDC92 | 0.70 | 0.0000 |
| ENSG00000119280 | C1orf198 | 0.69 | 0.0000 |
| ENSG00000119326 | CTNNAL1 | 0.45 | 0.0000 |
| ENSG00000119383 | PPP2R4 | 0.55 | 0.0000 |
| ENSG00000119402 | FBXW2 | 0.20 | 0.0010 |
| ENSG00000119632 | IFI27L2 | 0.26 | 0.0000 |
| ENSG00000119718 | EIF2B2 | 0.39 | 0.0000 |
| ENSG00000119801 | YPEL5 | 0.24 | 0.0001 |
| ENSG00000119862 | LGALSL | 0.57 | 0.0000 |
| ENSG00000119899 | SLC17A5 | 0.45 | 0.0000 |
| ENSG00000120008 | WDR11 | 0.29 | 0.0000 |
| ENSG00000120063 | GNA13 | 0.50 | 0.0000 |
| ENSG00000120159 | CAAP1 | 0.18 | 0.0037 |
| ENSG00000120265 | PCMT1 | 0.64 | 0.0000 |
| ENSG00000120594 | PLXDC2 | 0.70 | 0.0000 |
| ENSG00000120727 | PAIP2 | 0.32 | 0.0000 |
| ENSG00000120885 | CLU | 0.74 | 0.0000 |
| ENSG00000120903 | CHRNA2 | 0.43 | 0.0000 |
| ENSG00000120915 | EPHX2 | 0.25 | 0.0000 |
| ENSG00000120992 | LYPLA1 | 0.58 | 0.0000 |
| ENSG00000121579 | NAA50 | 0.27 | 0.0000 |
| ENSG00000121749 | TBC1D15 | 0.27 | 0.0000 |
| ENSG00000121766 | ZCCHC17 | 0.32 | 0.0000 |
| ENSG00000121848 | RNF115 | 0.40 | 0.0000 |
| ENSG00000121964 | GTDC1 | 0.58 | 0.0000 |
| ENSG00000122203 | KIAA1191 | 0.65 | 0.0000 |
| ENSG00000122218 | COPA | 0.54 | 0.0000 |
| ENSG00000122299 | ZC3H7A | 0.18 | 0.0033 |
| ENSG00000122359 | ANXA11 | 0.67 | 0.0000 |
| ENSG00000122490 | PQLC1 | 0.37 | 0.0000 |
| ENSG00000122515 | ZMIZ2 | 0.37 | 0.0000 |
| ENSG00000122545 | SEPT7 | 0.21 | 0.0006 |
| ENSG00000122566 | HNRNPA2B1 | 0.19 | 0.0018 |
| ENSG00000122643 | NT5C3A | 0.32 | 0.0000 |
| ENSG00000122786 | CALD1 | 0.51 | 0.0000 |
| ENSG00000122862 | SRGN | 0.48 | 0.0000 |
| ENSG00000123091 | RNF11 | 0.72 | 0.0000 |
| ENSG00000123104 | ITPR2 | 0.30 | 0.0000 |
| ENSG00000123124 | WWP1 | 0.41 | 0.0000 |
| ENSG00000123159 | GIPC1 | 0.38 | 0.0000 |
| ENSG00000123405 | NFE2 | 0.56 | 0.0000 |
| ENSG00000123416 | TUBA1B | 0.69 | 0.0000 |
| ENSG00000123472 | ATPAF1 | 0.47 | 0.0000 |
| ENSG00000123505 | AMD1 | 0.30 | 0.0000 |
| ENSG00000123739 | PLA2G12A | 0.72 | 0.0000 |
| ENSG00000123908 | AGO2 | 0.23 | 0.0002 |
| ENSG00000124151 | NCOA3 | 0.20 | 0.0014 |
| ENSG00000124164 | VAPB | 0.44 | 0.0000 |
| ENSG00000124193 | SRSF6 | 0.42 | 0.0000 |
| ENSG00000124209 | RAB22A | 0.17 | 0.0053 |
| ENSG00000124214 | STAU1 | 0.32 | 0.0000 |
| ENSG00000124302 | CHST8 | 0.27 | 0.0000 |
| ENSG00000124333 | VAMP7 | 0.63 | 0.0000 |
| ENSG00000124406 | ATP8A1 | 0.21 | 0.0008 |
| ENSG00000124422 | USP22 | 0.42 | 0.0000 |
| ENSG00000124486 | USP9X | 0.40 | 0.0000 |
| ENSG00000124491 | F13A1 | 0.74 | 0.0000 |
| ENSG00000124532 | MRS2 | 0.26 | 0.0000 |
| ENSG00000124535 | WRNIP1 | 0.73 | 0.0000 |
| ENSG00000124570 | SERPINB6 | 0.44 | 0.0000 |
| ENSG00000124588 | NQO2 | 0.21 | 0.0006 |
| ENSG00000124635 | HIST1H2BJ | 0.30 | 0.0000 |
| ENSG00000124702 | KLHDC3 | 0.29 | 0.0000 |
| ENSG00000124733 | MEA1 | 0.47 | 0.0000 |
| ENSG00000124762 | CDKN1A | 0.66 | 0.0000 |
| ENSG00000124772 | CPNE5 | 0.66 | 0.0000 |
| ENSG00000124831 | LRRFIP1 | 0.19 | 0.0019 |
| ENSG00000125257 | ABCC4 | 0.62 | 0.0000 |
| ENSG00000125354 | SEPT6 | 0.66 | 0.0000 |
| ENSG00000125375 | ATP5S | 0.30 | 0.0000 |
| ENSG00000125388 | GRK4 | 0.23 | 0.0002 |
| ENSG00000125457 | MIF4GD | 0.59 | 0.0000 |
| ENSG00000125534 | PPDPF | 0.46 | 0.0000 |
| ENSG00000125676 | THOC2 | 0.22 | 0.0003 |
| ENSG00000125733 | TRIP10 | 0.39 | 0.0000 |
| ENSG00000125734 | GPR108 | 0.59 | 0.0000 |
| ENSG00000125744 | RTN2 | 0.57 | 0.0000 |
| ENSG00000125753 | VASP | 0.37 | 0.0000 |
| ENSG00000125779 | PANK2 | 0.40 | 0.0000 |
| ENSG00000125814 | NAPB | 0.27 | 0.0000 |
| ENSG00000125827 | TMX4 | 0.24 | 0.0001 |
| ENSG00000125863 | MKKS | 0.20 | 0.0012 |
| ENSG00000125868 | DSTN | 0.45 | 0.0000 |
| ENSG00000125869 | LAMP5 | 0.32 | 0.0000 |
| ENSG00000125875 | TBC1D20 | 0.56 | 0.0000 |
| ENSG00000125898 | FAM110A | 0.36 | 0.0000 |
| ENSG00000125952 | MAX | 0.61 | 0.0000 |
| ENSG00000125970 | RALY | 0.62 | 0.0000 |
| ENSG00000126088 | UROD | 0.20 | 0.0012 |
| ENSG00000126091 | ST3GAL3 | 0.75 | 0.0000 |
| ENSG00000126217 | MCF2L | 0.26 | 0.0000 |
| ENSG00000126247 | CAPNS1 | 0.44 | 0.0000 |
| ENSG00000126391 | FRMD8 | 0.25 | 0.0000 |
| ENSG00000126432 | PRDX5 | 0.26 | 0.0000 |
| ENSG00000126458 | RRAS | 0.46 | 0.0000 |
| ENSG00000126581 | BECN1 | 0.65 | 0.0000 |
| ENSG00000126903 | SLC10A3 | 0.72 | 0.0000 |
| ENSG00000127249 | ATP13A4 | 0.25 | 0.0000 |
| ENSG00000127252 | HRASLS | 0.41 | 0.0000 |
| ENSG00000127314 | RAP1B | 0.53 | 0.0000 |
| ENSG00000127511 | SIN3B | 0.29 | 0.0000 |
| ENSG00000127526 | SLC35E1 | 0.61 | 0.0000 |
| ENSG00000127527 | EPS15L1 | 0.56 | 0.0000 |
| ENSG00000127824 | TUBA4A | 0.69 | 0.0000 |
| ENSG00000127831 | VIL1 | 0.74 | 0.0000 |
| ENSG00000127838 | PNKD | 0.60 | 0.0000 |
| ENSG00000127870 | RNF6 | 0.37 | 0.0000 |
| ENSG00000127920 | GNG11 | 0.30 | 0.0000 |
| ENSG00000127947 | PTPN12 | 0.47 | 0.0000 |
| ENSG00000128245 | YWHAH | 0.48 | 0.0000 |
| ENSG00000128266 | GNAZ | 0.72 | 0.0000 |
| ENSG00000128272 | ATF4 | 0.44 | 0.0000 |
| ENSG00000128294 | TPST2 | 0.81 | 0.0000 |
| ENSG00000128309 | MPST | 0.57 | 0.0000 |
| ENSG00000128311 | TST | 0.25 | 0.0000 |
| ENSG00000128578 | STRIP2 | 0.39 | 0.0000 |
| ENSG00000128595 | CALU | 0.61 | 0.0000 |
| ENSG00000128609 | NDUFA5 | 0.22 | 0.0003 |
| ENSG00000128731 | HERC2 | 0.18 | 0.0041 |
| ENSG00000128791 | TWSG1 | 0.37 | 0.0000 |
| ENSG00000128923 | FAM63B | 0.25 | 0.0000 |
| ENSG00000128989 | ARPP19 | 0.24 | 0.0001 |
| ENSG00000129187 | DCTD | 0.21 | 0.0008 |
| ENSG00000129292 | PHF20L1 | 0.51 | 0.0000 |
| ENSG00000129353 | SLC44A2 | 0.86 | 0.0000 |
| ENSG00000129354 | AP1M2 | 0.50 | 0.0000 |
| ENSG00000129355 | CDKN2D | 0.59 | 0.0000 |
| ENSG00000129521 | EGLN3 | 0.49 | 0.0000 |
| ENSG00000129636 | ITFG1 | 0.72 | 0.0000 |
| ENSG00000129657 | SEC14L1 | 0.60 | 0.0000 |
| ENSG00000129691 | ASH2L | 0.34 | 0.0000 |
| ENSG00000129925 | TMEM8A | 0.41 | 0.0000 |
| ENSG00000129968 | ABHD17A | 0.68 | 0.0000 |
| ENSG00000130066 | SAT1 | 0.40 | 0.0000 |
| ENSG00000130119 | GNL3L | 0.25 | 0.0000 |
| ENSG00000130176 | CNN1 | 0.46 | 0.0000 |
| ENSG00000130177 | CDC16 | 0.18 | 0.0038 |
| ENSG00000130201 | EXOC3L2 | 0.36 | 0.0000 |
| ENSG00000130227 | XPO7 | 0.64 | 0.0000 |
| ENSG00000130340 | SNX9 | 0.49 | 0.0000 |
| ENSG00000130402 | ACTN4 | 0.20 | 0.0010 |
| ENSG00000130429 | ARPC1B | 0.64 | 0.0000 |
| ENSG00000130703 | OSBPL2 | 0.28 | 0.0000 |
| ENSG00000130734 | ATG4D | 0.17 | 0.0072 |
| ENSG00000130779 | CLIP1 | 0.23 | 0.0002 |
| ENSG00000130830 | MPP1 | 0.74 | 0.0000 |
| ENSG00000130958 | SLC35D2 | 0.65 | 0.0000 |
| ENSG00000130985 | UBA1 | 0.24 | 0.0001 |
| ENSG00000131069 | ACSS2 | 0.34 | 0.0000 |
| ENSG00000131100 | ATP6V1E1 | 0.46 | 0.0000 |
| ENSG00000131165 | CHMP1A | 0.43 | 0.0000 |
| ENSG00000131171 | SH3BGRL | 0.31 | 0.0000 |
| ENSG00000131188 | PRR7 | 0.54 | 0.0000 |
| ENSG00000131196 | NFATC1 | 0.38 | 0.0000 |
| ENSG00000131236 | CAP1 | 0.76 | 0.0000 |
| ENSG00000131374 | TBC1D5 | 0.19 | 0.0019 |
| ENSG00000131389 | SLC6A6 | 0.41 | 0.0000 |
| ENSG00000131408 | NR1H2 | 0.63 | 0.0000 |
| ENSG00000131504 | DIAPH1 | 0.62 | 0.0000 |
| ENSG00000131634 | TMEM204 | 0.38 | 0.0000 |
| ENSG00000131653 | TRAF7 | 0.43 | 0.0000 |
| ENSG00000131711 | MAP1B | 0.30 | 0.0000 |
| ENSG00000131725 | WDR44 | 0.41 | 0.0000 |
| ENSG00000131778 | CHD1L | 0.71 | 0.0000 |
| ENSG00000131781 | FMO5 | 0.37 | 0.0000 |
| ENSG00000131791 | PRKAB2 | 0.32 | 0.0000 |
| ENSG00000131966 | ACTR10 | 0.49 | 0.0000 |
| ENSG00000132128 | LRRC41 | 0.23 | 0.0002 |
| ENSG00000132155 | RAF1 | 0.31 | 0.0000 |
| ENSG00000132376 | INPP5K | 0.51 | 0.0000 |
| ENSG00000132471 | WBP2 | 0.81 | 0.0000 |
| ENSG00000132478 | UNK | 0.23 | 0.0002 |
| ENSG00000132670 | PTPRA | 0.74 | 0.0000 |
| ENSG00000132819 | RBM38 | 0.51 | 0.0000 |
| ENSG00000132824 | SERINC3 | 0.61 | 0.0000 |
| ENSG00000132906 | CASP9 | 0.49 | 0.0000 |
| ENSG00000132912 | DCTN4 | 0.29 | 0.0000 |
| ENSG00000132970 | WASF3 | 0.32 | 0.0000 |
| ENSG00000133030 | MPRIP | 0.17 | 0.0046 |
| ENSG00000133069 | TMCC2 | 0.56 | 0.0000 |
| ENSG00000133193 | FAM104A | 0.34 | 0.0000 |
| ENSG00000133243 | BTBD2 | 0.49 | 0.0000 |
| ENSG00000133275 | CSNK1G2 | 0.17 | 0.0058 |
| ENSG00000133317 | LGALS12 | 0.59 | 0.0000 |
| ENSG00000133318 | RTN3 | 0.67 | 0.0000 |
| ENSG00000133393 | FOPNL | 0.42 | 0.0000 |
| ENSG00000133606 | MKRN1 | 0.70 | 0.0000 |
| ENSG00000133627 | ACTR3B | 0.57 | 0.0000 |
| ENSG00000133872 | TMEM66 | 0.25 | 0.0001 |
| ENSG00000134070 | IRAK2 | 0.31 | 0.0000 |
| ENSG00000134108 | ARL8B | 0.23 | 0.0002 |
| ENSG00000134198 | TSPAN2 | 0.43 | 0.0000 |
| ENSG00000134202 | GSTM3 | 0.39 | 0.0000 |
| ENSG00000134243 | SORT1 | 0.41 | 0.0000 |
| ENSG00000134278 | SPIRE1 | 0.33 | 0.0000 |
| ENSG00000134291 | TMEM106C | 0.43 | 0.0000 |
| ENSG00000134297 | PLEKHA8P1 | 0.35 | 0.0000 |
| ENSG00000134308 | YWHAQ | 0.64 | 0.0000 |
| ENSG00000134313 | KIDINS220 | 0.35 | 0.0000 |
| ENSG00000134318 | ROCK2 | 0.43 | 0.0000 |
| ENSG00000134352 | IL6ST | 0.53 | 0.0000 |
| ENSG00000134452 | FBXO18 | 0.27 | 0.0000 |
| ENSG00000134548 | C12orf39 | 0.64 | 0.0000 |
| ENSG00000134602 | | 0.39 | 0.0000 |
| ENSG00000134668 | SPOCD1 | 0.51 | 0.0000 |
| ENSG00000134779 | TPGS2 | 0.58 | 0.0000 |
| ENSG00000134824 | FADS2 | 0.40 | 0.0000 |
| ENSG00000134882 | UBAC2 | 0.72 | 0.0000 |
| ENSG00000134900 | TPP2 | 0.21 | 0.0006 |
| ENSG00000134909 | ARHGAP32 | 0.47 | 0.0000 |
| ENSG00000134986 | NREP | 0.52 | 0.0000 |
| ENSG00000134996 | OSTF1 | 0.22 | 0.0004 |
| ENSG00000135070 | ISCA1 | 0.44 | 0.0000 |
| ENSG00000135083 | CCNJL | 0.51 | 0.0000 |
| ENSG00000135090 | TAOK3 | 0.46 | 0.0000 |
| ENSG00000135148 | TRAFD1 | 0.18 | 0.0040 |
| ENSG00000135218 | CD36 | 0.66 | 0.0000 |
| ENSG00000135334 | AKIRIN2 | 0.37 | 0.0000 |
| ENSG00000135404 | CD63 | 0.22 | 0.0004 |
| ENSG00000135604 | STX11 | 0.40 | 0.0000 |
| ENSG00000135709 | KIAA0513 | 0.66 | 0.0000 |
| ENSG00000135776 | ABCB10 | 0.31 | 0.0000 |
| ENSG00000135821 | GLUL | 0.61 | 0.0000 |
| ENSG00000135838 | NPL | 0.43 | 0.0000 |
| ENSG00000135862 | LAMC1 | 0.28 | 0.0000 |
| ENSG00000135919 | SERPINE2 | 0.39 | 0.0000 |
| ENSG00000135926 | TMBIM1 | 0.79 | 0.0000 |
| ENSG00000135932 | CAB39 | 0.52 | 0.0000 |
| ENSG00000136003 | ISCU | 0.30 | 0.0000 |
| ENSG00000136021 | SCYL2 | 0.60 | 0.0000 |
| ENSG00000136048 | DRAM1 | 0.27 | 0.0000 |
| ENSG00000136156 | ITM2B | 0.58 | 0.0000 |
| ENSG00000136205 | TNS3 | 0.18 | 0.0041 |
| ENSG00000136231 | IGF2BP3 | 0.25 | 0.0000 |
| ENSG00000136238 | RAC1 | 0.25 | 0.0000 |
| ENSG00000136247 | ZDHHC4 | 0.52 | 0.0000 |
| ENSG00000136279 | DBNL | 0.81 | 0.0000 |
| ENSG00000136280 | CCM2 | 0.36 | 0.0000 |
| ENSG00000136295 | TTYH3 | 0.54 | 0.0000 |
| ENSG00000136404 | TM6SF1 | 0.43 | 0.0000 |
| ENSG00000136478 | TEX2 | 0.35 | 0.0000 |
| ENSG00000136731 | UGGT1 | 0.20 | 0.0009 |
| ENSG00000136754 | ABI1 | 0.43 | 0.0000 |
| ENSG00000136758 | YME1L1 | 0.17 | 0.0055 |
| ENSG00000136811 | ODF2 | 0.20 | 0.0008 |
| ENSG00000136854 | STXBP1 | 0.34 | 0.0000 |
| ENSG00000136856 | SLC2A8 | 0.31 | 0.0000 |
| ENSG00000136861 | CDK5RAP2 | 0.18 | 0.0029 |
| ENSG00000136878 | USP20 | 0.24 | 0.0001 |
| ENSG00000137075 | RNF38 | 0.30 | 0.0000 |
| ENSG00000137076 | TLN1 | 0.62 | 0.0000 |
| ENSG00000137145 | DENND4C | 0.49 | 0.0000 |
| ENSG00000137198 | GMPR | 0.75 | 0.0000 |
| ENSG00000137207 | YIPF3 | 0.46 | 0.0000 |
| ENSG00000137225 | CAPN11 | 0.48 | 0.0000 |
| ENSG00000137266 | SLC22A23 | 0.50 | 0.0000 |
| ENSG00000137312 | FLOT1 | 0.19 | 0.0018 |
| ENSG00000137449 | CPEB2 | 0.31 | 0.0000 |
| ENSG00000137486 | ARRB1 | 0.73 | 0.0000 |
| ENSG00000137672 | TRPC6 | 0.42 | 0.0000 |
| ENSG00000137801 | THBS1 | 0.73 | 0.0000 |
| ENSG00000137817 | PARP6 | 0.25 | 0.0000 |
| ENSG00000137822 | TUBGCP4 | 0.46 | 0.0000 |
| ENSG00000137831 | UACA | 0.24 | 0.0001 |
| ENSG00000137845 | ADAM10 | 0.35 | 0.0000 |
| ENSG00000137941 | TTLL7 | 0.44 | 0.0000 |
| ENSG00000137942 | FNBP1L | 0.40 | 0.0000 |
| ENSG00000138031 | ADCY3 | 0.60 | 0.0000 |
| ENSG00000138071 | ACTR2 | 0.19 | 0.0018 |
| ENSG00000138107 | ACTR1A | 0.25 | 0.0000 |
| ENSG00000138279 | ANXA7 | 0.62 | 0.0000 |
| ENSG00000138293 | NCOA4 | 0.55 | 0.0000 |
| ENSG00000138434 | SSFA2 | 0.58 | 0.0000 |
| ENSG00000138443 | ABI2 | 0.64 | 0.0000 |
| ENSG00000138449 | SLC40A1 | 0.49 | 0.0000 |
| ENSG00000138594 | TMOD3 | 0.42 | 0.0000 |
| ENSG00000138642 | HERC6 | 0.18 | 0.0036 |
| ENSG00000138698 | RAP1GDS1 | 0.41 | 0.0000 |
| ENSG00000138722 | MMRN1 | 0.71 | 0.0000 |
| ENSG00000138735 | PDE5A | 0.50 | 0.0000 |
| ENSG00000138756 | BMP2K | 0.31 | 0.0000 |
| ENSG00000138757 | G3BP2 | 0.47 | 0.0000 |
| ENSG00000138758 | SEPT11 | 0.33 | 0.0000 |
| ENSG00000138794 | CASP6 | 0.43 | 0.0000 |
| ENSG00000138796 | HADH | 0.45 | 0.0000 |
| ENSG00000138798 | EGF | 0.78 | 0.0000 |
| ENSG00000138801 | PAPSS1 | 0.41 | 0.0000 |
| ENSG00000138821 | SLC39A8 | 0.23 | 0.0002 |
| ENSG00000138835 | RGS3 | 0.21 | 0.0007 |
| ENSG00000138867 | GUCD1 | 0.47 | 0.0000 |
| ENSG00000139083 | ETV6 | 0.46 | 0.0000 |
| ENSG00000139323 | POC1B | 0.42 | 0.0000 |
| ENSG00000139433 | GLTP | 0.20 | 0.0008 |
| ENSG00000139597 | N4BP2L1 | 0.40 | 0.0000 |
| ENSG00000139644 | TMBIM6 | 0.48 | 0.0000 |
| ENSG00000139722 | VPS37B | 0.39 | 0.0000 |
| ENSG00000139835 | GRTP1 | 0.36 | 0.0000 |
| ENSG00000139946 | PELI2 | 0.53 | 0.0000 |
| ENSG00000139970 | RTN1 | 0.58 | 0.0000 |
| ENSG00000139990 | DCAF5 | 0.46 | 0.0000 |
| ENSG00000140022 | STON2 | 0.53 | 0.0000 |
| ENSG00000140299 | BNIP2 | 0.35 | 0.0000 |
| ENSG00000140374 | ETFA | 0.48 | 0.0000 |
| ENSG00000140416 | TPM1 | 0.60 | 0.0000 |
| ENSG00000140443 | IGF1R | 0.33 | 0.0000 |
| ENSG00000140474 | ULK3 | 0.19 | 0.0019 |
| ENSG00000140479 | PCSK6 | 0.84 | 0.0000 |
| ENSG00000140497 | SCAMP2 | 0.23 | 0.0002 |
| ENSG00000140553 | UNC45A | 0.76 | 0.0000 |
| ENSG00000140564 | FURIN | 0.59 | 0.0000 |
| ENSG00000140632 | GLYR1 | 0.40 | 0.0000 |
| ENSG00000140682 | TGFB1I1 | 0.76 | 0.0000 |
| ENSG00000140830 | TXNL4B | 0.47 | 0.0000 |
| ENSG00000140848 | CPNE2 | 0.52 | 0.0000 |
| ENSG00000140854 | KATNB1 | 0.57 | 0.0000 |
| ENSG00000140859 | KIFC3 | 0.71 | 0.0000 |
| ENSG00000140931 | CMTM3 | 0.41 | 0.0000 |
| ENSG00000140932 | CMTM2 | 0.30 | 0.0000 |
| ENSG00000140941 | MAP1LC3B | 0.35 | 0.0000 |
| ENSG00000141027 | NCOR1 | 0.43 | 0.0000 |
| ENSG00000141030 | COPS3 | 0.27 | 0.0000 |
| ENSG00000141179 | PCTP | 0.46 | 0.0000 |
| ENSG00000141198 | TOM1L1 | 0.45 | 0.0000 |
| ENSG00000141279 | NPEPPS | 0.49 | 0.0000 |
| ENSG00000141429 | GALNT1 | 0.32 | 0.0000 |
| ENSG00000141452 | C18orf8 | 0.52 | 0.0000 |
| ENSG00000141503 | MINK1 | 0.65 | 0.0000 |
| ENSG00000141580 | WDR45B | 0.50 | 0.0000 |
| ENSG00000141759 | TXNL4A | 0.32 | 0.0000 |
| ENSG00000141854 | | 0.18 | 0.0034 |
| ENSG00000141959 | PFKL | 0.54 | 0.0000 |
| ENSG00000142002 | DPP9 | 0.27 | 0.0000 |
| ENSG00000142046 | TMEM91 | 0.24 | 0.0001 |
| ENSG00000142192 | APP | 0.53 | 0.0000 |
| ENSG00000142208 | AKT1 | 0.40 | 0.0000 |
| ENSG00000142327 | RNPEPL1 | 0.63 | 0.0000 |
| ENSG00000142657 | PGD | 0.74 | 0.0000 |
| ENSG00000142669 | SH3BGRL3 | 0.45 | 0.0000 |
| ENSG00000142694 | EVA1B | 0.34 | 0.0000 |
| ENSG00000142794 | NBPF3 | 0.30 | 0.0000 |
| ENSG00000142875 | PRKACB | 0.45 | 0.0000 |
| ENSG00000142892 | PIGK | 0.24 | 0.0001 |
| ENSG00000142949 | PTPRF | 0.33 | 0.0000 |
| ENSG00000142961 | MOB3C | 0.46 | 0.0000 |
| ENSG00000143149 | ALDH9A1 | 0.32 | 0.0000 |
| ENSG00000143158 | MPC2 | 0.27 | 0.0000 |
| ENSG00000143162 | CREG1 | 0.55 | 0.0000 |
| ENSG00000143164 | DCAF6 | 0.49 | 0.0000 |
| ENSG00000143226 | FCGR2A | 0.47 | 0.0000 |
| ENSG00000143321 | HDGF | 0.59 | 0.0000 |
| ENSG00000143324 | XPR1 | 0.25 | 0.0000 |
| ENSG00000143353 | LYPLAL1 | 0.23 | 0.0002 |
| ENSG00000143363 | PRUNE | 0.76 | 0.0000 |
| ENSG00000143409 | FAM63A | 0.79 | 0.0000 |
| ENSG00000143418 | CERS2 | 0.81 | 0.0000 |
| ENSG00000143437 | ARNT | 0.27 | 0.0000 |
| ENSG00000143499 | SMYD2 | 0.67 | 0.0000 |
| ENSG00000143545 | RAB13 | 0.51 | 0.0000 |
| ENSG00000143549 | TPM3 | 0.64 | 0.0000 |
| ENSG00000143595 | AQP10 | 0.68 | 0.0000 |
| ENSG00000143612 | C1orf43 | 0.53 | 0.0000 |
| ENSG00000143622 | RIT1 | 0.37 | 0.0000 |
| ENSG00000143641 | GALNT2 | 0.34 | 0.0000 |
| ENSG00000143727 | ACP1 | 0.26 | 0.0000 |
| ENSG00000143761 | ARF1 | 0.76 | 0.0000 |
| ENSG00000143776 | CDC42BPA | 0.34 | 0.0000 |
| ENSG00000143797 | MBOAT2 | 0.66 | 0.0000 |
| ENSG00000143889 | HNRNPLL | 0.45 | 0.0000 |
| ENSG00000143891 | GALM | 0.49 | 0.0000 |
| ENSG00000143952 | VPS54 | 0.22 | 0.0003 |
| ENSG00000143995 | MEIS1 | 0.44 | 0.0000 |
| ENSG00000144118 | RALB | 0.23 | 0.0001 |
| ENSG00000144455 | SUMF1 | 0.23 | 0.0002 |
| ENSG00000144468 | RHBDD1 | 0.61 | 0.0000 |
| ENSG00000144560 | VGLL4 | 0.59 | 0.0000 |
| ENSG00000144567 | FAM134A | 0.40 | 0.0000 |
| ENSG00000144579 | CTDSP1 | 0.25 | 0.0001 |
| ENSG00000144677 | CTDSPL | 0.66 | 0.0000 |
| ENSG00000144746 | ARL6IP5 | 0.27 | 0.0000 |
| ENSG00000144893 | MED12L | 0.47 | 0.0000 |
| ENSG00000145022 | TCTA | 0.70 | 0.0000 |
| ENSG00000145335 | SNCA | 0.60 | 0.0000 |
| ENSG00000145431 | PDGFC | 0.50 | 0.0000 |
| ENSG00000145685 | LHFPL2 | 0.46 | 0.0000 |
| ENSG00000145687 | SSBP2 | 0.41 | 0.0000 |
| ENSG00000145703 | IQGAP2 | 0.43 | 0.0000 |
| ENSG00000145730 | PAM | 0.35 | 0.0000 |
| ENSG00000145740 | SLC30A5 | 0.52 | 0.0000 |
| ENSG00000145916 | RMND5B | 0.18 | 0.0042 |
| ENSG00000145982 | FARS2 | 0.21 | 0.0005 |
| ENSG00000146094 | DOK3 | 0.44 | 0.0000 |
| ENSG00000146112 | PPP1R18 | 0.43 | 0.0000 |
| ENSG00000146376 | ARHGAP18 | 0.52 | 0.0000 |
| ENSG00000146416 | AIG1 | 0.50 | 0.0000 |
| ENSG00000146535 | GNA12 | 0.60 | 0.0000 |
| ENSG00000146540 | C7orf50 | 0.36 | 0.0000 |
| ENSG00000146834 | MEPCE | 0.49 | 0.0000 |
| ENSG00000146858 | ZC3HAV1L | 0.27 | 0.0000 |
| ENSG00000146859 | TMEM140 | 0.41 | 0.0000 |
| ENSG00000146963 | C7orf55-LUC7L2 | 0.37 | 0.0000 |
| ENSG00000147036 | LANCL3 | 0.54 | 0.0000 |
| ENSG00000147065 | MSN | 0.68 | 0.0000 |
| ENSG00000147394 | ZNF185 | 0.83 | 0.0000 |
| ENSG00000147400 | CETN2 | 0.38 | 0.0000 |
| ENSG00000147443 | DOK2 | 0.72 | 0.0000 |
| ENSG00000147526 | TACC1 | 0.47 | 0.0000 |
| ENSG00000147535 | PPAPDC1B | 0.19 | 0.0022 |
| ENSG00000147650 | LRP12 | 0.45 | 0.0000 |
| ENSG00000147804 | SLC39A4 | 0.24 | 0.0001 |
| ENSG00000147853 | AK3 | 0.54 | 0.0000 |
| ENSG00000147854 | UHRF2 | 0.39 | 0.0000 |
| ENSG00000147862 | NFIB | 0.45 | 0.0000 |
| ENSG00000148120 | C9orf3 | 0.45 | 0.0000 |
| ENSG00000148175 | STOM | 0.79 | 0.0000 |
| ENSG00000148180 | GSN | 0.78 | 0.0000 |
| ENSG00000148248 | SURF4 | 0.21 | 0.0005 |
| ENSG00000148341 | SH3GLB2 | 0.27 | 0.0000 |
| ENSG00000148343 | FAM73B | 0.60 | 0.0000 |
| ENSG00000148426 | PROSER2 | 0.51 | 0.0000 |
| ENSG00000148484 | RSU1 | 0.46 | 0.0000 |
| ENSG00000148488 | ST8SIA6 | 0.24 | 0.0001 |
| ENSG00000148498 | PARD3 | 0.60 | 0.0000 |
| ENSG00000148700 | ADD3 | 0.66 | 0.0000 |
| ENSG00000148834 | GSTO1 | 0.46 | 0.0000 |
| ENSG00000148908 | RGS10 | 0.32 | 0.0000 |
| ENSG00000149084 | HSD17B12 | 0.42 | 0.0000 |
| ENSG00000149091 | DGKZ | 0.29 | 0.0000 |
| ENSG00000149131 | SERPING1 | 0.32 | 0.0000 |
| ENSG00000149177 | PTPRJ | 0.48 | 0.0000 |
| ENSG00000149179 | C11orf49 | 0.52 | 0.0000 |
| ENSG00000149218 | ENDOD1 | 0.68 | 0.0000 |
| ENSG00000149243 | KLHL35 | 0.27 | 0.0000 |
| ENSG00000149357 | LAMTOR1 | 0.61 | 0.0000 |
| ENSG00000149476 | DAK | 0.36 | 0.0000 |
| ENSG00000149485 | FADS1 | 0.46 | 0.0000 |
| ENSG00000149564 | ESAM | 0.83 | 0.0000 |
| ENSG00000149600 | COMMD7 | 0.44 | 0.0000 |
| ENSG00000149781 | FERMT3 | 0.85 | 0.0000 |
| ENSG00000149925 | ALDOA | 0.66 | 0.0000 |
| ENSG00000149932 | TMEM219 | 0.46 | 0.0000 |
| ENSG00000150054 | MPP7 | 0.17 | 0.0072 |
| ENSG00000150093 | ITGB1 | 0.79 | 0.0000 |
| ENSG00000150403 | TMCO3 | 0.41 | 0.0000 |
| ENSG00000150637 | CD226 | 0.62 | 0.0000 |
| ENSG00000150681 | RGS18 | 0.23 | 0.0002 |
| ENSG00000150712 | MTMR12 | 0.59 | 0.0000 |
| ENSG00000150867 | PIP4K2A | 0.56 | 0.0000 |
| ENSG00000150991 | UBC | 0.20 | 0.0009 |
| ENSG00000150995 | ITPR1 | 0.17 | 0.0057 |
| ENSG00000151136 | BTBD11 | 0.21 | 0.0007 |
| ENSG00000151148 | UBE3B | 0.54 | 0.0000 |
| ENSG00000151247 | EIF4E | 0.39 | 0.0000 |
| ENSG00000151327 | FAM177A1 | 0.20 | 0.0010 |
| ENSG00000151414 | NEK7 | 0.40 | 0.0000 |
| ENSG00000151502 | VPS26B | 0.50 | 0.0000 |
| ENSG00000151665 | PIGF | 0.17 | 0.0056 |
| ENSG00000151690 | MFSD6 | 0.46 | 0.0000 |
| ENSG00000151693 | ASAP2 | 0.44 | 0.0000 |
| ENSG00000151702 | FLI1 | 0.43 | 0.0000 |
| ENSG00000151743 | AMN1 | 0.27 | 0.0000 |
| ENSG00000151748 | SAV1 | 0.53 | 0.0000 |
| ENSG00000151779 | NBAS | 0.28 | 0.0000 |
| ENSG00000152061 | RABGAP1L | 0.34 | 0.0000 |
| ENSG00000152128 | TMEM163 | 0.28 | 0.0000 |
| ENSG00000152229 | PSTPIP2 | 0.58 | 0.0000 |
| ENSG00000152256 | PDK1 | 0.51 | 0.0000 |
| ENSG00000152291 | TGOLN2 | 0.32 | 0.0000 |
| ENSG00000152332 | UHMK1 | 0.31 | 0.0000 |
| ENSG00000152484 | USP12 | 0.39 | 0.0000 |
| ENSG00000152556 | PFKM | 0.57 | 0.0000 |
| ENSG00000152601 | MBNL1 | 0.61 | 0.0000 |
| ENSG00000152620 | NADK2 | 0.22 | 0.0003 |
| ENSG00000152642 | GPD1L | 0.44 | 0.0000 |
| ENSG00000152952 | PLOD2 | 0.61 | 0.0000 |
| ENSG00000153064 | BANK1 | 0.38 | 0.0000 |
| ENSG00000153071 | DAB2 | 0.47 | 0.0000 |
| ENSG00000153162 | BMP6 | 0.60 | 0.0000 |
| ENSG00000153214 | TMEM87B | 0.18 | 0.0029 |
| ENSG00000153317 | ASAP1 | 0.41 | 0.0000 |
| ENSG00000153561 | RMND5A | 0.42 | 0.0000 |
| ENSG00000153815 | CMIP | 0.32 | 0.0000 |
| ENSG00000153827 | TRIP12 | 0.50 | 0.0000 |
| ENSG00000154122 | ANKH | 0.19 | 0.0025 |
| ENSG00000154127 | UBASH3B | 0.39 | 0.0000 |
| ENSG00000154146 | NRGN | 0.52 | 0.0000 |
| ENSG00000154188 | ANGPT1 | 0.27 | 0.0000 |
| ENSG00000154229 | PRKCA | 0.28 | 0.0000 |
| ENSG00000154310 | TNIK | 0.37 | 0.0000 |
| ENSG00000154917 | RAB6B | 0.47 | 0.0000 |
| ENSG00000154978 | VOPP1 | 0.41 | 0.0000 |
| ENSG00000155096 | AZIN1 | 0.63 | 0.0000 |
| ENSG00000155099 | TMEM55A | 0.46 | 0.0000 |
| ENSG00000155115 | GTF3C6 | 0.26 | 0.0000 |
| ENSG00000155158 | TTC39B | 0.47 | 0.0000 |
| ENSG00000155366 | RHOC | 0.48 | 0.0000 |
| ENSG00000155729 | KCTD18 | 0.43 | 0.0000 |
| ENSG00000155849 | ELMO1 | 0.38 | 0.0000 |
| ENSG00000155975 | VPS37A | 0.35 | 0.0000 |
| ENSG00000155984 | TMEM185A | 0.72 | 0.0000 |
| ENSG00000156011 | PSD3 | 0.44 | 0.0000 |
| ENSG00000156026 | MCU | 0.48 | 0.0000 |
| ENSG00000156052 | GNAQ | 0.72 | 0.0000 |
| ENSG00000156136 | DCK | 0.20 | 0.0012 |
| ENSG00000156206 | C15orf26 | 0.53 | 0.0000 |
| ENSG00000156381 | ANKRD9 | 0.69 | 0.0000 |
| ENSG00000156504 | FAM122B | 0.40 | 0.0000 |
| ENSG00000156515 | HK1 | 0.73 | 0.0000 |
| ENSG00000156535 | CD109 | 0.45 | 0.0000 |
| ENSG00000156639 | ZFAND3 | 0.51 | 0.0000 |
| ENSG00000156642 | NPTN | 0.59 | 0.0000 |
| ENSG00000156860 | FBRS | 0.47 | 0.0000 |
| ENSG00000156875 | HIAT1 | 0.22 | 0.0003 |
| ENSG00000156931 | VPS8 | 0.47 | 0.0000 |
| ENSG00000157045 | NTAN1 | 0.51 | 0.0000 |
| ENSG00000157216 | SSBP3 | 0.27 | 0.0000 |
| ENSG00000157303 | SUSD3 | 0.58 | 0.0000 |
| ENSG00000157514 | TSC22D3 | 0.63 | 0.0000 |
| ENSG00000157538 | DSCR3 | 0.50 | 0.0000 |
| ENSG00000157570 | TSPAN18 | 0.46 | 0.0000 |
| ENSG00000157600 | TMEM164 | 0.33 | 0.0000 |
| ENSG00000157837 | SPPL3 | 0.70 | 0.0000 |
| ENSG00000157978 | LDLRAP1 | 0.74 | 0.0000 |
| ENSG00000158019 | BRE | 0.45 | 0.0000 |
| ENSG00000158109 | TPRG1L | 0.48 | 0.0000 |
| ENSG00000158290 | CUL4B | 0.18 | 0.0033 |
| ENSG00000158457 | TSPAN33 | 0.81 | 0.0000 |
| ENSG00000158552 | ZFAND2B | 0.42 | 0.0000 |
| ENSG00000158560 | DYNC1I1 | 0.46 | 0.0000 |
| ENSG00000158604 | TMED4 | 0.34 | 0.0000 |
| ENSG00000158710 | TAGLN2 | 0.75 | 0.0000 |
| ENSG00000158793 | NIT1 | 0.21 | 0.0005 |
| ENSG00000158796 | DEDD | 0.64 | 0.0000 |
| ENSG00000158856 | DMTN | 0.62 | 0.0000 |
| ENSG00000158869 | FCER1G | 0.23 | 0.0001 |
| ENSG00000158985 | CDC42SE2 | 0.29 | 0.0000 |
| ENSG00000159023 | EPB41 | 0.17 | 0.0071 |
| ENSG00000159069 | FBXW5 | 0.81 | 0.0000 |
| ENSG00000159176 | CSRP1 | 0.57 | 0.0000 |
| ENSG00000159202 | UBE2Z | 0.32 | 0.0000 |
| ENSG00000159335 | PTMS | 0.29 | 0.0000 |
| ENSG00000159339 | PADI4 | 0.23 | 0.0001 |
| ENSG00000159346 | ADIPOR1 | 0.74 | 0.0000 |
| ENSG00000159348 | CYB5R1 | 0.76 | 0.0000 |
| ENSG00000159363 | ATP13A2 | 0.29 | 0.0000 |
| ENSG00000159461 | AMFR | 0.69 | 0.0000 |
| ENSG00000159592 | GPBP1L1 | 0.28 | 0.0000 |
| ENSG00000159593 | NAE1 | 0.20 | 0.0009 |
| ENSG00000159625 | CCDC135 | 0.41 | 0.0000 |
| ENSG00000159658 | EFCAB14 | 0.45 | 0.0000 |
| ENSG00000159692 | CTBP1 | 0.24 | 0.0001 |
| ENSG00000159720 | ATP6V0D1 | 0.28 | 0.0000 |
| ENSG00000159840 | ZYX | 0.77 | 0.0000 |
| ENSG00000160013 | PTGIR | 0.59 | 0.0000 |
| ENSG00000160014 | CALM3 | 0.41 | 0.0000 |
| ENSG00000160058 | BSDC1 | 0.68 | 0.0000 |
| ENSG00000160145 | KALRN | 0.35 | 0.0000 |
| ENSG00000160188 | RSPH1 | 0.32 | 0.0000 |
| ENSG00000160190 | SLC37A1 | 0.58 | 0.0000 |
| ENSG00000160211 | G6PD | 0.47 | 0.0000 |
| ENSG00000160221 | C21orf33 | 0.19 | 0.0020 |
| ENSG00000160298 | C21orf58 | 0.49 | 0.0000 |
| ENSG00000160310 | PRMT2 | 0.69 | 0.0000 |
| ENSG00000160410 | SHKBP1 | 0.27 | 0.0000 |
| ENSG00000160445 | ZER1 | 0.60 | 0.0000 |
| ENSG00000160446 | ZDHHC12 | 0.22 | 0.0003 |
| ENSG00000160613 | PCSK7 | 0.33 | 0.0000 |
| ENSG00000160691 | SHC1 | 0.37 | 0.0000 |
| ENSG00000160703 | NLRX1 | 0.47 | 0.0000 |
| ENSG00000160714 | UBE2Q1 | 0.42 | 0.0000 |
| ENSG00000160789 | LMNA | 0.80 | 0.0000 |
| ENSG00000160796 | NBEAL2 | 0.17 | 0.0062 |
| ENSG00000160948 | VPS28 | 0.26 | 0.0000 |
| ENSG00000160991 | ORAI2 | 0.70 | 0.0000 |
| ENSG00000160999 | SH2B2 | 0.32 | 0.0000 |
| ENSG00000161011 | SQSTM1 | 0.69 | 0.0000 |
| ENSG00000161013 | MGAT4B | 0.69 | 0.0000 |
| ENSG00000161202 | DVL3 | 0.17 | 0.0058 |
| ENSG00000161203 | AP2M1 | 0.87 | 0.0000 |
| ENSG00000161547 | SRSF2 | 0.29 | 0.0000 |
| ENSG00000161570 | CCL5 | 0.30 | 0.0000 |
| ENSG00000161911 | TREML1 | 0.74 | 0.0000 |
| ENSG00000161921 | CXCL16 | 0.17 | 0.0051 |
| ENSG00000161999 | JMJD8 | 0.53 | 0.0000 |
| ENSG00000162368 | CMPK1 | 0.73 | 0.0000 |
| ENSG00000162434 | JAK1 | 0.23 | 0.0002 |
| ENSG00000162511 | LAPTM5 | 0.17 | 0.0067 |
| ENSG00000162517 | PEF1 | 0.43 | 0.0000 |
| ENSG00000162521 | RBBP4 | 0.25 | 0.0000 |
| ENSG00000162704 | ARPC5 | 0.49 | 0.0000 |
| ENSG00000162722 | TRIM58 | 0.77 | 0.0000 |
| ENSG00000162852 | CNST | 0.40 | 0.0000 |
| ENSG00000162909 | CAPN2 | 0.78 | 0.0000 |
| ENSG00000162923 | WDR26 | 0.52 | 0.0000 |
| ENSG00000163041 | H3F3A | 0.18 | 0.0029 |
| ENSG00000163110 | PDLIM5 | 0.50 | 0.0000 |
| ENSG00000163297 | ANTXR2 | 0.24 | 0.0001 |
| ENSG00000163320 | CGGBP1 | 0.32 | 0.0000 |
| ENSG00000163344 | PMVK | 0.20 | 0.0014 |
| ENSG00000163349 | HIPK1 | 0.18 | 0.0027 |
| ENSG00000163359 | COL6A3 | 0.53 | 0.0000 |
| ENSG00000163374 | YY1AP1 | 0.39 | 0.0000 |
| ENSG00000163430 | FSTL1 | 0.61 | 0.0000 |
| ENSG00000163444 | TMEM183A | 0.19 | 0.0022 |
| ENSG00000163466 | ARPC2 | 0.18 | 0.0031 |
| ENSG00000163536 | SERPINI1 | 0.23 | 0.0002 |
| ENSG00000163634 | THOC7 | 0.18 | 0.0034 |
| ENSG00000163681 | SLMAP | 0.29 | 0.0000 |
| ENSG00000163703 | CRELD1 | 0.37 | 0.0000 |
| ENSG00000163734 | CXCL3 | 0.36 | 0.0000 |
| ENSG00000163735 | CXCL5 | 0.46 | 0.0000 |
| ENSG00000163736 | PPBP | 0.41 | 0.0000 |
| ENSG00000163737 | PF4 | 0.26 | 0.0000 |
| ENSG00000163738 | MTHFD2L | 0.38 | 0.0000 |
| ENSG00000163743 | RCHY1 | 0.27 | 0.0000 |
| ENSG00000163812 | ZDHHC3 | 0.42 | 0.0000 |
| ENSG00000163898 | LIPH | 0.40 | 0.0000 |
| ENSG00000163930 | BAP1 | 0.43 | 0.0000 |
| ENSG00000163932 | PRKCD | 0.64 | 0.0000 |
| ENSG00000163950 | SLBP | 0.38 | 0.0000 |
| ENSG00000164088 | PPM1M | 0.17 | 0.0048 |
| ENSG00000164096 | C4orf3 | 0.17 | 0.0058 |
| ENSG00000164116 | GUCY1A3 | 0.68 | 0.0000 |
| ENSG00000164118 | CEP44 | 0.27 | 0.0000 |
| ENSG00000164120 | HPGD | 0.25 | 0.0000 |
| ENSG00000164171 | ITGA2 | 0.36 | 0.0000 |
| ENSG00000164181 | ELOVL7 | 0.65 | 0.0000 |
| ENSG00000164305 | CASP3 | 0.41 | 0.0000 |
| ENSG00000164506 | STXBP5 | 0.39 | 0.0000 |
| ENSG00000164574 | GALNT10 | 0.50 | 0.0000 |
| ENSG00000164659 | KIAA1324L | 0.23 | 0.0001 |
| ENSG00000164924 | YWHAZ | 0.66 | 0.0000 |
| ENSG00000165006 | UBAP1 | 0.56 | 0.0000 |
| ENSG00000165025 | SYK | 0.31 | 0.0000 |
| ENSG00000165119 | HNRNPK | 0.40 | 0.0000 |
| ENSG00000165169 | DYNLT3 | 0.39 | 0.0000 |
| ENSG00000165233 | C9orf89 | 0.41 | 0.0000 |
| ENSG00000165244 | ZNF367 | 0.41 | 0.0000 |
| ENSG00000165280 | VCP | 0.58 | 0.0000 |
| ENSG00000165309 | ARMC3 | 0.29 | 0.0000 |
| ENSG00000165389 | SPTSSA | 0.29 | 0.0000 |
| ENSG00000165406 | MARCH8 | 0.22 | 0.0003 |
| ENSG00000165434 | PGM2L1 | 0.40 | 0.0000 |
| ENSG00000165458 | INPPL1 | 0.43 | 0.0000 |
| ENSG00000165475 | CRYL1 | 0.66 | 0.0000 |
| ENSG00000165476 | REEP3 | 0.17 | 0.0070 |
| ENSG00000165637 | VDAC2 | 0.29 | 0.0000 |
| ENSG00000165646 | SLC18A2 | 0.50 | 0.0000 |
| ENSG00000165682 | CLEC1B | 0.42 | 0.0000 |
| ENSG00000165702 | GFI1B | 0.67 | 0.0000 |
| ENSG00000165775 | FUNDC2 | 0.33 | 0.0000 |
| ENSG00000165914 | TTC7B | 0.86 | 0.0000 |
| ENSG00000165929 | TC2N | 0.39 | 0.0000 |
| ENSG00000165948 | IFI27L1 | 0.31 | 0.0000 |
| ENSG00000165959 | CLMN | 0.21 | 0.0007 |
| ENSG00000166035 | LIPC | 0.34 | 0.0000 |
| ENSG00000166086 | JAM3 | 0.65 | 0.0000 |
| ENSG00000166091 | CMTM5 | 0.63 | 0.0000 |
| ENSG00000166165 | CKB | 0.31 | 0.0000 |
| ENSG00000166171 | DPCD | 0.36 | 0.0000 |
| ENSG00000166311 | SMPD1 | 0.38 | 0.0000 |
| ENSG00000166337 | TAF10 | 0.25 | 0.0001 |
| ENSG00000166340 | TPP1 | 0.87 | 0.0000 |
| ENSG00000166452 | AKIP1 | 0.27 | 0.0000 |
| ENSG00000166483 | WEE1 | 0.25 | 0.0000 |
| ENSG00000166501 | PRKCB | 0.87 | 0.0000 |
| ENSG00000166557 | TMED3 | 0.42 | 0.0000 |
| ENSG00000166681 | NGFRAP1 | 0.24 | 0.0001 |
| ENSG00000166710 | B2M | 0.18 | 0.0038 |
| ENSG00000166831 | RBPMS2 | 0.59 | 0.0000 |
| ENSG00000166848 | TERF2IP | 0.37 | 0.0000 |
| ENSG00000166887 | VPS39 | 0.38 | 0.0000 |
| ENSG00000166912 | MTMR10 | 0.38 | 0.0000 |
| ENSG00000166925 | TSC22D4 | 0.19 | 0.0021 |
| ENSG00000166946 | CCNDBP1 | 0.38 | 0.0000 |
| ENSG00000166963 | MAP1A | 0.66 | 0.0000 |
| ENSG00000166974 | MAPRE2 | 0.73 | 0.0000 |
| ENSG00000166979 | EVA1C | 0.45 | 0.0000 |
| ENSG00000167004 | PDIA3 | 0.21 | 0.0005 |
| ENSG00000167005 | NUDT21 | 0.28 | 0.0000 |
| ENSG00000167081 | PBX3 | 0.46 | 0.0000 |
| ENSG00000167100 | SAMD14 | 0.62 | 0.0000 |
| ENSG00000167110 | GOLGA2 | 0.29 | 0.0000 |
| ENSG00000167112 | TRUB2 | 0.34 | 0.0000 |
| ENSG00000167113 | COQ4 | 0.38 | 0.0000 |
| ENSG00000167114 | SLC27A4 | 0.42 | 0.0000 |
| ENSG00000167210 | LOXHD1 | 0.35 | 0.0000 |
| ENSG00000167220 | HDHD2 | 0.19 | 0.0021 |
| ENSG00000167323 | STIM1 | 0.61 | 0.0000 |
| ENSG00000167414 | GNG8 | 0.18 | 0.0027 |
| ENSG00000167460 | TPM4 | 0.73 | 0.0000 |
| ENSG00000167461 | RAB8A | 0.60 | 0.0000 |
| ENSG00000167468 | GPX4 | 0.40 | 0.0000 |
| ENSG00000167491 | GATAD2A | 0.48 | 0.0000 |
| ENSG00000167522 | ANKRD11 | 0.56 | 0.0000 |
| ENSG00000167553 | TUBA1C | 0.62 | 0.0000 |
| ENSG00000167632 | TRAPPC9 | 0.56 | 0.0000 |
| ENSG00000167642 | SPINT2 | 0.76 | 0.0000 |
| ENSG00000167645 | YIF1B | 0.73 | 0.0000 |
| ENSG00000167657 | DAPK3 | 0.36 | 0.0000 |
| ENSG00000167671 | UBXN6 | 0.42 | 0.0000 |
| ENSG00000167705 | RILP | 0.40 | 0.0000 |
| ENSG00000167740 | CYB5D2 | 0.54 | 0.0000 |
| ENSG00000167972 | ABCA3 | 0.58 | 0.0000 |
| ENSG00000167996 | FTH1 | 0.22 | 0.0003 |
| ENSG00000168002 | POLR2G | 0.26 | 0.0000 |
| ENSG00000168066 | SF1 | 0.24 | 0.0001 |
| ENSG00000168067 | MAP4K2 | 0.75 | 0.0000 |
| ENSG00000168118 | RAB4A | 0.63 | 0.0000 |
| ENSG00000168172 | HOOK3 | 0.21 | 0.0005 |
| ENSG00000168175 | MAPK1IP1L | 0.22 | 0.0003 |
| ENSG00000168256 | NKIRAS2 | 0.59 | 0.0000 |
| ENSG00000168300 | PCMTD1 | 0.31 | 0.0000 |
| ENSG00000168374 | ARF4 | 0.47 | 0.0000 |
| ENSG00000168385 | SEPT2 | 0.74 | 0.0000 |
| ENSG00000168405 | CMAHP | 0.30 | 0.0000 |
| ENSG00000168461 | RAB31 | 0.39 | 0.0000 |
| ENSG00000168497 | SDPR | 0.64 | 0.0000 |
| ENSG00000168610 | STAT3 | 0.56 | 0.0000 |
| ENSG00000168615 | ADAM9 | 0.66 | 0.0000 |
| ENSG00000168710 | AHCYL1 | 0.17 | 0.0068 |
| ENSG00000168734 | PKIG | 0.53 | 0.0000 |
| ENSG00000168765 | GSTM4 | 0.53 | 0.0000 |
| ENSG00000168883 | USP39 | 0.55 | 0.0000 |
| ENSG00000168904 | LRRC28 | 0.36 | 0.0000 |
| ENSG00000168958 | MFF | 0.34 | 0.0000 |
| ENSG00000168994 | PXDC1 | 0.40 | 0.0000 |
| ENSG00000169032 | MAP2K1 | 0.24 | 0.0001 |
| ENSG00000169057 | MECP2 | 0.19 | 0.0025 |
| ENSG00000169129 | AFAP1L2 | 0.33 | 0.0000 |
| ENSG00000169241 | SLC50A1 | 0.61 | 0.0000 |
| ENSG00000169247 | SH3TC2 | 0.51 | 0.0000 |
| ENSG00000169313 | P2RY12 | 0.28 | 0.0000 |
| ENSG00000169398 | PTK2 | 0.74 | 0.0000 |
| ENSG00000169490 | TM2D2 | 0.45 | 0.0000 |
| ENSG00000169504 | CLIC4 | 0.49 | 0.0000 |
| ENSG00000169554 | ZEB2 | 0.25 | 0.0000 |
| ENSG00000169704 | GP9 | 0.47 | 0.0000 |
| ENSG00000169756 | LIMS1 | 0.61 | 0.0000 |
| ENSG00000169764 | UGP2 | 0.51 | 0.0000 |
| ENSG00000169813 | HNRNPF | 0.31 | 0.0000 |
| ENSG00000169891 | REPS2 | 0.37 | 0.0000 |
| ENSG00000169925 | BRD3 | 0.62 | 0.0000 |
| ENSG00000170035 | UBE2E3 | 0.69 | 0.0000 |
| ENSG00000170043 | TRAPPC1 | 0.44 | 0.0000 |
| ENSG00000170100 | ZNF778 | 0.18 | 0.0036 |
| ENSG00000170113 | NIPA1 | 0.34 | 0.0000 |
| ENSG00000170242 | USP47 | 0.39 | 0.0000 |
| ENSG00000170271 | FAXDC2 | 0.84 | 0.0000 |
| ENSG00000170275 | CRTAP | 0.33 | 0.0000 |
| ENSG00000170315 | UBB | 0.19 | 0.0020 |
| ENSG00000170365 | SMAD1 | 0.20 | 0.0010 |
| ENSG00000170525 | PFKFB3 | 0.17 | 0.0053 |
| ENSG00000170542 | SERPINB9 | 0.20 | 0.0011 |
| ENSG00000171033 | PKIA | 0.33 | 0.0000 |
| ENSG00000171148 | TADA3 | 0.71 | 0.0000 |
| ENSG00000171159 | C9orf16 | 0.35 | 0.0000 |
| ENSG00000171161 | ZNF672 | 0.37 | 0.0000 |
| ENSG00000171206 | TRIM8 | 0.31 | 0.0000 |
| ENSG00000171314 | PGAM1 | 0.31 | 0.0000 |
| ENSG00000171552 | BCL2L1 | 0.67 | 0.0000 |
| ENSG00000171611 | PTCRA | 0.46 | 0.0000 |
| ENSG00000171720 | HDAC3 | 0.44 | 0.0000 |
| ENSG00000171735 | CAMTA1 | 0.18 | 0.0040 |
| ENSG00000171843 | MLLT3 | 0.36 | 0.0000 |
| ENSG00000171928 | TVP23B | 0.31 | 0.0000 |
| ENSG00000172037 | LAMB2 | 0.43 | 0.0000 |
| ENSG00000172057 | ORMDL3 | 0.55 | 0.0000 |
| ENSG00000172115 | CYCS | 0.20 | 0.0009 |
| ENSG00000172159 | FRMD3 | 0.29 | 0.0000 |
| ENSG00000172164 | SNTB1 | 0.47 | 0.0000 |
| ENSG00000172270 | BSG | 0.65 | 0.0000 |
| ENSG00000172375 | C2CD2L | 0.31 | 0.0000 |
| ENSG00000172426 | RSPH9 | 0.38 | 0.0000 |
| ENSG00000172432 | GTPBP2 | 0.63 | 0.0000 |
| ENSG00000172466 | ZNF24 | 0.20 | 0.0011 |
| ENSG00000172493 | AFF1 | 0.21 | 0.0008 |
| ENSG00000172543 | CTSW | 0.33 | 0.0000 |
| ENSG00000172572 | PDE3A | 0.25 | 0.0001 |
| ENSG00000172578 | KLHL6 | 0.44 | 0.0000 |
| ENSG00000172667 | ZMAT3 | 0.46 | 0.0000 |
| ENSG00000172725 | CORO1B | 0.56 | 0.0000 |
| ENSG00000172757 | CFL1 | 0.53 | 0.0000 |
| ENSG00000172794 | RAB37 | 0.85 | 0.0000 |
| ENSG00000172819 | RARG | 0.57 | 0.0000 |
| ENSG00000172889 | EGFL7 | 0.67 | 0.0000 |
| ENSG00000172893 | DHCR7 | 0.51 | 0.0000 |
| ENSG00000172927 | MYEOV | 0.22 | 0.0002 |
| ENSG00000172965 | MIR4435-1HG | 0.23 | 0.0001 |
| ENSG00000172992 | DCAKD | 0.61 | 0.0000 |
| ENSG00000173064 | HECTD4 | 0.19 | 0.0016 |
| ENSG00000173083 | HPSE | 0.47 | 0.0000 |
| ENSG00000173210 | ABLIM3 | 0.82 | 0.0000 |
| ENSG00000173264 | GPR137 | 0.64 | 0.0000 |
| ENSG00000173542 | MOB1B | 0.40 | 0.0000 |
| ENSG00000173598 | NUDT4 | 0.30 | 0.0000 |
| ENSG00000173660 | UQCRH | 0.24 | 0.0001 |
| ENSG00000173757 | STAT5B | 0.45 | 0.0000 |
| ENSG00000173812 | EIF1 | 0.23 | 0.0002 |
| ENSG00000173852 | DPY19L1 | 0.61 | 0.0000 |
| ENSG00000173960 | UBXN2A | 0.30 | 0.0000 |
| ENSG00000173992 | CCS | 0.19 | 0.0020 |
| ENSG00000174083 | PIK3R6 | 0.34 | 0.0000 |
| ENSG00000174099 | MSRB3 | 0.52 | 0.0000 |
| ENSG00000174175 | SELP | 1.00 | 0.0000 |
| ENSG00000174365 | SNHG11 | 0.40 | 0.0000 |
| ENSG00000174437 | ATP2A2 | 0.40 | 0.0000 |
| ENSG00000174456 | C12orf76 | 0.43 | 0.0000 |
| ENSG00000174574 | AKIRIN1 | 0.57 | 0.0000 |
| ENSG00000174788 | PCP2 | 0.33 | 0.0000 |
| ENSG00000174915 | PTDSS2 | 0.35 | 0.0000 |
| ENSG00000175063 | UBE2C | 0.26 | 0.0000 |
| ENSG00000175115 | PACS1 | 0.48 | 0.0000 |
| ENSG00000175155 | YPEL2 | 0.31 | 0.0000 |
| ENSG00000175161 | CADM2 | 0.18 | 0.0043 |
| ENSG00000175166 | PSMD2 | 0.72 | 0.0000 |
| ENSG00000175203 | DCTN2 | 0.68 | 0.0000 |
| ENSG00000175215 | CTDSP2 | 0.52 | 0.0000 |
| ENSG00000175216 | CKAP5 | 0.24 | 0.0001 |
| ENSG00000175220 | ARHGAP1 | 0.26 | 0.0000 |
| ENSG00000175221 | MED16 | 0.39 | 0.0000 |
| ENSG00000175224 | ATG13 | 0.35 | 0.0000 |
| ENSG00000175294 | CATSPER1 | 0.20 | 0.0013 |
| ENSG00000175324 | LSM1 | 0.33 | 0.0000 |
| ENSG00000175348 | TMEM9B | 0.40 | 0.0000 |
| ENSG00000175387 | SMAD2 | 0.78 | 0.0000 |
| ENSG00000175470 | PPP2R2D | 0.38 | 0.0000 |
| ENSG00000175471 | MCTP1 | 0.71 | 0.0000 |
| ENSG00000175567 | UCP2 | 0.39 | 0.0000 |
| ENSG00000175582 | RAB6A | 0.72 | 0.0000 |
| ENSG00000175662 | TOM1L2 | 0.38 | 0.0000 |
| ENSG00000175727 | MLXIP | 0.61 | 0.0000 |
| ENSG00000175826 | CTDNEP1 | 0.25 | 0.0000 |
| ENSG00000175854 | SWI5 | 0.27 | 0.0000 |
| ENSG00000175931 | UBE2O | 0.48 | 0.0000 |
| ENSG00000175984 | DENND2C | 0.24 | 0.0001 |
| ENSG00000176108 | CHMP6 | 0.60 | 0.0000 |
| ENSG00000176170 | SPHK1 | 0.65 | 0.0000 |
| ENSG00000176171 | BNIP3 | 0.23 | 0.0001 |
| ENSG00000176407 | KCMF1 | 0.45 | 0.0000 |
| ENSG00000176463 | SLCO3A1 | 0.35 | 0.0000 |
| ENSG00000176783 | RUFY1 | 0.43 | 0.0000 |
| ENSG00000176871 | WSB2 | 0.54 | 0.0000 |
| ENSG00000176953 | NFATC2IP | 0.19 | 0.0021 |
| ENSG00000177076 | ACER2 | 0.43 | 0.0000 |
| ENSG00000177119 | ANO6 | 0.68 | 0.0000 |
| ENSG00000177156 | TALDO1 | 0.59 | 0.0000 |
| ENSG00000177324 | BEND2 | 0.47 | 0.0000 |
| ENSG00000177370 | TIMM22 | 0.29 | 0.0000 |
| ENSG00000177425 | PAWR | 0.19 | 0.0023 |
| ENSG00000177479 | ARIH2 | 0.21 | 0.0005 |
| ENSG00000177565 | TBL1XR1 | 0.22 | 0.0003 |
| ENSG00000177663 | IL17RA | 0.21 | 0.0006 |
| ENSG00000177697 | CD151 | 0.70 | 0.0000 |
| ENSG00000177731 | FLII | 0.69 | 0.0000 |
| ENSG00000177885 | GRB2 | 0.33 | 0.0000 |
| ENSG00000177963 | RIC8A | 0.41 | 0.0000 |
| ENSG00000177981 | ASB8 | 0.48 | 0.0000 |
| ENSG00000178057 | NDUFAF3 | 0.50 | 0.0000 |
| ENSG00000178585 | CTNNBIP1 | 0.52 | 0.0000 |
| ENSG00000178927 | C17orf62 | 0.37 | 0.0000 |
| ENSG00000178980 | SEPW1 | 0.21 | 0.0008 |
| ENSG00000179051 | RCC2 | 0.22 | 0.0003 |
| ENSG00000179348 | GATA2 | 0.47 | 0.0000 |
| ENSG00000179361 | ARID3B | 0.47 | 0.0000 |
| ENSG00000179364 | PACS2 | 0.58 | 0.0000 |
| ENSG00000179526 | SHARPIN | 0.56 | 0.0000 |
| ENSG00000179632 | MAF1 | 0.74 | 0.0000 |
| ENSG00000180190 | TDRP | 0.64 | 0.0000 |
| ENSG00000180233 | ZNRF2 | 0.34 | 0.0000 |
| ENSG00000180354 | MTURN | 0.59 | 0.0000 |
| ENSG00000180448 | HMHA1 | 0.65 | 0.0000 |
| ENSG00000180573 | HIST1H2AC | 0.51 | 0.0000 |
| ENSG00000180596 | HIST1H2BC | 0.29 | 0.0000 |
| ENSG00000180628 | PCGF5 | 0.48 | 0.0000 |
| ENSG00000180694 | TMEM64 | 0.57 | 0.0000 |
| ENSG00000180879 | SSR4 | 0.34 | 0.0000 |
| ENSG00000181016 | LSMEM1 | 0.32 | 0.0000 |
| ENSG00000181061 | HIGD1A | 0.43 | 0.0000 |
| ENSG00000181104 | F2R | 0.59 | 0.0000 |
| ENSG00000181458 | TMEM45A | 0.23 | 0.0002 |
| ENSG00000181704 | YIPF6 | 0.34 | 0.0000 |
| ENSG00000181804 | SLC9A9 | 0.26 | 0.0000 |
| ENSG00000182048 | TRPC2 | 0.48 | 0.0000 |
| ENSG00000182054 | IDH2 | 0.20 | 0.0011 |
| ENSG00000182093 | WRB | 0.46 | 0.0000 |
| ENSG00000182134 | TDRKH | 0.24 | 0.0001 |
| ENSG00000182149 | IST1 | 0.17 | 0.0055 |
| ENSG00000182179 | UBA7 | 0.54 | 0.0000 |
| ENSG00000182208 | MOB2 | 0.56 | 0.0000 |
| ENSG00000182220 | ATP6AP2 | 0.53 | 0.0000 |
| ENSG00000182287 | AP1S2 | 0.34 | 0.0000 |
| ENSG00000182400 | TRAPPC6B | 0.21 | 0.0006 |
| ENSG00000182446 | NPLOC4 | 0.20 | 0.0014 |
| ENSG00000182500 | ORAI1 | 0.49 | 0.0000 |
| ENSG00000182551 | ADI1 | 0.56 | 0.0000 |
| ENSG00000182568 | SATB1 | 0.23 | 0.0001 |
| ENSG00000182732 | RGS6 | 0.66 | 0.0000 |
| ENSG00000182934 | SRPR | 0.61 | 0.0000 |
| ENSG00000183044 | ABAT | 0.44 | 0.0000 |
| ENSG00000183137 | CEP57L1 | 0.21 | 0.0005 |
| ENSG00000183255 | PTTG1IP | 0.67 | 0.0000 |
| ENSG00000183283 | DAZAP2 | 0.59 | 0.0000 |
| ENSG00000183386 | FHL3 | 0.22 | 0.0003 |
| ENSG00000183576 | SETD3 | 0.34 | 0.0000 |
| ENSG00000183597 | TANGO2 | 0.77 | 0.0000 |
| ENSG00000183688 | FAM101B | 0.23 | 0.0002 |
| ENSG00000183690 | EFHC2 | 0.51 | 0.0000 |
| ENSG00000183726 | TMEM50A | 0.30 | 0.0000 |
| ENSG00000183963 | SMTN | 0.48 | 0.0000 |
| ENSG00000184007 | PTP4A2 | 0.55 | 0.0000 |
| ENSG00000184009 | ACTG1 | 0.71 | 0.0000 |
| ENSG00000184178 | SCFD2 | 0.47 | 0.0000 |
| ENSG00000184216 | IRAK1 | 0.23 | 0.0002 |
| ENSG00000184489 | PTP4A3 | 0.41 | 0.0000 |
| ENSG00000184500 | PROS1 | 0.82 | 0.0000 |
| ENSG00000184602 | SNN | 0.50 | 0.0000 |
| ENSG00000184640 | SEPT9 | 0.18 | 0.0035 |
| ENSG00000184702 | SEPT5 | 0.85 | 0.0000 |
| ENSG00000184743 | ATL3 | 0.19 | 0.0017 |
| ENSG00000184792 | OSBP2 | 0.55 | 0.0000 |
| ENSG00000184840 | TMED9 | 0.21 | 0.0005 |
| ENSG00000184900 | SUMO3 | 0.37 | 0.0000 |
| ENSG00000185010 | F8 | 0.34 | 0.0000 |
| ENSG00000185015 | CA13 | 0.36 | 0.0000 |
| ENSG00000185052 | SLC24A3 | 0.69 | 0.0000 |
| ENSG00000185236 | RAB11B | 0.66 | 0.0000 |
| ENSG00000185245 | GP1BA | 0.44 | 0.0000 |
| ENSG00000185305 | ARL15 | 0.32 | 0.0000 |
| ENSG00000185340 | GAS2L1 | 0.75 | 0.0000 |
| ENSG00000185418 | TARSL2 | 0.37 | 0.0000 |
| ENSG00000185420 | SMYD3 | 0.24 | 0.0001 |
| ENSG00000185513 | L3MBTL1 | 0.46 | 0.0000 |
| ENSG00000185532 | PRKG1 | 0.56 | 0.0000 |
| ENSG00000185621 | LMLN | 0.21 | 0.0007 |
| ENSG00000185624 | P4HB | 0.57 | 0.0000 |
| ENSG00000185630 | PBX1 | 0.38 | 0.0000 |
| ENSG00000185787 | MORF4L1 | 0.28 | 0.0000 |
| ENSG00000185825 | BCAP31 | 0.58 | 0.0000 |
| ENSG00000185896 | LAMP1 | 0.46 | 0.0000 |
| ENSG00000185909 | KLHDC8B | 0.45 | 0.0000 |
| ENSG00000185963 | BICD2 | 0.61 | 0.0000 |
| ENSG00000185989 | RASA3 | 0.76 | 0.0000 |
| ENSG00000186063 | AIDA | 0.41 | 0.0000 |
| ENSG00000186088 | GSAP | 0.61 | 0.0000 |
| ENSG00000186111 | PIP5K1C | 0.50 | 0.0000 |
| ENSG00000186162 | CIDECP | 0.39 | 0.0000 |
| ENSG00000186314 | PRELID2 | 0.26 | 0.0000 |
| ENSG00000186470 | BTN3A2 | 0.26 | 0.0000 |
| ENSG00000186480 | INSIG1 | 0.50 | 0.0000 |
| ENSG00000186501 | TMEM222 | 0.17 | 0.0064 |
| ENSG00000186591 | UBE2H | 0.54 | 0.0000 |
| ENSG00000186642 | PDE2A | 0.43 | 0.0000 |
| ENSG00000186716 | BCR | 0.69 | 0.0000 |
| ENSG00000186815 | TPCN1 | 0.29 | 0.0000 |
| ENSG00000187010 | RHD | 0.35 | 0.0000 |
| ENSG00000187097 | ENTPD5 | 0.36 | 0.0000 |
| ENSG00000187098 | MITF | 0.54 | 0.0000 |
| ENSG00000187109 | NAP1L1 | 0.42 | 0.0000 |
| ENSG00000187231 | SESTD1 | 0.68 | 0.0000 |
| ENSG00000187266 | EPOR | 0.70 | 0.0000 |
| ENSG00000187667 | WHAMMP3 | 0.21 | 0.0008 |
| ENSG00000187699 | C2orf88 | 0.41 | 0.0000 |
| ENSG00000187764 | SEMA4D | 0.50 | 0.0000 |
| ENSG00000187800 | PEAR1 | 0.70 | 0.0000 |
| ENSG00000188076 | SCGB1C1 | 0.21 | 0.0006 |
| ENSG00000188191 | PRKAR1B | 0.41 | 0.0000 |
| ENSG00000188229 | TUBB4B | 0.73 | 0.0000 |
| ENSG00000188554 | NBR1 | 0.31 | 0.0000 |
| ENSG00000188641 | DPYD | 0.24 | 0.0001 |
| ENSG00000188677 | PARVB | 0.66 | 0.0000 |
| ENSG00000188921 | PTPLAD2 | 0.44 | 0.0000 |
| ENSG00000188986 | NELFB | 0.40 | 0.0000 |
| ENSG00000189308 | LIN54 | 0.22 | 0.0003 |
| ENSG00000189403 | HMGB1 | 0.24 | 0.0001 |
| ENSG00000196182 | STK40 | 0.76 | 0.0000 |
| ENSG00000196187 | TMEM63A | 0.44 | 0.0000 |
| ENSG00000196230 | TUBB | 0.54 | 0.0000 |
| ENSG00000196233 | LCOR | 0.45 | 0.0000 |
| ENSG00000196459 | TRAPPC2 | 0.31 | 0.0000 |
| ENSG00000196526 | AFAP1 | 0.21 | 0.0008 |
| ENSG00000196547 | MAN2A2 | 0.71 | 0.0000 |
| ENSG00000196611 | MMP1 | 0.49 | 0.0000 |
| ENSG00000196704 | AMZ2 | 0.33 | 0.0000 |
| ENSG00000196776 | CD47 | 0.34 | 0.0000 |
| ENSG00000196914 | ARHGEF12 | 0.58 | 0.0000 |
| ENSG00000196923 | PDLIM7 | 0.65 | 0.0000 |
| ENSG00000196924 | FLNA | 0.71 | 0.0000 |
| ENSG00000197006 | METTL9 | 0.18 | 0.0037 |
| ENSG00000197122 | SRC | 0.68 | 0.0000 |
| ENSG00000197147 | LRRC8B | 0.45 | 0.0000 |
| ENSG00000197183 | C20orf112 | 0.32 | 0.0000 |
| ENSG00000197226 | TBC1D9B | 0.60 | 0.0000 |
| ENSG00000197321 | SVIL | 0.18 | 0.0043 |
| ENSG00000197324 | LRP10 | 0.66 | 0.0000 |
| ENSG00000197386 | HTT | 0.32 | 0.0000 |
| ENSG00000197415 | VEPH1 | 0.58 | 0.0000 |
| ENSG00000197442 | MAP3K5 | 0.65 | 0.0000 |
| ENSG00000197461 | PDGFA | 0.20 | 0.0009 |
| ENSG00000197535 | MYO5A | 0.21 | 0.0005 |
| ENSG00000197586 | ENTPD6 | 0.28 | 0.0000 |
| ENSG00000197746 | PSAP | 0.58 | 0.0000 |
| ENSG00000197798 | FAM118B | 0.28 | 0.0000 |
| ENSG00000197858 | GPAA1 | 0.33 | 0.0000 |
| ENSG00000197879 | MYO1C | 0.76 | 0.0000 |
| ENSG00000197903 | HIST1H2BK | 0.39 | 0.0000 |
| ENSG00000197959 | DNM3 | 0.47 | 0.0000 |
| ENSG00000197971 | MBP | 0.36 | 0.0000 |
| ENSG00000198055 | GRK6 | 0.59 | 0.0000 |
| ENSG00000198168 | SVIP | 0.21 | 0.0005 |
| ENSG00000198356 | ASNA1 | 0.50 | 0.0000 |
| ENSG00000198431 | TXNRD1 | 0.18 | 0.0039 |
| ENSG00000198467 | TPM2 | 0.20 | 0.0009 |
| ENSG00000198478 | SH3BGRL2 | 0.75 | 0.0000 |
| ENSG00000198513 | ATL1 | 0.53 | 0.0000 |
| ENSG00000198586 | TLK1 | 0.38 | 0.0000 |
| ENSG00000198589 | LRBA | 0.60 | 0.0000 |
| ENSG00000198626 | RYR2 | 0.38 | 0.0000 |
| ENSG00000198668 | CALM1 | 0.22 | 0.0004 |
| ENSG00000198682 | PAPSS2 | 0.37 | 0.0000 |
| ENSG00000198730 | CTR9 | 0.17 | 0.0066 |
| ENSG00000198752 | CDC42BPB | 0.37 | 0.0000 |
| ENSG00000198753 | PLXNB3 | 0.59 | 0.0000 |
| ENSG00000198805 | PNP | 0.48 | 0.0000 |
| ENSG00000198814 | GK | 0.45 | 0.0000 |
| ENSG00000198833 | UBE2J1 | 0.70 | 0.0000 |
| ENSG00000198836 | OPA1 | 0.21 | 0.0005 |
| ENSG00000198843 | | 0.64 | 0.0000 |
| ENSG00000198858 | R3HDM4 | 0.68 | 0.0000 |
| ENSG00000198873 | GRK5 | 0.51 | 0.0000 |
| ENSG00000198876 | DCAF12 | 0.28 | 0.0000 |
| ENSG00000198898 | CAPZA2 | 0.65 | 0.0000 |
| ENSG00000198911 | SREBF2 | 0.53 | 0.0000 |
| ENSG00000198925 | ATG9A | 0.67 | 0.0000 |
| ENSG00000198948 | MFAP3L | 0.36 | 0.0000 |
| ENSG00000198951 | NAGA | 0.19 | 0.0018 |
| ENSG00000198960 | ARMCX6 | 0.29 | 0.0000 |
| ENSG00000203485 | INF2 | 0.62 | 0.0000 |
| ENSG00000203666 | EFCAB2 | 0.29 | 0.0000 |
| ENSG00000203879 | GDI1 | 0.74 | 0.0000 |
| ENSG00000204136 | GGTA1P | 0.36 | 0.0000 |
| ENSG00000204272 | | 0.22 | 0.0004 |
| ENSG00000204308 | RNF5 | 0.34 | 0.0000 |
| ENSG00000204310 | AGPAT1 | 0.71 | 0.0000 |
| ENSG00000204323 | SMIM5 | 0.25 | 0.0000 |
| ENSG00000204406 | MBD5 | 0.24 | 0.0001 |
| ENSG00000204420 | C6orf25 | 0.86 | 0.0000 |
| ENSG00000204424 | LY6G6F | 0.52 | 0.0000 |
| ENSG00000204428 | LY6G5C | 0.18 | 0.0037 |
| ENSG00000204525 | HLA-C | 0.32 | 0.0000 |
| ENSG00000204590 | GNL1 | 0.35 | 0.0000 |
| ENSG00000204592 | HLA-E | 0.85 | 0.0000 |
| ENSG00000204843 | DCTN1 | 0.17 | 0.0049 |
| ENSG00000205038 | PKHD1L1 | 0.63 | 0.0000 |
| ENSG00000205126 | ACCSL | 0.39 | 0.0000 |
| ENSG00000205133 | TRIQK | 0.34 | 0.0000 |
| ENSG00000205309 | NT5M | 0.80 | 0.0000 |
| ENSG00000205531 | NAP1L4 | 0.56 | 0.0000 |
| ENSG00000205581 | HMGN1 | 0.33 | 0.0000 |
| ENSG00000205593 | DENND6B | 0.36 | 0.0000 |
| ENSG00000205639 | MFSD2B | 0.84 | 0.0000 |
| ENSG00000206052 | DOK6 | 0.32 | 0.0000 |
| ENSG00000206503 | HLA-A | 0.24 | 0.0001 |
| ENSG00000206549 | PRSS50 | 0.37 | 0.0000 |
| ENSG00000206560 | ANKRD28 | 0.47 | 0.0000 |
| ENSG00000207939 | MIR223 | 0.19 | 0.0023 |
| ENSG00000211456 | SACM1L | 0.37 | 0.0000 |
| ENSG00000212694 | | 0.32 | 0.0000 |
| ENSG00000213246 | SUPT4H1 | 0.27 | 0.0000 |
| ENSG00000213281 | NRAS | 0.18 | 0.0029 |
| ENSG00000213366 | GSTM2 | 0.21 | 0.0005 |
| ENSG00000213465 | ARL2 | 0.38 | 0.0000 |
| ENSG00000213625 | LEPROT | 0.56 | 0.0000 |
| ENSG00000213639 | PPP1CB | 0.28 | 0.0000 |
| ENSG00000213654 | GPSM3 | 0.44 | 0.0000 |
| ENSG00000213672 | NCKIPSD | 0.71 | 0.0000 |
| ENSG00000213719 | CLIC1 | 0.32 | 0.0000 |
| ENSG00000213889 | PPM1N | 0.28 | 0.0000 |
| ENSG00000214941 | ZSWIM7 | 0.33 | 0.0000 |
| ENSG00000215039 | CD27-AS1 | 0.36 | 0.0000 |
| ENSG00000215302 | | 0.17 | 0.0057 |
| ENSG00000222041 | LINC00152 | 0.19 | 0.0026 |
| ENSG00000223380 | SEC22B | 0.21 | 0.0006 |
| ENSG00000223482 | NUTM2A-AS1 | 0.21 | 0.0007 |
| ENSG00000223519 | KIF28P | 0.32 | 0.0000 |
| ENSG00000223773 | CD99P1 | 0.17 | 0.0055 |
| ENSG00000224616 | | 0.46 | 0.0000 |
| ENSG00000224914 | LINC00863 | 0.22 | 0.0004 |
| ENSG00000225205 | | 0.22 | 0.0003 |
| ENSG00000225484 | | 0.31 | 0.0000 |
| ENSG00000225733 | FGD5-AS1 | 0.53 | 0.0000 |
| ENSG00000225936 | | 0.37 | 0.0000 |
| ENSG00000226777 | KIAA0125 | 0.20 | 0.0012 |
| ENSG00000226824 | | 0.25 | 0.0000 |
| ENSG00000227355 | | 0.18 | 0.0033 |
| ENSG00000228215 | | 0.26 | 0.0000 |
| ENSG00000228409 | CCT6P1 | 0.33 | 0.0000 |
| ENSG00000228651 | | 0.18 | 0.0032 |
| ENSG00000229666 | MAST4-AS1 | 0.23 | 0.0002 |
| ENSG00000229754 | CXCR2P1 | 0.55 | 0.0000 |
| ENSG00000231925 | TAPBP | 0.17 | 0.0068 |
| ENSG00000233093 | LINC00892 | 0.18 | 0.0026 |
| ENSG00000233276 | GPX1 | 0.55 | 0.0000 |
| ENSG00000233369 | | 0.39 | 0.0000 |
| ENSG00000233452 | STXBP5-AS1 | 0.24 | 0.0001 |
| ENSG00000233527 | | 0.23 | 0.0001 |
| ENSG00000233614 | DDX11L10 | 0.30 | 0.0000 |
| ENSG00000234231 | | 0.51 | 0.0000 |
| ENSG00000234585 | CCT6P3 | 0.38 | 0.0000 |
| ENSG00000234745 | HLA-B | 0.22 | 0.0004 |
| ENSG00000234810 | | 0.34 | 0.0000 |
| ENSG00000235162 | C12orf75 | 0.50 | 0.0000 |
| ENSG00000235257 | | 0.36 | 0.0000 |
| ENSG00000235609 | | 0.57 | 0.0000 |
| ENSG00000236279 | CLEC2L | 0.47 | 0.0000 |
| ENSG00000236304 | | 0.21 | 0.0006 |
| ENSG00000236397 | DDX11L2 | 0.43 | 0.0000 |
| ENSG00000236875 | DDX11L5 | 0.28 | 0.0000 |
| ENSG00000236936 | | 0.18 | 0.0033 |
| ENSG00000237419 | | 0.29 | 0.0000 |
| ENSG00000237803 | LINC00211 | 0.41 | 0.0000 |
| ENSG00000237805 | | 0.21 | 0.0008 |
| ENSG00000237854 | LINC00674 | 0.51 | 0.0000 |
| ENSG00000238201 | | 0.20 | 0.0011 |
| ENSG00000239213 | | 0.26 | 0.0000 |
| ENSG00000239445 | ST3GAL6-AS1 | 0.25 | 0.0000 |
| ENSG00000241685 | ARPC1A | 0.36 | 0.0000 |
| ENSG00000241973 | PI4KA | 0.26 | 0.0000 |
| ENSG00000243317 | C7orf73 | 0.22 | 0.0004 |
| ENSG00000244509 | APOBEC3C | 0.29 | 0.0000 |
| ENSG00000245552 | | 0.40 | 0.0000 |
| ENSG00000246448 | | 0.19 | 0.0019 |
| ENSG00000246889 | | 0.30 | 0.0000 |
| ENSG00000247271 | ZBED5-AS1 | 0.27 | 0.0000 |
| ENSG00000247556 | OIP5-AS1 | 0.24 | 0.0001 |
| ENSG00000248242 | | 0.20 | 0.0009 |
| ENSG00000248636 | | 0.37 | 0.0000 |
| ENSG00000249684 | | 0.35 | 0.0000 |
| ENSG00000249898 | | 0.23 | 0.0002 |
| ENSG00000250334 | LINC00989 | 0.31 | 0.0000 |
| ENSG00000250348 | | 0.27 | 0.0000 |
| ENSG00000250878 | METTL21EP | 0.35 | 0.0000 |
| ENSG00000251600 | | 0.23 | 0.0001 |
| ENSG00000253394 | LINC00534 | 0.36 | 0.0000 |
| ENSG00000253819 | LINC01151 | 0.40 | 0.0000 |
| ENSG00000253982 | | 0.37 | 0.0000 |
| ENSG00000254087 | LYN | 0.48 | 0.0000 |
| ENSG00000254138 | | 0.17 | 0.0058 |
| ENSG00000254786 | | 0.24 | 0.0001 |
| ENSG00000254999 | BRK1 | 0.33 | 0.0000 |
| ENSG00000255002 | | 0.24 | 0.0001 |
| ENSG00000255240 | | 0.23 | 0.0001 |
| ENSG00000255325 | | 0.34 | 0.0000 |
| ENSG00000255364 | | 0.43 | 0.0000 |
| ENSG00000257103 | LSM14A | 0.31 | 0.0000 |
| ENSG00000257261 | | 0.27 | 0.0000 |
| ENSG00000257267 | ZNF271 | 0.34 | 0.0000 |
| ENSG00000257923 | CUX1 | 0.81 | 0.0000 |
| ENSG00000258999 | | 0.31 | 0.0000 |
| ENSG00000259719 | | 0.65 | 0.0000 |
| ENSG00000260032 | LINC00657 | 0.25 | 0.0000 |
| ENSG00000261253 | | 0.37 | 0.0000 |
| ENSG00000263563 | UBBP4 | 0.28 | 0.0000 |
| ENSG00000264964 | | 0.24 | 0.0001 |
| ENSG00000265148 | BZRAP1-AS1 | 0.67 | 0.0000 |
| ENSG00000267243 | | 0.28 | 0.0000 |
| ENSG00000268555 | | 0.29 | 0.0000 |
| ENSG00000270055 | | 0.32 | 0.0000 |
| ENSG00000272168 | CASC15 | 0.33 | 0.0000 |
| ENSG00000272369 | | 0.18 | 0.0036 |
| ENSG00000273143 | | 0.31 | 0.0000 |

### EXAMPLES

### Example 1

### General Materials and Methods

### Study Design and Sample Selection

Peripheral whole blood was drawn by venipuncture from cancer patients, patients with inflammatory and other non-cancerous conditions, and asymptomatic individuals at the VU University Medical Center, Amsterdam. The Netherlands, the Dutch Cancer Institute (NKI/AvL), Amsterdam, The Netherlands, the Academical Medical Center, Amsterdam, The Netherlands, the Utrecht Medical Center, Utrecht, The Netherlands, the University Hospital of Umeå, Umeå, Sweden, the Hospital Germans Trias i Pujol, Barcelona, Spain, The University Hospital of Pisa, Pisa, Italy, and Massachusetts General Hospital, Boston, USA. Whole blood was collected in 4-, 6-, or 10-mL EDTA-coated purple-capped BD Vacutainers containing the anti-coagulant EDTA. Cancer patients were diagnosed by clinical, radiological and pathological examination, and were confirmed to have at moment of blood collection detectable tumor load. 106 NSCLC samples included were follow-up samples of the same patient, collected weeks to months after the first blood collection. Age-matching was performed retrospectively using a custom script in MATLAB, iteratively matching samples by excluding and including Non-cancer and NSCLC samples aiming at a similar median age and age-range between both groups. Samples for both training, evaluation, and validation cohorts were collected and processed similarly and simultaneously. A detailed overview of the included samples, demographic characteristics, the hospital of origin, time between blood collection and platelet isolation (blood storage time), and for which analyses and classifiers were used is provided in Table 4. Asymptomatic individuals were at the moment of blood collection, or previously, not diagnosed with cancer, but were not subjected to additional tests confirming the absence of cancer. The patients with inflammatory or other non-cancerous conditions did not have a diagnosed malignant tumor at the moment of blood collection. This study was conducted in accordance with the principles of the Declaration of Helsinki. Approval for this study was obtained from the institutional review board and the ethics committee at each participating hospital. Clinical follow-up of asymptomatic individuals is not available due to anonymization of these samples according to the ethical rules of the hospitals.

### Clinical Data Annotation

For collection and annotation of clinical data, patient records were manually queried for demographic variables. i.e. age, gender, smoking, type of tumor, metastases, details of current and prior treatments, and co-morbidities. In case of transgender individuals, the new gender was stated (n=1). Platelet samples were collected before start of (a new) treatment or during treatment, respectively baseline and follow-up samples. Response assessment of patients treated with nivolumab (FIG. 2) was performed by CT-imaging at baseline, 6-8 weeks, 3 months and 6 months after start of treatment. For the nivolumab response prediction algorithm, samples that were collected up to one month before start of treatment were annotated as baseline samples. Treatment response was assessed according to the updated RECIST version 1.1 criteria and scored as progressive disease (PD), stable disease (SD), partial response (PR), or complete response (CR) (Eisenhauer et al., 2009, European Journal of Cancer, 45: 228-247; Schwartz et al., 2016, European journal of cancer 62: 132-137). See FIG. 2a for a detailed schematic representation. Our aim was to identify those patients with disease control to therapy. Hence, for the nivolumab response prediction analysis, we grouped patients with progressive disease as the most optimal response in the non-responding group, totaling 60 samples. Patients with partial response at any response assessment time point as most optimal response or stable disease at 6 months response assessment were annotated as responders, totaling 44 samples. All clinical data was anonymized and stored in a secured database.

### Confounding Variable Analysis

To estimate the contribution of the variables 1) patient age (in years) at moment of blood collection, 2) whole blood storage time, 3) gender, and 4) smoking (current, former, never), we summarized the available data from Supplemental Table S1A-C and Supplemental FIG. S2C of our previous study (Best et al., 2015, Cancer Cell, 28: 666-676), and performed logistic regression analyses in the statistical software module SAS (v.13.0.0; SAS Institute Inc., 100 SAS Campus Drive, Cary, N.C. 27513-2414, USA). Blood storage time was defined as the time between blood collection and the start of platelet isolation by differential centrifugation, stratified into a <12 hours group and a >12 hours group. For variables of samples of which data was missing, that particular value of the particular samples was excluded from the calculation. The joint predictive power of patient age, blood storage time, and the predictive strength of the diagnostics classifier for NSCLC, was assessed using a measure of logistic regression with nominal response, by selecting disease state as the Role Variable Y. and adding patient age, blood storage time, gender, smoking, and predictive strength for NSCLC as the model effects. All additional settings were set at default.

TABLE 4
Comprehensive overview of the study cohort and statistical contribution to the classifiers.

| | | | | | | | | | Statistical predictive contribution | | | | |
|---|---|---|---|---|---|---|---|---|---|---|---|---|---|
| | | | | | | | | | Likelihood-ratio chi-square value (p-value) | | | | |
| | | | | | | | | Blood | | | | | thrombo- |
| | | | | | | n | Median | Storage | | | | | Seq |
| | | | | | AUC | Inflam- | age | (% < | Patient | Blood | | | classifi- |
| | Cohort | Group | n | Acc. | (95%-Cl) | matory | (IQR) | 12 h) | age | storage | Gender | Smoking | cation |
| Un- | Training | Healthy | 39 | 92% | 0.99 | 0 | 40 | 100% | 9.8 | 2.9 | 0.8 | n.a. | 29.5 |
| matched | (un- | | | | (0.97-1.00) | n.a. | (22.25) | | (p = 0.002) | (p = 0.09) | (p = 0.38) | | (p < |
| cohort | matched) | NSCLC | 36 | | | | 59 | 61% | | | | | 0.0001) |
| (Best | | | | | | | (13.25) | | | | | | |
| et.al. | Validation | Healthy | 16 | 98% | 0.98 | 0 | 32.5 | 100% | 0.004 | 0.01 | 3.5 | n.a. | 21.6 |
| 2015) | (un- | | | | (0.93-1.00) | | (26.25) | | (p = 0.95) | (p = 0.90) | (p = 0.06) | | (p < |
| | matched) | NSCLC | 24 | | | n.a. | 62 | 58% | | | | | 0.0001) |
| | | | | | | | (14.25) | | | | | | |
| Matched | Training | Non- | 44 | 77% | 0.84 | 36 | 62 | 100% | 2.4 | n.a. | 0.03 | 5.7 | 30.7 |
| cohort | (matched) | cancer | | | (0.75-0.92) | | (18.5) | | (p = 0.12) | | (p = 0.87) | (p = 0.12) | (p < |
| (this | | NSCLC | 49 | | | n.a. | 59 | 100% | | | | | 0.0001) |
| study) | | | | | | | (9) | | | | | | |
| Genes: | Evaluation | Non- | 20 | 85% | 0.91 | 4 | 61 | 100% | 4.1 | n.a. | 0.05 | 6.0 | 32.0 |
| n= 830 | (matched) | cancer | | | (0.62.1.00) | | (10.25) | | (p = 0.12) | | (p = 0.80) | (p = 0.11) | (p < |
| | | NSCLC | 20 | | | n.a. | 58 | 100% | | | | | 0.0001) |
| | | | | | | | (24) | | | | | | |
| | Validation | Non- | 40 | 91% | 0.95 | 9 | 56 | 100% | 3.7 | n.a. | 0.1 | 14.7 | 76.2 |
| | (matched) | cancer | | | (0.91-0.99) | | (9.25) | | (p = 0.06) | | (p = 0.95) | (p = 0.002) | (p < |
| | | NSCLC | 90 | | | n.a. | 63 | 100% | | | | | 0.0001) |
| | | | | | | | (14) | | | | | | |
| Full | Training | Non- | 60 | 84% | 0.90 | 30 | 59 | 100% | <0.0001 | n.a. | 3.4 | 2.7 | 58.7 |
| cohort | (matched) | cancer | | | (0.84-0.95) | | (9.25) | | (p = 0.99) | | (p = 0.18) | (p = 0.43) | (p < |
| (this | | NSCLC | 60 | | | n.a. | 61 | 100% | | | | | 0.0001) |
| study) | | | | | | | (13.25) | | | | | | |
| Genes: | Evaluation | Non- | 44 | 91% | 0.93 | 19 | 58 | 100% | 0.62 | n.a. | 1.1 | 9.9 | 55.0 |
| n = 1000 | (matched) | cancer | | | (0.87-0.99) | | (15.5) | | (p = 0.43) | | (p = 0.30) | (p = 0.02) | (p < |
| | | NSCLC | 44 | | | n.a. | 62 | 100% | | | | | 0.0001) |
| | | | | | | | (13) | | | | | | |
| | Validation | Non- | 268 | 89% | 0.94 | 91 | 39 | 3% | 42.4 | 0.05 | 0.23 | 28.0 | 87.7 |
| | (un- | cancer | | | (0.93-0.96) | | (19) | | (p < | (p = 0.83) | (p = 0.63) | (p < 0.0001) | (p < |
| | matched) | NSCLC | 248 | | | n.a. | 64 | 25% | 0.0001) | | | | 0.0001) |
| | | | | | | | (14) | | | | | | |

### Blood Processing and Platelet Isolation

Whole blood samples in 4-, 6-, or 10-mL EDTA-coated Vacutainer tubes were processed using standardized protocols within 48 hours as described previously (Best et al., 2015. Cancer Cell 28: 666-676; Nilsson et al., 2011. Blood 118: 3680-3683). Whole blood collected in the VU University Medical Center, the Dutch Cancer Institute, the Utrecht Medical Center, the University Hospital of Umeå, the Hospital Germans Trias I Pujol, and the University Hospital of Pisa was subjected to platelet isolation within 12 hours after blood collection. Whole blood samples collected at Massachusetts General Hospital Boston and the Academical Medical Center Amsterdam were stored overnight and processed after 24 hours. To isolate platelets, platelet rich plasma (PRP) was separated from nucleated blood cells by a 20-minute 120×g centrifugation step, after which the platelets were pelleted by a 20-minute 360×g centrifugation step. Removal of 9/10th of the PRP has to be performed carefully to reduce the risk of contamination of the platelet preparation with nucleated cells, pelleted in the buffy coat. Centrifugations were performed at room temperature. Platelet pellets were carefully resuspended in RNAlater (Life Technologies) and after overnight incubation at 4° C. frozen at −80° C.

### Flow Cytometric Analysis of Platelet Activation

To assess the relative platelet activation during our platelet isolations, we measured the surface protein expression levels of the constitutively expressed platelet marker CD41 (APC anti-human, clone: HIP8) and platelet activation-dependent markers P-selectin (CD62P, PE anti-human, clone: AK4, Biolegend) and CD63 (FITC anti-human, clone: H5C6, Biolegend), using a BD FACSCalibur flow cytometer. We collected five 6-mL EDTA-coated Vacutainers tubes from each of six healthy donors, and determined the platelet activation state at baseline (0 hours), 8 hours, 24 hours, 48 hours, and 72 hours. As a negative control, we isolated at time point zero platelets from whole blood using a standardized platelet isolation protocol from citrate-anticoagulated whole blood that has been validated for inducing minimal platelet activation. This protocol consisted of a step of OptiPrep (Sigma) density gradient centrifugation (350×g, 15 minutes) after collection of platelet rich plasma. This was followed by two washing steps first with Hepes, followed by a washing step in SSP+ (Macopharma) buffer. We used 400 nM prostaglandin I2 (Sigma-Aldrich) before every centrifugation step to prevent platelet activation during the isolation procedure. As a positive control, we included platelets activated by 20 μM TRAP (TRAPtest, Roche). Platelet pellets were after isolation prefixed in 0.5% formaldehyde (Roth), stained, and stored in 1% formaldehyde for flow cytometric analysis. Relative activation and mean fluorescent intensity (MFI) values were analyzed with FlowJo. Hence, absence of platelet activation during blood collection and storage was confirmed by stable levels of P-selectin and CD63 platelet surface markers (FIG. 4b).

### Total RNA Isolation, SMARTer Amplification, and Truseq Library Preparation

Preparation of samples for sequencing was performed in batches, and included per batch a mixture of clinical conditions. For platelet RNA isolation, frozen platelets were thawed on ice and total RNA was isolated using the mirVana miRNA isolation kit (Ambion. Thermo Scientific, AM1560). Platelet RNA was eluated in 30 μL elution buffer. We evaluated the platelet RNA quality using the RNA 6000 Picochip (Bioanalyzer 2100, Agilent), and included as a quality standard for subsequent experiments only platelet RNA samples with a RIN-value >7 and/or distinctive rRNA curves. All Bioanalyzer 2100 quality and quantity measures were collected from the automatically generated Bioanalyzer result reports using default settings, and after critical assessment of the reference ladder (quantity, appearance, and slope). The Truseq cDNA labelling protocol for Illumina sequencing (see below) requires ˜1 μg of input cDNA. Since a single platelet contains an estimated ˜2 femtogram of RNA (Teruel-Montoya et al., 2014. PLuS ONE 9(7): e102259), assuming an average platelet count of 300×106 per mL of whole blood and highly efficient platelet isolation and RNA extraction, the estimated optimal yield of platelets from clinically relevant blood volumes (6 mL) is about 3.6 microg. The average total RNA obtained from our blood samples is 146 ng (standard deviation: 130 ng, n=237 samples, see FIG. 4c). Measurement of total platelet RNA yield of whole blood collected in 6 mL EDTA-coated Vacutainer tubes between Non-cancer individuals (n=86) and NSCLC patients (n=151) resulted in a minor but significant increase in total RNA in platelets of NSCLC patients (p=0.0014, Student's t-test. FIG. 4c), which was attributed to a potential difference in the platelet turnover in NSCLC patients (see also Example 3). To have sufficient platelet cDNA for robust RNA-seq library preparation, the samples were subjected to cDNA synthesis and amplification using the SMARTer Ultra Low RNA Kit for Illumina Sequencing v3 (Clontech, cat. nr. 634853). Prior to amplification, all samples were diluted to ˜500 pg/microL total RNA and again the quality was determined and quantified using the Bioanalyzer Picochip. For samples with a stock yield below 400 pg/microL, a volume of two or more microliters of total RNA (up to ˜500 pg total RNA) was used as input for the SMARTer amplification. Quality control of amplified cDNA was measured using the Bioanalyzer 2100 with DNA High Sensitivity chip (Agilent). All SMARTer cDNA synthesis and amplifications were performed together with a negative control, which was required to be negative by Bioanalyzer analysis. Samples with detectable fragments in the 300-7500 bp region were selected for further processing. To measure the average cDNA length, we selected in the Bioanalyzer software the region from 200-9000 base pairs and recorded the average length. For labelling of platelet cDNA for sequencing, all amplified platelet cDNA was first subjected to nucleic acid shearing by sonication (Covaris Inc) and subsequently labelled with single index barcodes for Illumina sequencing using the Truseq Nano DNA Sample Prep Kit (Illumina, cat nr. FC-121-4001). To account for the low platelet cDNA input concentration, all bead clean-up steps were performed using a 15-minute bead-cDNA binding step and a 10-cycle enrichment PCR. All other steps were according to manufacturers protocol. Labelled platelet DNA library quality and quantity was measured using the DNA 7500 chip or DNA High Sensitivity chip (Agilent). To correlate total RNA input for SMARTer amplification, SMARTer amplification cDNA yield, and Truseq cDNA yield (FIG. 4d, e) all samples with matched data available were subjected to a Pearson correlation test (correlation test function in R). High-quality samples with product sizes between 300-500 bp were pooled (12-19 samples per pool) in equimnolar concentrations for shallow thromboSeq and submitted for 100 bp Single Read sequencing on the Illumina Hiseq 2500 platform using version 4 sequencing reagents. For the deep thromboSeq experiment (see FIG. 4l), we pooled 12 identically prepared platelet samples, and sequenced the same pool on four lanes of a Hiseq 2500 flowcell. Subsequently, four separate FASTQ-files per sample were merged in silico.

### Processing of Raw RNA-Sequencing Data

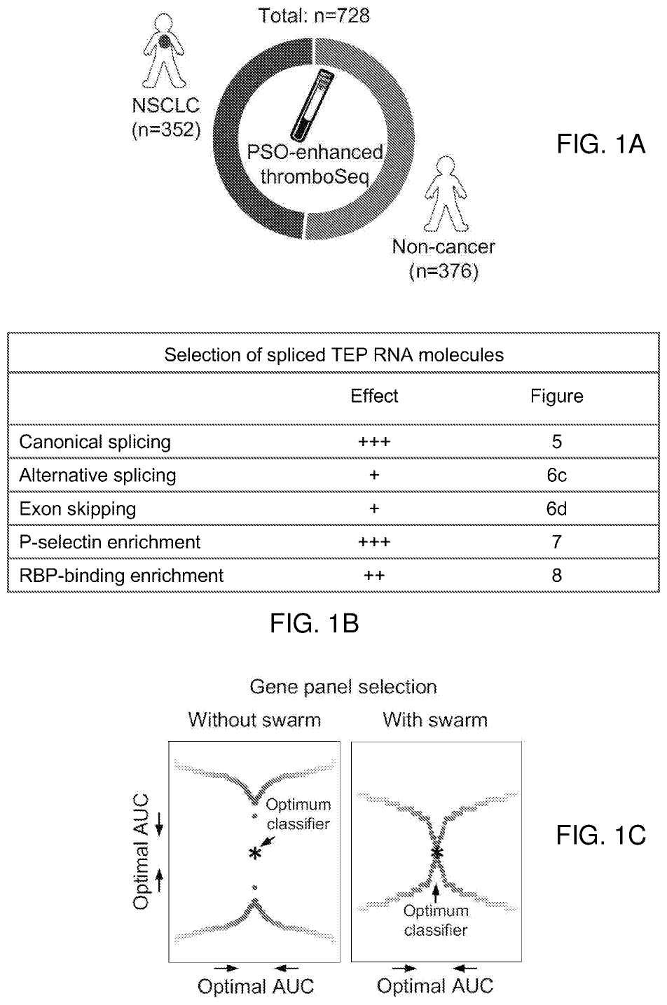

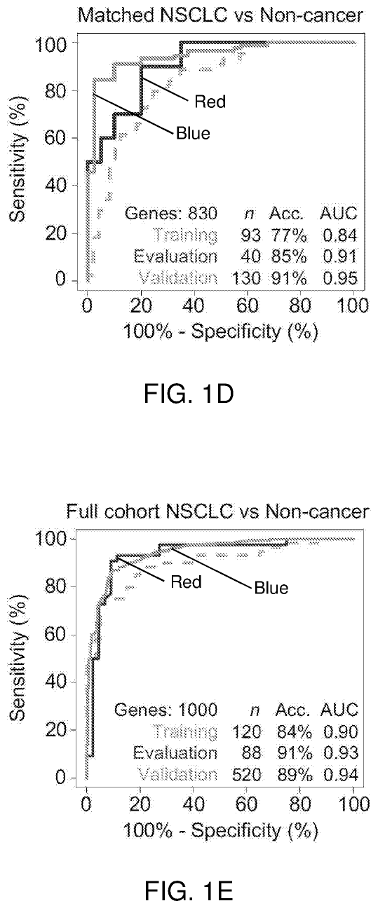

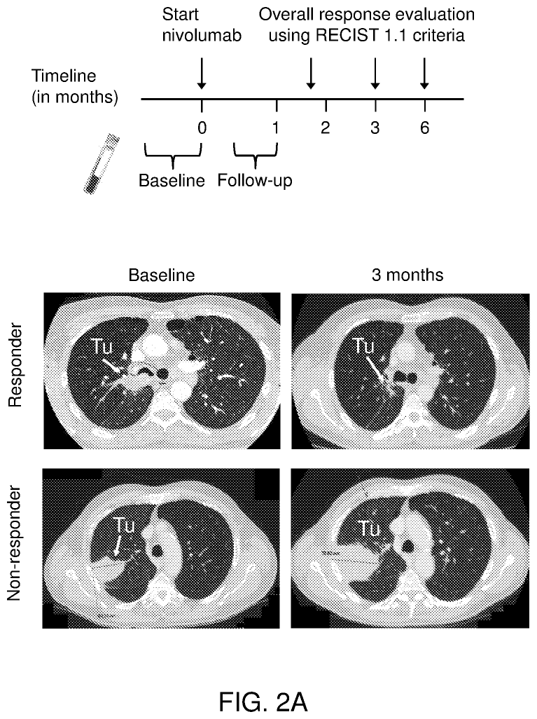

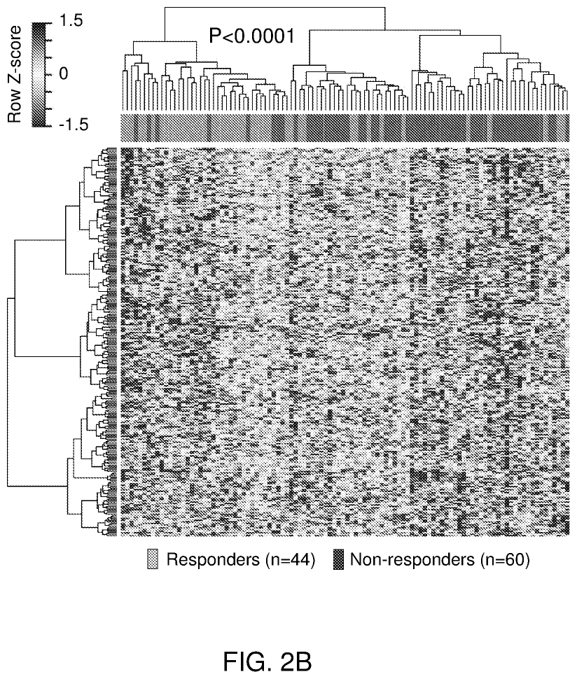

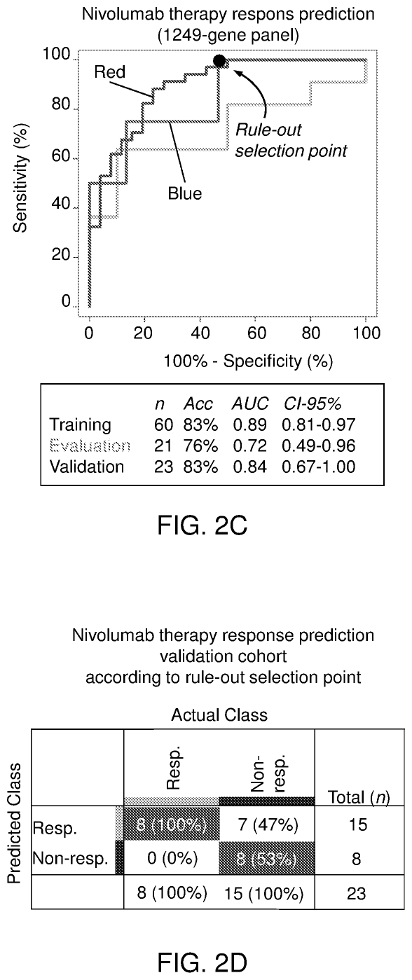

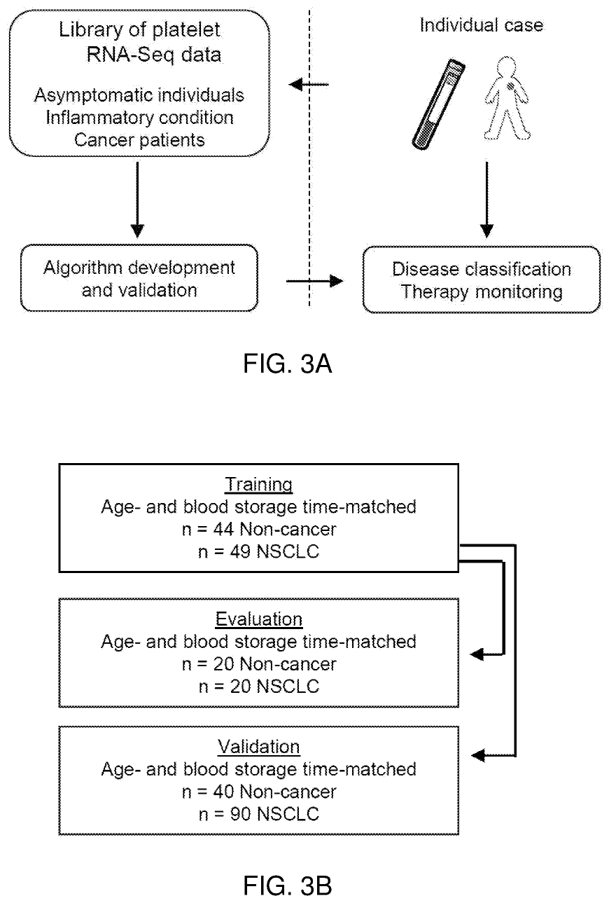

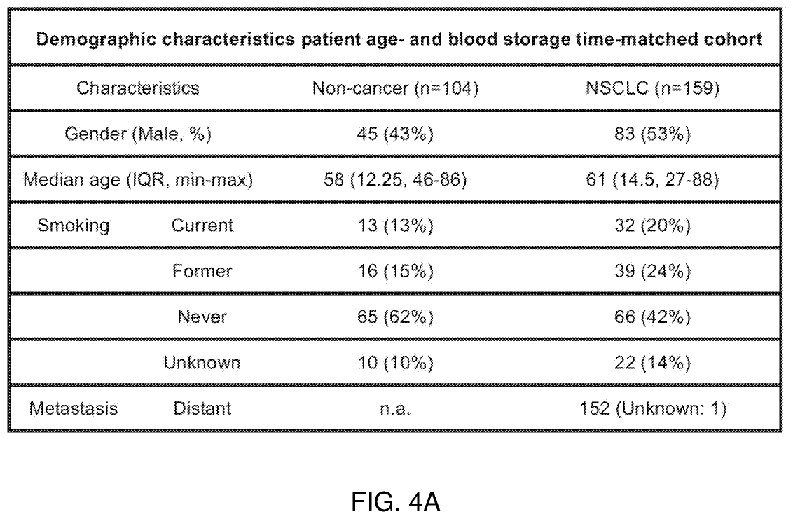

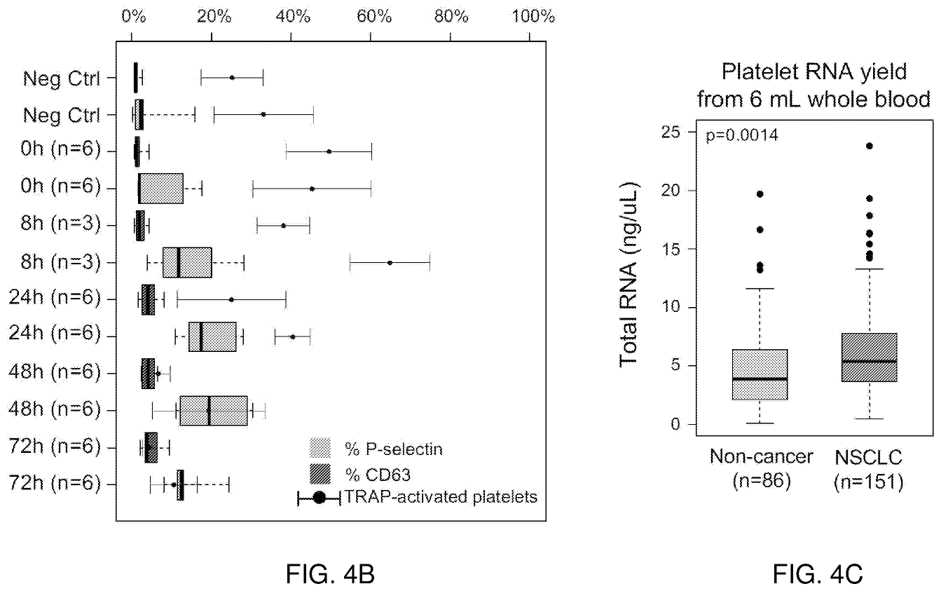

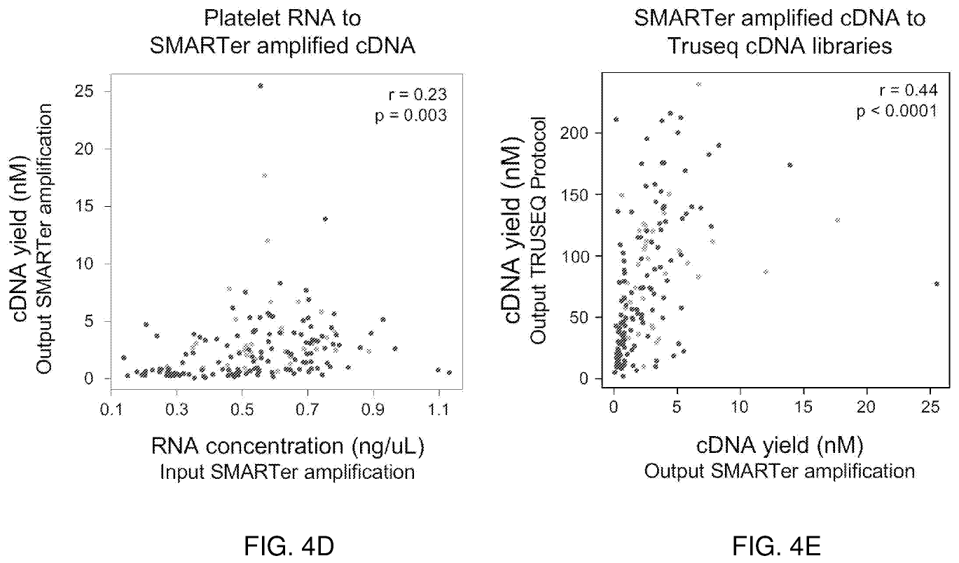

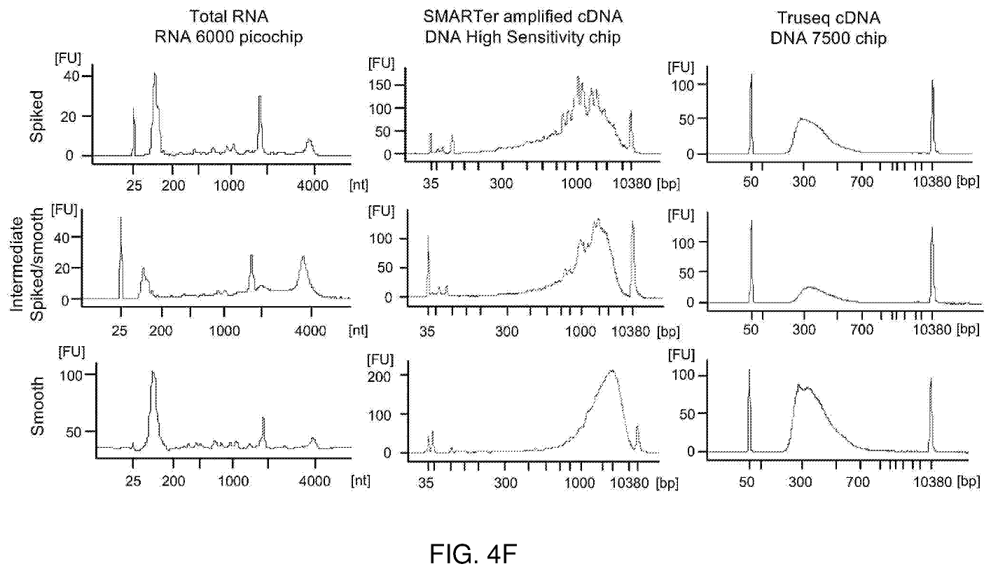

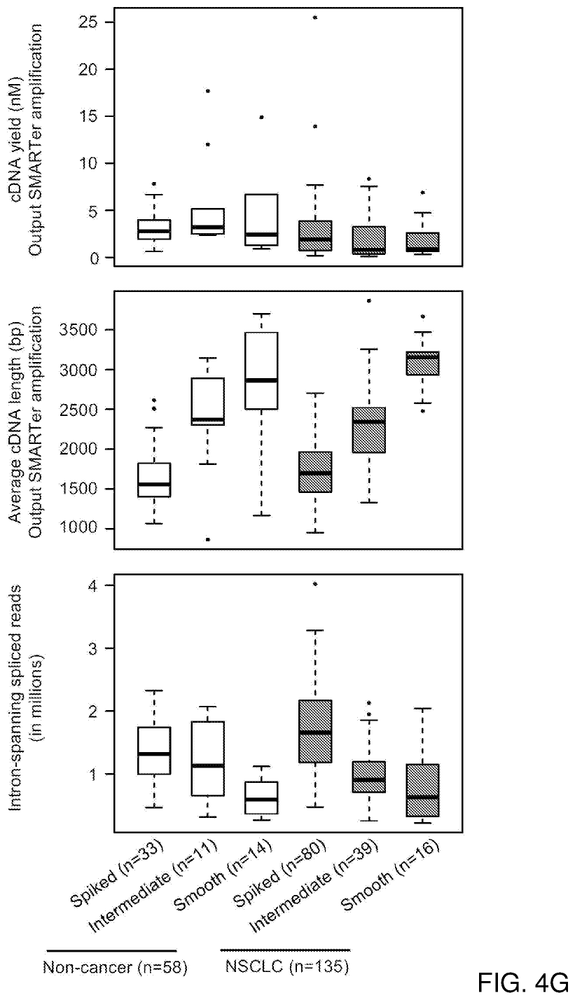

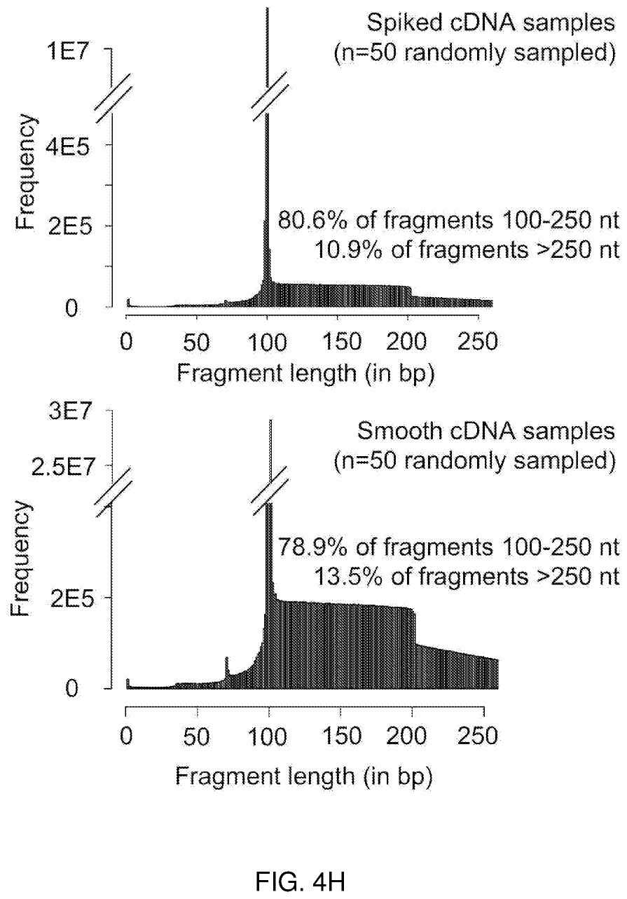

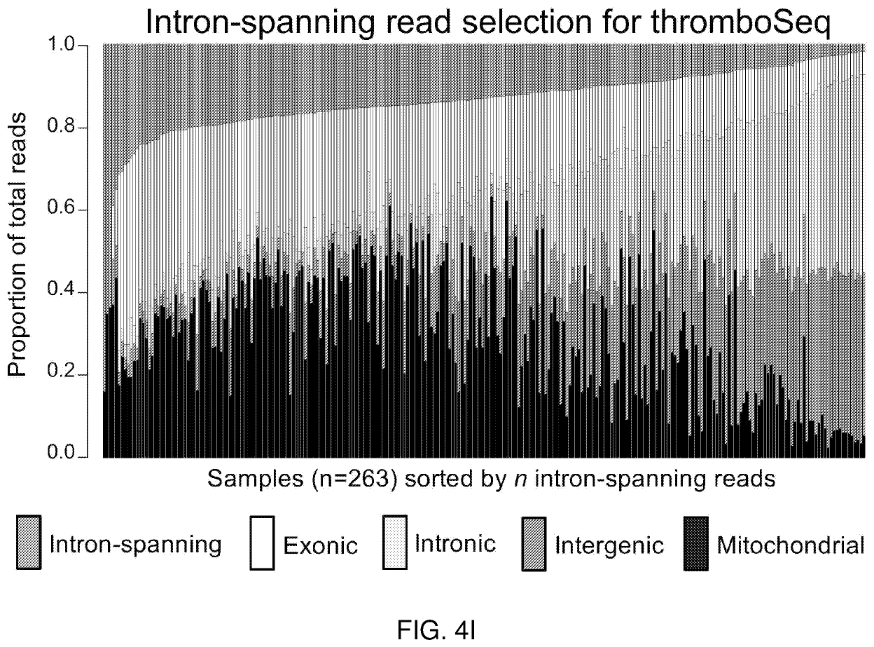

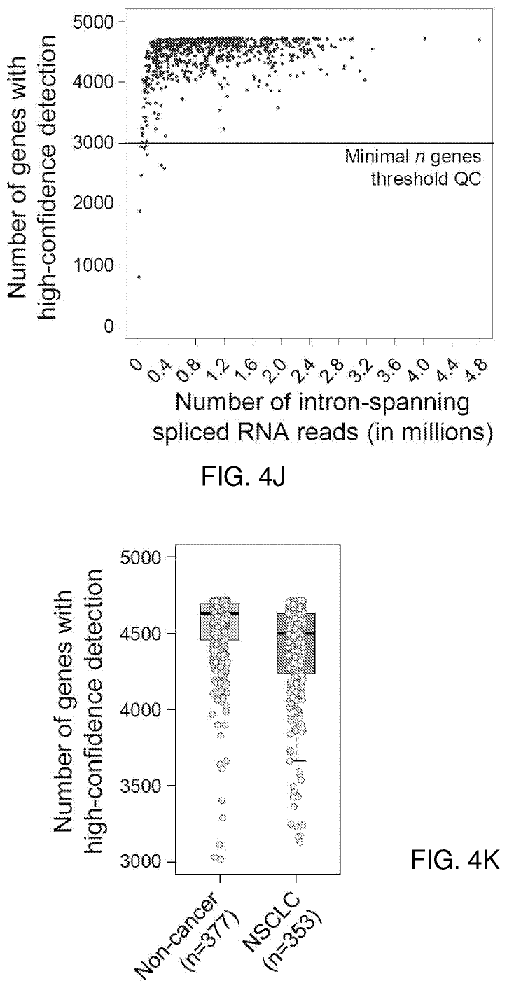

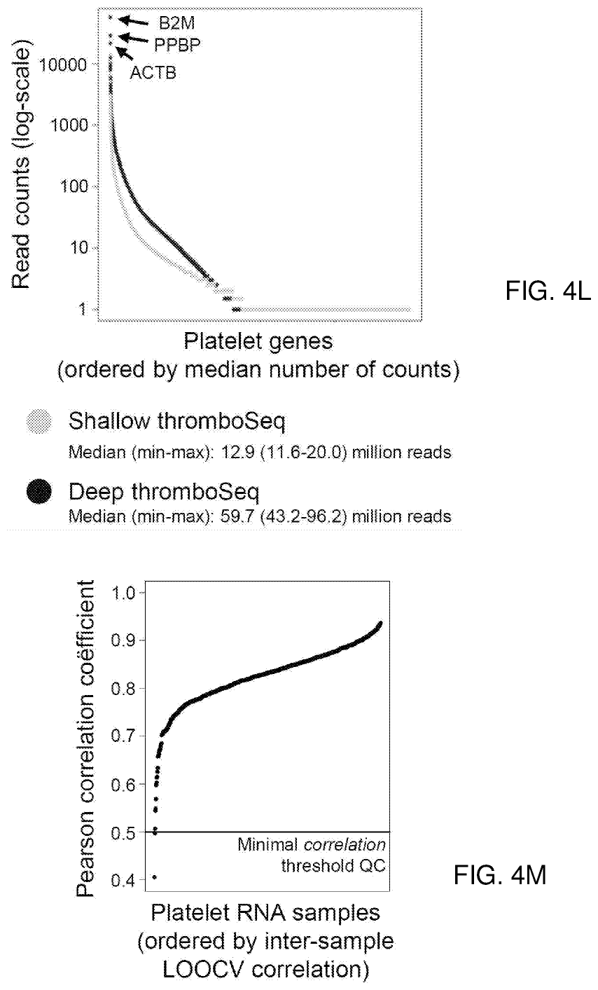

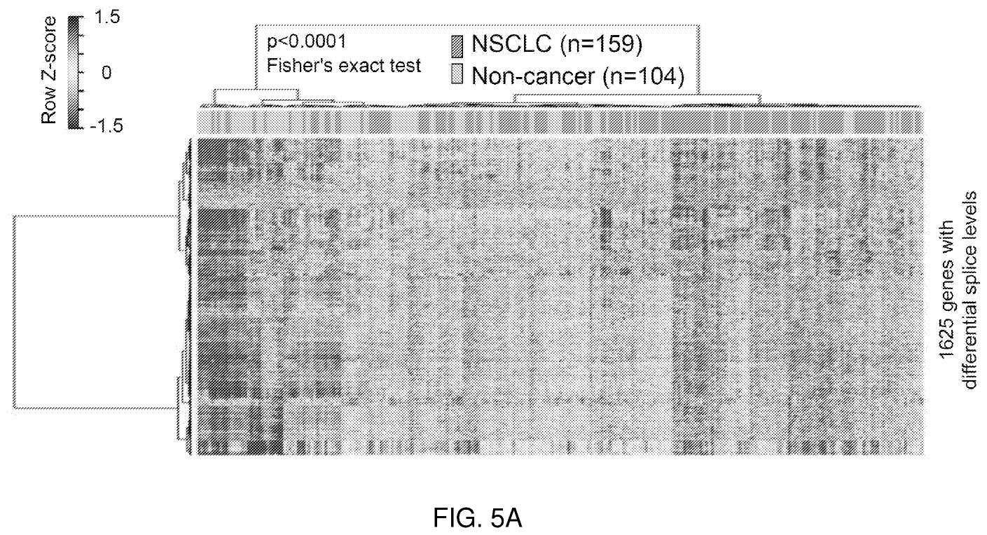

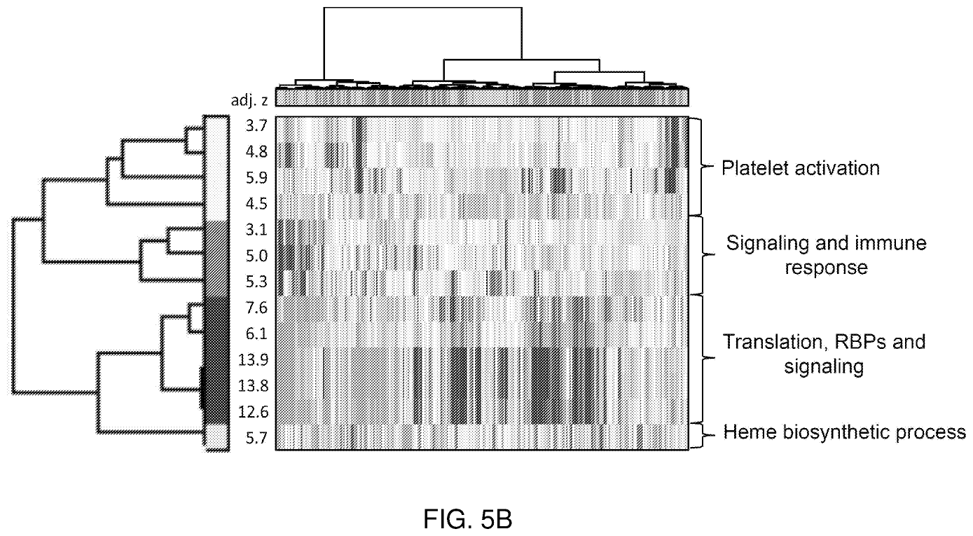

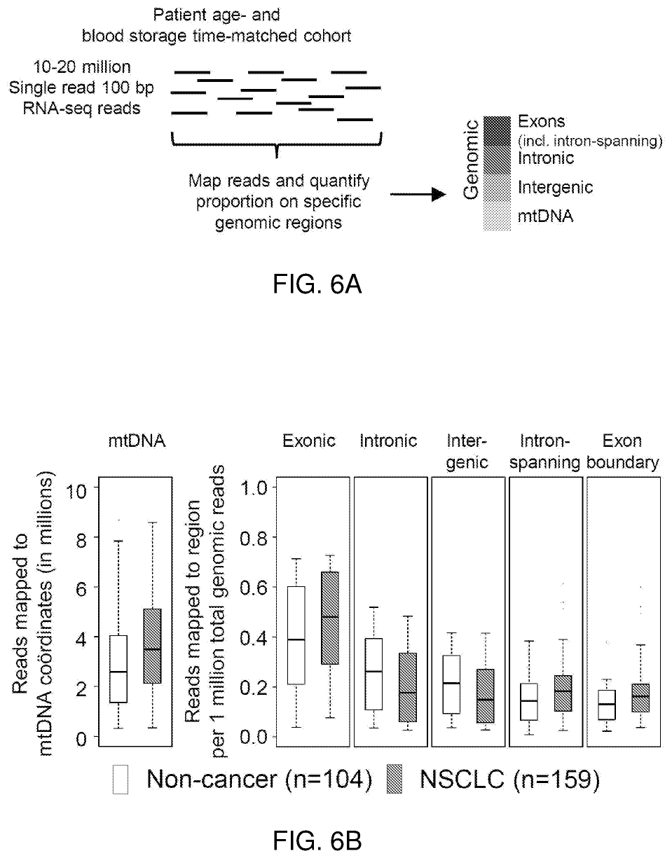

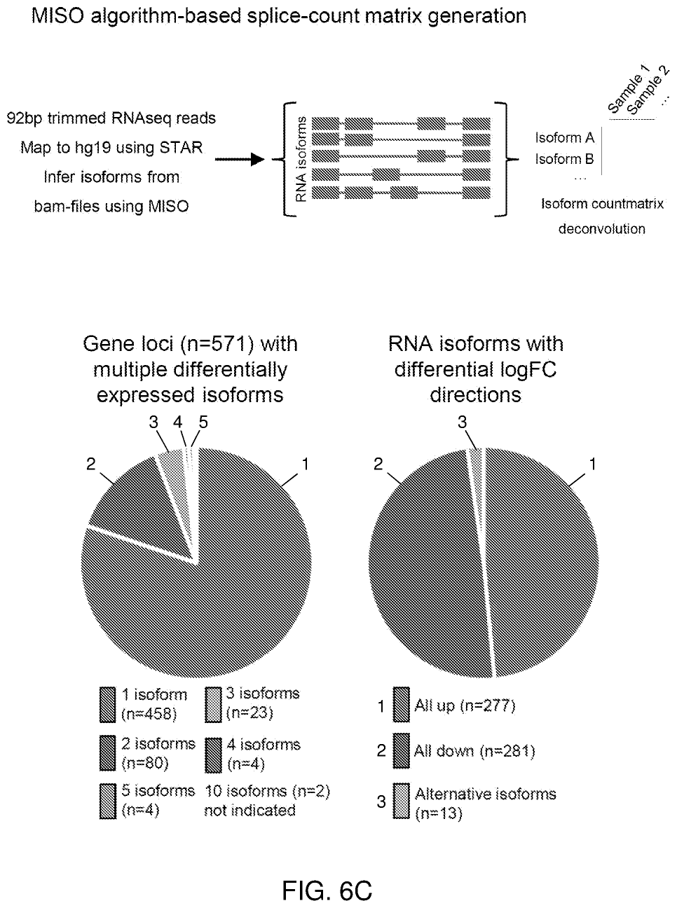

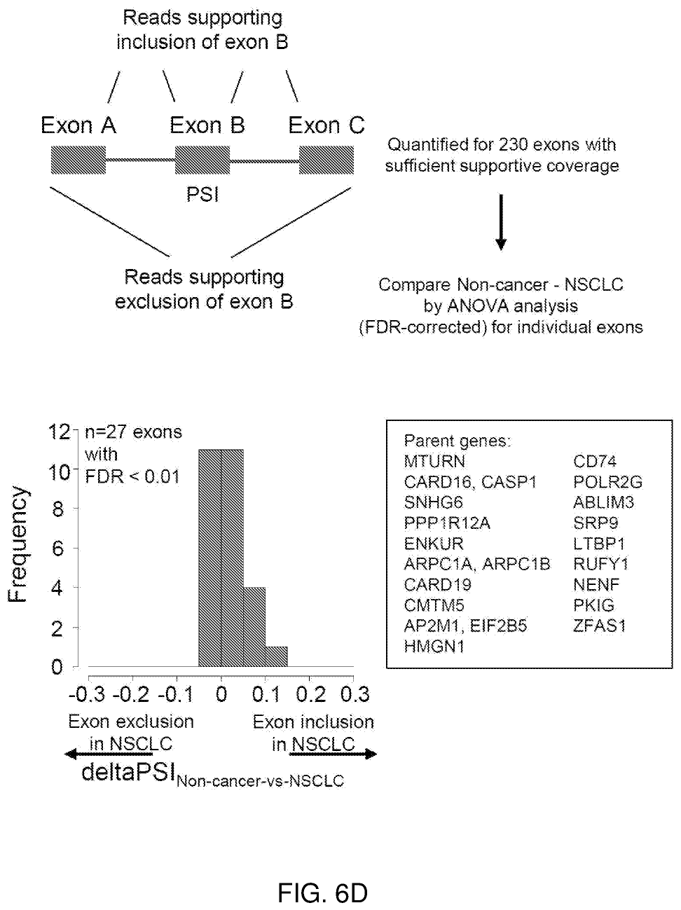

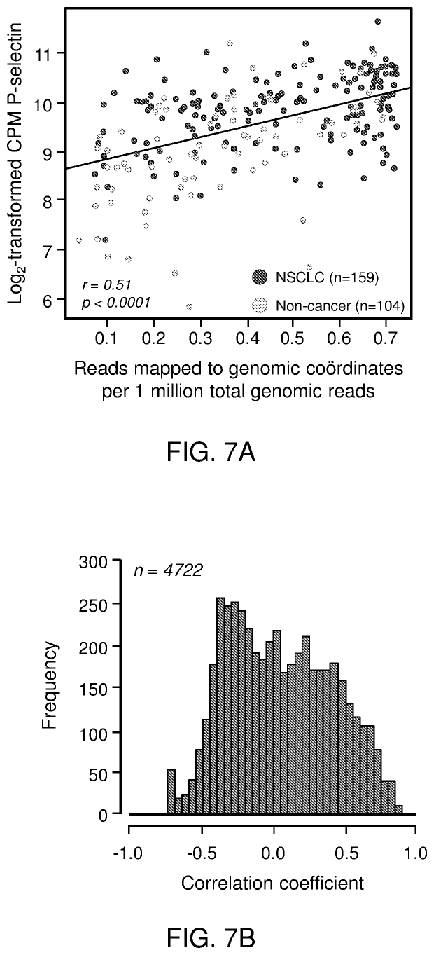

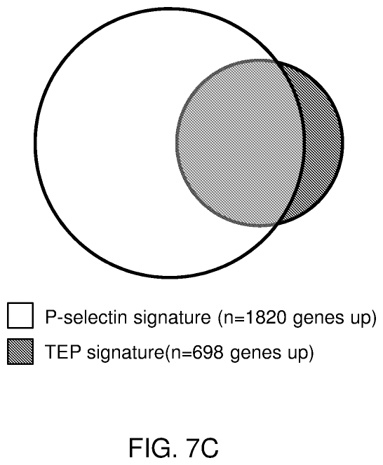

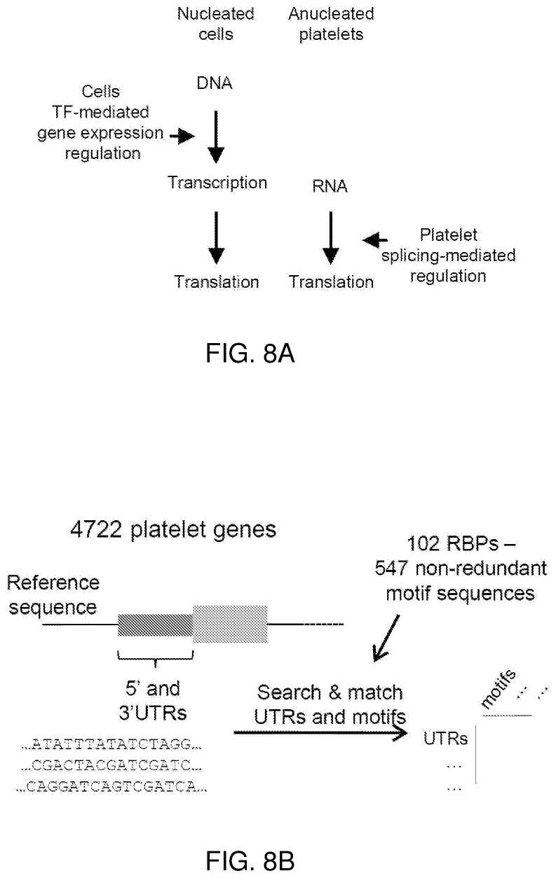

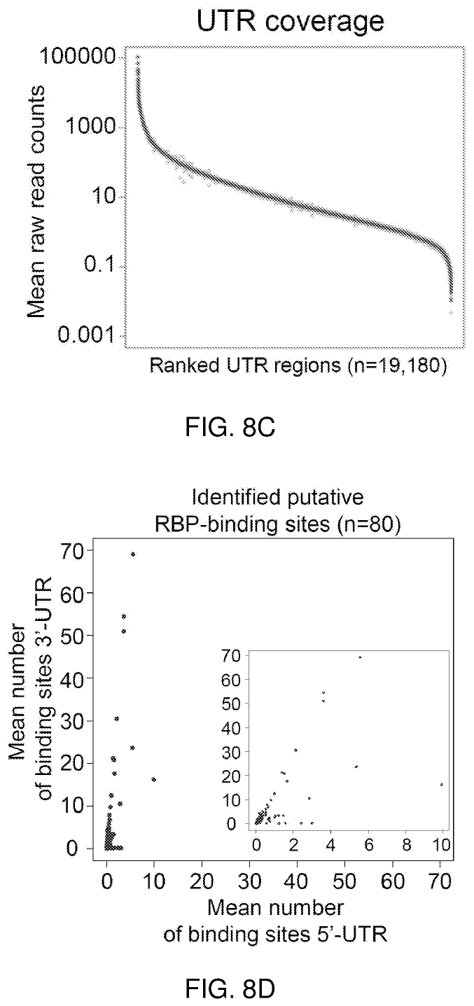

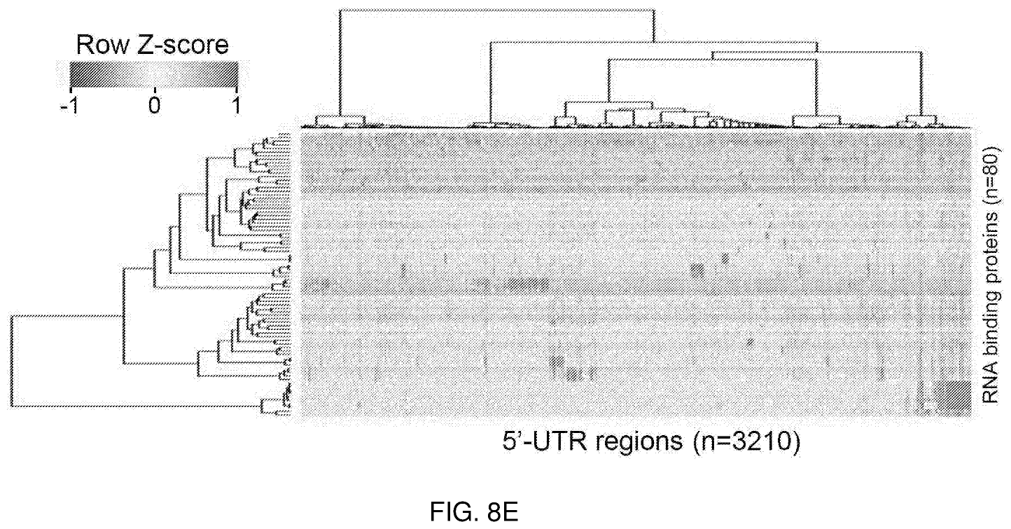

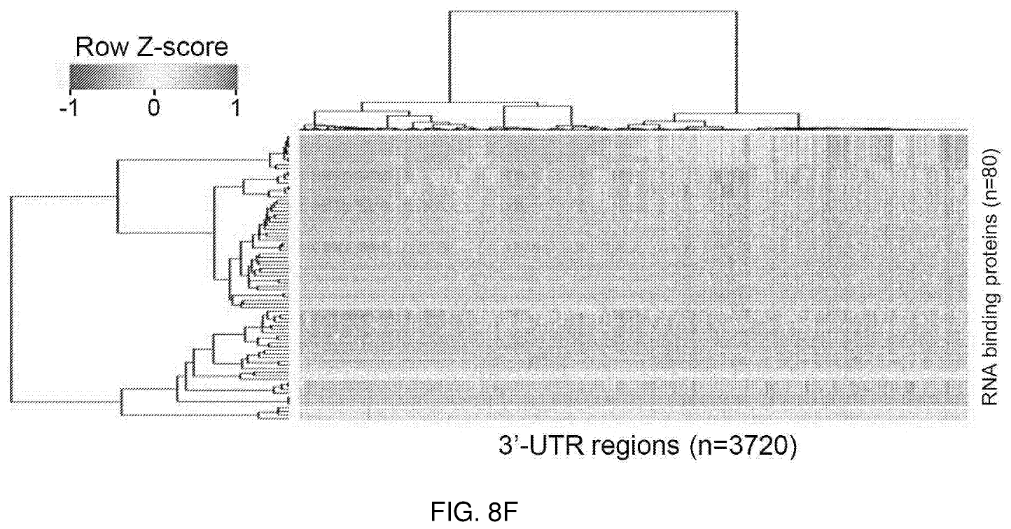

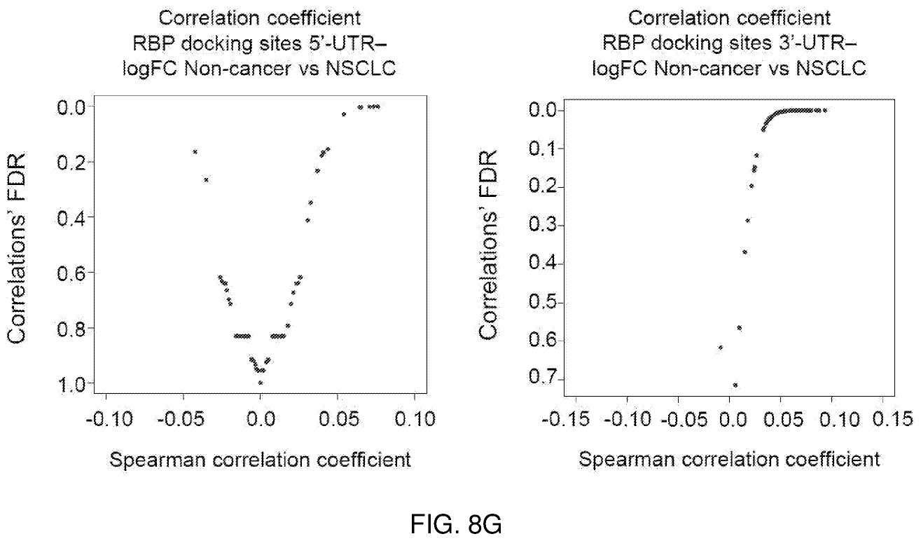

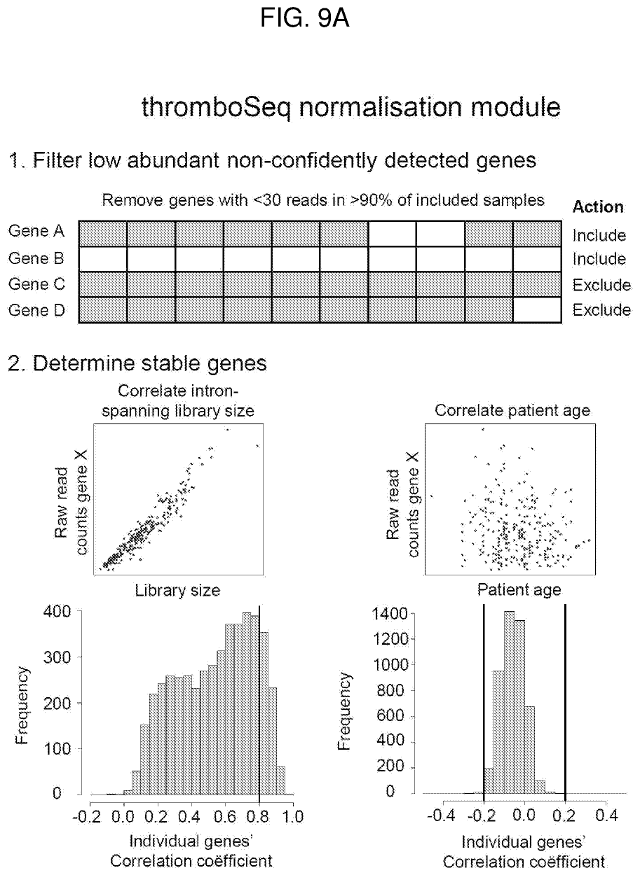

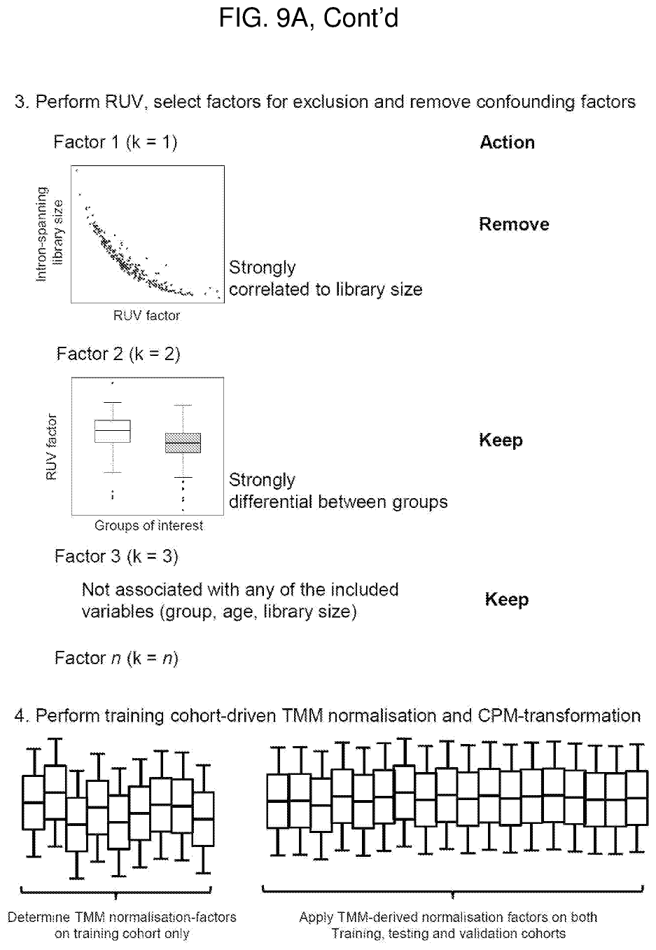

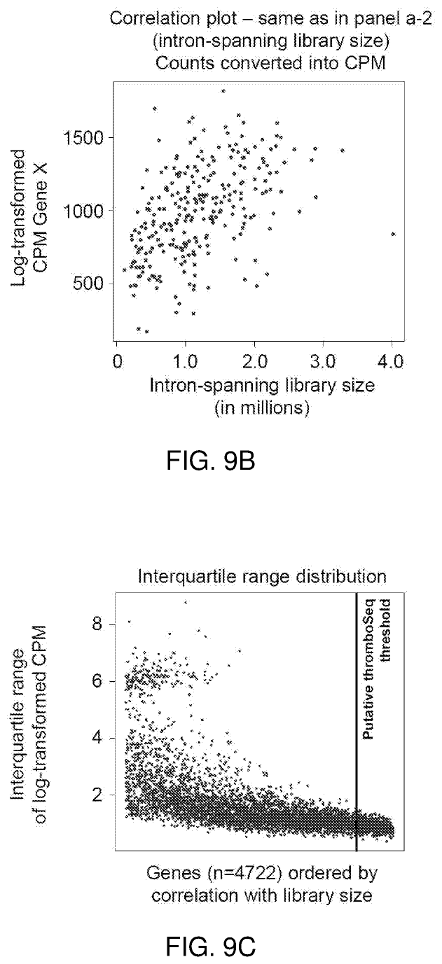

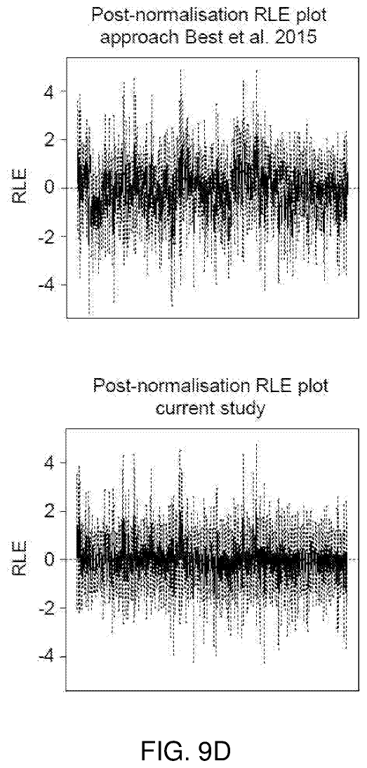

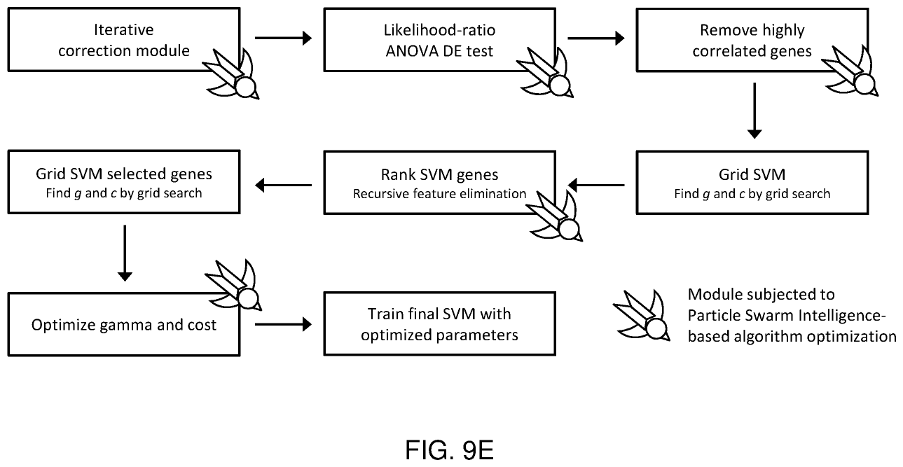

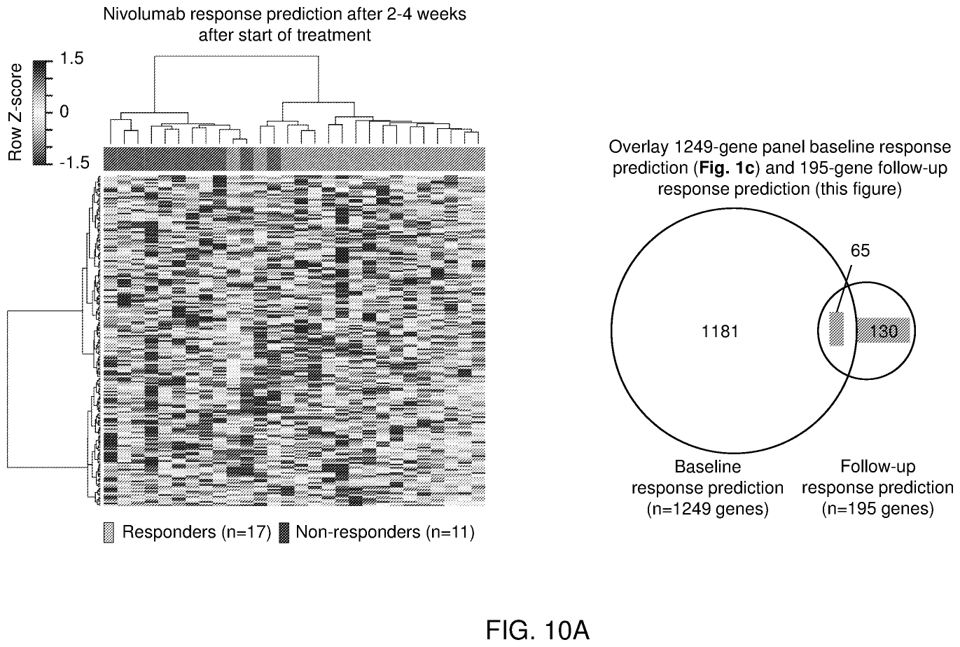

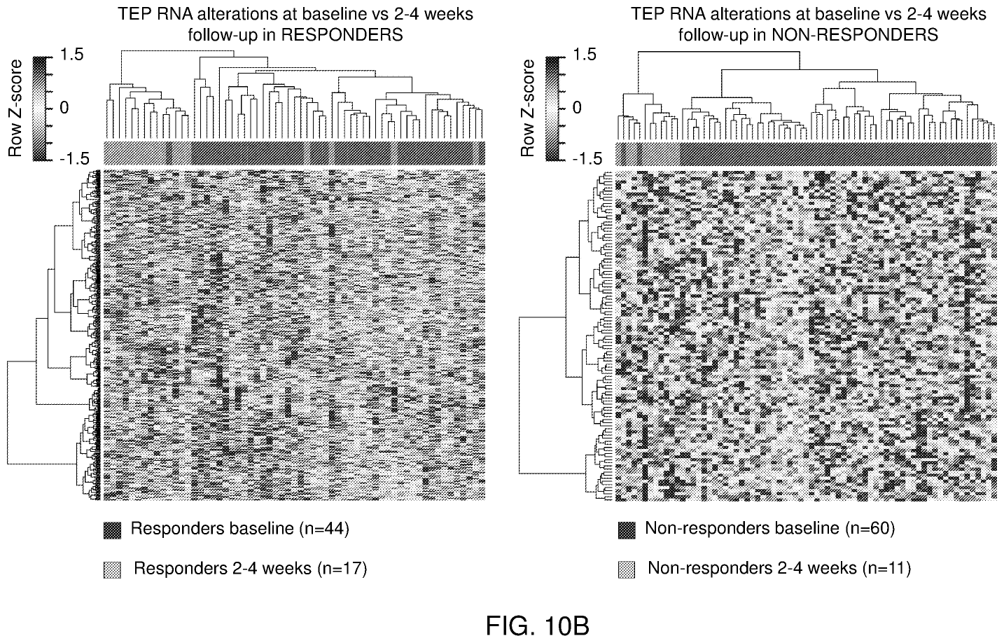

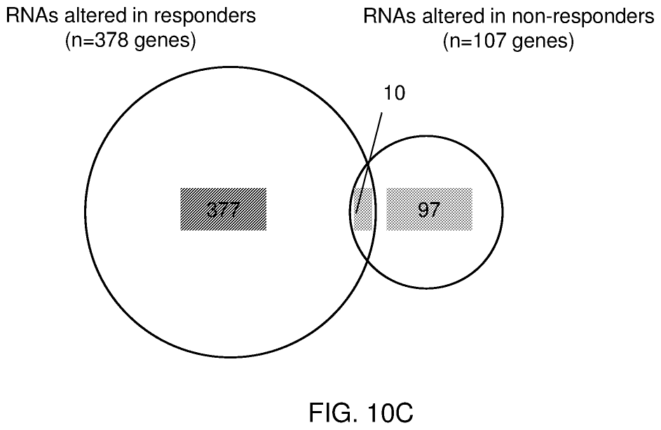

Raw RNA sequence data of platelets encoded in FASTQ-files were subjected to a standardized RNA-seq alignment pipeline, as described previously (Best et al., 2015. Cancer Cell 28: 666-676). In summary, RNA-sequence reads were subjected to trimming and clipping of sequence adapters by Trimmomatic (v. 0.22) (Bolger et al., 2014. Bioinformatics 30: 2114-2120), mapped to the human reference genome (hg19) using STAR (v. 2.3.0) (Dobin et al., 2013. Bioinformatics 29: 15-21), and summarized using HTSeq (v. 0.6.1), which was guided by the Ensembl gene annotation version 75 (Anders et al., 2014. Bioinformatics 31: 166-169). All subsequent statistical and analytical analyses were performed in R (version 3.3.0) and R-studio (version 0.99.902). Of samples that yielded less than 0.2×10E6 intron-spanning reads in total after sequencing, we again sequenced the original Truseq preparation of the sample and merged the read counts generated from the two individual FASTQ-files after HTSeq count summarization (performed for n=52 samples). Genes encoded on the mitochondrial DNA and the Y-chromosome were excluded from downstream analyses, except for the analyses in FIG. 6b. As expected, after sequencing of polyadenylated RNA we measured a significant enrichment of platelet sequence reads mapping to exonic regions (FIG. 6b). Sample filtering was performed by assessing the library complexity, which is partially associated with the intron-spanning reads library size (FIG. 4j). First, we excluded the genes that yielded <30 intron-spanning reads in >90% of the cohort for all platelet samples that were sequenced (n=740 Total, n=385 Non-cancer and n=355 NSCLC). This resulted in this platelet RNA-seq library in 4722 different genes detected with sufficient coverage. For each sample, we quantified the number of genes for which at least one intron-spanning read was mapped, and excluded samples with <3000 detected genes (˜1% lowerbound. FIG. 4j). Hereby we excluded 10 samples (n=8 (2.1% of total) Non-cancer, n=2 (0.6% of total) NSCLC). Next, to exclude platelet samples that show low inter-sample correlation, we performed a leave-one-sample-out cross-correlation analysis (FIG. 4m). Following data normalisation (see section ‘Data normalisation and RUV-mediated factor correction’ in Example 1), for each sample in the cohort, all samples except the ‘test sample’ were used to calculate the median counts-per-million expression for each gene (reference profile). Following, the comparability of the test sample to the reference set was determined by Pearson's correlation. Samples with a correlation <0.5 were excluded (n=2), and the remaining 728 samples were included in this study (FIG. 1a). Of note, we observed delicate differences in the Bioanalyzer cDNA profiles (spiked/smooth patterns), irrespective of patient group, but with a significant correlation to average cDNA length (FIG. 4f, g). This observation is discussed in more detail in Example 2. We measured the average length of concatenated reads mapped to intergenic regions for spiked and smooth samples separately using Bedtools (v. 2.17.0, Bedtools merge following Bedtools intersection), and observed that the majority of reads (>10.9% for spiked samples and >13.5% for smooth samples, n=50 samples each) had an average fragment length (concatenated reads) of <250 nt, with a peak at 100-200 nt. We attribute the differences in cDNA profiles at least partly to ‘contaminating’ plasma DNA retained during the platelet isolation procedure (FIG. 4h and Example 2). To prevent potential plasma DNA from contributing to our computational platelet RNA analyses, we only selected spliced intron-spanning RNA reads (FIG. 1b, FIG. 4i).

Assessment of the Technical Performance of thromboSeq

We observed in the platelet RNA a rich repertoire of spliced RNAs (FIG. 4k), including 4000-5000 different messenger and non-coding RNAs. The spliced platelet RNA diversity is in agreement with previous observations of platelet RNA profiles (Best et al., 2015. Cancer Cell 28: 666-676; Rowley et al., 2011. Blood 118: e101-11; Bray et al., 2013. BMC Genomics 14:1; Gnatenko et al., 2003. Blood 101: 2285-2293). To estinmate the efficiency of detecting the repertoire of 4000-5000 platelet RNAs from ˜500 pg of total platelet RNA input (FIG. 4k), we summarized all gene tags with at least 30 non-normalized intron-spanning read counts. We investigated whether collection of more single-read 100 bp RNA-seq reads (˜5× deeper: deep thromboSeq) of the platelet cDNA libraries (n=12 healthy donors) yielded in detection of more low-abundant RNAs (FIG. 4l). For this, we selected the gene tags that had more than 10 raw intron-spanning reads in at least one sample. This was performed separately for shallow and deep thromboSeq. For visualization purposes, we calculated the median raw intron-spanning read counts, log-transformed the counts (after adding one count to all tags), and plotted the 20,000 gene tags with highest count numbers. Again, this was performed separately for shallow and deep thromboSeq data. Increasing the average coverage of shallow thromboSeq ˜5× does not yield in significantly enriched detection of low-abundant platelet genes.

### Differential Splicing Analysis

Prior to differential splicing analyses the data was subjected to the iterative correction-module as described in the section ‘Data normalisation and RUV-mediated factor correction’ in Example 1 (age correlation threshold 0.2, library size correlation threshold 0.8 (Non-cancer/NSCLC, FIG. 5a) or 0.95 (nivolumab therapy response signature, FIG. 4b)). Corrected read counts were converted to counts-per-million, log-transformed, and multiplied by the TMM-normalization factor calculated by the calcNormFactors-function of the R-package edgeR (Robinson et al., 2010. Bioinformatics 26: 139-140). For generation of differential spliced gene sets, the after fitting of negative binominal models and both common, tag-wise and trended dispersion estimates were obtained, differentially expressed transcripts were determined using a generalized linear model (GLM) likelihood ratio test, as implemented in the edgeR-package. For data signal purposes, we performed differential expression analyses with post-hoc gene ontology interpretation using the corrected read counts as input for differential splicing analyses, whereas for reproducibility of the data during classification tasks we used the non-corrected raw read counts as input. Genes with less than three logarithmic counts per million (log CPM) were removed from the spliced RNA gene lists. RNAs with a p-value corrected for multiple hypothesis testing (FDR) below 0.01 were considered as statistically significant. For the nivolumab response prediction signature development using differential splicing analysis (FIG. 2b) and the classification algorithm (FIG. 2c), we used p-value statistics for gene selection. The nivolumab response prediction signature threshold was determined by swarm-intelligence, using the p-value calculated by Fisher's exact test of the column dendrogram (Ward clustering) as the performance parameter (see also section ‘Performance measurement of the swarm-enhanced thromboSeq algorithm’ in Example 1. Unsupervised hierarchical clustering of heatmap row and column dendrograms was performed by Ward clustering and Pearson distances. Non-random partitioning and the corresponding p-value of unsupervised hierarchical clustering was determined using a Fisher's exact test (fisher.test-function in R). To determine differentially splicing levels between platelets of Non-cancer individuals and NSCLC patients (FIG. 5), we included only samples assigned to the patient age- and blood storage time-matched cohort (training, and validation, n=263 in total, see also FIGS. 3c and 4a).

### Analysis of RNA-Seq Read Distribution

Distribution of mapped RNA-seq reads of platelet cDNA, and thus the origin of the RNA fragments, was investigated in samples assigned to the patient age- and blood storage time-matched NSCLC/Non-cancer cohort (training, evaluation, and validation, totaling 263 samples). The mitochondrial genome and human genome, of which the latter includes exonic, intronic, and intergenic regions were quantified separately (FIG. 6a). Read quantification was performed using the Samtools View algorithm (v. 1.2, options -q 30, -c enabled). For quantification of exonic reads, we only selected reads that mapped fully to an exon by performing a Bedtools Intersect filter step (-abam, -wa. -f 1, v. 2.17.0) prior to Samtools View quantification. We used bed-files of exonic, intronic, and intergenic regions annotated in Ensembl gene annotation version 37 and hg19 as a reference. Spliced RNAs were filtered from the aligned reads by selection of a cigar-tag in the bam-file, and reads mapping to the mitochondrial genome were selected by only quantifying reads mapping to ‘chrM’. We determined the ratios of reads mapping to the specific genomic regions by calculating the proportion of reads as compared to the total number of quantified reads per sample. Independent Student's t-test was performed using the t.test function in R. A detailed description of the results and data interpretation is provided in Example 3.

### P-Selectin Signature

To determine the correlation between p-selection levels and exonic read counts, we compared the P-selectin (SELP, ENSG00000174175) counts-per-million values of 263 patient age- and blood storage time-matched individuals to the number of reads mapping to exons (FIG. 7a). P-selectin expression levels were collected from log 2-transformed. TMM-normalised, and counts-per-million transformed read counts, subjected to RUV-mediated correction (see section ‘Data normalisation and RUV-mediated factor correction’ in Example 1, age correlation threshold 0.2, library size correlation threshold 0.9). Exonic read counts to P-selectin expression levels correlation analysis was performed using Pearson's correlation. To identify gene expression co-correlated to P-selectin enrichment, we calculated Pearson's correlations of all individual genes (n=4722 in total) to the P-selectin expression levels. Data was summarized in a histogram, and we compiled a P-selectin signature by selecting positively (r>0) and most significantly (FDR<0.01, adjusted for multiple hypothesis testing) correlated genes. The P-selectin signature was compared with all differentially and increasingly spliced genes between Non-cancer and NSCLC (FIG. 5a), and summarized in a Venn diagram (VennDiagram-package in R).

### Alternative Spliced Isoform and Exon Skipping Events Analyses

We employed the MISO algorithm (Katz et al., 2010. Nature methods 7: 1009-15) for alternative splicing analysis in our 100 bp single read RNA-seq data. Briefly, the MISO algorithm quantifies the number of reads favouring inclusion or exclusion of a particular annotated event, such as exon skipping, or RNA isoforms. By scoring reads supporting either one variant or the other (on/off) and scoring reads supporting both isoforms, the algorithm infers the ratio of inclusion, and thereby the percent spliced in (PSI). A detailed description of the alternative splicing analysis in TEPs and interpretation of the results is provided in Example 3.

Processing of Raw mRNA Sequencing Data for MISO Splicing Analysis

For the MISO RNA splicing analyses (FIGS. 6c and d). FASTQ-files of the patient age- and blood storage time-matched NSCLC/Non-cancer cohort were again subjected to Trimmomatic trimming and clipping, and STAR read mapping (see also section ‘Processing of raw RNA-sequencing data’ in Example 1). To create an uniform read length of all inputted reads, as required by the MISO algorithm, trimmed reads were cropped to 92 bp and reads below a read length of 92 bp were excluded from analysis. After addition of read groups using Picard tools (AddOrReplaceReadGroups function, v. 1.115), MISO sam-to-bam conversion was performed, and the indexed bam files were subjected to the MISO algorithm (v. 0.5.3) using hg19 and the indexed Ensembl gene annotation version 65 as reference. MISO output files were summarized using the summarize_miso-function. Summarized MISO files of isoforms and skipped exons were subsequently converted into ‘psi’ count matrices and ‘assigned counts’ count matrices using a custom script in MATLAB.

### Identification of Alternatively Spliced Isoforms

For alternative isoform analysis, we narrowed the analysis to the 4722 genes identified with confident intron-spanning expression levels in platelets (see also section ‘Processing of raw RNA-sequencing data’ in Example 1). For each annotated Ensemble transcript ID, available in the MISO summary output files, the assigned read counts (reads assigned to the particular RNA isoform) were summarized in a count matrix. A schematic overview of the procedure is presented in FIG. 6c. To ensure proper detection of the isoform, we excluded RNA isoforms with <10 reads in >90% of the sample cohort, and applied TMM- and counts-per-million normalisation. Next, differential expression analysis among annotated Ensembl transcripts was performed, and the most significant hits (FDR<0.01, log CPM>1) were selected. For details regarding the differential expression analysis, see section ‘Differential splicing analysis’ in Example 1. For identification of multiple RNA isoforms per parent gene locus, we matched the Ensembl transcript IDs (enst) with Ensembl gene IDs (ensg) and calculated the frequency metrics of the ensg-tags for the significant enst-tags. Distribution of alternatively spliced isoforms was assessed by including all enst-tags per parent gene locus, and comparing the median expression values for both Non-cancer and NSCLC samples. Isoforms that showed in all cases increased or decreased levels were scored as non-alternatively spliced. Isoforms that exhibited enrichment in either group but a decrease in the other, and the opposite for at least one other isoform were scored as alternatively spliced RNAs.

### Identification of Exon Skipping Events

For analysis of exon skipping events, we developed a custom analysis pipeline summarizing reads supporting inclusion or exclusion of annotated exons and scoring the relative contribution in groups of interest, i.e. Non-cancer versus NSCLC. The input for the algorithm is a PSI-values count matrix and an ‘assigned counts’ count matrix, as generated from summary output files generated by MISO. The former count matrix is required to calculate the relative PSI-values and distribution per group, the latter count matrix is required to only include exons with sufficient coverage in the RNA-seq data (i.e. >10 reads in >60% of the samples, which support both inclusion (1.0) and exclusion (0,1) of the variant, see also Katz et al.,). The coverage selector dowvnscaled the available exons for analysis to 230 exons (FIG. 6d). To select differential levels of skipping exon events. PSI-values were compared among Non-cancer and NSCLC using an independent student's t-test including post-hoc false discovery rate (FDR) correction (t.test and p.adjust function in R). Events with an FDR<0.01 were considered as potential skipped exon events. The deltaPSI-value was calculated by subtracting per skipping event the median PSI-value of Non-cancer from the median PSI-value NSCLC.

### RNA Binding Protein Motif Enrichment Analysis—RBP-thromboSearch Engine

To identify RNA-binding protein (RBP) profiles associated with the TEP signatures in NSCLC patients (FIG. 8), we designed and developed the RBP-thromboSearch engine. The rationale of this algorithm is that enriched binding sites for particular RBPs in the untranslated regions (UTRs) of genes is correlated to stabilization or regulation of splicing of that specific RNA. The algorithm identifies the number of matching RBP binding motifs in the genomic UTH sequences of genes confidently identified in platelets. Subsequently, it correlates for each included RBP the n binding sites to the logarithmic fold-change (log FC) of each individual gene, and significant correlations are ranked as potentially involved RBPs. For this analysis, we collected previously well-characterized RBP binding motifs from literature (Ray et al., 2013. Nature 499:172-177). The algorithm exploits the following assumptions: 1) more binding sites for a particular RBP in a UTR region predicts increased regulation of the gene either by stabilization or destabilization of the pre-mRNA molecule (Oikonomou et al., 2014. Cell Reports 7: 281-292), 2) the functions in 1) are primarily driven by a single RBP and not in combinations or synergy with multiple RBPs or miRNAs. or other cis or trans regulatory elements, and 3) the included RBPs are present in platelets of Non-cancer individuals and/or NSCLC patients. In order to determine the n RBP binding sites-log FC correlations, the algorithm performs the following calculations and quality measure steps:

(i) The algorithm selects of all inputted genes the annotated RNA isoforms and identifies genomic regions of the annotated RNA isoforms that are associated with either the 5′-UTR or 3′-UTR. The genomic coding sequence is extracted from the human hg19 reference genome using the getfasta function in Bedtools (v. 2.17.0). For this study, we used the Ensembl annotation version 75.
(ii) All characterized motif sequences extracted from literature (102 in total, Supplementary Table 3 of Ray et al., (Ray et al., 2013. Nature 499: 172-177), filtered for Homo Sapiens) are reduced to 547 non-redundant (‘A’, ‘G’, ‘C’, and ‘T’-sequence) annotations according to the IUPAC motif annotation. These non-redundant motif sequences serve as the representative motif sequences for the initial search.
(iii) In an iterative manner, per RBP the associated non-redundant RBP motif sequences are matched with all identified and included UTR sequences (using the str_count function of the seqinr package in R).
(iv) The algorithm identifies the number of reads mapping to each UTR region per sample using Samtools View (q 30, -c enabled. FIG. 8b). UTR sequences with no or minimal coverage were considered to be non-confident for presence in platelets. To account for the minimal bias introduced by oligo-dT-primed mRNA amplification (Ramskold et al., 2012. Nature Biotech 30: 777-782), we set the threshold of number of reads for the 3′-UTR at five reads, and for the 5′-UTR at three reads.
(v) For all 5′- and 3′-UTRs with sufficient coverage associated with the same parent gene (ensg), all matched UTR-non-redundant motif hits were summed, and summarized in a gene-motif matrix. Non-redundant motifs were converted to RBP-ids by overlaying all possible RBP-motif matches. This matrix is used for downstream analyses, data interpretation, and visualization.

We confirmed 3′- and 5′-UTR enrichment of particular RBPs (FIG. 8d), and observed UTR-clusters of co-involved RBPs (FIG. 8e,f). Correlations between log FC and n RBP binding sites were determined for all RBPs using Pearson's correlation, and summarized in a volcano plot (FIG. 8g). For a detailed description and interpretation of the results, see Example 4.

### Data Normalisation and RUV-Mediated Factor Correction

We identified two variables, i.e. blood storage time and patient age, that potentially influence the classifiers predictive strength (Table 4). To reduce the influence of confounding factors participating in the classification model, we applied the following novel approach for iterative RNA-sequencing data correction (see also schematic representation in FIG. 9a). The correction module is based on the remove unwanted variation (RUV) method, proposed by Risso et al., (Risso et al., 2014. Nature Biotech 32: 896-902; Peixoto et al., 2015. Nucleic Acids Res 43: 7664-7674), supplemented by selection of ‘stable genes’ (independent of the confounding variables), and an iterative and automated approach for removal and inclusion of respectively unwanted and wanted variation. The RUV correction approach exploits a generalized linear model, and estimates the contribution of covariates of interest and unwanted variation using singular value decomposition (Risso et al., 2014, Nature Biotech 32: 896-902). In principle, this approach is applicable to any RNA-seq dataset and allows for investigation of many potentially confounding variables in parallel. Of note, the iterative correction algorithm is agnostic for the group to which a particular sample belongs, in this case NSCLC or Non-cancer, and the necessary stable gene panels are only calculated by samples included in the training cohort. The algorithm performs the following multiple filtering, selection, and normalisation steps, i.e.:

(i) Filtering of genes with low abundance, i.e. less than 30 intron-spanning spliced RNA reads in more than 90% of the sample cohort (also included in the general QC-module, see section ‘Processing of raw RNA-sequencing data’).
(ii) Determination of genes showing least variability among confounding variables. For this, the non-normalized raw reads counts of each gene that passed the initial filter in (i) were correlated using Pearson's correlation to either the total intron-spanning library size (as calculated by the DGEList-function of the edgeR package in R) or the age of the individuals. Genes with a high Pearson correlation (towards 1) show the least variability after counts-per-million normalisation (see FIG. 9b,c), and were thus designated as stable genes.
(iii) Raw read counts of the training cohort were subjected to the RUVg-function from the RUVSeq-package in R. The stable genes identified among the confounding variables were used as ‘negative control genes’. Following, the individual estimated factors for each sample identified by RUVg are correlated to potential confounding factors (in the current study: library size, age of the individual) or the group of interest (for example Non-cancer versus NSCLC). The continuous (confounding) variables are correlated to the estimated variance of the samples. Dichotomous variables (e.g. group) are compared using a Student's t-test. In both instances, the p-value was used as a significance surrogate between the RUVg variable and the (confounding) variable. Of note, to prevent removal of a variable likely correlated to group, we applied two rules prior to matching a variable to a (confounding) factor. i.e. a) the p-value between RUVg variable and group should be at least >1e−5, and b) the p-value between RUVg variable and the other variable should be at least <0.01. Raw non-normalized reads were corrected for RUVg variable x in case this variable was correlated to a confounding factor. Finally, the total intron-spanning library size per sample was adjusted by calculating the sum of the RUVg-corrected read counts per sample.
(iv) RUVg-normalized read counts are subjected to counts-per-million normalization, log-transformation, and multiplication using a TMM-normalisation factor. The latter normalisation factor was calculated using a custom function, implemented from the calcNornmFactors-function in the edgeR package in R. Here, the eligible samples for TMM-reference sample selection can be narrowed to a subset of the cohort. i.e. for this study the samples assigned to the training cohort, and the selected reference sample was locked. We applied this iterative correction module to all analyses in this work. The estimated RUVg number of factors of unwanted variation (k) was 3. We directly compared the performance of our previous normalisation module and the iterative correction module presented in this study using relative log intensity (RLE) plots (FIG. 9d), and observed superior removal of variation within the expression data. RLE-plots were generated using the plotRLE-function of the EDASeq package. Significance of the reduction of inter-sample variability (FIG. 9d) was determined by calculating the absolute difference of the samples' median RLE counts to the overall median RLE counts for all samples for each sample with and without RUV-mediated factor correction.

### Support Vector Machine (SVM)-Based Algorithm Development and Particle Swarm-Driven SVM-Parameter Optimalisation

The swarm-enhanced thromboSeq algorithm implements multiple improvements over the previously published thromboSeq algorithm (Best et at, 2015. Cancer Cell 28: 68-676). An overview of the swarm-enhanced thromboSeq classification algorithm is provided in FIG. 9e. First, we improved algorithm optimization and training evaluation by implementing a training-evaluation approach. A total of 93 samples for the matched cohort (FIG. 1d) and 120 samples for the full cohort (FIG. 1e) assigned for training-evaluation were used as an internal training cohort. These samples served as reference samples for the iterative correction module (see ‘Data normalisation and RUV-mediated factor correction’-section in Example 1), initial gene panel selection by a likelihood ratio ANOVA test (see ‘Differential splicing analysis’-section in Example 1), SVM-parameter optimization, and final algorithm training and locking (selection of support vectors). Second, after the likelihood ratio ANOVA analysis we removed genes with high internal correlation (findCorrelations function in the R-package caret), as these were previously suggested to contribute to unwanted noise in SVM-models. Third, we implemented a recursive feature elimination (RFE) algorithm, previously proposed by Guyon et al., (Guyon et al., 2002. Machine Learning 46: 389-422), to enrich the gene panels for genes most relevant and contributing to the SVM classifiers. Fourth, following the final SVM cost and gamma parameter grid search (see FIG. 9e), we performed additional refinement of the cost and gamma parameters, by enabling an internal, second particle swarm algorithm (cv.particle_swarm-function in the R-package Optunity). This internal particle swarm algorithm was employed to investigate and pinpoint neighbouring values of the optimal gamma and cost parameters determined by the SVM grid search for more optimal internal SVM performance. Fifth, the entire SVM classification algorithm was subjected to a particle swarm optimization algorithm (PSO), implemented by the ppso-package in R (optim_ppso_robust-function) (Tolson and Shoemaker, 2007. Water Resources Research 43: W01413). Particle swarm intelligence is based on the position and velocity of particles in a search-space that are seeking for the best solution to a problem. Upon iterative recalibration of the particles based on its local best solution and overall best solution, a more refined estimate of the input parameters and algorithm settings can be achieved (FIG. 1c). The implemented algorithm allows for realtime visualization of the particle swarms, optimization of multiple parameters in parallel, and deployment of the iterative ‘function-calls’ using multiple computational cores, thereby advancing implementation of large classifiers on large-sized computer clusters. The PSO-algorithm aims to minimize the ‘1-AUC’-score. We employed for our matched NSCLC/Non-cancer cohort classifier 100 particles with 10 iterations and for the full NSCLC/Non-cancer cohort classifier 200 particles with 7 iterations. We optimized four steps of the generic classification algorithms, i.e. (i) the iterative correction module threshold used for selection of genes identified as stable genes among the library size (see also FIG. 9a), (ii) the FDR-threshold included in the differential splicing filter applied to the results of the likelihood ANOVA test, (iii) the exclusion of highly correlated genes selected after the likelihood ANOVA test, and (iv) number of genes passing the RFE-algorithm. Predefined ranges were submitted to the PSO-algorithm for every classification task presented in the this study. Training of SVM algorithms was performed using a two-times internal cross validation, and an initial gamma and cost parameter range for the grid search of respectively 2\hat{ }(−20:0) and 2\hat{ }(0:20). To account for undetected genes in the validation cohort, potentially hampering normalization of the data and reducing algorithm performance, genes with counts between zero and 12 (matched cohort) and 2 (full cohort) were replaced by the median counts of the training cohort, for that particular gene.

Performance Measurement of the Swarm-Enhanced thromboSeq Algorithm

We assessed the performance, stability, and reproducibility of the swarm-enhanced thromboSeq platform using multiple training, evaluation, and validation cohorts. A schematic overview of the cohorts used for assessment of the performance of the platform in patient age- and blood storage-matched cohorts is provided in FIG. 3b. A detailed description of the samples used for classification and assignment to the different cohorts is provided in Table 5. Demographic and clinical characteristics of the cohorts are summarized in Table 4, FIG. 4a, and Table 5. All classification experiments were performed with the swarm-enhanced thromboSeq algorithm, using parameters optimized by particle swarm intelligence. We assigned for the matched cohort (FIG. 1d) 133 samples for training-evaluation, of which 93 were used for RUV-correction, gene panel selection, and SVM training, and 40 were used for gene panel optimization. The full cohort (FIG. 1e) contained 208 samples for training-evaluation, of which 120 were used for RUV-correction, gene panel selection, and SMV training, and 88 were used for gene panel optimization. The nivolumab response prediction cohort contained randomly samples cohorts consisting of 60 training samples, 21 evaluation samples, and 23 independent validation samples. All random selection procedures were performed using the sample-function as implemented in R. For assignment of samples per cohort to the training and evaluation subcohorts, only the number of samples per clinical group was balanced, whereas other potentially contributing variables were not stratified at this stage (assuming random distribution among the groups). Performance of the training cohort was assessed by a leave-one-out cross validation approach (LOOCV, see also Best et al., (Best et al., 2015. Cancer Cell 28: 666-676)). During a LOOCV procedure, all samples minus one (‘left-out sample’) are used for training of the algorithm, after which the response status of the left-out sample is classified. Each sample is predicted once, resulting in the same number of predictions as samples in the training cohort. The list of stable genes among the initial training cohort, determined RUV-factors for removal, and final gene panel determined by swarm-optimization of the training-evaluation cohort were used as input for the LOOCV procedure. As a control for internal reproducibility, we randomly sampled training and evaluation cohorts, while maintaining the validation cohorts and the swarm-guided gene panel of the original classifier, and perform 100 (nivolumab response prediction) or 1000 (matched and full cohort NSCLC/Non-cancer) training and classification procedures. As a control for random classification, class labels of the samples used by the SVM-algorithm for training of the support vectors were randomly permutated, while maintaining the swarm-guided gene list of the original classifier. This process was performed 1000 for the matched and full NSCLC/Non-cancer cohort classifiers, and 100 for the nivolumab response prediction classifier. P-values were calculated accordingly, as described previously (Best et al., 2015. Cancer Cell 28: 666-676). Results were presented in receiver operating characteristics (ROC)-curves, and summarized using area under the curve (AUC)-values, as determined by the ROCR-package in R. AUC 95% confidence intervals were calculated according to the method of Delong using the ci.auc-function of the pROC-package in R (Delong et al., 1988. Biometrics 44: 837-45).

### Gene Ontology Analysis

For the gene ontology analysis, we investigated co-associated gene clusters using the PAGODA functions implemented in version 1.99 of the scde R-package (http://pklab.med.harvard.edu/scde/). PAGODA allows for clustering of redundant heterogeneity patterns and the identification of de novo gene clusters through pathway and gene set overdispersion analysis (Fan et al., 2016. Nature Methods 13: 241-244). In particular, the ability to identify de novo gene clusters is of interest for the analysis of platelet RNA-seq data, as platelet biological functions are potentially unannotated and can only be inferred by unbiased cluster analysis. Gene IDs as selected by differential splicing analysis (n=1622, FIG. 5a) were used as input to generate gene ontology library files. We used a distance threshold of 0.9 for the PAGODA redundancy reduction, and identification of de novo gene options was enabled. Remaining steps in the analysis were according to instructions from the PAGODA authors. PAGODA analysis revealed four major clusters (one existing and three de novo gene clusters) of co-regulated genes that were correlated to disease state. We selected clusters with a significantly enriched multiple hypothesis testing corrected z-score (adjusted z-score). The de novo clusters were further curated manually using the PANTHER Classification System (http://pantherdb.org/) on the 26 Sep. 2016.

### Example 2

By analysis of platelet RNA samples after SMARTer amplification we observed delicate differences in the SMARTer cDNA profiles (FIG. 4f), as measured by a Bioanalyzer DNA High Sensitivity chip. The slopes of the cDNA products can be subdivided in spiked, smooth, and intermediate spiked/smooth profiles, and do not tend to be disease-specific (FIG. 4g). The spiked pattern, which is the most abundantly observed slope (59% in both Non-cancer as NSCLC cohort) is possibly related to the relative small diversity of RNA molecules (˜4000-5000 different RNAs measured) in platelets. The remaining samples are characterized by a smooth or intermediate spiked/smooth cDNA product profile. Of note, the Picochip RNA profiles and DNA 7500 Truseq cDNA profiles are similar among the three SMARTer groups (FIG. 4f), and none of the SMARTer groups was enriched in low-quality RNA samples. The average cDNA length can be correlated to the SMARTer profiles, whereas the cDNA yield following SMARTer amplification was comparable. Notably, the samples with a more smooth-like pattern resulted in reduced total counts of intron-spanning spliced RNA reads, and a concomitant increase in reads mapping to intergenic regions (FIG. 4i). RNA-seq reads mapping to intergenic regions are considered to be derived from unannotated genes resulting in stacks of multiple (spliced) reads, or (genomic) DNA contamination resulting in scattered reads. By analysis of small bins of intergenic regions (1 kb each), we observed that a minority of these reads can be attributed to potential unannotated genes (data not shown). Analysis of the average length distribution of concatenated read fragment mapping to intergenic regions (see Example 1), revealed a median fragment size of ˜100-200 bp with a distinct peak at 100 bp, which might be derived from fragments of cell-free DNA (FIG. 4h) (Newman et al., 2014. Nature Med 20: 548-554; Jiang and Lo, 2016. Trends Gen 32: 360-371). We previously estimated the contribution of nucleated cells in the platelet isolation procedure (n=7 randomly selected platelet isolations), potentially explaining traces of genomic DNA, but observed only minor contamination of these nucleated (white blood) cell (Best et al., 2015. Cancer Cell 28: 666-676). Notably, the time between whole blood collection and start of the platelet isolation procedure is likely to be correlated to the SMARTer cDNA slopes. Samples that have been stored as whole blood for more than 24 hours showed a spiked pattern in virtually all cases, whereas platelets isolated directly after blood collection showed a smooth pattern in most of the cases. Cell-free DNA is rather unstable in whole blood collected in EDTA-coated tubes, and most traces of cell-free DNA are likely degraded after more than 12-24 hours of incubation. Therefore, we anticipated that the whole blood samples subjected to the platelet isolation protocol—immediately or within 12 hours after blood collection—were possibly contaminated by residual plasma-derived cell-free DNA, of which traces remain in the isolated platelet pellet. The contamination with ‘unwanted’ cell-free DNA in the platelet RNA profiles can be circumvented by selection of intron-spanning RNA-seq reads, since exon-to-exon reads are specifically RNA-derived. Standardization of sample collection by starting the platelet isolation within 4-24 hours after blood collection, is therefore suggested.

### Example 3

RNA-seq data offers an opportunity to quantify nearly any region of the transcriptome at high resolution. Hence we investigated the distribution of RNA species in the platelet RNA profiles. The platelets analyzed in this study make up a snapshot of all platelets circulating in the blood stream at moment of blood collection, and may be influenced by variables such as total platelet counts, medication, bleeding disorders, injuries, activities or sports, and circadian rhythm. For the following analyses, in order to reduce the influence of factors highly suspected of confounding the platelets profiles (Table 4), we selected 263 patient age- and blood storage time-matched individuals. Based on intron-spanning read count analysis we identified 1625 spliced platelet genes with significantly differentially spliced levels (FDR<0.01, 698 genes with enhanced splicing in platelets of NSCLC patients and 927 genes with decreased splicing in platelets of NSCLC patients), which is in line with previous findings (Best et al., 2015. Cancer Cell 28: 666-676; Calverley et al., 2010. Clinical and Transl Science 3: 227-232).

Based on unsupervised hierarchical clustering of intron-spanning reads the Non-cancer and NSCLC samples separated into two distinct groups (p<0.0001, Fisher's exact test, FIG. 5a). Next, we quantified the number of confidently mapped RNA-seq reads for each separate region of the mitochondrial genome and human genome, i.e. exonic, intronic, and intergenic fractions (see Example 1). We observed an on average increase in the number of reads mapping to the mitochondrial genome in NSCLC patients as compared to cancer-free individuals (FIG. 6b). Follow-up analysis revealed an increased number of normalized reads (the reads per one million total genomic reads) mapping to exonic fractions in NSCLC patients, whereas for intronic and intergenic fractions the opposite was observed (FIG. 6b). We further observed that, for samples with a larger proportion of reads mapping as intron-spanning spliced RNA reads, the contribution of reads mapping to the mitochondrial genome and intergenic regions was lower, whereas samples with low intron-spanning spliced RNA reads showed the opposite (FIGS. 4i and 6b).

Next, we investigated the contribution of alternative splicing events to the platelet RNA repertoire, since alternative splicing events might influence the number of spliced HNA reads used for the diagnostic classifiers. For characterization of transcriptome-wide alternative isoforms and splicing events, we implemented the previously published MISO algorithm (Katz et al., 2010. Nature Methods 7: 1009-1015) for the quantification and summarization of annotated RNA isoforms. From this, we inferred a count matrix, which contains the number of reads supporting each included RNA isoform per sample (FIG. 6c, see Example 1 for additional details). Next, we performed differential expression analysis between the RNA isoforms, and selected differential RNA isoforms between Non-cancer individuals (n=104) and NSCLC patients (n=159). Differential RNA isoform analysis between Non-cancer individuals and NSCLC patients revealed 743 RNA isoforms to be significantly enriched (n=359) or depleted (n=384) in TEPs of NSCLC patients. In 20% ( 113/571) of the genes, we identified multiple isoforms associated with the same gene locus (FIG. 6c). However, in only 13/571 (2.3%) of the genes we observed potential alternative splicing of the isoforms, although the differences between these particular RNA isoform were minor (data not shown). Altogether these results suggest that alternatively spliced RNA isoforms only have a minor-to-moderate contribution to the TEP profiles (FIG. 1b).

Next, we investigated alternative splicing events within genes. i.e. exon skipping. Here, we again applied the MISO algorithm (Katz et al., 2010. Nature Methods 7: 1009-1015) to profile 38327 annotated exons, and to infer the fraction of reads supporting either inclusion or exclusion of the particular exon as compared to neighbouring exons (schematic representation in FIG. 6d). In addition, the algorithm provides for each event a percent spliced in (PSI) value, quantifying the estimated fraction of reads supporting either inclusion or exclusion of a particular exon. For exon skipping analysis, 230 exons remained eligible for analysis after filtering for exons with low coverage. We applied ANOVA statistics, including correction for multiple hypothesis testing (FDR), for each included exon. By applying a threshold (ANOVA FDR<0.01), we identified 27 exon skipping events that were statistically significantly different in PSI value between Non-cancer and NSCLC samples (n=15 skipped in Non-cancer, n=12 skipped in NSCLC), and we observed a general trend towards exon inclusion in NSCLC (FIG. 6d). The putative exon skipping events are present in genes like SNHG6, CD74, and SRP9 (FIG. 6d). Hence, analysis of alternative splicing in platelets suggests a minor-to-moderate contribution to the TEP splicing profiles (FIG. 1b).

We also observed multiple variables converging, i.e. 1) platelets of NSCLC patients have an higher RNA yield on average (FIG. 4c), 2) platelets of NSCLC patients show on average a lower variety of processed and spliced RNAs, indicating reduced activity (FIG. 4k), and 5) platelets of NSCLC patients show an increased expression of reads mapping to exons and intron-spanning reads (FIG. 6b), whereas the reads spanning exon boundaries (potential unspliced RNAs) have similar levels in Non-cancer and NSCLC. In line with these findings, and supported by literature reports (Dymicka-Piekarska and Kemona, 2008. Thrombosis Res 122: 141-143; Dymicka-Piekarska et al., 2006. Advances Med Sciences 51: 304-308; Stone et al., 2012. New England J Med 366: 610-618; Watrowski et al., 2016. Tumour Biol 37: 12079-12087), the platelet fraction of cancer patients seems to be enriched with younger reticulated platelets. Reticulated platelets are newborn platelets (<1 day old), and contain considerably enriched levels of RNAs, as measured by thiazole orange staining (Hoffmann, 2014. Clinical Chem Lab Med 52: 1107-1117; Harrison et al., 1997. Platelets, 8: 379-383; Ingram and Coopersmith, 1969. British J Haematol 17: 225-229). Reticulated platelets were estimated to have an enriched RNA content of 20-40 fold (Angénieux et al., 2016. PloS one 11: e0148064). Hence, we hypothesized that the platelet RNA of NSCLC patients could be enriched with RNAs associated with younger platelets, including P-selectin (CD62) (Bernlochner et al., 2016. Platelets 27: 796-804). We indeed observed a highly significant positive correlation between exonic read coverage and P-selectin RNA-seq read counts (n=263, r=0.51, p<0.0001, Pearson's correlation, FIG. 7a). Next, we calculated an RNA signature correlated to P-selectin, and defined a profile of 2797 confidently detected and P-selectin co-correlating genes (FDR<0.01, FIG. 7b). The P-selectin signature was enriched for markers like CASP3, previously implicated in megakaryocyte-mediated pro-platelet formation (Morishima and Nakanishi, 2016. Genes Cells 21: 798-806). MMP1 and TIMP1, previously shown to be sorted to platelets (Ceechetti et al., 2011. Blood 118: 1903-1911), and ACTB, previously detected in reticulated platelets (Angénieux et al., 2016. PloS One 11: e0148064), providing validity of the P-selectin reticulated platelet signature. We observed that 77% of genes in the P-selectin signature were also identified as significantly enriched in the TEPs of NSCLC patients (FIG. 7c). Hence, we estimated that the contribution of the younger reticulated platelets to the TEP RNA profiles of NSCLC patients is significant (FIG. 1b and FIG. 7c).

### Example 4

Platelets are anucleated cell fragments. They contain, however, a functional spliceosome and several splice factor proteins (Denis et al., 2005. Cell 122: 379-391). Therefore, platelets retain their capacity to initiate pre-mRNA splicing. Several experiments have demonstrated that platelets are able to splice pre-mRNA upon environmental queues (Rondina et al., 2011. Journal Thromb Haemostasis 9: 748-758; Schwertz et al., 2006. J Exp Med 203: 2433-2340; Denis et al., 2005. Cell 122: 379-391), and that they have the ability to translate RNA into proteins (Weyrich et al., 1998. Proceedings of the National Academy of Sciences 95: 5556-5561). As platelets lack a nucleus, but are packaged with ˜20-40 femtograms of RNA (Angénieux et al., 2016. PloS One 11: e0148064) and circulate for 7-10 days, the (pre-)mRNA needs to be properly curated. The inability of platelets to transcribe chromosomal DNA, as opposed to nucleated cells, prevents the platelets from transcription factor-mediated gene regulation, hinting at post-transcriptional regulation of the RNA pool (FIG. 8a), possibly by RNA binding proteins (RBPs) (Zimmerman and Weyrich, 2008. Arteriosel Thromb Vasc Biol 28: s17-24). Indeed, the SF2/ASF- (SRSF1-) RBP has previously been implicated in the initiation of splicing of tissue factor mRNA in the platelets of healthy individuals (Schwertz et al., 2006. J Exp Med 203: 2433-2440). In general, RBPs are implicated in multiple co- and post-transcriptional processes associated with gene expression, such as RNA splicing, poly-adenylation, stabilization, and localisation (Glisovic et al., 2008. FEBS Letters 582: 1977-1986). A co-assembly of multiple RBPs with RNA molecules results in heterogeneous nuclear ribonucleoproteins (hnRNPs), which can define the fate of the pre-mRNA molecules. The 5′- and 3′-UTR are considered to be the most prominent regulatory regions for pre-mRNAs (Ray et al., 2013. Nature 499 172-177), whereas intronic regions primarily mediate alternative splicing events such as exon skipping. SAGE analyses of platelet RNA lysates have shown that the platelets contain genes with an on average longer 3′-UTR length (Dittrich et al., 2006. Thromb Haemostasis 95: 643-651). Therefore, we hypothesized that differential binding of RBPs to the UTR regions of platelet RNAs might explain the differential splicing patterns observed in TEPs. We developed an algorithm that scans for RBP binding motifs in UTR regions, and which identifies correlations between the number of binding sites and the log fold-change of the particular gene. We termed the algorithm the RBP-thromboSearch engine (FIG. 8b, see details in Example 1). We included 102 RBPs of which the binding motifs were previously identified (Ray et al., 2013. Nature 499: 172-177). We only included UTR regions with sufficient read coverage in the RNA-seq data (FIG. 8c, see Example 1). We first identified RBPs with enriched tropism for either the 5′-UTR or 3′-UTR, and indeed observed that RBM8A, FUS, and PPRC1 were primarily targeted towards the 5-ULTR, whereas IGF2BP2. ZC3H14, and RALY showed an enriched binding repertoire for the 3′-UTR (FIG. 8d). These enrichments were reported previously (Ray et al., 2013. Nature 499: 172-177), supporting the specificity of our matching-approach. All UTRs had at least one binding site for one of the RBPs. By analysis of the 3210 5′-UTR regions and 3720 3′-UTR regions, we observed that the number of RBP binding sites per UTR region showed a bimodal distribution, indicating controlled regulation of specific RBPs for specific UTR regions (FIG. 8e, f). To assess whether the RNAs in the NSCLC TEP RNA signatures are co-regulated by specific RBP binding sites, we correlated the log FC-values of either the 5′-UTR or 3′-UTR of the genes to the number of matching binding sides on either of these regions for each RBP. This resulted in 5 significant correlations for the 5′-UTR (FDR<0.01, RBM4, RBM8A, PPRC1, FUS, SAMD4A) and 69 for the 3′-UTR (FDR<0.01, top 5 is PCBP1/2, SRSF1, RBM28 LIN28A. and CPEB2, FIG. 8g). The significant correlations between n RBP binding sites and the log FC of the signature genes were positive for all significantly enriched RBPs, suggesting that enhanced binding sites might lead to enhanced splicing. Possibly, upon platelet activation, RBPs are released from specific granules into the platelet cytosol, thereby starting the splicing process. Alternatively, RBPs are controlled by protein kinases, such as Clk, that regulated RBP phosphorylation (Denis et al., 2005. Cell 122: 379-391; Schwertz et al., 2006. J Exp Med 203: 2433-2440), and thereby its intracellular localization (Colwill et al., 1996. EMBO J 15: 265-275). Thus, we conclude that differential RBP binding signatures might at least partially contribute to the specific TEP signatures, although further experimental validation is warranted.

### Example 5

### Development of Classification Signature

Blood platelets act as local and systemic responders during tumorigenesis and cancer metastasis (McAllister and Weinberg 2014. Nature Cell Biol 16: 717-27), thereby being exposed to tumor-mediated platelet education, and resulting in altered platelet behaviour (Labelle et al., 2011. Cancer Cell 20: 576-590; Schumacher et al., 2013. Cancer Cell 24: 130-137; Kerr et al., 2013. Oncogene 32: 4319-4324). We have previously demonstrated that platelet RNA can function as a biomarker trove to detect and classify cancer from blood via self-learning support vector machine (SVM)-based algorithms (Best et al., 2015. Cancer Cell 28: 666-676)(FIG. 3a). For platelet RNA biomarker selection and computational analyses, the isolated platelet RNA is first subjected to SMARTer cDNA synthesis and amplification. Truseq library preparation, and Illumina Hiseq sequencing (FIG. 4d-e, Example 1). We termed this highly multiplexed biomarker signature detection platform thromboSeq. Extrinsic factors can be of influence in the selection process and read-out of the platelet RNA biomarkers (Diamandis, 2016. Cancer Cell 29: 141-142; Joosse and Pantel, 2015. Cancer Cell 28: 552-554; Feller and Lewitzky, 2016. Cell Communication and Signaling 14: 24), and by statistical modeling of publicly available data (Best et al., 2015. Cancer Cell 28: 666-676), we were able to confirm that age of the individual and blood storage time can influence the platelet classification score (Table 4). Hence, we assembled cohorts of blood platelet samples from patients with NSCLC (n=159) and individuals with no known cancer (n=104), matched for age (median age (interquartile range: IQR) of 61 (14.5) and 58 (12.25) years respectively, FIG. 4a), and blood storage time (platelet isolation within 12 hours of blood collection). This matched cohort is part of a larger cohort of NSCLC patients (n=352) and individuals with no known cancer, but not excluding individuals with inflammatory diseases (n=376) (FIG. 1a, Table 4, Table 5, FIG. 4a). The matched NSCLC/Non-cancer cohort enabled us to first assess the contribution of potential technical and biological variables, i.e. platelet activation, platelet RNA yield, platelet maturation, and circulating DNA contamination (FIGS. 4-5, Example 2), and to investigate the platelet RNA profiles and RNA processing pathways (FIG. 1b, FIGS. 5-8, Examples 3-4). In addition, we investigated the platelet RNA sequencing efficiency using the thromboSeq platform (FIG. 4) Altogether, our results demonstrate that selection of intron-spanning spliced RNA reads eliminates potential undesired contribution of DNA contamination in the platelet RNA biomarker selection process, and that per sample a repertoire of at least 3000 different genes has to be detected prior to inclusion for diagnostic algorithm development (FIG. 4). In addition, the spliced platelet RNA profiles in patients with NSCLC seem to be predominantly altered by canonical splicing events and RNA-binding protein activity during platelet education and maturation in response to tumor growth (FIG. 1b, FIGS. 4-8, Examples 2-4). Next, we employed the matched NSCLC/Non-cancer platelet cohort to develop a NSCLC diagnostics classification algorithm (FIG. 1). We first improved the robustness of the data normalisation procedure of our previously developed SVM-based thromboSeq classification algorithm (Best et al., 2015. Cancer Cell 28: 666-676) by introduction of a RUV-based (Risso et al., 2014. Nature Biotech 32: 896-902) iterative correction module, thereby considerably reducing the relative intersample variability (p<0.0001, two-sided Student's t-test, FIG. 9a-d). Second, we implemented a PSO-driven meta-algorithm for selection of the most contributive genes used for classification (FIG. 1c, FIG. 9e). The PSO-driven algorithm leverages the use of many candidate solutions (i.e. particles), and by adopting swarm intelligence and particle velocity the algorithm continuously searches for more optimal solutions, ultimately reaching the most optimal fit (Kennedy et al., 2001. The Morgan Kaufmann Series in Evolutionary Computation. Ed: David B. Fogel; Bonyadi and Michalewicz 2016. Evolutionary computation: 1-54). Finally, we tested and validated the PSO-driven thromboSeq algorithm using the NSCLC/Non-cancer cohorts matched for patient age and blood storage time (n=263 in total). We summarized the predictive measures of the PSO-enhanced thromboSeq platform in a receiver operating characteristics (ROC) curve. We observed that this NSCLC classification algorithm has significant predictive power in patient age- and blood storage time-matched evaluation (accuracy: 85%, AUC: 0.91, 95%-CI: 0.82-1.00, n=40, red line. FIG. 1d) and validation cohorts (accuracy: 91%, AUC: 0.95, 95%-CI: 0.91-0.99, n=130, blue line, FIG. 1d). Post hoc leave-one-out cross validation (LOOCV) analysis of the training cohort suggests reduced performance (accuracy: 77%, AUC of 0.84, 95%-CI: 0.75-0.92, n=93, dashed grey line, FIG. 1d), as compared to the ‘matched’ evaluation (85% accuracy) and validation cohort (91% accuracy). This may be explained by the different classification techniques used, and optimization of the gene panel towards the evaluation cohort at cost of classification power in the training cohort. Following swarm-enhanced gene panel selection, the performance metrics of the training, evaluation and validation cohorts suggest that the algorithm has not been overfitted, a common pitfall of machine learning tasks (Lever et al., 2016. Nature Methods 13: 703-704). The contribution of patient age and blood storage time to the cancer classification was negligible as compared to the predictive power attributed to platelet RNA (Table 4). Of note, random selection of 1000 other patient age- and blood storage time-matched training cohorts from the same sample library (n=93 each) showed similar classification strength (median AUC ‘validation cohort’: 0.85, IQR: 0.05), as opposed to random classification (median AUC ‘validation cohort’: 0.55, IQR: 0.01, p<0.001). Subsequently, we included all samples of the full non-matched NSCLC/Non-cancer cohort (n=352 and n=376, respectively) and developed a new classification algorithm. For development of the algorithm training cohort, we summed all matched patient age and blood storage time samples and assigned 120 samples for swarm-guided gene list selection and SVM training, and 88 samples for swarm-based optimization. Hence, again the training cohort of the NSCLC diagnostics classifier was not confounded by patient age or blood storage time (Table 4). A total of 520 samples (patient age- and/or blood storage time-unmatched), including samples collected in multiple hospitals and from different clinical cohorts (Table 5), remained for validation of the algorithm, and were predicted by the algorithms while the algorithms' classification parameters were locked. We again summarized the predictive measures of the PSO-enhanced thromboSeq platform in a HOC curve, for evaluation (accuracy: 91%, AUC: 0.93, 95%-CI: 0.87-0.99, n=88, red line. FIG. 1e) and validation (accuracy: 89%, AUC: 0.94, 95%-CI: 0.93-0.96, n=520, blue line. FIG. 1e). Post-hoc LOOCV analysis of the training cohort again resulted in reduced performance (accuracy: 84%, AUC: 0.90, 95%-CI: 0.84-0.95, n=120, dashed grey line, FIG. 1e), as compared to the ‘full’ evaluation (91% accuracy) and validation cohort. (89% accuracy). Random selection of other training cohorts (n=120 each) while locking the gene panel resulted in similar classification strength (n=1000, median AUC ‘validation cohort’: 0.89. IQR: 0.05), whereas for random classification algorithm performance diminished (median AUC ‘validation cohort’: 0.5, IQR: 0.03, p<0.001). Therefore, we conclude that the PSO-driven thromboSeq platform allows for robust biomarker selection for blood-based cancer diagnostics, independent of bias introduced by age of the individual, blood storage time, and certain inflammatory diseases.

### Example 6 Development of Response Signature

Next, we investigated the clinical utility of swarm-modulated TEP biomarker signatures for therapy response prediction in patients with NSCLC. For this, we prospectively included patients with NSCLC that were selected for treatment with the PD-1 monoclonal antibody nivolumab that is associated with an objective response rate of approximately 20% in unselected NSCLC cohorts in the second line setting (Borghaei et al., 2015. New England J Med 373: 1627-1639; Brahmer et al., 2015. New England J Med 373: 123-135). Currently, stratification of patients for anti-PD-(L)1 targeted therapy is hampered by limited accuracy and concordance of available biomarkers, including PD-L1 immunohistochemistry of tumor tissue. Studies have identified correlations between tumor tissue mutational load, presence of neo-antigens, infiltration of immune cells, and response to anti-PD-(L)1 immunotherapy (Rizvi et al., 2015. Science 348: 124-128; McGranahan et al., 2016. Science 351: 1463-1469). Identification of patients with a low likelihood of response to anti-PD-(L)1 immunotherapy, while still correctly identifying individuals who most likely benefit from this therapy, might prevent unnecessary treatment and concomitant costs, and potential exposure of patients to serious immunological adverse events. Platelets can behave as immunomodulators in inflammatory conditions (Boilard et al., 2010. Science 327: 580-583), and are therefore potentially also involved in the immune response towards a tumor. To this end, we collected platelet samples before start of nivolumab treatment (n=64). These samples are part of the cohort presented in FIG. 1a. Response assessment of patients treated with nivolumab was performed by computed tomography (CT)-imaging at baseline, 6-8 weeks, 3 months and 6 months after start of treatment (FIG. 2a). Treatment response was assessed according to the updated Response Evaluation Criteria in Solid Tumours (RECIST) version 1.1. NSCLC patients with disease control (i.e. complete and partial responders, and patients with stable disease at six months after start of nivolumab treatment), were assigned to the responders group. For thromboSeq analysis, we selected baseline blood samples of 64 NSCLC patients treated with nivolumab (n=44 responders and n=60 non-responders), aiming at relatively balanced group sizes for optimal development of the PSO-driven nivolumab response prediction algorithm (FIG. 2a). First, we observed significant non-random clustering of differentially spliced RNAs in platelets of 44 responders and 60 patients not responding to nivolumab (gene panel optimized by swarm-intelligence, p<0.0001 by Fisher's exact test, FIG. 2b). Next, we re-applied swarm-intelligence for nivolumab response prediction signature identification. For this, we randomly selected a 60-sample training, 21-sample dependent evaluation, and 23-sample validation cohorts. The PSO-enhanced thromboSeq classification algorithm reached, using a 1246-gene nivolumab response prediction panel, an accuracy of 76% in the dependent evaluation cohort (AUC: 0.72, 95%-CI: 0.49-0.96, n=21, grey line. FIG. 2c). We next observed that the 1246-gene nivolumab response prediction algorithm has significant predictive power in an independent validation cohort. (accuracy: 83%, AUC: 0.89, 95%-CI: 0.67-1.00, n=23, blue line, FIG. 2c). Post hoc leave-one-out cross validation (LOOCV) analysis of the training cohort, during which each samples of the 60-samples training cohort is left out for algorithm training and subsequently predicted, resulted in high-accuracy classifications (accuracy: 83%, AUC: 0.89, 95%-CI: 0.81-0.97, red line, FIG. 2c). We confirmed the sensitivity of the nivolumab response prediction classifier by randomly selecting other training and dependent evaluation cohorts with similar sample sizes (n=1000 iterations, median AUC: 0.78, IQR: 0.09). In addition, we confirmed the specificity by randomly shuffling class labels (permutations) during the training process, resulting in random classifications (n=1000, median AUC: 0.30, min-max: 0.2-0.31, p<0.0001. FIG. 2c). Selection of an algorithm threshold at which all responders are correctly assigned for nivolumab treatment (100% sensitivity) using this 1246-gene classifier results in correct assignment in 53% of the cases in non-responders (53% specificity, FIG. 2d).

Assuming a 20% response rate to nivolumab among an unselected population of NSCLC patients (Borghaei et al., 2015. New Engl J Med 373: 1627-1639; Brahmer et al., 2015. New Engl J Med 373: 123-135), 42% of the full population will safely be withheld nivolumab treatment. We noted that classification of the n28-Follow-up-cohort (collected 2-4 weeks after start of treatment) in the 1246-genes nivolumab response prediction algorithm yielded in random classification (data not shown). However, we observed similar distinctive power in TEP RNA profiles at 2-4 weeks following start of treatment when analyzed separately (FIG. 10a), indicating that for a response predictor, during nivolumab treatment a separate classifier has to be build. We also noted that the TEP RNA profiles alter while patient are treated with nivolumab (FIG. 10b,c).

Altogether, we provide evidence that TEPs could potentially serve as a diagnostics platform for cancer detection and therapy selection. The PSO-driven thromboSeq algorithm development approach allowed for efficient biomarker selection and may be applicable to other diagnostics biosources and indications. A further increase in the classification power of swarm-enhanced thromboSeq may be achieved by 1) training of the swarm-enhanced self-learning algorithms on significantly more patient age- and blood storage time-matched samples, 2) including analysis of small RNA-seq (e.g. miRNAs), 3) including non-human RNAs, and/or 4) combining multiple blood-based biosources, such as TEP RNA, exosomal RNA, cell-free RNA, and cell-free DNA. By nature, swarm intelligence allows for self-reorganization and re-evaluation, enabling continuous algorithm optimization (FIG. 3a). At present, large scale validation of TEPs for the (early) detection of NSCLC and nivolumab response prediction is warranted.

### Example 7 Patient Cases

A 60-years-old male presents at the general practitioner (GP). He complains about sputum mixed with blood, tiredness, shortness of breath, and loss of weight. Upon physical examination the GP notices enlargement of clavicular lymph nodes. The GP suspects the patient of localized or metastasized lung cancer. He orders a platelet-RNA-based diagnostic test (thromboSeq). The patient is subjected to a venipuncture, and whole blood is collected in a EDTA-coated tube. The EDTA-coated tube with blood is send via medical transport to a sequencing facility, compatible with the thromboSeq system. Upon arrival of the blood tube at the sequencing facility the EDTA-coated tube is subjected to the standardized platelet isolation protocol, and from the resulting platelet pellet total RNA isolation is performed. The total RNA is quantified, quality-controlled, and ˜500 pg RNA is subjected to the standardized SMARTer cDNA amplification protocol. Resulting cDNA is labelled for Illumina sequencing, and the sample is sequenced using the Illumina sequencing platform.

Following sequencing, the samples' FASTQ-file is processed using the thromboSeq bioinformatics pipeline, consisting of read mapping, quantification, normalization, and correction, and classified using the swarm-enhanced NSCLC Dx signature-based support vector machine (SVM) classifier. The classification result is send to the GP.

A 66-years-old female is diagnosed with a stage IV non-small cell lung cancer (NSCLC), with multiple metastases to the brain. The medical doctors decide to investigate the sensitivity of the primary tumor for anti-PD(L)1-targeted treatment, especially nivolumab treatment. They draw blood using a regular venipuncture procedure, and collect the whole blood in EDTA-coated vacutainer tubes. The EDTA-coated tube with blood is send via medical transport to a sequencing facility, compatible with the thromboSeq system. Upon arrival of the blood tube at the sequencing facility the EDTA-coated tube is subjected to the standardized platelet isolation protocol, and from the resulting platelet pellet total RNA isolation is performed. The total RNA is quantified, quality-controlled, and ˜500 pg RNA is subjected to the standardized SMARTer cDNA amplification protocol. Resulting cDNA is labelled for Illumina sequencing, and the sample is sequenced using the Illumina sequencing platform. Following sequencing, the samples' FASTQ-file is processed using the thromboSeq bioinformatics pipeline, consisting roughly of read mapping, quantification, normalization, and correction, and classified using the swarm-enhanced nivolumab therapy response signature-based SVM classifier. The classification result, which contains a predicted response efficacy to nivolumab, is send to the medical team.

### Example 8 Minimal Biomarker Panels

### NSCLC Diagnostics Gene Panel

To select a minimal biomarker gene panel for TEP-RNA NSCLC diagnostics, a NSCLC diagnostics score was calculated. The NSCLC/Non-cancer RNA-sequencing dataset (n=779 samples) was first subjected to the RUV-normalization module (lib-size threshold: 0.418, as determined by PSO). The genes with stable expression levels among the cohort and the factors for RUV-correction were determined using the training cohort only (n=120 samples). Next, ANOVA differential expression analysis using only the samples assigned to the age-, gender-, EDTA-, and smoking-matched NSCLC/Non-cancer training cohort was performed. Following, an iterative biomarker gene panel selection algorithm, which adds per iteration a new gene according to a ranked FDR- or p-value-ranked ANOVA list, was employed. The biomarker gene panel is composed of genes with a positive logarithmic fold change. The NSCLC diagnostics score was calculated per iteration by selecting the median 2-log counts-per-million value for each sample for the genes in the biomarker gene panel. For each biomarker set, the AUC-value of a ROC-curve of the biomarker gene in an evaluation cohort (n=88) was evaluated. This was performed for a biomarker gene panel ranging from 2 genes up to and including 500 genes.

The evaluation cohort (n=88 samples) showed the highest AUC-value in the ROC-curve of the NSCLC diagnostics score in a 60-gene biomarker gene panel (AUC-value: 0.86, classification accuracy: 81%). Subsequent locking of the 60-genes biomarker gene panel and ROC-curve evaluation of an independent NSCLC late-stage validation cohort (n=518, n=245 NSCLC and n=273 non-cancer) resulted in an AUC-value of 0.80 (95%-CI: 0.77-0.84) and an classification accuracy of 73%, and an independent NSCLC locally-advanced validation cohort (n=106, n=53 NSCLC and n=53 non-cancer) resulted in an AUC-value of 0.74 (95%-CI: 0.64-0.84) and an classification accuracy of 69%.

Before the biomarker gene panel was reduced to 10 genes, the 60-genes biomarker gene panel was filtered for genes that were also selected by PSO (see above). 45 out of 60 genes were present in both gene panels and thus selected for further analyses. The 45 genes resulted in an AUC-value of 0.77 (95%-CI: 0.73-0.81) and a classification accuracy of 77% in an independent, late stage validation set (n=518 samples). The AUC-value was 0.74 (95%-CI: 0.65-0.83), with a classification accuracy of 70% in an early stage validation set (n=106 samples). Following, random 10-gene panel biomarker gene panels from these 45 candidate biomarkers were selected (n=1000 iterations), and the classification accuracy in the evaluation cohort (n=88) was determined. The randomly selected biomarker gene panel (n=10 genes) with highest AUC-value and classification accuracy (respectively 0.87 and 81%) was selected for validation in the independent early and late-stage validation cohort (early-stage cohort: n=106, AUC-value: 0.69 (95%-CI: 0.59-0.79), classification accuracy 65%, late-stage cohort: n=518, AUC-value: 0.74 (95%-CI: 0.70-0.77), classification accuracy 68%).

### P-Selectin Panel for NSCLC Diagnostics and Nivolumab Response Prediction

The p-selectin 5-gene signature was selected using a similar approach. First, all genes correlated to the expression level of p-selectin RNA were selected and sorted according to the correlation coefficient and FDR-value. Next, the sorted p-selectin correlating genes were filtered for those with a positive logarithmic fold change in the non-cancer versus NSCLC ANOVA. Again, the p-selectin gene panel was iteratively increased by adding in each iteration one additional gene, according to the FDR-ranked p-selectin co-correlating gene list. This was performed for two up till and including 50 genes. For each biomarker set the samples in the evaluation cohort were evaluated for AUC-value and classification accuracy, and the p-selectin gene panel with the best AUC-value and classification accuracy was selected (n=5 genes, AUC: 0.74, classification accuracy: 70%). The resulting 5 gene panel classified the independent NSCLC late-stage validation samples, resulting in an AUC-value of 0.58 (95%-CI: 0.53-0.62) and classification accuracy of 57% (n=518 samples). The early-stage NSCLC were classified with an AUC-value of 0.66 (95%-CI: 0.55-0.76) and classification accuracy of 65% (n=106 samples).

### Nivolumab Response Prediction Gene Panel

A minimal gene panel for nivolumab response prediction was selected using a similar approach. Platelet samples were collected up to one month before start of treatment (baseline, n=179 samples). Response assessment of patients treated with nivolumab was performed by CT-imaging at baseline, 6-8 weeks, 3 months and 6 months after start of treatment. Treatment response was assessed according to the updated RECIST version 1.1 criteria (Eisenhauer et al., 2009. Europ J Cancer 45: 228-247; Schwartz et al., 2016. Eur J Cancer 62: 132-7), and scored as progressive disease (PD), stable disease (SD), partial response (PR), or complete response (CR). The main aim was to identify those patients who showed control of disease in response to therapy versus non-responders. Hence, for the nivolumab response prediction analysis, patients were grouped who showed progressive disease as the most optimal response in the non-responding group, totaling 179 samples. Patients with partial response at any response assessment time point as most optimal response or stable disease at 6 months response assessment were annotated as responders, totaling 91 samples. To select and validate the nivolumab biomarker gene panel, 91 responders and 91 non-responders matched for age and gender were selected randomly, to enable for equal group sizes. 55 responders and non-responders were assigned to the training cohort (n=110 in total), 25 responders and non-responders were assigned to the evaluation cohort (n=50 in total), and 11 responders and non-responders remained for independent validation (n=22 in total). We first subjected the cohort to the RUV-normalization module (Jacob et al., 2016. Biostatistics 17: 16-28). For this analysis, genes were selected that showed expression levels which correlated to sample library sizes (calculated by Pearson's correlation) and hospital of sample collection (calculated by ANOVA statistics), and subjected the samples to RUV-correction. This enables for correction of the read counts for confounding factors in the RNA-sequencing data. The stable genes were determined using the training cohort only. Next, we performed trimmed mean of M values-normalization (TMM-normalization; Robinson and Oshlack, 2010. Genome Biol 11: R25) and subjected the TMM-normalized log-2-transformed counts-per-million reads to per-gene wilcoxon differential expression analysis. For this, only the samples assigned to the training cohort were included. The gene list resulting from the wilcoxon differential expression analysis sorted by p-value served as an input for an iterative biomarker gene panel selection algorithm as described above. The direction of the differential expression was calculated by subtracting the median counts from the non-responders from the responders (delta_median-value). The nivolumab response prediction score was determined by subtracting per sample the median counts of genes that showed decreased expression from those that showed increased expression. During each iteration of the iterative biomarker gene panel selection algorithm both an increased and decreased RNA was added. For each biomarker set, the AUC-value of a HOC-curve of the biomarker gene was evaluated in an evaluation cohort (n=50 samples). This was performed for a biomarker gene panel ranging from 4 up till and including 1600 genes. The evaluation cohort reached the highest AUC-value in the ROC-curve of the nivolumab response prediction score in a 4-gene biomarker gene panel (AUC-value: 0.69, classification accuracy: 70%). Subsequent locking of the 4-gene biomarker gene panel and HOC-curve analysis of classification of an independent validation cohort (n=22, n=11 responders, n=11 non-responders) resulted in an AUC-value of 0.70 (95%-CI: 0.47-0.94) and an classification accuracy of 73%. Additional evaluation of a 6-gene biomarker gene panel, selected using the three most significantly increase and the three most significantly decreased differentially expressed RNAs resulted in an classification accuracy of (0% in the evaluation cohort (AUC: 0.60, n=50 samples) and classification accuracy of 64% in the validation cohort (AUC: 0.61, 95%-CI: 0.36-0.86, n=22 samples).

TABLE 5
Patient characteristics

| | | | | | | | | | | Time of | | | |
|---|---|---|---|---|---|---|---|---|---|---|---|---|---|
| | | | | | | | | | Response | collection | | | Nivol- |
| | Classification | | Storage | | | Smok- | Meta- | | to | Nivol- | Matched | Full | umab |
| Number | group | Hospital | time | Age | Gender | ing | stasis | Treatment | treatment | umab | cohort | cohort | cohort |
| 1 | nonCancer | VUMC | <12 h | 68 | F | N | NA | NA | NA | NA | evaluation | evaluation | NA |
| 2 | nonCancer | VUMC | <12 h | 65 | F | N | NA | NA | NA | NA | evaluation | evaluation | NA |
| 3 | nonCancer | VUMC | <12 h | 65 | M | N | NA | NA | NA | NA | evaluation | evaluation | NA |
| 4 | nonCancer | VUMC | <12 h | 56 | F | N | NA | NA | NA | NA | evaluation | training | NA |
| 5 | nonCancer | VUMC | <12 h | 54 | F | N | NA | NA | NA | NA | evaluation | training | NA |
| 6 | nonCancer | VUMC | <12 h | 62 | M | N | NA | NA | NA | NA | evaluation | training | NA |
| 7 | nonCancer | VUMC | <12 h | 51 | M | N | NA | NA | NA | NA | evaluation | evaluation | NA |
| 8 | nonCancer | VUMC | <12 h | 60 | F | N | NA | NA | NA | NA | evaluation | training | NA |
| 9 | nonCancer | VUMC | <12 h | 56 | F | N | NA | NA | NA | NA | evaluation | training | NA |
| 10 | nonCancer | VUMC | <12 h | 59 | M | F | NA | NA | NA | NA | evaluation | evaluation | NA |
| 11 | nonCancer | VUMC | <12 h | 63 | F | F | NA | NA | NA | NA | evaluation | training | NA |
| 12 | nonCancer | VUMC | <12 h | 55 | M | N | NA | NA | NA | NA | evaluation | evaluation | NA |
| 13 | nonCancer | VUMC | <12 h | 54 | F | N | NA | NA | NA | NA | evaluation | evaluation | NA |
| 14 | nonCancer | VUMC | <12 h | 62 | F | N | NA | NA | NA | NA | evaluation | evaluation | NA |
| 15 | nonCancer | VUMC | <12 h | 53 | F | N | NA | NA | NA | NA | evaluation | training | NA |
| 16 | nonCancer | VUMC | <12 h | 71 | M | NA | NA | NA | NA | NA | evaluation | training | NA |
| 17 | nonCancer | VUMC | <12 h | 48 | F | N | NA | NA | NA | NA | validation | evaluation | NA |
| 18 | nonCancer | VUMC | <12 h | 55 | F | N | NA | NA | NA | NA | validation | training | NA |
| 19 | nonCancer | VUMC | <12 h | 60 | F | N | NA | NA | NA | NA | validation | training | NA |
| 20 | nonCancer | VUMC | <12 h | 56 | M | N | NA | NA | NA | NA | validation | training | NA |
| 21 | nonCancer | VUMC | <12 h | 58 | M | N | NA | NA | NA | NA | validation | evaluation | NA |
| 22 | nonCancer | VUMC | <12 h | 54 | M | F | NA | NA | NA | NA | validation | evaluation | NA |
| 23 | nonCancer | VUMC | <12 h | 46 | F | N | NA | NA | NA | NA | validation | training | NA |
| 24 | nonCancer | VUMC | <12 h | 53 | F | N | NA | NA | NA | NA | validation | evaluation | NA |
| 25 | nonCancer | VUMC | <12 h | 52 | F | N | NA | NA | NA | NA | validation | evaluation | NA |
| 26 | nonCancer | VUMC | <12 h | 54 | F | N | NA | NA | NA | NA | validation | evaluation | NA |
| 27 | nonCancer | VUMC | <12 h | 64 | F | Y | NA | NA | NA | NA | validation | training | NA |
| 28 | nonCancer | VUMC | <12 h | 46 | M | N | NA | NA | NA | NA | validation | training | NA |
| 29 | nonCancer | VUMC | <12 h | 63 | M | N | NA | NA | NA | NA | validation | training | NA |
| 30 | nonCancer | VUMC | <12 h | 47 | M | N | NA | NA | NA | NA | validation | training | NA |
| 31 | nonCancer | VUMC | <12 h | 76 | F | N | NA | NA | NA | NA | validation | training | NA |
| 32 | nonCancer | VUMC | <12 h | 53 | M | N | NA | NA | NA | NA | validation | evaluation | NA |
| 33 | nonCancer | VUMC | <12 h | 54 | F | Y | NA | NA | NA | NA | validation | evaluation | NA |
| 34 | nonCancer | VUMC | <12 h | 56 | M | N | NA | NA | NA | NA | validation | training | NA |
| 35 | nonCancer | VUMC | <12 h | 56 | M | N | NA | NA | NA | NA | validation | training | NA |
| 36 | nonCancer | VUMC | <12 h | 55 | F | N | NA | NA | NA | NA | validation | training | NA |
| 37 | nonCancer | VUMC | <12 h | 55 | F | N | NA | NA | NA | NA | validation | training | NA |
| 38 | nonCancer | VUMC | <12 h | 53 | M | N | NA | NA | NA | NA | validation | evaluation | NA |
| 39 | nonCancer | VUMC | <12 h | 55 | F | Y | NA | NA | NA | NA | validation | evaluation | NA |
| 40 | nonCancer | VUMC | <12 h | 59 | M | N | NA | NA | NA | NA | validation | training | NA |
| 41 | nonCancer | VUMC | <12 h | 56 | F | N | NA | NA | NA | NA | validation | training | NA |
| 42 | nonCancer | VUMC | <12 h | 86 | M | N | NA | NA | NA | NA | validation | evaluation | NA |
| 43 | nonCancer | VUMC | <12 h | 69 | F | N | NA | NA | NA | NA | validation | evaluation | NA |
| 44 | nonCancer | VUMC | <12 h | 54 | M | N | NA | NA | NA | NA | validation | training | NA |
| 45 | nonCancer | VUMC | <12 h | 56 | F | N | NA | NA | NA | NA | validation | training | NA |
| 46 | nonCancer | VUMC | <12 h | 60 | M | N | NA | NA | NA | NA | validation | training | NA |
| 47 | nonCancer | VUMC | <12 h | 62 | F | N | NA | NA | NA | NA | validation | training | NA |
| 48 | nonCancer | VUMC | <12 h | 50 | F | N | NA | NA | NA | NA | training | training | NA |
| 49 | nonCancer | VUMC | <12 h | 51 | M | Y | NA | NA | NA | NA | training | evaluation | NA |
| 50 | nonCancer | VUMC | <12 h | 50 | F | N | NA | NA | NA | NA | training | evaluation | NA |
| 51 | nonCancer | VUMC | <12 h | 47 | F | N | NA | NA | NA | NA | training | training | NA |
| 52 | nonCancer | VUMC | <12 h | 51 | M | N | NA | NA | NA | NA | training | evaluation | NA |
| 53 | nonCancer | VUMC | <12 h | 49 | F | N | NA | NA | NA | NA | training | evaluation | NA |
| 54 | nonCancer | VUMC | <12 h | 52 | F | N | NA | NA | NA | NA | training | evaluation | NA |
| 55 | nonCancer | VUMC | <12 h | 57 | F | N | NA | NA | NA | NA | training | training | NA |
| 56 | nonCancer | VUMC | <12 h | NA | NA | NA | NA | NA | NA | NA | NA | validation | NA |
| 57 | nonCancer | UMCU | <12 h | 54 | F | Y | NA | NA | NA | NA | NA | validation | NA |
| 58 | nonCancer | UMCU | <12 h | 53 | M | N | NA | NA | NA | NA | NA | validation | NA |
| 59 | nonCancer | UMCU | <12 h | 56 | M | F | NA | NA | NA | NA | NA | validation | NA |
| 60 | nonCancer | UMCU | <12 h | 48 | M | N | NA | NA | NA | NA | NA | validation | NA |
| 61 | nonCancer | UMCU | <12 h | 53 | M | N | NA | NA | NA | NA | NA | validation | NA |
| 62 | nonCancer | UMCU | <12 h | 41 | M | F | NA | NA | NA | NA | NA | validation | NA |
| 63 | nonCancer | UMCU | <12 h | 43 | M | N | NA | NA | NA | NA | NA | validation | NA |
| 64 | nonCancer | UMCU | <12 h | 41 | M | N | NA | NA | NA | NA | NA | validation | NA |
| 65 | nonCancer | UMCU | <12 h | 40 | F | N | NA | NA | NA | NA | NA | validation | NA |
| 66 | nonCancer | UMCU | <12 h | 47 | M | N | NA | NA | NA | NA | NA | validation | NA |
| 67 | nonCancer | UMCU | <12 h | 48 | M | N | NA | NA | NA | NA | NA | validation | NA |
| 68 | nonCancer | UMCU | <12 h | 53 | M | N | NA | NA | NA | NA | NA | validation | NA |
| 69 | nonCancer | UMCU | <12 h | 53 | M | F | NA | NA | NA | NA | NA | validation | NA |
| 70 | nonCancer | UMCU | <12 h | 57 | M | F | NA | NA | NA | NA | NA | validation | NA |
| 71 | nonCancer | UMCU | <12 h | 51 | F | N | NA | NA | NA | NA | NA | validation | NA |
| 72 | nonCancer | VUMC | <12 h | 35 | F | N | NA | NA | NA | NA | NA | validation | NA |
| 73 | nonCancer | VUMC | <12 h | 38 | F | N | NA | NA | NA | NA | NA | validation | NA |
| 74 | nonCancer | VUMC | <12 h | 38 | F | N | NA | NA | NA | NA | NA | validation | NA |
| 75 | nonCancer | VUMC | <12 h | 39 | F | F | NA | NA | NA | NA | NA | validation | NA |
| 76 | nonCancer | VUMC | <12 h | 42 | F | N | NA | NA | NA | NA | NA | validation | NA |
| 77 | nonCancer | VUMC | <12 h | 42 | F | N | NA | NA | NA | NA | NA | validation | NA |
| 78 | nonCancer | VUMC | <12 h | 42 | F | N | NA | NA | NA | NA | NA | validation | NA |
| 79 | nonCancer | VUMC | <12 h | 44 | F | N | NA | NA | NA | NA | NA | validation | NA |
| 80 | nonCancer | VUMC | <12 h | 40 | F | N | NA | NA | NA | NA | NA | validation | NA |
| 81 | nonCancer | VUMC | <12 h | 39 | F | N | NA | NA | NA | NA | NA | validation | NA |
| 82 | nonCancer | VUMC | <12 h | 40 | F | N | NA | NA | NA | NA | NA | validation | NA |
| 83 | nonCancer | VUMC | <12 h | 37 | F | N | NA | NA | NA | NA | NA | validation | NA |
| 84 | nonCancer | VUMC | <12 h | 45 | F | Y | NA | NA | NA | NA | NA | validation | NA |
| 85 | nonCancer | VUMC | <12 h | 40 | F | Y | NA | NA | NA | NA | NA | validation | NA |
| 86 | nonCancer | VUMC | <12 h | 45 | F | N | NA | NA | NA | NA | NA | validation | NA |
| 87 | nonCancer | VUMC | <12 h | 59 | F | N | NA | NA | NA | NA | NA | validation | NA |
| 88 | nonCancer | VUMC | <12 h | 36 | F | N | NA | NA | NA | NA | NA | validation | NA |
| 89 | nonCancer | AMC | >12 h | 23 | M | N | NA | NA | NA | NA | NA | validation | NA |
| 90 | nonCancer | AMC | >12 h | 20 | F | Y | NA | NA | NA | NA | NA | validation | NA |
| 91 | nonCancer | AMC | >12 h | 21 | F | N | NA | NA | NA | NA | NA | validation | NA |
| 92 | nonCancer | AMC | >12 h | 21 | F | N | NA | NA | NA | NA | NA | validation | NA |
| 93 | nonCancer | AMC | >12 h | 21 | F | N | NA | NA | NA | NA | NA | validation | NA |
| 94 | nonCancer | AMC | >12 h | 22 | F | N | NA | NA | NA | NA | NA | validation | NA |
| 95 | nonCancer | AMC | >12 h | 30 | F | Y | NA | NA | NA | NA | NA | validation | NA |
| 96 | nonCancer | AMC | >12 h | 24 | M | N | NA | NA | NA | NA | NA | validation | NA |
| 97 | nonCancer | AMC | >12 h | 42 | F | N | NA | NA | NA | NA | NA | validation | NA |
| 98 | nonCancer | VUMC | <12 h | 33 | F | N | NA | NA | NA | NA | NA | validation | NA |
| 99 | nonCancer | VUMC | <12 h | 34 | M | N | NA | NA | NA | NA | NA | validation | NA |
| 100 | nonCancer | VUMC | <12 h | 35 | M | N | NA | NA | NA | NA | NA | validation | NA |
| 101 | nonCancer | VUMC | <12 h | 24 | F | Y | NA | NA | NA | NA | NA | validation | NA |
| 102 | nonCancer | VUMC | <12 h | 26 | M | N | NA | NA | NA | NA | NA | validation | NA |
| 103 | nonCancer | VUMC | <12 h | 23 | F | N | NA | NA | NA | NA | NA | validation | NA |
| 104 | nonCancer | VUMC | <12 h | 27 | F | N | NA | NA | NA | NA | NA | validation | NA |
| 105 | nonCancer | VUMC | <12 h | 21 | F | N | NA | NA | NA | NA | NA | validation | NA |
| 106 | nonCancer | VUMC | <12 h | 22 | F | N | NA | NA | NA | NA | NA | validation | NA |
| 107 | nonCancer | VUMC | <12 h | 21 | M | N | NA | NA | NA | NA | NA | validation | NA |
| 108 | nonCancer | VUMC | <12 h | 29 | F | N | NA | NA | NA | NA | NA | validation | NA |
| 109 | nonCancer | VUMC | <12 h | 32 | F | N | NA | NA | NA | NA | NA | validation | NA |
| 110 | nonCancer | VUMC | <12 h | 35 | M | N | NA | NA | NA | NA | NA | validation | NA |
| 111 | nonCancer | VUMC | <12 h | 29 | M | N | NA | NA | NA | NA | NA | validation | NA |
| 112 | nonCancer | VUMC | <12 h | 32 | F | N | NA | NA | NA | NA | NA | validation | NA |
| 113 | nonCancer | VUMC | <12 h | 33 | F | N | NA | NA | NA | NA | NA | validation | NA |
| 114 | nonCancer | VUMC | <12 h | 25 | M | N | NA | NA | NA | NA | NA | validation | NA |
| 115 | nonCancer | VUMC | <12 h | 25 | F | N | NA | NA | NA | NA | NA | validation | NA |
| 116 | nonCancer | VUMC | <12 h | 27 | F | N | NA | NA | NA | NA | NA | validation | NA |
| 117 | nonCancer | VUMC | <12 h | 30 | M | N | NA | NA | NA | NA | NA | validation | NA |
| 118 | nonCancer | VUMC | <12 h | 34 | M | N | NA | NA | NA | NA | NA | validation | NA |
| 119 | nonCancer | VUMC | <12 h | 32 | F | N | NA | NA | NA | NA | NA | validation | NA |
| 120 | nonCancer | VUMC | <12 h | 24 | F | N | NA | NA | NA | NA | NA | validation | NA |
| 121 | nonCancer | VUMC | <12 h | 33 | F | N | NA | NA | NA | NA | NA | validation | NA |
| 122 | nonCancer | VUMC | <12 h | 24 | M | Y | NA | NA | NA | NA | NA | validation | NA |
| 123 | nonCancer | VUMC | <12 h | 29 | M | N | NA | NA | NA | NA | NA | validation | NA |
| 124 | nonCancer | VUMC | <12 h | 32 | M | N | NA | NA | NA | NA | NA | validation | NA |
| 125 | nonCancer | VUMC | <12 h | 23 | F | Y | NA | NA | NA | NA | NA | validation | NA |
| 126 | nonCancer | VUMC | <12 h | 20 | F | N | NA | NA | NA | NA | NA | validation | NA |
| 127 | nonCancer | VUMC | <12 h | 18 | M | N | NA | NA | NA | NA | NA | validation | NA |
| 128 | nonCancer | VUMC | <12 h | 18 | M | N | NA | NA | NA | NA | NA | validation | NA |
| 129 | nonCancer | VUMC | <12 h | 41 | M | N | NA | NA | NA | NA | NA | validation | NA |
| 130 | nonCancer | VUMC | <12 h | 33 | M | Y | NA | NA | NA | NA | NA | validation | NA |
| 131 | nonCancer | VUMC | <12 h | 26 | M | Y | NA | NA | NA | NA | NA | validation | NA |
| 132 | nonCancer | VUMC | <12 h | 32 | F | N | NA | NA | NA | NA | NA | validation | NA |
| 133 | nonCancer | VUMC | <12 h | 26 | F | N | NA | NA | NA | NA | NA | validation | NA |
| 134 | nonCancer | VUMC | <12 h | 32 | F | N | NA | NA | NA | NA | NA | validation | NA |
| 135 | nonCancer | VUMC | <12 h | 26 | F | N | NA | NA | NA | NA | NA | validation | NA |
| 136 | nonCancer | VUMC | <12 h | 26 | F | N | NA | NA | NA | NA | NA | validation | NA |
| 137 | nonCancer | VUMC | <12 h | 37 | M | N | NA | NA | NA | NA | NA | validation | NA |
| 138 | nonCancer | VUMC | <12 h | 26 | M | N | NA | NA | NA | NA | NA | validation | NA |
| 139 | nonCancer | VUMC | <12 h | 43 | F | N | NA | NA | NA | NA | NA | validation | NA |
| 140 | nonCancer | VUMC | <12 h | 27 | F | N | NA | NA | NA | NA | NA | validation | NA |
| 141 | nonCancer | VUMC | <12 h | 39 | F | Y | NA | NA | NA | NA | NA | validation | NA |
| 142 | nonCancer | VUMC | <12 h | 36 | F | N | NA | NA | NA | NA | NA | validation | NA |
| 143 | nonCancer | VUMC | <12 h | 29 | F | N | NA | NA | NA | NA | NA | validation | NA |
| 144 | nonCancer | VUMC | <12 h | 27 | F | Y | NA | NA | NA | NA | NA | validation | NA |
| 145 | nonCancer | VUMC | <12 h | 38 | F | N | NA | NA | NA | NA | NA | validation | NA |
| 146 | nonCancer | VUMC | <12 h | 22 | F | N | NA | NA | NA | NA | NA | validation | NA |
| 147 | nonCancer | VUMC | <12 h | 21 | F | N | NA | NA | NA | NA | NA | validation | NA |
| 148 | nonCancer | VUMC | <12 h | 33 | F | N | NA | NA | NA | NA | NA | validation | NA |
| 149 | nonCancer | VUMC | <12 h | 49 | F | NA | NA | NA | NA | NA | NA | validation | NA |
| 150 | nonCancer | VUMC | <12 h | 43 | F | NA | NA | NA | NA | NA | NA | validation | NA |
| 151 | nonCancer | VUMC | <12 h | 41 | F | N | NA | NA | NA | NA | NA | validation | NA |
| 152 | nonCancer | VUMC | <12 h | 64 | F | NA | NA | NA | NA | NA | NA | validation | NA |
| 153 | nonCancer | VUMC | <12 h | 29 | M | N | NA | NA | NA | NA | NA | validation | NA |
| 154 | nonCancer | VUMC | <12 h | 34 | M | N | NA | NA | NA | NA | NA | validation | NA |
| 155 | nonCancer | VUMC | <12 h | 27 | M | N | NA | NA | NA | NA | NA | validation | NA |
| 156 | nonCancer | VUMC | <12 h | 45 | M | N | NA | NA | NA | NA | NA | validation | NA |
| 157 | nonCancer | VUMC | <12 h | 24 | F | F | NA | NA | NA | NA | NA | validation | NA |
| 158 | nonCancer | VUMC | <12 h | 45 | M | N | NA | NA | NA | NA | NA | validation | NA |
| 159 | nonCancer | VUMC | <12 h | 26 | M | N | NA | NA | NA | NA | NA | validation | NA |
| 160 | nonCancer | VUMC | <12 h | 21 | M | N | NA | NA | NA | NA | NA | validation | NA |
| 161 | nonCancer | VUMC | <12 h | 27 | F | Y | NA | NA | NA | NA | NA | validation | NA |
| 162 | nonCancer | VUMC | <12 h | 43 | M | N | NA | NA | NA | NA | NA | validation | NA |
| 163 | nonCancer | VUMC | <12 h | 29 | M | N | NA | NA | NA | NA | NA | validation | NA |
| 164 | nonCancer | VUMC | <12 h | 24 | F | N | NA | NA | NA | NA | NA | validation | NA |
| 165 | nonCancer | VUMC | <12 h | 40 | M | N | NA | NA | NA | NA | NA | validation | NA |
| 166 | nonCancer | VUMC | <12 h | 43 | M | N | NA | NA | NA | NA | NA | validation | NA |
| 167 | nonCancer | VUMC | <12 h | 37 | M | Y | NA | NA | NA | NA | NA | validation | NA |
| 168 | nonCancer | VUMC | <12 h | 29 | M | Y | NA | NA | NA | NA | NA | validation | NA |
| 169 | nonCancer | VUMC | <12 h | 29 | M | N | NA | NA | NA | NA | NA | validation | NA |
| 170 | nonCancer | VUMC | <12 h | 41 | F | N | NA | NA | NA | NA | NA | validation | NA |
| 171 | nonCancer | VUMC | <12 h | NA | NA | NA | NA | NA | NA | NA | NA | validation | NA |
| 172 | nonCancer | VUMC | <12 h | NA | NA | NA | NA | NA | NA | NA | NA | validation | NA |
| 173 | nonCancer | VUMC | <12 h | NA | NA | NA | NA | NA | NA | NA | NA | validation | NA |
| 174 | nonCancer | VUMC | <12 h | NA | NA | NA | NA | NA | NA | NA | NA | validation | NA |
| 175 | nonCancer | VUMC | <12 h | 64 | F | NA | NA | NA | NA | NA | NA | validation | NA |
| 176 | nonCancer | VUMC | <12 h | 49 | F | NA | NA | NA | NA | NA | NA | validation | NA |
| 177 | nonCancer | VUMC | <12 h | 64 | F | NA | NA | NA | NA | NA | NA | validation | NA |
| 178 | nonCancer | UMEA | <12 h | NA | M | NA | NA | NA | NA | NA | NA | validation | NA |
| 179 | nonCancer | UMEA | <12 h | NA | M | NA | NA | NA | NA | NA | NA | validation | NA |
| 180 | nonCancer | UMEA | <12 h | NA | M | NA | NA | NA | NA | NA | NA | validation | NA |
| 181 | nonCancer | UMEA | <12 h | NA | M | NA | NA | NA | NA | NA | NA | validation | NA |
| 182 | nonCancer | UMEA | <12 h | NA | M | NA | NA | NA | NA | NA | NA | validation | NA |
| 183 | nonCancer | UMEA | <12 h | NA | M | NA | NA | NA | NA | NA | NA | validation | NA |
| 184 | nonCancer | UMEA | <12 h | NA | M | NA | NA | NA | NA | NA | NA | validation | NA |
| 185 | nonCancer | UMEA | <12 h | NA | M | NA | NA | NA | NA | NA | NA | validation | NA |
| 186 | nonCancer | UMEA | <12 h | NA | M | NA | NA | NA | NA | NA | NA | validation | NA |
| 187 | nonCancer | UMEA | <12 h | NA | M | NA | NA | NA | NA | NA | NA | validation | NA |
| 188 | nonCancer | VUMC | <12 h | 35 | M | Y | NA | NA | NA | NA | NA | validation | NA |
| 189 | nonCancer | VUMC | <12 h | 49 | M | N | NA | NA | NA | NA | NA | validation | NA |
| 190 | nonCancer | VUMC | <12 h | 51 | M | N | NA | NA | NA | NA | NA | validation | NA |
| 191 | nonCancer | VUMC | <12 h | 22 | M | N | NA | NA | NA | NA | NA | validation | NA |
| 192 | nonCancer | VUMC | <12 h | 61 | M | N | NA | NA | NA | NA | NA | validation | NA |
| 193 | nonCancer | VUMC | <12 h | 36 | M | N | NA | NA | NA | NA | NA | validation | NA |
| 194 | nonCancer | VUMC | <12 h | 26 | M | N | NA | NA | NA | NA | NA | validation | NA |
| 195 | nonCancer | VUMC | <12 h | 24 | M | N | NA | NA | NA | NA | NA | validation | NA |
| 196 | nonCancer | VUMC | <12 h | 62 | M | Y | NA | NA | NA | NA | NA | validation | NA |
| 197 | nonCancer | VUMC | <12 h | 53 | F | N | NA | NA | NA | NA | NA | validation | NA |
| 198 | nonCancer | VUMC | <12 h | 31 | M | N | NA | NA | NA | NA | NA | validation | NA |
| 199 | nonCancer | VUMC | <12 h | 44 | M | N | NA | NA | NA | NA | NA | validation | NA |
| 200 | nonCancer | VUMC | <12 h | 57 | F | N | NA | NA | NA | NA | NA | validation | NA |
| 201 | nonCancer | VUMC | <12 h | 53 | M | N | NA | NA | NA | NA | NA | validation | NA |
| 202 | nonCancer | VUMC | <12 h | 34 | M | Y | NA | NA | NA | NA | NA | validation | NA |
| 203 | nonCancer | VUMC | <12 h | 35 | M | Y | NA | NA | NA | NA | NA | validation | NA |
| 204 | nonCancer | VUMC | <12 h | 29 | M | N | NA | NA | NA | NA | NA | validation | NA |
| 205 | nonCancer | VUMC | <12 h | 23 | M | N | NA | NA | NA | NA | NA | validation | NA |
| 206 | nonCancer | VUMC | <12 h | 28 | M | N | NA | NA | NA | NA | NA | validation | NA |
| 207 | nonCancer | VUMC | <12 h | 25 | M | N | NA | NA | NA | NA | NA | validation | NA |
| 208 | nonCancer | VUMC | <12 h | 53 | M | N | NA | NA | NA | NA | NA | validation | NA |
| 209 | nonCancer | VUMC | <12 h | 57 | M | N | NA | NA | NA | NA | NA | validation | NA |
| 210 | nonCancer | VUMC | <12 h | 51 | M | N | NA | NA | NA | NA | NA | validation | NA |
| 211 | nonCancer | VUMC | <12 h | 50 | M | N | NA | NA | NA | NA | NA | validation | NA |
| 212 | nonCancer | VUMC | <12 h | 42 | F | N | NA | NA | NA | NA | NA | validation | NA |
| 213 | nonCancer | VUMC | <12 h | 42 | F | N | NA | NA | NA | NA | NA | validation | NA |
| 214 | nonCancer | VUMC | <12 h | 52 | M | Y | NA | NA | NA | NA | NA | validation | NA |
| 215 | nonCancer | VUMC | <12 h | 50 | M | N | NA | NA | NA | NA | NA | validation | NA |
| 216 | nonCancer | VUMC | <12 h | 26 | M | Y | NA | NA | NA | NA | NA | validation | NA |
| 217 | nonCancer | VUMC | <12 h | 49 | M | N | NA | NA | NA | NA | NA | validation | NA |
| 218 | nonCancer | VUMC | <12 h | 44 | F | N | NA | NA | NA | NA | NA | validation | NA |
| 219 | nonCancer | VUMC | <12 h | 47 | F | N | NA | NA | NA | NA | NA | validation | NA |
| 220 | nonCancer | VUMC | <12 h | 67 | M | F | NA | NA | NA | NA | NA | validation | NA |
| 221 | nonCancer | VUMC | <12 h | 58 | F | Y | NA | NA | NA | NA | NA | validation | NA |
| 222 | nonCancer | VUMC | <12 h | 55 | F | N | NA | NA | NA | NA | NA | validation | NA |
| 223 | nonCancer | VUMC | <12 h | 63 | F | N | NA | NA | NA | NA | NA | validation | NA |
| 224 | nonCancer | VUMC | <12 h | 52 | F | N | NA | NA | NA | NA | NA | validation | NA |
| 225 | nonCancer | VUMC | <12 h | 50 | F | N | NA | NA | NA | NA | NA | validation | NA |
| 226 | nonCancer | VUMC | <12 h | 48 | F | Y | NA | NA | NA | NA | NA | validation | NA |
| 227 | nonCancer | VUMC | <12 h | 47 | F | N | NA | NA | NA | NA | NA | validation | NA |
| 228 | nonCancer | VUMC | <12 h | 44 | M | N | NA | NA | NA | NA | NA | validation | NA |
| 229 | nonCancer | VUMC | <12 h | 52 | F | F | NA | NA | NA | NA | NA | validation | NA |
| 230 | nonCancer | VUMC | <12 h | 51 | F | Y | NA | NA | NA | NA | NA | validation | NA |
| 231 | nonCancer | VUMC | <12 h | 59 | F | N | NA | NA | NA | NA | NA | validation | NA |
| 232 | nonCancer | VUMC | <12 h | 55 | F | N | NA | NA | NA | NA | NA | validation | NA |
| 233 | nonCancer | VUMC | <12 h | 50 | F | N | NA | NA | NA | NA | NA | validation | NA |
| 234 | nonCancer | VUMC | <12 h | 51 | F | Y | NA | NA | NA | NA | NA | validation | NA |
| 235 | nonCancer | VUMC | <12 h | 52 | F | N | NA | NA | NA | NA | NA | validation | NA |
| 236 | nonCancer | VUMC | <12 h | 48 | M | N | NA | NA | NA | NA | NA | validation | NA |
| 237 | nonCancer | VUMC | <12 h | 69 | M | N | NA | NA | NA | NA | training | training | NA |
| 238 | nonCancer | PISA | <12 h | NA | NA | NA | NA | NA | NA | NA | NA | validation | NA |
| 239 | nonCancer | VUMC | <12 h | 60 | F | N | NA | NA | NA | NA | NA | validation | NA |
| 240 | nonCancer | VUMC | <12 h | NA | NA | NA | NA | NA | NA | NA | NA | validation | NA |
| 241 | nonCancer | VUMC | <12 h | NA | NA | NA | NA | NA | NA | NA | NA | validation | NA |
| 242 | nonCancer | VUMC | <12 h | 46 | M | N | NA | NA | NA | NA | validation | training | NA |
| 243 | nonCancer | VUMC | <12 h | 58 | M | Y | NA | NA | NA | NA | training | training | NA |
| 244 | nonCancer | VUMC | <12 h | 51 | M | F | NA | NA | NA | NA | training | evaluation | NA |
| 245 | nonCancer | VUMC | <12 h | 53 | M | NA | NA | NA | NA | NA | training | evaluation | NA |
| 246 | nonCancer | VUMC | <12 h | 54 | F | NA | NA | NA | NA | NA | training | training | NA |
| 247 | nonCancer | VUMC | <12 h | 62 | M | NA | NA | NA | NA | NA | training | training | NA |
| 248 | nonCancer | VUMC | <12 h | 27 | F | NA | NA | NA | NA | NA | NA | validation | NA |
| 249 | nonCancer | VUMC | <12 h | 18 | M | NA | NA | NA | NA | NA | NA | validation | NA |
| 250 | nonCancer | VUMC | <12 h | 45 | M | F | NA | NA | NA | NA | NA | validation | NA |
| 251 | nonCancer | VUMC | <12 h | 21 | F | NA | NA | NA | NA | NA | NA | validation | NA |
| 252 | nonCancer | VUMC | <12 h | 39 | M | F | NA | NA | NA | NA | NA | validation | NA |
| 253 | nonCancer | VUMC | <12 h | 22 | F | N | NA | NA | NA | NA | NA | validation | NA |
| 254 | nonCancer | VUMC | <12 h | 23 | M | N | NA | NA | NA | NA | NA | validation | NA |
| 255 | nonCancer | VUMC | <12 h | 32 | F | NA | NA | NA | NA | NA | NA | validation | NA |
| 256 | nonCancer | VUMC | <12 h | 21 | M | NA | NA | NA | NA | NA | NA | validation | NA |
| 257 | nonCancer | VUMC | <12 h | 18 | F | N | NA | NA | NA | NA | NA | validation | NA |
| 258 | nonCancer | VUMC | <12 h | 25 | F | N | NA | NA | NA | NA | NA | validation | NA |
| 259 | nonCancer | VUMC | <12 h | 19 | F | F | NA | NA | NA | NA | NA | validation | NA |
| 260 | nonCancer | VUMC | <12 h | 41 | M | N | NA | NA | NA | NA | NA | validation | NA |
| 261 | nonCancer | VUMC | <12 h | 24 | M | Y | NA | NA | NA | NA | NA | validation | NA |
| 262 | nonCancer | VUMC | <12 h | 28 | F | NA | NA | NA | NA | NA | NA | validation | NA |
| 263 | nonCancer | VUMC | <12 h | 49 | F | NA | NA | NA | NA | NA | validation | training | NA |
| 264 | nonCancer | VUMC | <12 h | 51 | F | NA | NA | NA | NA | NA | validation | training | NA |
| 265 | nonCancer | VUMC | <12 h | 47 | F | NA | NA | NA | NA | NA | training | evaluation | NA |
| 266 | nonCancer | VUMC | <12 h | 57 | M | NA | NA | NA | NA | NA | training | training | NA |
| 267 | nonCancer | VUMC | <12 h | 61 | F | NA | NA | NA | NA | NA | training | training | NA |
| 268 | nonCancer | VUMC | <12 h | 62 | M | NA | NA | NA | NA | NA | training | evaluation | NA |
| 269 | nonCancer | VUMC | <12 h | 39 | F | NA | NA | NA | NA | NA | NA | validation | NA |
| 270 | nonCancer | VUMC | <12 h | 43 | F | NA | NA | NA | NA | NA | NA | validation | NA |
| 271 | nonCancer | VUMC | <12 h | 39 | M | NA | NA | NA | NA | NA | NA | validation | NA |
| 272 | nonCancer | VUMC | <12 h | 40 | F | NA | NA | NA | NA | NA | NA | validation | NA |
| 273 | nonCancer | VUMC | <12 h | 46 | F | NA | NA | NA | NA | NA | NA | validation | NA |
| 274 | nonCancer | VUMC | <12 h | 44 | F | NA | NA | NA | NA | NA | NA | validation | NA |
| 275 | nonCancer | VUMC | <12 h | 37 | M | NA | NA | NA | NA | NA | NA | validation | NA |
| 276 | nonCancer | VUMC | <12 h | 46 | F | NA | NA | NA | NA | NA | NA | validation | NA |
| 277 | nonCancer | VUMC | <12 h | 40 | F | NA | NA | NA | NA | NA | NA | validation | NA |
| 278 | nonCancer | VUMC | <12 h | 39 | F | NA | NA | NA | NA | NA | NA | validation | NA |
| 279 | nonCancer | VUMC | <12 h | 43 | F | NA | NA | NA | NA | NA | NA | validation | NA |
| 280 | nonCancer | VUMC | <12 h | 42 | F | NA | NA | NA | NA | NA | NA | validation | NA |
| 281 | nonCancer | VUMC | <12 h | 34 | M | NA | NA | NA | NA | NA | NA | validation | NA |
| 282 | nonCancer | VUMC | <12 h | 41 | F | NA | NA | NA | NA | NA | NA | validation | NA |
| 283 | nonCancer | VUMC | <12 h | 42 | F | NA | NA | NA | NA | NA | NA | validation | NA |
| 284 | nonCancer | VUMC | <12 h | 47 | M | NA | NA | NA | NA | NA | NA | validation | NA |
| 285 | nonCancer | VUMC | <12 h | 35 | M | NA | NA | NA | NA | NA | NA | validation | NA |
| 286 | nonCancer | VUMC | <12 h | 68 | M | NA | NA | NA | NA | NA | NA | validation | NA |
| 287 | nonCancer | VUMC | <12 h | 41 | F | NA | NA | NA | NA | NA | NA | validation | NA |
| 288 | nonCancer | VUMC | <12 h | 48 | F | NA | NA | NA | NA | NA | NA | validation | NA |
| 289 | nonCancer | VUMC | <12 h | 43 | F | NA | NA | NA | NA | NA | NA | validation | NA |
| 290 | nonCancer | VUMC | <12 h | 45 | F | NA | NA | NA | NA | NA | NA | validation | NA |
| 291 | nonCancer | VUMC | <12 h | 42 | F | NA | NA | NA | NA | NA | NA | validation | NA |
| 292 | nonCancer | VUMC | <12 h | 42 | M | NA | NA | NA | NA | NA | NA | validation | NA |
| 293 | nonCancer | VUMC | <12 h | 35 | F | NA | NA | NA | NA | NA | NA | validation | NA |
| 294 | nonCancer | VUMC | <12 h | 54 | F | NA | NA | NA | NA | NA | NA | validation | NA |
| 295 | nonCancer | VUMC | <12 h | 39 | F | NA | NA | NA | NA | NA | NA | validation | NA |
| 296 | nonCancer | VUMC | <12 h | 56 | F | NA | NA | NA | NA | NA | NA | validation | NA |
| 297 | nonCancer | VUMC | <12 h | 59 | F | NA | NA | NA | NA | NA | NA | validation | NA |
| 298 | nonCancer | VUMC | <12 h | 61 | F | NA | NA | NA | NA | NA | NA | validation | NA |
| 299 | nonCancer | VUMC | <12 h | 53 | F | NA | NA | NA | NA | NA | NA | validation | NA |
| 300 | nonCancer | VUMC | <12 h | 49 | M | NA | NA | NA | NA | NA | NA | validation | NA |
| 301 | nonCancer | VUMC | <12 h | 44 | M | NA | NA | NA | NA | NA | NA | validation | NA |
| 302 | nonCancer | VUMC | <12 h | 48 | F | NA | NA | NA | NA | NA | NA | validation | NA |
| 303 | nonCancer | VUMC | <12 h | 42 | F | NA | NA | NA | NA | NA | NA | validation | NA |
| 304 | nonCancer | VUMC | <12 h | 51 | F | NA | NA | NA | NA | NA | NA | validation | NA |
| 305 | nonCancer | VUMC | <12 h | 30 | F | NA | NA | NA | NA | NA | NA | validation | NA |
| 306 | nonCancer | VUMC | <12 h | 49 | M | NA | NA | NA | NA | NA | NA | validation | NA |
| 307 | nonCancer | VUMC | <12 h | 61 | M | NA | NA | NA | NA | NA | NA | validation | NA |
| 308 | nonCancer | VUMC | <12 h | 42 | F | NA | NA | NA | NA | NA | NA | validation | NA |
| 309 | nonCancer | VUMC | <12 h | 47 | F | NA | NA | NA | NA | NA | NA | validation | NA |
| 310 | nonCancer | VUMC | <12 h | 53 | M | NA | NA | NA | NA | NA | NA | validation | NA |
| 311 | nonCancer | VUMC | <12 h | 52 | F | NA | NA | NA | NA | NA | NA | validation | NA |
| 312 | nonCancer | VUMC | <12 h | 68 | M | NA | NA | NA | NA | NA | NA | validation | NA |
| 313 | nonCancer | VUMC | <12 h | 30 | M | NA | NA | NA | NA | NA | NA | validation | NA |
| 314 | nonCancer | VUMC | <12 h | 52 | M | NA | NA | NA | NA | NA | NA | validation | NA |
| 315 | nonCancer | VUMC | <12 h | 45 | F | NA | NA | NA | NA | NA | NA | validation | NA |
| 316 | nonCancer | VUMC | <12 h | 64 | F | NA | NA | NA | NA | NA | NA | validation | NA |
| 317 | nonCancer | VUMC | <12 h | 32 | F | NA | NA | NA | NA | NA | NA | validation | NA |
| 318 | nonCancer | VUMC | <12 h | 52 | F | NA | NA | NA | NA | NA | NA | validation | NA |
| 319 | nonCancer | VUMC | <12 h | 49 | F | NA | NA | NA | NA | NA | NA | validation | NA |
| 320 | nonCancer | VUMC | <12 h | 52 | F | NA | NA | NA | NA | NA | NA | validation | NA |
| 321 | nonCancer | UMCU | <12 h | 64 | M | N | NA | NA | NA | NA | evaluation | evaluation | NA |
| 322 | nonCancer | UMCU | <12 h | 48 | F | Y | NA | NA | NA | NA | training | training | NA |
| 323 | nonCancer | UMCU | <12 h | 71 | F | N | NA | NA | NA | NA | training | training | NA |
| 324 | nonCancer | UMCU | <12 h | 63 | F | N | NA | NA | NA | NA | training | training | NA |
| 325 | nonCancer | UMCU | <12 h | 63 | F | N | NA | NA | NA | NA | training | evaluation | NA |
| 326 | nonCancer | UMCU | <12 h | NA | NA | NA | NA | NA | NA | NA | NA | validation | NA |
| 327 | nonCancer | UMCU | <12 h | NA | NA | NA | NA | NA | NA | NA | NA | validation | NA |
| 328 | nonCancer | UMCU | <12 h | NA | NA | NA | NA | NA | NA | NA | NA | validation | NA |
| 329 | nonCancer | UMCU | <12 h | NA | NA | NA | NA | NA | NA | NA | NA | validation | NA |
| 330 | nonCancer | UMCU | <12 h | NA | NA | NA | NA | NA | NA | NA | NA | validation | NA |
| 331 | nonCancer | UMCU | <12 h | NA | NA | NA | NA | NA | NA | NA | NA | validation | NA |
| 332 | nonCancer | UMCU | <12 h | NA | NA | NA | NA | NA | NA | NA | NA | validation | NA |
| 333 | nonCancer | VUMC | <12 h | 81 | F | F | NA | NA | NA | NA | evaluation | training | NA |
| 334 | nonCancer | VUMC | <12 h | 69 | F | F | NA | NA | NA | NA | validation | training | NA |
| 335 | nonCancer | VUMC | <12 h | 51 | F | N | NA | NA | NA | NA | validation | evaluation | NA |
| 336 | nonCancer | VUMC | <12 h | 65 | F | F | NA | NA | NA | NA | validation | training | NA |
| 337 | nonCancer | VUMC | <12 h | 66 | F | N | NA | NA | NA | NA | validation | evaluation | NA |
| 338 | nonCancer | VUMC | <12 h | 62 | F | F | NA | NA | NA | NA | training | training | NA |
| 339 | nonCancer | VUMC | <12 h | 71 | F | F | NA | NA | NA | NA | training | evaluation | NA |
| 340 | nonCancer | VUMC | <12 h | 69 | F | Y | NA | NA | NA | NA | training | training | NA |
| 341 | nonCancer | VUMC | <12 h | 63 | M | F | NA | NA | NA | NA | training | training | NA |
| 342 | nonCancer | VUMC | <12 h | 58 | M | Y | NA | NA | NA | NA | training | evaluation | NA |
| 343 | nonCancer | VUMC | <12 h | 75 | M | N | NA | NA | NA | NA | training | evaluation | NA |
| 344 | nonCancer | VUMC | <12 h | 74 | F | N | NA | NA | NA | NA | training | training | NA |
| 345 | nonCancer | VUMC | <12 h | 80 | M | F | NA | NA | NA | NA | training | evaluation | NA |
| 346 | nonCancer | VUMC | <12 h | 63 | M | F | NA | NA | NA | NA | training | training | NA |
| 347 | nonCancer | VUMC | <12 h | 72 | M | F | NA | NA | NA | NA | training | evaluation | NA |
| 348 | nonCancer | VUMC | <12 h | 72 | M | Y | NA | NA | NA | NA | training | evaluation | NA |
| 349 | nonCancer | VUMC | <12 h | 71 | F | F | NA | NA | NA | NA | training | evaluation | NA |
| 350 | nonCancer | VUMC | <12 h | 69 | F | F | NA | NA | NA | NA | training | evaluation | NA |
| 351 | nonCancer | VUMC | <12 h | 79 | M | F | NA | NA | NA | NA | training | training | NA |
| 352 | nonCancer | VUMC | <12 h | 67 | F | N | NA | NA | NA | NA | training | training | NA |
| 353 | nonCancer | VUMC | <12 h | 77 | F | N | NA | NA | NA | NA | training | training | NA |
| 354 | nonCancer | VUMC | <12 h | 51 | M | N | NA | NA | NA | NA | training | training | NA |
| 355 | nonCancer | VUMC | <12 h | 76 | F | N | NA | NA | NA | NA | training | training | NA |
| 356 | nonCancer | VUMC | <12 h | 29 | F | F | NA | NA | NA | NA | NA | validation | NA |
| 357 | nonCancer | VUMC | <12 h | 35 | M | N | NA | NA | NA | NA | NA | validation | NA |
| 358 | nonCancer | VUMC | <12 h | 40 | F | N | NA | NA | NA | NA | NA | validation | NA |
| 359 | nonCancer | VUMC | <12 h | 43 | F | N | NA | NA | NA | NA | NA | validation | NA |
| 360 | nonCancer | VUMC | <12 h | 34 | F | Y | NA | NA | NA | NA | NA | validation | NA |
| 361 | nonCancer | VUMC | <12 h | 17 | M | N | NA | NA | NA | NA | NA | validation | NA |
| 362 | nonCancer | VUMC | <12 h | 39 | F | NA | NA | NA | NA | NA | NA | validation | NA |
| 363 | nonCancer | VUMC | <12 h | 45 | F | N | NA | NA | NA | NA | NA | validation | NA |
| 364 | nonCancer | VUMC | <12 h | 36 | F | Y | NA | NA | NA | NA | NA | validation | NA |
| 365 | nonCancer | VUMC | <12 h | 24 | F | N | NA | NA | NA | NA | NA | validation | NA |
| 366 | nonCancer | VUMC | <12 h | 43 | F | NA | NA | NA | NA | NA | NA | validation | NA |
| 367 | nonCancer | UMCU | <12 h | 52 | F | Y | NA | NA | NA | NA | evaluation | training | NA |
| 368 | nonCancer | UMCU | <12 h | 71 | F | Y | NA | NA | NA | NA | evaluation | evaluation | NA |
| 369 | nonCancer | UMCU | <12 h | 63 | F | N | NA | NA | NA | NA | validation | training | NA |
| 370 | nonCancer | UMCU | <12 h | 73 | M | N | NA | NA | NA | NA | validation | training | NA |
| 371 | nonCancer | UMCU | <12 h | 65 | M | N | NA | NA | NA | NA | training | evaluation | NA |
| 372 | nonCancer | UMCU | <12 h | 39 | F | Y | NA | NA | NA | NA | NA | validation | NA |
| 373 | nonCancer | UMCU | <12 h | 55 | M | Y | NA | NA | NA | NA | training | training | NA |
| 374 | nonCancer | UMCU | <12 h | 70 | M | N | NA | NA | NA | NA | training | evaluation | NA |
| 375 | nonCancer | UMCU | <12 h | 48 | M | Y | NA | NA | NA | NA | training | training | NA |
| 376 | nonCancer | UMCU | <12 h | 39 | M | N | NA | NA | NA | NA | NA | validation | NA |
| 377 | NSCLC | VUMC | <12 h | 55 | M | NA | N | NA | NA | NA | evaluation | training | NA |
| 378 | NSCLC | VUMC | <12 h | 55 | F | F | Y | Vinorelbine | PD | NA | evaluation | validation | NA |
| 379 | NSCLC | VUMC | <12 h | 55 | F | F | Y | Vinorelbine | PD | NA | evaluation | validation | NA |
| 380 | NSCLC | VUMC | <12 h | 78 | M | N | Y | NA | NA | NA | evaluation | training | NA |
| 381 | NSCLC | VUMC | <12 h | 44 | M | N | Y | NA | NA | NA | evaluation | evaluation | NA |
| 382 | NSCLC | VUMC | <12 h | 39 | F | N | Y | NA | NA | NA | evaluation | evaluation | NA |
| 383 | NSCLC | VUMC | <12 h | 65 | F | F | Y | NA | NA | NA | evaluation | evaluation | NA |
| 384 | NSCLC | VUMC | <12 h | 42 | M | NA | Y | NA | NA | NA | evaluation | evaluation | NA |
| 385 | NSCLC | VUMC | <12 h | 61 | M | F | Y | Crizotinib | PR | NA | evaluation | training | NA |
| 386 | NSCLC | VUMC | <12 h | 61 | F | N | Y | NA | NA | NA | evaluation | evaluation | NA |
| 387 | NSCLC | VUMC | <12 h | 79 | F | N | Y | NA | NA | NA | evaluation | training | NA |
| 388 | NSCLC | VUMC | <12 h | 49 | F | Y | Y | Crizotinib | MR | NA | evaluation | training | NA |
| 389 | NSCLC | VUMC | <12 h | 73 | F | N | Y | NA | NA | NA | evaluation | validation | NA |
| 390 | NSCLC | VUMC | <12 h | 55 | M | N | Y | Ceritinib | PR | NA | evaluation | training | NA |
| 391 | NSCLC | VUMC | <12 h | 72 | M | N | Y | NA | NA | NA | evaluation | training | NA |
| 392 | NSCLC | VUMC | <12 h | 39 | M | N | Y | NA | NA | NA | evaluation | evaluation | NA |
| 393 | NSCLC | VUMC | <12 h | 84 | F | N | Y | NA | NA | NA | evaluation | training | NA |
| 394 | NSCLC | VUMC | <12 h | 74 | M | N | Y | NA | NA | NA | evaluation | validation | NA |
| 395 | NSCLC | VUMC | <12 h | 67 | M | N | Y | NA | NA | NA | evaluation | validation | NA |
| 396 | NSCLC | VUMC | <12 h | 46 | F | F | Y | NA | NA | NA | evaluation | evaluation | NA |
| 397 | NSCLC | VUMC | <12 h | 52 | M | Y | Y | NA | NA | NA | validation | validation | NA |
| 398 | NSCLC | VUMC | <12 h | 61 | F | N | Y | Vemurafenib | SD | NA | validation | training | NA |
| 399 | NSCLC | VUMC | <12 h | 54 | M | F | Y | Crizotinib | PD | NA | validation | training | NA |
| 400 | NSCLC | VUMC | <12 h | 68 | M | F | Y | Dabrafenib + | PR | NA | validation | evaluation | NA |
| | | | | | | | | Trametinib | | | | | |
| 401 | NSCLC | VUMC | <12 h | 60 | F | Y | Y | Dabrafenib | SD | NA | validation | training | NA |
| 402 | NSCLC | VUMC | <12 h | 43 | M | N | Y | Ceritinib | SD | NA | validation | evaluation | NA |
| 403 | NSCLC | VUMC | <12 h | 63 | M | Y | Y | Crizotinib | CR | NA | validation | evaluation | NA |
| 404 | NSCLC | VUMC | <12 h | 62 | M | N | Y | Crizotinib | PR | NA | validation | training | NA |
| 405 | NSCLC | VUMC | <12 h | 62 | M | F | Y | NA | NA | NA | validation | training | NA |
| 406 | NSCLC | VUMC | <12 h | 73 | F | N | Y | NA | NA | NA | validation | validation | NA |
| 407 | NSCLC | VUMC | <12 h | 81 | F | Y | Y | NA | NA | NA | validation | training | NA |
| 408 | NSCLC | VUMC | <12 h | 63 | M | F | Y | NA | NA | NA | validation | evaluation | NA |
| 409 | NSCLC | VUMC | <12 h | 49 | M | NA | Y | NA | NA | NA | validation | validation | NA |
| 410 | NSCLC | VUMC | <12 h | 65 | M | Y | Y | Crizotinib | SD | NA | validation | training | NA |
| 411 | NSCLC | VUMC | <12 h | 55 | F | N | Y | NA | NA | NA | validation | training | NA |
| 412 | NSCLC | VUMC | <12 h | 47 | M | N | Y | Pemetrexed | PD | NA | validation | validation | NA |
| 413 | NSCLC | VUMC | <12 h | 67 | M | F | Y | NA | NA | NA | validation | training | NA |
| 414 | NSCLC | VUMC | <12 h | 68 | F | N | Y | NA | NA | NA | validation | training | NA |
| 415 | NSCLC | VUMC | <12 h | 53 | F | N | Y | NA | NA | NA | validation | evaluation | NA |
| 416 | NSCLC | VUMC | <12 h | 60 | M | NA | Y | Crizotinib | NA | NA | validation | training | NA |
| 417 | NSCLC | VUMC | <12 h | 83 | F | N | Y | NA | NA | NA | validation | training | NA |
| 418 | NSCLC | VUMC | <12 h | 61 | F | N | NA | NA | NA | NA | validation | evaluation | NA |
| 419 | NSCLC | VUMC | <12 h | 55 | F | N | Y | NA | NA | NA | validation | evaluation | NA |
| 420 | NSCLC | VUMC | <12 h | 56 | F | N | Y | Crizotinib | PR | NA | validation | validation | NA |
| 421 | NSCLC | VUMC | <12 h | 66 | M | N | Y | Crizotinib | PR | NA | validation | evaluation | NA |
| 422 | NSCLC | VUMC | <12 h | 59 | M | F | Y | Gefitinib | PD | NA | validation | validation | NA |
| 423 | NSCLC | VUMC | <12 h | 53 | M | Y | Y | Crizotinib | PR | NA | validation | evaluation | NA |
| 424 | NSCLC | VUMC | <12 h | 81 | F | N | Y | Crizotinib | SD | NA | validation | validation | NA |
| 425 | NSCLC | VUMC | <12 h | 59 | F | N | Y | NA | NA | NA | validation | training | NA |
| 426 | NSCLC | VUMC | <12 h | 47 | M | N | Y | NA | PD | NA | validation | training | NA |
| 427 | NSCLC | VUMC | <12 h | 49 | M | Y | Y | NA | NA | NA | validation | evaluation | NA |
| 428 | NSCLC | VUMC | <12 h | 43 | M | N | Y | NA | NA | NA | validation | validation | NA |
| 429 | NSCLC | VUMC | <12 h | 62 | F | F | Y | Crizotinib | PD | NA | validation | validation | NA |
| 430 | NSCLC | VUMC | <12 h | 71 | F | F | Y | NA | NA | NA | validation | validation | NA |
| 431 | NSCLC | VUMC | <12 h | 61 | F | Y | Y | NA | NA | NA | validation | evaluation | NA |
| 432 | NSCLC | VUMC | <12 h | 66 | M | Y | Y | Crizotinib | PR | NA | validation | evaluation | NA |
| 433 | NSCLC | VUMC | <12 h | 64 | M | F | Y | Crizotinib | MR | NA | validation | validation | NA |
| 434 | NSCLC | VUMC | <12 h | 50 | F | NA | Y | NA | NA | NA | validation | training | NA |
| 435 | NSCLC | VUMC | <12 h | 67 | M | NA | Y | Sorafenib + | NA | NA | validation | training | NA |
| | | | | | | | | Metformin | | | | | |
| 436 | NSCLC | VUMC | <12 h | 54 | M | Y | Y | Sorafenib + | SD | NA | validation | evaluation | NA |
| | | | | | | | | Metformin | | | | | |
| 437 | NSCLC | VUMC | <12 h | 49 | F | N | Y | Sorafenib + | SD | NA | validation | validation | NA |
| | | | | | | | | Metformin | | | | | |
| 438 | NSCLC | VUMC | <12 h | 56 | F | Y | Y | Sorafenib | PD | NA | validation | training | NA |
| 439 | NSCLC | VUMC | <12 h | 44 | M | Y | Y | Sorafenib + | PR | NA | validation | training | NA |
| | | | | | | | | Metformin | | | | | |
| 440 | NSCLC | VUMC | <12 h | 50 | F | N | Y | Sorafenib + | PD | NA | validation | training | NA |
| | | | | | | | | Metformin | | | | | |
| 441 | NSCLC | VUMC | <12 h | 66 | M | NA | Y | NA | NA | NA | validation | evaluation | NA |
| 442 | NSCLC | VUMC | <12 h | 66 | F | NA | Y | Vemurafenib | PD | NA | validation | training | NA |
| 443 | NSCLC | VUMC | <12 h | 53 | F | F | Y | Dabrafenib | PR | NA | validation | training | NA |
| 444 | NSCLC | VUMC | <12 h | 66 | M | N | Y | Crizotinib | PR | NA | validation | training | NA |
| 445 | NSCLC | VUMC | <12 h | 83 | F | N | Y | NA | NA | NA | validation | training | NA |
| 446 | NSCLC | VUMC | <12 h | 54 | F | Y | Y | Crizo inib | PR | NA | validation | training | NA |
| 447 | NSCLC | VUMC | <12 h | 65 | F | F | Y | Vemurafenib | PR | NA | validation | validation | NA |
| 448 | NSCLC | VUMC | <12 h | 66 | F | NA | Y | Vemurafenib | PD | NA | validation | validation | NA |
| 449 | NSCLC | VUMC | <12 h | 68 | F | N | Y | Dabrafenib + | PR | NA | validation | validation | NA |
| | | | | | | | | Trametinib | | | | | |
| 450 | NSCLC | VUMC | <12 h | 73 | M | NA | Y | Cisplatin + | PR | NA | validation | training | NA |
| | | | | | | | | Pemetrexed | | | | | |
| 451 | NSCLC | VUMC | <12 h | 62 | F | F | Y | Dabrafenib + | PR | NA | validation | evaluation | NA |
| | | | | | | | | Trametinib | | | | | |
| 452 | NSCLC | VUMC | <12 h | 55 | M | NA | N | NA | NA | NA | validation | evaluation | NA |
| 453 | NSCLC | VUMC | <12 h | 56 | F | F | Y | NA | NA | NA | validation | validation | NA |
| 454 | NSCLC | VUMC | <12 h | 64 | M | NA | Y | NA | NA | NA | validation | validation | NA |
| 455 | NSCLC | VUMC | <12 h | 88 | M | Y | Y | Dabrafenib | SD | NA | validation | evaluation | NA |
| 456 | NSCLC | VUMC | <12 h | 27 | M | N | Y | Cisplatin + | PR | NA | validation | training | NA |
| | | | | | | | | Pemetrexed | | | | | |
| 457 | NSCLC | VUMC | <12 h | 72 | M | N | Y | NA | NA | NA | validation | validation | NA |
| 458 | NSCLC | VUMC | <12 h | 62 | F | F | Y | Dabrafenib | SD | NA | validation | evaluation | NA |
| 459 | NSCLC | VUMC | <12 h | 68 | M | Y | N | NA | NA | NA | validation | validation | NA |
| 460 | NSCLC | VUMC | <12 h | 71 | M | N | Y | NA | NA | NA | validation | evaluation | NA |
| 461 | NSCLC | VUMC | <12 h | 40 | M | N | Y | NA | NA | NA | validation | training | NA |
| 462 | NSCLC | VUMC | <12 h | 53 | F | F | Y | NA | NA | NA | validation | validation | NA |
| 463 | NSCLC | VUMC | <12 h | 73 | F | N | Y | NA | NA | NA | validation | evaluation | NA |
| 464 | NSCLC | VUMC | <12 h | 48 | M | NA | Y | NA | NA | NA | validation | evaluation | NA |
| 465 | NSCLC | VUMC | <12 h | 55 | M | N | Y | NA | NA | NA | validation | validation | NA |
| 466 | NSCLC | VUMC | <12 h | 65 | M | NA | Y | NA | NA | NA | validation | validation | NA |
| 467 | NSCLC | VUMC | <12 h | 64 | F | N | Y | NA | NA | NA | validation | validation | NA |
| 468 | NSCLC | VUMC | <12 h | 39 | F | N | Y | NA | NA | NA | validation | training | NA |
| 469 | NSCLC | VUMC | <12 h | 63 | M | F | Y | NA | NA | NA | validation | training | NA |
| 470 | NSCLC | VUMC | <12 h | 63 | M | N | Y | NA | NA | NA | validation | validation | NA |
| 471 | NSCLC | VUMC | <12 h | 78 | M | F | Y | NA | NA | NA | validation | training | NA |
| 472 | NSCLC | VUMC | <12 h | 76 | F | N | Y | NA | NA | NA | validation | training | NA |
| 473 | NSCLC | VUMC | <12 h | 59 | F | Y | Y | Garboplatin + | MR | NA | validation | training | NA |
| | | | | | | | | Gemcitabine | | | | | |
| 474 | NSCLC | VUMC | <12 h | 72 | M | N | Y | NA | NA | NA | validation | evaluation | NA |
| 475 | NSCLC | VUMC | <12 h | 74 | F | F | Y | Dabrafenib + | PR | NA | validation | training | NA |
| | | | | | | | | Trametinib | | | | | |
| 476 | NSCLC | VUMC | <12 h | 71 | F | F | Y | NA | NA | NA | validation | evaluation | NA |
| 477 | NSCLC | VUMC | <12 h | 68 | F | N | Y | Dabrafenib + | PR | NA | validation | validation | NA |
| | | | | | | | | Trametinib | | | | | |
| 478 | NSCLC | VUMC | <12 h | 53 | F | F | Y | NA | NA | NA | validation | training | NA |
| 479 | NSCLC | VUMC | <12 h | 69 | M | NA | Y | NA | NA | NA | validation | validation | NA |
| 480 | NSCLC | VUMC | <12 h | 73 | M | NA | Y | Cisplatin + | SD | NA | validation | training | NA |
| | | | | | | | | Pemetrexed | | | | | |
| 481 | NSCLC | VUMC | <12 h | 68 | F | N | Y | Dabrafenib + | PR | NA | validation | validation | NA |
| | | | | | | | | Trametinib | | | | | |
| 482 | NSCLC | VUMC | <12 h | 69 | M | N | Y | Nivolumab | PR | NA | validation | validation | NA |
| 483 | NSCLC | VUMC | <12 h | 47 | M | F | Y | Nivolumab | NA | NA | validation | training | NA |
| 484 | NSCLC | VUMC | <12 h | 75 | M | NA | Y | Nivolumab | PR | NA | validation | validation | NA |
| 485 | NSCLC | VUMC | <12 h | 75 | M | NA | Y | Nivolumab | PR | FollowUp | validation | validation | NA |
| 486 | NSCLC | VUMC | <12 h | 40 | M | Y | Y | Nivolumab | PD | FollowUp | validation | training | NA |
| 487 | NSCLC | VUMC | <12 h | 63 | F | F | Y | Erlotinib | SD | NA | training | validation | NA |
| 488 | NSCLC | VUMC | <12 h | 53 | M | Y | Y | NA | NA | NA | training | validation | NA |
| 489 | NSCLC | VUMC | <12 h | 55 | F | N | Y | NA | NA | NA | training | validation | NA |
| 490 | NSCLC | VUMC | <12 h | 61 | F | N | Y | NA | NA | NA | training | evaluation | NA |
| 491 | NSCLC | VUMC | <12 h | 59 | F | N | Y | NA | NA | NA | training | validation | NA |
| 492 | NSCLC | VUMC | <12 h | 63 | M | F | Y | NA | NA | NA | training | training | NA |
| 493 | NSCLC | VUMC | <12 h | 61 | M | Y | Y | Nivolumab | PD | FollowUp | training | training | NA |
| 494 | NSCLC | VUMC | <12 h | 53 | M | Y | Y | Crizotinib | NA | NA | training | validation | NA |
| 495 | NSCLC | VUMC | <12 h | 63 | M | N | Y | Crizotinib | PR | NA | training | evaluation | NA |
| 496 | NSCLC | VUMC | <12 h | 55 | M | Y | Y | NA | NA | NA | training | training | NA |
| 497 | NSCLC | VUMC | <12 h | 48 | M | N | Y | Pemetrexed | PD | NA | training | validation | NA |
| 498 | NSCLC | VUMC | <12 h | 42 | M | NA | Y | NA | NA | NA | training | evaluation | NA |
| 499 | NSCLC | VUMC | <12 h | 63 | M | Y | Y | NA | NA | NA | training | training | NA |
| 500 | NSCLC | VUMC | <12 h | 61 | F | N | Y | NA | NA | NA | training | validation | NA |
| 501 | NSCLC | VUMC | <12 h | 64 | M | N | Y | NA | NA | NA | training | validation | NA |
| 502 | NSCLC | VUMC | <12 h | 54 | M | NA | N | NA | NA | NA | training | evaluation | NA |
| 503 | NSCLC | VUMC | <12 h | 65 | F | F | Y | NA | NA | NA | training | evaluation | NA |
| 504 | NSCLC | VUMC | <12 h | 53 | M | F | Y | Nivolumab | PR | Baseline | training | validation | Training |
| 505 | NSCLC | VUMC | <12 h | 61 | F | Y | Y | Crizotinib | PR | NA | training | training | NA |
| 506 | NSCLC | VUMC | <12 h | 56 | F | N | Y | NA | NA | NA | training | training | NA |
| 507 | NSCLC | VUMC | <12 h | 62 | F | F | Y | Dabrafenib + | PR | NA | training | training | NA |
| | | | | | | | | Trametinib | | | | | |
| 508 | NSCLC | VUMC | <12 h | 59 | F | N | Y | NA | NA | NA | training | training | NA |
| 509 | NSCLC | VUMC | <12 h | 39 | M | Y | Y | Nivolumab | MR | NA | training | training | NA |
| 510 | NSCLC | VUMC | <12 h | 56 | F | Y | N | Nivolumab | PR | NA | training | validation | NA |
| 511 | NSCLC | VUMC | <12 h | 43 | M | N | Y | NA | NA | NA | training | training | NA |
| 512 | NSCLC | VUMC | <12 h | 73 | M | NA | Y | Cisplatin + | SD | NA | training | evaluation | NA |
| | | | | | | | | Pemetrexed | | | | | |
| 513 | NSCLC | VUMC | <12 h | 47 | F | F | Y | NA | NA | NA | training | validation | NA |
| 514 | NSCLC | VUMC | <12 h | 56 | F | Y | N | Nivolumab | PR | Baseline | training | evaluation | Training |
| 515 | NSCLC | VUMC | <12 h | 48 | M | N | Y | NA | NA | NA | training | training | NA |
| 516 | NSCLC | VUMC | <12 h | 81 | F | N | Y | NA | NA | NA | training | validation | NA |
| 517 | NSCLC | VUMC | <12 h | 53 | M | F | Y | Nivolumab | PR | FollowUp | training | evaluation | NA |
| 518 | NSCLC | VUMC | <12 h | 74 | F | N | Y | NA | NA | NA | training | evaluation | NA |
| 519 | NSCLC | VUMC | <12 h | 75 | M | Y | Y | Nivolumab | PR | FollowUp | training | validation | NA |
| 520 | NSCLC | VUMC | <12 h | 79 | M | N | Y | NA | NA | NA | training | validation | NA |
| 521 | NSCLC | VUMC | <12 h | 63 | F | F | Y | Erlotinib | SD | NA | training | evaluation | NA |
| 522 | NSCLC | VUMC | <12 h | 54 | F | Y | Y | Crizotinib | PR | NA | training | validation | NA |
| 523 | NSCLC | VUMC | <12 h | 67 | M | Y | Y | NA | NA | NA | training | validation | NA |
| 524 | NSCLC | VUMC | <12 h | 63 | F | F | Y | Ceritinib | PD | NA | training | evaluation | NA |
| 525 | NSCLC | VUMC | <12 h | 44 | M | N | Y | NA | NA | NA | training | validation | NA |
| 526 | NSCLC | VUMC | <12 h | 39 | M | N | Y | NA | NA | NA | training | evaluation | NA |
| 527 | NSCLC | VUMC | <12 h | 54 | F | Y | Y | NA | NA | NA | training | training | NA |
| 528 | NSCLC | VUMC | <12 h | 61 | F | N | Y | NA | NA | NA | training | validation | NA |
| 529 | NSCLC | VUMC | <12 h | 78 | M | N | Y | NA | NA | NA | training | training | NA |
| 530 | NSCLC | VUMC | <12 h | 57 | F | F | Y | NA | NA | NA | training | validation | NA |
| 531 | NSCLC | VUMC | <12 h | 54 | F | F | Y | NA | NA | NA | training | training | NA |
| 532 | NSCLC | VUMC | <12 h | 61 | F | Y | Y | CLDK-2201 | PR | NA | training | training | NA |
| 533 | NSCLC | VUMC | <12 h | 74 | F | F | Y | Dabrafenib + | PR | NA | training | evaluation | NA |
| | | | | | | | | Trametinib | | | | | |
| 534 | NSCLC | VUMC | <12 h | 56 | F | F | Y | NA | NA | NA | training | validation | NA |
| 535 | NSCLC | VUMC | <12 h | 78 | M | NA | Y | Nivolumab | SD | Baseline | training | evaluation | Training |
| 536 | NSCLC | MGH | >12 h | 32 | M | NA | Y | NA | NA | NA | NA | validation | NA |
| 537 | NSCLC | MGH | >12 h | 58 | M | NA | Y | NA | NA | NA | NA | validation | NA |
| 538 | NSCLC | MGH | >12 h | 82 | F | NA | Y | NA | NA | NA | NA | validation | NA |
| 539 | NSCLC | MGH | >12 h | 74 | F | NA | Y | NA | NA | NA | NA | validation | NA |
| 540 | NSCLC | MGH | >12 h | 36 | M | N | Y | NA | NA | NA | NA | validation | NA |
| 541 | NSCLC | MGH | >12 h | 33 | M | NA | Y | NA | NA | NA | NA | validation | NA |
| 542 | NSCLC | MGH | >12 h | 63 | M | NA | Y | NA | NA | NA | NA | validation | NA |
| 543 | NSCLC | MGH | >12 h | 77 | F | NA | Y | NA | NA | NA | NA | validation | NA |
| 544 | NSCLC | MGH | >12 h | 37 | F | NA | Y | NA | NA | NA | NA | validation | NA |
| 545 | NSCLC | MGH | >12 h | 69 | M | NA | Y | NA | NA | NA | NA | validation | NA |
| 546 | NSCLC | MGH | >12 h | 67 | F | NA | Y | NA | NA | NA | NA | validation | NA |
| 547 | NSCLC | MGH | >12 h | 73 | M | NA | Y | NA | NA | NA | NA | validation | NA |
| 548 | NSCLC | MGH | >12 h | 48 | F | NA | Y | NA | NA | NA | NA | validation | NA |
| 549 | NSCLC | MGH | >12 h | 54 | M | NA | Y | NA | NA | NA | NA | validation | NA |
| 550 | NSCLC | MGH | >12 h | 55 | F | NA | Y | NA | NA | NA | NA | validation | NA |
| 551 | NSCLC | MGH | >12 h | 55 | M | NA | Y | NA | NA | NA | NA | validation | NA |
| 552 | NSCLC | MGH | >12 h | 68 | F | NA | Y | NA | NA | NA | NA | validation | NA |
| 553 | NSCLC | MGH | >12 h | 68 | F | NA | Y | NA | NA | NA | NA | validation | NA |
| 554 | NSCLC | MGH | >12 h | 62 | M | NA | Y | NA | NA | NA | NA | validation | NA |
| 555 | NSCLC | MGH | >12 h | 49 | F | NA | Y | NA | NA | NA | NA | validation | NA |
| 556 | NSCLC | MGH | >12 h | 67 | M | NA | Y | NA | NA | NA | NA | validation | NA |
| 557 | NSCLC | MGH | >12 h | 82 | F | NA | Y | NA | NA | NA | NA | validation | NA |
| 558 | NSCLC | MGH | >12 h | 62 | F | NA | Y | NA | NA | NA | NA | validation | NA |
| 559 | NSCLC | MGH | >12 h | 53 | F | NA | Y | NA | NA | NA | NA | validation | NA |
| 560 | NSCLC | MGH | >12 h | 60 | M | NA | Y | NA | NA | NA | NA | validation | NA |
| 561 | NSCLC | MGH | >12 h | 64 | F | NA | Y | NA | NA | NA | NA | validation | NA |
| 562 | NSCLC | MGH | >12 h | 50 | F | NA | Y | NA | NA | NA | NA | validation | NA |
| 563 | NSCLC | MGH | >12 h | 64 | F | NA | Y | NA | NA | NA | NA | validation | NA |
| 564 | NSCLC | MGH | >12 h | 64 | F | NA | Y | NA | NA | NA | NA | validation | NA |
| 565 | NSCLC | MGH | >12 h | 68 | F | NA | Y | NA | NA | NA | NA | validation | NA |
| 566 | NSCLC | MGH | >12 h | 78 | M | NA | Y | NA | NA | NA | NA | validation | NA |
| 567 | NSCLC | MGH | >12 h | 86 | M | NA | Y | NA | NA | NA | NA | validation | NA |
| 568 | NSCLC | HGTP | <12 h | 72 | M | N | Y | NA | NA | NA | NA | validation | NA |
| 569 | NSCLC | HGTP | <12 h | 36 | F | Y | Y | NA | NA | NA | NA | validation | NA |
| 570 | NSCLC | MGH | >12 h | 65 | F | NA | Y | NA | NA | NA | NA | validation | NA |
| 571 | NSCLC | MGH | >12 h | 64 | M | NA | Y | NA | NA | NA | NA | validation | NA |
| 572 | NSCLC | MGH | >12 h | 61 | M | NA | Y | NA | NA | NA | NA | validation | NA |
| 573 | NSCLC | MGH | >12 h | 56 | M | NA | Y | NA | NA | NA | NA | validation | NA |
| 574 | NSCLC | MGH | >12 h | 70 | M | NA | Y | NA | NA | NA | NA | validation | NA |
| 575 | NSCLC | MGH | >12 h | 59 | M | NA | Y | NA | NA | NA | NA | validation | NA |
| 576 | NSCLC | MGH | >12 h | 58 | M | NA | Y | NA | NA | NA | NA | validation | NA |
| 577 | NSCLC | MGH | >12 h | 70 | F | NA | Y | NA | NA | NA | NA | validation | NA |
| 578 | NSCLC | MGH | >12 h | 42 | F | NA | Y | NA | NA | NA | NA | validation | NA |
| 579 | NSCLC | MGH | >12 h | 55 | F | NA | Y | NA | NA | NA | NA | validation | NA |
| 580 | NSCLC | MGH | >12 h | 74 | M | NA | Y | NA | NA | NA | NA | validation | NA |
| 581 | NSCLC | MGH | >12 h | 63 | F | NA | Y | NA | NA | NA | NA | validation | NA |
| 582 | NSCLC | MGH | >12 h | 54 | F | NA | Y | NA | NA | NA | NA | validation | NA |
| 583 | NSCLC | NKI | <12 h | 54 | M | F | Y | NA | NA | NA | NA | validation | NA |
| 584 | NSCLC | NKI | <12 h | 69 | M | F | Y | NA | NA | NA | NA | validation | NA |
| 585 | NSCLC | NKI | <12 h | 74 | M | F | Y | NA | NA | NA | NA | validation | NA |
| 586 | NSCLC | NKI | <12 h | 54 | M | F | Y | NA | NA | NA | NA | validation | NA |
| 587 | NSCLC | NKI | <12 h | 73 | F | F | Y | NA | NA | NA | NA | validation | NA |
| 588 | NSCLC | NKI | <12 h | 73 | F | F | Y | NA | NA | NA | NA | validation | NA |
| 589 | NSCLC | NKI | <12 h | 67 | F | F | Y | Nivolumab | PD | NA | NA | validation | Training |
| 590 | NSCLC | NKI | <12 h | 73 | F | F | Y | NA | NA | NA | NA | validation | NA |
| 591 | NSCLC | NKI | <12 h | 67 | M | F | Y | Nivolumab | PD | NA | NA | validation | NA |
| 592 | NSCLC | NKI | <12 h | 73 | F | F | Y | NA | NA | NA | NA | validation | NA |
| 593 | NSCLC | NKI | <12 h | 69 | M | F | Y | NA | NA | NA | NA | validation | NA |
| 594 | NSCLC | NKI | <12 h | 74 | M | F | Y | NA | NA | NA | NA | validation | NA |
| 595 | NSCLC | NKI | <12 h | 67 | M | Y | Y | Nivolumab | SD | NA | NA | validation | NA |
| 596 | NSCLC | NKI | <12 h | 74 | M | F | Y | NA | NA | NA | NA | validation | NA |
| 597 | NSCLC | NKI | <12 h | 73 | F | F | Y | NA | NA | NA | NA | validation | NA |
| 598 | NSCLC | NKI | <12 h | 67 | F | F | Y | NA | NA | NA | NA | validation | NA |
| 599 | NSCLC | NKI | <12 h | 74 | M | F | Y | NA | NA | NA | NA | validation | NA |
| 600 | NSCLC | NKI | <12 h | 74 | M | F | Y | NA | NA | NA | NA | validation | NA |
| 601 | NSCLC | NKI | <12 h | 41 | M | F | Y | NA | NA | NA | NA | validation | NA |
| 602 | NSCLC | NKI | <12 h | 73 | F | F | Y | NA | NA | NA | NA | validation | NA |
| 603 | NSCLC | NKI | <12 h | 54 | M | F | Y | NA | NA | NA | NA | validation | NA |
| 604 | NSCLC | NKI | <12 h | 67 | M | Y | Y | NA | NA | NA | NA | validation | NA |
| 605 | NSCLC | NKI | <12 h | 74 | M | F | Y | NA | NA | NA | NA | validation | NA |
| 606 | NSCLC | NKI | <12 h | 54 | M | F | Y | NA | NA | NA | NA | validation | NA |
| 607 | NSCLC | NKI | <12 h | 67 | M | Y | Y | NA | NA | NA | NA | validation | NA |
| 608 | NSCLC | NKI | <12 h | 52 | M | N | Y | NA | NA | NA | NA | validation | NA |
| 609 | NSCLC | NKI | <12 h | 52 | F | N | Y | Nivolumab | SD | NA | NA | validation | Evaluation |
| 610 | NSCLC | NKI | <12 h | 49 | F | F | Y | Nivolumab | PR | NA | NA | validation | NA |
| 611 | NSCLC | NKI | <12 h | 49 | F | F | Y | Nivolumab | PR | NA | NA | validation | NA |
| 612 | NSCLC | NKI | <12 h | 69 | M | F | Y | NA | NA | NA | NA | validation | NA |
| 613 | NSCLC | NKI | <12 h | 74 | M | F | Y | NA | NA | NA | NA | validation | NA |
| 614 | NSCLC | NKI | <12 h | 54 | M | F | Y | NA | NA | NA | NA | validation | NA |
| 615 | NSCLC | NKI | <12 h | 67 | M | Y | Y | NA | NA | NA | NA | validation | NA |
| 616 | NSCLC | NKI | <12 h | 69 | M | F | Y | NA | NA | NA | NA | validation | NA |
| 617 | NSCLC | MGH | >12 h | 50 | F | NA | Y | NA | NA | NA | NA | validation | NA |
| 618 | NSCLC | MGH | >12 h | 51 | F | NA | Y | NA | NA | NA | NA | validation | NA |
| 619 | NSCLC | MGH | >12 h | 70 | F | NA | Y | NA | NA | NA | NA | validation | NA |
| 620 | NSCLC | MGH | >12 h | 49 | F | NA | Y | NA | NA | NA | NA | validation | NA |
| 621 | NSCLC | MGH | >12 h | 68 | F | NA | Y | NA | NA | NA | NA | validation | NA |
| 622 | NSCLC | MGH | >12 h | 71 | F | NA | Y | NA | NA | NA | NA | validation | NA |
| 623 | NSCLC | MGH | >12 h | 55 | F | NA | Y | NA | NA | NA | NA | validation | NA |
| 624 | NSCLC | MGH | >12 h | 65 | M | NA | Y | NA | NA | NA | NA | validation | NA |
| 625 | NSCLC | MGH | >12 h | 58 | F | NA | Y | NA | NA | NA | NA | validation | NA |
| 626 | NSCLC | MGH | >12 h | 58 | F | NA | Y | NA | NA | NA | NA | validation | NA |
| 627 | NSCLC | MGH | >12 h | 53 | M | NA | Y | NA | NA | NA | NA | validation | NA |
| 628 | NSCLC | MGH | >12 h | 53 | M | NA | N | NA | NA | NA | NA | validation | NA |
| 629 | NSCLC | MGH | >12 h | 58 | M | NA | Y | NA | NA | NA | NA | validation | NA |
| 630 | NSCLC | MGH | >12 h | 72 | F | NA | Y | NA | NA | NA | NA | validation | NA |
| 631 | NSCLC | MGH | >12 h | 67 | F | NA | Y | NA | NA | NA | NA | validation | NA |
| 632 | NSCLC | MGH | >12 h | 63 | M | NA | Y | NA | NA | NA | NA | validation | NA |
| 633 | NSCLC | MGH | >12 h | 60 | F | NA | Y | NA | NA | NA | NA | validation | NA |
| 634 | NSCLC | MGH | >12 h | 68 | M | NA | N | NA | NA | NA | NA | validation | NA |
| 635 | NSCLC | VUMC | <12 h | 88 | M | Y | Y | Dabrafenib | SD | NA | NA | validation | NA |
| 636 | NSCLC | VUMC | <12 h | 88 | M | Y | Y | Dabrafenib | SD | NA | NA | validation | NA |
| 637 | NSCLC | NKI | <12 h | 74 | M | Y | Y | Nivolumab | PD | FollowUp | NA | validation | NA |
| 638 | NSCLC | NKI | <12 h | 75 | M | F | Y | NA | NA | NA | NA | validation | NA |
| 639 | NSCLC | NKI | <12 h | 66 | F | F | Y | Nivolumab | PD | Baseline | NA | validation | Training |
| 640 | NSCLC | NKI | <12 h | 68 | M | Y | Y | Nivolumab | PD | NA | NA | validation | Training |
| 641 | NSCLC | NKI | <12 h | 58 | M | Y | Y | Nivolumab | PR | FollowUp | NA | validation | NA |
| 642 | NSCLC | NKI | <12 h | 69 | M | F | Y | Nivolumab | PD | NA | NA | validation | Training |
| 643 | NSCLC | NKI | <12 h | 69 | M | F | Y | Nivolumab | PD | FollowUp | NA | validation | NA |
| 644 | NSCLC | NKI | <12 h | 74 | M | N | Y | Nivol umab | PD | Baseline | NA | validation | Validation |
| 645 | NSCLC | NKI | <12 h | 58 | M | F | Y | Nivolumab | PD | Baseline | NA | validation | Evaluation |
| 646 | NSCLC | NKI | <12 h | 57 | F | Y | Y | Nivolumab | PR | NA | NA | validation | Training |
| 647 | NSCLC | NKI | <12 h | 66 | F | F | Y | Nivolumab | SD | NA | NA | validation | Evaluation |
| 648 | NSCLC | NKI | <12 h | 67 | M | F | Y | Nivolumab | PD | FollowUp | NA | validation | NA |
| 649 | NSCLC | NKI | <12 h | 75 | M | F | Y | Nivolumab | SD | NA | NA | validation | NA |
| 650 | NSCLC | NKI | <12 h | 74 | M | F | Y | Nivolumab | SD | NA | NA | validation | Training |
| 651 | NSCLC | NKI | <12 h | 63 | M | F | Y | Nivolumab | PR | NA | NA | validation | Training |
| 652 | NSCLC | NKI | <12 h | 58 | F | Y | Y | Nivolumab | PR | NA | NA | validation | Training |
| 653 | NSCLC | NKI | <12 h | 68 | M | Y | Y | Nivolumab | PD | NA | NA | validation | Training |
| 654 | NSCLC | NKI | <12 h | 65 | F | Y | Y | Nivolumab | PD | NA | NA | validation | Evaluation |
| 655 | NSCLC | NKI | <12 h | 70 | M | Y | Y | Nivolumab | PR | NA | NA | validation | Training |
| 656 | NSCLC | VUMC | <12 h | 62 | M | N | Y | Nivolumab | PR | Baseline | NA | validation | Training |
| 657 | NSCLC | VUMC | <12 h | 61 | M | N | Y | NA | NA | NA | NA | validation | NA |
| 658 | NSCLC | VUMC | <12 h | 55 | M | Y | Y | Nivolumab | PR | Baseline | NA | validation | Training |
| 659 | NSCLC | NKI | <12 h | 74 | M | Y | Y | Nivolumab | PD | NA | NA | validation | Training |
| 660 | NSCLC | NKI | <12 h | 53 | M | Y | Y | Nivolumab | PR | FollowUp | NA | validation | NA |
| 661 | NSCLC | NKI | <12 h | 68 | F | F | Y | Nivolumab | PD | FollowUp | NA | validation | NA |
| 662 | NSCLC | NKI | <12 h | 68 | F | F | Y | Nivolumab | SD | FollowUp | NA | validation | NA |
| 663 | NSCLC | NKI | <12 h | 59 | F | F | Y | Nivolumab | PD | FollowUp | NA | validation | NA |
| 664 | NSCLC | NKI | <12 h | 67 | M | F | Y | Nivolumab | PD | Baseline | NA | validation | Training |
| 665 | NSCLC | NKI | <12 h | 59 | F | F | Y | Nivolumab | PD | NA | NA | validation | Training |
| 666 | NSCLC | NKI | <12 h | 61 | F | F | Y | Nivolumab | PR | NA | NA | validation | Training |
| 667 | NSCLC | NKI | <12 h | 68 | F | F | Y | Nivolumab | SD | NA | NA | validation | Training |
| 668 | NSCLC | NKI | <12 h | 65 | M | F | Y | Nivolumab | PR | NA | NA | validation | Training |
| 669 | NSCLC | NKI | <12 h | 74 | M | F | Y | Nivolumab | SD | FollowUp | NA | validation | NA |
| 670 | NSCLC | NKI | <12 h | 69 | M | F | Y | Nivolumab | NA | NA | NA | validation | NA |
| 671 | NSCLC | NKI | <12 h | 69 | M | F | Y | Nivolumab | PD | Baseline | NA | validation | Training |
| 672 | NSCLC | NKI | <12 h | 58 | M | Y | Y | Nivolumab | MR | NA | NA | validation | NA |
| 673 | NSCLC | NKI | <12 h | NA | M | F | Y | Nivolumab | PR | NA | NA | validation | Training |
| 674 | NSCLC | NKI | <12 h | 72 | F | N | Y | Nivolumab | SD | NA | NA | validation | NA |
| 675 | NSCLC | NKI | <12 h | 73 | M | F | Y | Nivolumab | PD | Baseline | NA | validation | Evaluation |
| 676 | NSCLC | NKI | <12 h | 58 | M | Y | Y | Nivolumab | PR | Baseline | NA | validation | Training |
| 677 | NSCLC | NKI | <12 h | 50 | F | F | Y | Nivolumab | SD | NA | NA | validation | NA |
| 678 | NSCLC | NKI | <12 h | 66 | F | F | Y | Nivolumab | PD | FollowUp | NA | validation | NA |
| 679 | NSCLC | NKI | <12 h | 73 | M | F | Y | Nivolumab | SD | NA | NA | validation | NA |
| 680 | NSCLC | VUMC | <12 h | 53 | F | F | N | Nivolumab | PR | Baseline | NA | validation | Evaluation |
| 681 | NSCLC | VUMC | <12 h | 65 | M | F | Y | Nivolumab | PD | NA | NA | validation | NA |
| 682 | NSCLC | NKI | <12 h | 57 | F | Y | Y | Nivolumab | PR | FollowUp | NA | validation | NA |
| 683 | NSCLC | NKI | <12 h | 65 | M | F | Y | Nivolumab | PR | FollowUp | NA | validation | NA |
| 684 | NSCLC | NKI | <12 h | 60 | F | Y | Y | Nivolumab | PD | NA | NA | validation | NA |
| 685 | NSCLC | NKI | <12 h | 38 | M | N | Y | Nivolumab | PD | NA | NA | validation | Training |
| 686 | NSCLC | NKI | <12 h | 68 | F | F | Y | Nivolumab | SD | FollowUp | NA | validation | NA |
| 687 | NSCLC | NKI | <12 h | 38 | M | N | Y | Nivolumab | PD | FollowUp | NA | validation | NA |
| 688 | NSCLC | NKI | <12 h | 65 | M | F | Y | Nivolumab | PR | FollowUp | NA | validation | NA |
| 689 | NSCLC | NKI | <12 h | 62 | F | N | Y | Nivolumab | SD | FollowUp | NA | validation | NA |
| 690 | NSCLC | NKI | <12 h | 74 | M | F | Y | Nivolumab | SD | FollowUp | NA | validation | NA |
| 691 | NSCLC | VUMC | <12 h | 73 | M | F | Y | Nivolumab | SD | NA | NA | validation | NA |
| 692 | NSCLC | VUMC | <12 h | 69 | M | N | Y | Nivolumab | PR | NA | NA | validation | NA |
| 693 | NSCLC | NKI | <12 h | 68 | M | Y | Y | Nivolumab | PD | FollowUp | NA | validation | NA |
| 694 | NSCLC | NKI | <12 h | 42 | M | F | Y | NA | NA | NA | NA | validation | NA |
| 695 | NSCLC | NKI | <12 h | 49 | F | F | Y | NA | NA | NA | NA | validation | NA |
| 696 | NSCLC | NKI | <12 h | 77 | F | F | Y | Nivolumab | PD | NA | NA | validation | Training |
| 697 | NSCLC | NKI | <12 h | 58 | M | F | Y | NA | NA | NA | NA | validation | NA |
| 698 | NSCLC | NKI | <12 h | 72 | F | Y | Y | Crizotinib | PD | NA | NA | validation | NA |
| 699 | NSCLC | NKI | <12 h | 75 | M | N | Y | Nivolumab | PD | Baseline | NA | validation | Training |
| 700 | NSCLC | NKI | <12 h | 56 | F | F | Y | Nivolumab | SD | NA | NA | validation | NA |
| 701 | NSCLC | NKI | <12 h | 63 | F | F | Y | Nivolumab | PD | NA | NA | validation | Validation |
| 702 | NSCLC | NKI | <12 h | 44 | F | Y | Y | Crizotinib | PD | NA | NA | validation | NA |
| 703 | NSCLC | NKI | <12 h | 49 | M | N | Y | Nivolumab | PD | Baseline | NA | validation | Training |
| 704 | NSCLC | NKI | <12 h | 55 | F | Y | Y | Nivolumab | PD | FollowUp | NA | validation | NA |
| 705 | NSCLC | NKI | <12 h | 73 | F | F | Y | Nivolumab | PR | NA | NA | validation | Training |
| 706 | NSCLC | NKI | <12 h | 67 | F | F | Y | Nivolumab | SD | NA | NA | validation | NA |
| 707 | NSCLC | NKI | <12 h | 68 | F | F | Y | Nivolumab | PR | Baseline | NA | validation | Validation |
| 708 | NSCLC | NKI | <12 h | 55 | F | F | Y | Nivolumab | PD | NA | NA | validation | Training |
| 709 | NSCLC | NKI | <12 h | 65 | M | Y | Y | Nivolumab | PR | NA | NA | validation | Evaluation |
| 710 | NSCLC | NKI | <12 h | 64 | F | N | Y | Nivolumab | PR | Baseline | NA | validation | Training |
| 711 | NSCLC | NKI | <12 h | 70 | F | F | Y | Nivolumab | PR | Baseline | NA | validation | Evaluation |
| 712 | NSCLC | NKI | <12 h | 77 | F | F | Y | Nivolumab | PD | Baseline | NA | validation | Evaluation |
| 713 | NSCLC | NKI | <12 h | 64 | F | Y | Y | Nivolumab | PD | Baseline | NA | validation | Training |
| 714 | NSCLC | NKI | <12 h | 60 | M | N | Y | Nivolumab | PD | Baseline | NA | validation | Validation |
| 715 | NSCLC | NKI | <12 h | 68 | F | F | Y | Nivolumab | SD | Baseline | NA | validation | Validation |
| 716 | NSCLC | NKI | <12 h | 60 | F | F | Y | Nivolumab | PD | Baseline | NA | validation | Validation |
| 717 | NSCLC | NKI | <12 h | 50 | F | F | Y | NA | NA | NA | NA | validation | NA |
| 718 | NSCLC | NKI | <12 h | 62 | M | F | Y | Nivolumab | PD | Baseline | NA | validation | Evaluation |
| 719 | NSCLC | NKI | <12 h | 50 | F | F | Y | NA | NA | NA | NA | validation | NA |
| 720 | NSCLC | NKI | <12 h | 68 | M | Y | Y | Nivolumab | PD | Baseline | NA | validation | Validation |
| 721 | NSCLC | NKI | <12 h | 30 | F | N | Y | Nivolumab | PD | Baseline | NA | validation | Evaluation |
| 722 | NSCLC | NKI | <12 h | 58 | M | F | Y | Nivolumab | PD | Baseline | NA | validation | Validation |
| 723 | NSCLC | NKI | <12 h | 46 | F | N | Y | Nivolumab | PD | Baseline | NA | validation | Evaluation |
| 724 | NSCLC | NKI | <12 h | 75 | M | F | Y | Nivolumab | PD | Baseline | NA | validation | Training |
| 725 | NSCLC | NKI | <12 h | 66 | F | Y | Y | Nivolumab | PD | Baseline | NA | validation | Evaluation |
| 726 | NSCLC | NKI | <12 h | 58 | M | F | Y | Nivolumab | PD | Baseline | NA | validation | Training |
| 727 | NSCLC | NKI | <12 h | 68 | M | F | Y | Nivolumab | PR | Baseline | NA | validation | NA |
| 728 | NSCLC | NKI | <12 h | 60 | M | N | Y | NA | NA | NA | NA | validation | NA |
| 729 | NSCLC | NKI | <12 h | 77 | F | F | Y | Nivolumab | PD | Baseline | NA | NA | Training |
| 730 | NSCLC | NKI | <12 h | 56 | F | F | Y | Nivolumab | PD | Baseline | NA | NA | Validation |
| 731 | NSCLC | NKI | <12 h | 63 | F | F | Y | Nivolumab | PD | Baseline | NA | NA | Validation |
| 732 | NSCLC | NKI | <12 h | 73 | F | F | Y | Nivolumab | PR | Baseline | NA | NA | Training |
| 733 | NSCLC | NKI | <12 h | 67 | F | F | Y | Nivolumab | PD | Baseline | NA | NA | Validation |
| 734 | NSCLC | NKI | <12 h | 55 | F | F | Y | Nivolumab | PD | Baseline | NA | NA | Training |
| 735 | NSCLC | NKI | <12 h | 65 | M | Y | Y | Nivolumab | PR | Baseline | NA | NA | Evaluation |
| 736 | NSCLC | NKI | <12 h | 74 | M | F | Y | Nivolumab | PD | Baseline | NA | NA | Validation |
| 737 | NSCLC | NKI | <12 h | 62 | M | N | N | Nivolumab | PD | Baseline | NA | NA | Evaluation |
| 738 | NSCLC | VUMC | <12 h | 61 | M | N | N | Nivolumab | PR | Baseline | NA | NA | Evaluation |
| 739 | NSCLC | VUMC | <12 h | 61 | F | N | N | Nivolumab | PR | Baseline | NA | NA | Training |
| 740 | NSCLC | VUMC | <12 h | 69 | M | N | N | Nivolumab | PR | Baseline | NA | NA | Training |
| 741 | NSCLC | VUMC | <12 h | 69 | F | N | N | Nivolumab | PR | Baseline | NA | NA | Evaluation |
| 742 | NSCLC | VUMC | <12 h | 54 | F | N | N | Nivolumab | PD | Baseline | NA | NA | Training |
| 743 | NSCLC | VUMC | <12 h | 68 | M | N | N | Nivolumab | PD | Baseline | NA | NA | Training |
| 744 | NSCLC | VUMC | <12 h | 69 | M | N | N | Nivolumab | PR | Baseline | NA | NA | Training |
| 745 | NSCLC | VUMC | <12 h | 67 | M | N | N | Nivolumab | PR | Baseline | NA | NA | Training |
| 746 | NSCLC | VUMC | <12 h | 62 | M | N | N | Nivolumab | PD | Baseline | NA | NA | Training |
| 747 | NSCLC | VUMC | <12 h | 72 | F | N | N | Nivolumab | PD | Baseline | NA | NA | Training |
| 748 | NSCLC | VUMC | <12 h | 56 | M | N | N | Nivolumab | PD | Baseline | NA | NA | Training |
| 749 | NSCLC | VUMC | <12 h | 56 | F | N | N | Nivolumab | PD | Baseline | NA | NA | Training |
| 750 | NSCLC | VUMC | <12 h | 65 | M | N | N | Nivolumab | PD | Baseline | NA | NA | Training |
| 751 | NSCLC | VUMC | <12 h | 69 | F | N | N | Nivolumab | PR | Baseline | NA | NA | Training |
| 752 | NSCLC | VUMC | <12 h | 69 | M | N | N | Nivolumab | PD | Baseline | NA | NA | Training |
| 753 | NSCLC | VUMC | <12 h | 60 | F | N | N | Nivolumab | PD | Baseline | NA | NA | Training |
| 754 | NSCLC | VUMC | <12 h | 70 | M | N | N | Nivolumab | PD | Baseline | NA | NA | Training |
| 755 | NSCLC | VUMC | <12 h | 58 | M | N | N | Nivolumab | PD | Baseline | NA | NA | Validation |
| 756 | NSCLC | VUMC | <12 h | 64 | M | N | N | Nivolumab | PD | Baseline | NA | NA | Evaluation |
| 757 | NSCLC | VUMC | <12 h | 69 | F | N | N | Nivolumab | PR | Baseline | NA | NA | Evaluation |
| 758 | NSCLC | NKI | <12 h | 65 | M | N | N | Nivolumab | PD | Baseline | NA | NA | Training |
| 759 | NSCLC | NKI | <12 h | 54 | F | N | N | Nivolumab | PR | Baseline | NA | NA | Validation |
| 760 | NSCLC | NKI | <12 h | 58 | M | N | N | Nivolumab | PR | Baseline | NA | NA | Validation |
| 761 | NSCLC | NKI | <12 h | 64 | F | N | N | Nivolumab | PR | Baseline | NA | NA | Validation |
| 762 | NSCLC | NKI | <12 h | 66 | M | N | N | Nivolumab | PR | Baseline | NA | NA | Training |
| 763 | NSCLC | NKI | <12 h | 73 | F | N | N | Nivolumab | PR | Baseline | NA | NA | Training |
| 764 | NSCLC | NKI | <12 h | 57 | M | N | N | Nivolumab | PD | Baseline | NA | NA | Training |
| 765 | NSCLC | NKI | <12 h | 68 | M | N | N | Nivolumab | PD | Baseline | NA | NA | Validation |
| 766 | NSCLC | NKI | <12 h | 73 | M | N | N | Nivolumab | PR | Baseline | NA | NA | Evaluation |
| 767 | NSCLC | NKI | <12 h | 68 | M | N | N | Nivolumab | PD | Baseline | NA | NA | Training |
| 768 | NSCLC | NKI | <12 h | 64 | M | N | N | Nivolumab | PD | Baseline | NA | NA | Validation |
| 769 | NSCLC | NKI | <12 h | 62 | M | N | N | Nivolumab | PD | Baseline | NA | NA | Training |
| 770 | NSCLC | NKI | <12 h | 51 | F | N | N | Nivolumab | PR | Baseline | NA | NA | Training |
| 771 | NSCLC | NKI | <12 h | 69 | F | N | N | Nivolumab | PD | Baseline | NA | NA | Validation |
| 772 | NSCLC | NKI | <12 h | 54 | M | N | N | Nivolumab | PD | Baseline | NA | NA | Training |
| 773 | NSCLC | NKI | <12 h | 60 | M | N | N | Nivolumab | PR | Baseline | NA | NA | Validation |
| 774 | NSCLC | NKI | <12 h | 38 | F | N | N | Nivolumab | PD | Baseline | NA | NA | Validation |
| 775 | NSCLC | NKI | <12 h | 79 | M | N | N | Nivolumab | PD | Baseline | NA | NA | Training |
| 776 | NSCLC | NKI | <12 h | 64 | M | N | N | Nivolumab | PD | Baseline | NA | NA | Evaluation |
| 777 | NSCLC | NKI | <12 h | 68 | F | N | N | Nivolumab | PR | Baseline | NA | NA | Validation |
| 778 | NSCLC | NKI | <12 h | 55 | F | Y | Y | Nivolumab | PD | FollowUp | NA | NA | NA |
| 779 | NSCLC | VUMC | <12 h | 47 | F | N | N | Nivolumab | PR | FollowUp | NA | NA | NA |
| 780 | NSCLC | VUMC | <12 h | 67 | F | N | N | Nivolumab | PR | FollowUp | NA | NA | NA |
| 781 | NSCLC | VUMC | <12 h | 60 | F | N | N | Nivolumab | PR | FollowUp | NA | NA | NA |
| 782 | NSCLC | VUMC | <12 h | 67 | F | N | N | Nivolumab | PR | FollowUp | NA | NA | NA |

1. A method of administering immunotherapy that modulates an interaction between PD-1 and its ligand, to a cancer patient, comprising the steps of:
providing a sample from the patient, the sample comprising mRNA products that are obtained from anucleated cells of said patient;
determining a gene expression level for at least four genes listed in Table 1;
comparing said determined gene expression level to a reference expression level of said genes in a reference sample;
typing the patient as a positive responder to said immunotherapy, or as a not-positive responder, based on the comparison with the reference; and
administering immunotherapy to a cancer patient that is typed as a positive responder.

2. The method according to claim 1, whereby said cancer patient is a lung cancer patient, preferably a non-small cell lung cancer patient.

3. The method according to claim 1, wherein the anucleated cell is a thrombocyte.

4. The method according to claim 1, comprising determining the gene expression level for at least 10 genes, preferably all genes, listed in Table 1.

5. The method according to claim 1, wherein the sample is obtained by isolating anucleated cells, preferably thrombocytes, from a blood sample of said patient and isolating mRNA from said isolated cells.

6. The method according to claim 1, wherein the gene expression level is determined by next generation sequencing.

7. The method according to claim 1, wherein the immunotherapy comprises nivolumab.

8. A method of typing a sample of a subject for the presence or absence of a cancer, comprising the steps of:
providing a sample from the subject, whereby the sample comprises mRNA products that are obtained from anucleated cells of said subject;
determining a gene expression level for at least five genes listed in Table 2;
comparing said determined gene expression level to a reference expression level of said genes in a reference sample; and
typing said sample for the presence or absence of a cancer on the basis of the comparison between the determined gene expression level and the reference gene expression level.

9. The method according to claim 8, wherein the cancer is a lung cancer, preferably a non-small cell lung cancer.

10. The method according to claim 8, comprising determining the gene expression level for at least 10 genes, preferably all genes, listed in Table 2.

11. The method according to claim 8, wherein the anucleated cells are thrombocytes.

12. The method according to claim 8, wherein the sample is obtained by isolating anucleated cells, preferably thrombocytes, from a blood sample of said subject and isolating mRNA from said isolated cells.

13. Immunotherapy that modulates an interaction between PD-1 and its ligand, for use in a method of treating a cancer patient, preferably a lung cancer patient, wherein said cancer patient is selected by:
typing a sample from the patient, the sample comprising mRNA products that are obtained from anucleated cells of said subject;
determining a gene expression level for at least four genes listed in Table 1;
comparing said determined gene expression level to an expression level of said genes in a reference sample;
typing the patient as a positive responder to said immunotherapy, or as a not-positive responder, based on the comparison with the reference; and
assigning immunotherapy to a cancer patient that is selected as a positive responder.

14. A method for obtaining a biomarker panel for typing of a sample from a subject, the method comprising
isolating anucleated cells, preferably thrombocytes, from a liquid sample of a subject having condition A;
isolating RNA from said isolated cells;
determining RNA expression levels for at least 100 genes in said isolated RNA;
determining RNA expression levels for said at least 100 genes in a control sample from a subject not having condition A; and
using particle swarm optimization-based algorithms to obtain a biomarker panel that discriminates between a subject having condition A and a subject not having condition A.

15. The method according to claim 14, wherein the subject having condition A is suffering from a cancer, preferably a lung cancer, or had a known response to a cancer treatment.
